(19) United States
(12) Patent Application Publication
(10) Pub. No.: US 2023/0023593 A1
(43) Pub. Date: Jan. 26, 2023

(54) POSITIVE RESIST COMPOSITION AND PATTERN FORMING PROCESS

(71) Applicant: Shin-Etsu Chemical Co., Ltd., Tokyo (JP)

(72) Inventors: Jun Hatakeyama, Joetsu-shi (JP); Takayuki Fujiwara, Joetsu-shi (JP)

(73) Assignee: Shin-Etsu Chemical Co., Ltd., Tokyo (JP)

(21) Appl. No.: 17/837,310

(22) Filed: Jun. 10, 2022

(30) Foreign Application Priority Data
Jun. 22, 2021 (JP) 2021-102960

(51) Int. Cl. G03F 7/039; C07C 317/24; C07C 317/06; C08G 63/06; G03F 7/20

(52) U.S. Cl. CPC G03F 7/0392; C07C 317/24; C07C 317/06; C08G 63/065; G03F 7/2004

(57) ABSTRACT

A positive resist composition is provided comprising (A) a specific sulfonium salt as quencher, (B) a sulfonium salt consisting of a fluorinated sulfonate anion and a sulfonium cation as acid generator, and (C) a base polymer comprising repeat units having an acid labile group. The resist composition has a high sensitivity and resolution, improved LWR or CDU, and a broad process window and forms a pattern of good profile after exposure.

### CROSS-REFERENCE TO RELATED APPLICATION

This non-provisional application claims priority under 35 U.S.C. § 119(a) on Patent Application No. 2021-102960 filed in Japan on Jun. 22, 2021, the entire contents of which are hereby incorporated by reference.

### TECHNICAL FIELD

This invention relates to a positive resist composition and a patterning process using the composition.

### BACKGROUND ART

To meet the demand for higher integration density and operating speed of LSIs, the effort to reduce the pattern rule is in rapid progress. In particular, the enlargement of the logic memory market to comply with the wide-spread use of smart phones drives forward the miniaturization technology. As the use of 5G high-speed communications and artificial intelligence (AI) is widely spreading, high-performance semiconductor devices are needed for their processing, with the progress of miniaturization being accelerated. As the advanced miniaturization technology, manufacturing of microelectronic devices at the 7-nm node by the ArF immersion lithography and devices at the 5-nm node by the EUV lithography has been implemented in a mass scale. The EUV lithography is one of the candidates for the manufacture of 3-nm node devices of the next generation and 2-nm node devices of the next-but-one generation.

The EUV lithography enables to form small size patterns because the wavelength (13.5 nm) of EUV is as short as 1/14.3 of the wavelength (193 nm) of ArF excimer laser light. However, since the number of photons available from EUV exposure is accordingly 1/14 of that from ArF excimer laser exposure, there arises the problem of shot noise that a variation in number of photons causes an increase in edge roughness (LWR) and a lowering of CDU (Non-Patent Document 1).

In addition to the variations due to shot noise, it is pointed out in Non-Patent Document 2 that the uneven distribution of acid generator and quencher components in a resist film causes a variation in feature size. In the EUV lithography for forming very small size patterns, there exists a need for a resist material of uniform distribution system.

### CITATION LIST

Non-Patent Document 1: SPIE Vol. 3331, p 531 (1998)
Non-Patent Document 2: SPIE Vol. 9776, p 97760V-1 (2016)

### DISCLOSURE OF INVENTION

An object of the invention is to provide a positive tone resist composition which exhibits a higher sensitivity and resolution than prior art positive resist compositions, improved LWR or CDU, and a broad process window and forms a pattern of good profile after exposure; and a pattern forming process using the same.

The inventors presumed that for obtaining a positive tone resist composition having a high sensitivity and resolution as desired in the recent market, improved LWR or CDU, and capable of avoiding the bridging phenomenon that lines are bridged like threading when lines are thickened, and preventing pattern collapse or film thickness loss when lines are thinned, it is necessary to prevent resist components, typically quencher from agglomerating together, to disperse or distribute the components uniformly, to minimize the swell of a resist film in alkaline developer, and to prevent pattern collapse during drying of rinse liquid.

It is believed effective for the purpose of preventing quencher agglomeration, to utilize the electrical repulsion force of fluorine atoms to prevent the relevant components from agglomeration. The inventors have found that when a sulfonium salt containing an anion of specific structure, hexafluoroalkoxide anion as the quencher is combined with a sulfonium salt containing a sulfonate anion having fluorine on the carbon atom at α- and/or β-position of the sulfo group as the acid generator, there is formulated a positive resist composition which forms a resist pattern having improved CDU or LWR and a broad process window.

In one aspect, the invention provides a positive resist composition comprising

(A) a quencher in the form of a sulfonium salt having the following formula (1),

(B) an acid generator in the form of a sulfonium salt consisting of a sulfonate anion having fluorine on the carbon atom at α- and/or β-position of the sulfo group and a sulfonium cation, and

(C) a base polymer comprising repeat units of at least one type selected from repeat units (a1) having a carboxy group whose hydrogen is substituted by an acid labile group and repeat units (a2) having a phenolic hydroxy group whose hydrogen is substituted by an acid labile group.

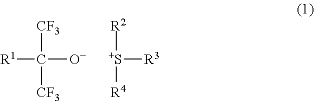

Herein R¹ is fluorine, a C₁-C₄ alkyl group, C₁-C₄ alkyloxy group, C₂-C₄ alkenyl group, C₂-C₄ alkynyl group, phenyl group, or C₁-C₂₀ hydrocarbyloxycarbonyl group, some or all of the hydrogen atoms in the alkyl, alkyloxy, alkenyl, alkynyl, and hydrocarbyloxycarbonyl groups may be substituted by fluorine, chlorine, bromine, iodine, trifluoromethyl, trifluoromethoxy, trifluorothio, hydroxy, cyano, nitro or sulfonyl moiety, some constituent —CH₂— in the alkyl, alkyloxy, alkenyl, alkynyl, and hydrocarbyloxycarbonyl groups may be replaced by an ester bond or ether bond, and some or all of the hydrogen atoms in the phenyl group may be substituted by fluorine, C₁-C₄ fluoroalkyl, C₁-C₄ fluoroalkyloxy, C₁-C₄ fluoroalkylthio, cyano or nitro moiety. R² to R⁴ are each independently halogen or a C₁-C₂₀ hydrocarbyl group which may contain at least one atom selected from oxygen, sulfur, nitrogen and halogen, R² and R³ may bond together to form a ring with the sulfur atom to which they are attached.

In a preferred embodiment, the sulfonate anion in the sulfonium salt (B) has the formula (2-1) or (2-2):

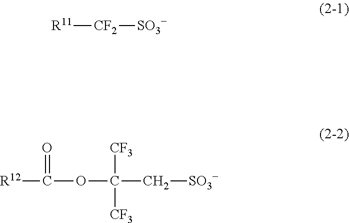

wherein R¹¹ is fluorine or a C₁-C₂₀ hydrocarbyl group which may contain a heteroatom, and R¹² is a C₁-C₂₀ hydrocarbyl group which may contain a heteroatom.

In a preferred embodiment, the sulfonate anion in the sulfonium salt (B) is an iodized sulfonate anion.

More preferably, the iodized sulfonate anion has the formula (2-3).

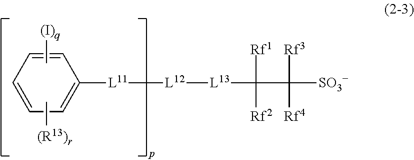

Herein p is an integer of 1 to 3, q is an integer of 1 to 5, r is an integer of 0 to 3, and 1≤q+r≤5. L¹¹ is a single bond, ether bond, ester bond, amide bond, imide bond, or C₁-C₆ saturated hydrocarbylene group in which some constituent —CH₂— may be replaced by an ether bond or ester bond. L¹² is a single bond or a C₁-C₂₀ hydrocarbylene group when p is 1, and a C₁-C₂₀ (p+1)-valent hydrocarbon group when p is 2 or 3, the hydrocarbylene group and (p+1)-valent hydrocarbon group may contain at least one atom selected from oxygen, sulfur and nitrogen. L¹³ is a single bond, ether bond or ester bond. R¹³ is a hydroxy group, carboxy group, fluorine, chlorine, bromine or amino group, or a C₁-C₂₀ hydrocarbyl group, C₁-C₂₀ hydrocarbyloxy group, C₂-C₂₀ hydrocarbylcarbonyl group, C₂-C₂₀ hydrocarbyloxycarbonyl group, C₂-C₂₀ hydrocarbylcarbonyloxy group, or C₁-C₂₀ hydrocarbylsulfonyloxy group, which may contain fluorine, chlorine, bromine, hydroxy, amino or ether bond, or —N(R^{13A})(R^{13B}), —N(R^{13C})—C(═O)—R^{13D}, or —N(R^{13C})—C(═O)—O—R^{13D}, R^{13A} and R^{13B} are each independently hydrogen or a C₁-C₆ saturated hydrocarbyl group, R^{13C} is hydrogen or a C₁-C₆ saturated hydrocarbyl group in which some or all of the hydrogen atoms may be substituted by halogen, hydroxy, C₁-C₆ saturated hydrocarbyloxy, C₂-C₆ saturated hydrocarbylcarbonyl or C₂-C₆ saturated hydrocarbylcarbonyloxy moiety, R^{13D} is a C₁-C₁₆ aliphatic hydrocarbyl group, C₆-C₁₂ aryl group or C₇-C₁₅ aralkyl group, in which some or all of the hydrogen atoms may be substituted by halogen, hydroxy, C₁-C₆ saturated hydrocarbyloxy, C₂-C₆ saturated hydrocarbylcarbonyl or C₂-C₆ saturated hydrocarbylcarbonyloxy moiety. Rf¹ to Rf⁴ are each independently hydrogen, fluorine or trifluoromethyl, at least one of Rf¹ to Rf⁴ is fluorine or trifluoromethyl, and Rf¹ and Rf², taken together, may form a carbonyl group.

In a preferred embodiment, repeat units (a1) have the formula (a1) and repeat units (a2) have the formula (a2).

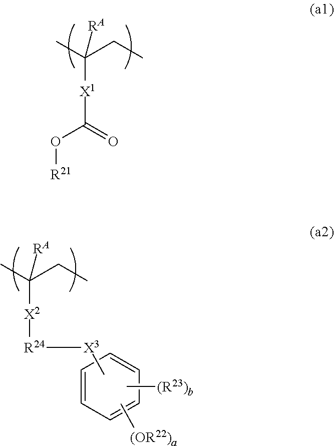

Herein R^{A} is each independently hydrogen or methyl, X¹ is a single bond, phenylene, naphthylene, or a C₁-C₁₂ linking group containing at least one moiety selected from ether bond, ester bond and lactone ring, X² is a single bond, ester bond or amide bond, X³ is a single bond, ether bond or ester bond, R²¹ and R²² are each independently an acid labile group, R²³ is fluorine, trifluoromethyl, cyano or a C₁-C₆ saturated hydrocarbyl group, R²⁴ is a single bond or a C₁-C₆ alkanediyl group in which some carbon may be replaced by an ether bond or ester bond, a is 1 or 2, b is an integer of 0 to 4, and 1≤a+b≤5.

In a preferred embodiment, the base polymer further comprises repeat units having an adhesive group selected from hydroxy, carboxy, lactone ring, carbonate, thiocarbonate, carbonyl, cyclic acetal, ether bond, ester bond, sulfonic ester bond, cyano, amide bond, —O—C(═O)—S—, and —O—C(═O)—NH—.

The resist composition may further comprise (D) an organic solvent and/or (E) a surfactant.

In another aspect, the invention provides a pattern forming process comprising the steps of applying the positive resist composition defined above onto a substrate to form a resist film thereon, exposing the resist film to high-energy radiation, and developing the exposed resist film in a developer.

The high-energy radiation is typically i-line, KrF excimer laser, ArF excimer laser, EB, or EUV of wavelength 3 to 15 nm.

### Advantageous Effects of Invention

The positive resist composition of the invention forms a resist film having a high sensitivity, improved LWR or CDU, and a broad process window because the acid generator and the quencher are uniformly distributed in the resist film. By virtue of these advantages, the resist composition is fully useful in commercial application and suited as a micropatterning material for the fabrication of VLSIs, micropatterning material for the fabrication of photomasks by EB writing, and micropatterning material adapted for EB or EUV lithography. The resist composition may be used not only in the lithography for forming semiconductor circuits, but also in the formation of mask circuit patterns, micromachines, and thin-film magnetic head circuits.

### DESCRIPTION OF EMBODIMENTS

As used herein, the singular forms “a,” “an” and “the” include plural referents unless the context clearly dictates otherwise. The notation (Cn-Cm) means a group containing from n to m carbon atoms per group. The term “halogenated (e.g., fluorinated) compound” indicates a compound containing halogen (e.g., fluorine), and the term “group” and “moiety” are interchangeable. In chemical formulae, the broken line designates a valence bond, and Me stands for methyl and Ac for acetyl.

The abbreviations and acronyms have the following meaning.

EB: electron beam

EUV: extreme ultraviolet

Mw: weight average molecular weight

Mn: number average molecular weight

Mw/Mn: molecular weight dispersity

GPC: gel permeation chromatography

PEB: post-exposure bake

PAG: photoacid generator

LWR: line width roughness

CDU: critical dimension uniformity

### Positive Resist Composition

The positive resist composition of the invention is defined as comprising (A) a quencher in the form of a sulfonium salt containing an anion of specific structure, hexafluoroalkoxide anion, (B) an acid generator in the form of a sulfonium salt consisting of a sulfonate anion having fluorine on the carbon atom at α- and/or β-position of the sulfo group and a sulfonium cation, and (C) a base polymer comprising repeat units of at least one type selected from repeat units (a1) having a carboxy group whose hydrogen is substituted by an acid labile group and repeat units (a2) having a phenolic hydroxy group whose hydrogen is substituted by an acid labile group. The electric repulsion of fluorine atoms in the sulfonium salt serving as the quencher or component (A) prevents the quencher from agglomerating together. Then the resist pattern after development is improved in LWR and CDU. Since the fluoroalcohol generated upon light exposure little swells in the alkaline developer, and has a large contact angle with water and hence, a weak capillary force, the stresses applied to the resist pattern during drying of rinsing pure water after alkaline development are reduced. This prevents pattern collapse.

### (A) Quencher

The quencher as component (A) is a sulfonium salt having the following formula (1).

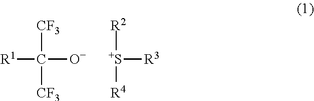

In formula (1), R¹ is fluorine, a C₁-C₄ alkyl group, C₁-C₄ alkyloxy group, C₂-C₄ alkenyl group, C₂-C₄ alkynyl group, phenyl group, or C₁-C₂₀ hydrocarbyloxycarbonyl group. Some or all of the hydrogen atoms in the alkyl, alkyloxy, alkenyl, alkynyl, and hydrocarbyloxycarbonyl groups may be substituted by fluorine, chlorine, bromine, iodine, trifluoromethyl, trifluoromethoxy, trifluorothio, hydroxy, cyano, nitro or sulfonyl moiety; some constituent —CH₂— in the alkyl, alkyloxy, alkenyl, alkynyl, and hydrocarbyloxycarbonyl groups may be replaced by an ester bond or ether bond; and some or all of the hydrogen atoms in the phenyl group may be substituted by fluorine, C₁-C₄ fluoroalkyl, C₁-C₄ fluoroalkyloxy, C₁-C₄ fluoroalkylthio, cyano or nitro moiety.

Examples of the C₁-C₄ alkyl group and alkyl moiety in the C₁-C₄ alkyloxy group include methyl, ethyl, n-propyl, isopropyl n-butyl, isobutyl, sec-butyl, and tert-butyl. Examples of the C₂-C₄ alkenyl group include vinyl, 1-propenyl, and 2-propenyl. Examples of the C₂-C₄ alkynyl group include ethynyl, 1-propynyl, and 2-propynyl.

The hydrocarbyl moiety in the C₁-C₂₀ hydrocarbyloxycarbonyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, and tert-butyl; C₃-C₂₀ cyclic saturated hydrocarbyl groups such as cyclopropyl, cyclobutyl, cyclopentyl, cyclohexyl, norbornyl, and adamantyl; C₆-C₂₀ aryl groups such as phenyl and naphthyl; and C₇-C₂₀ aralkyl groups such as benzyl and phenethyl.

The alkyl moiety in the C₁-C₄ fluoroalkyl, C₁-C₄ fluoroalkyloxy, and C₁-C₄ fluoroalkylthio groups is a C₁-C₄ alkyl group in which some or all of the hydrogen atoms are substituted by fluorine. Examples thereof include fluoromethyl, difluoromethyl, trifluoromethyl, 2,2,2-trifluoroethyl, pentafluoroethyl, and 1,1,1,3,3,3-hexafluoro-2-propyl.

Examples of the alkoxide anion in the sulfonium salt having formula (1) are shown below, but not limited thereto.

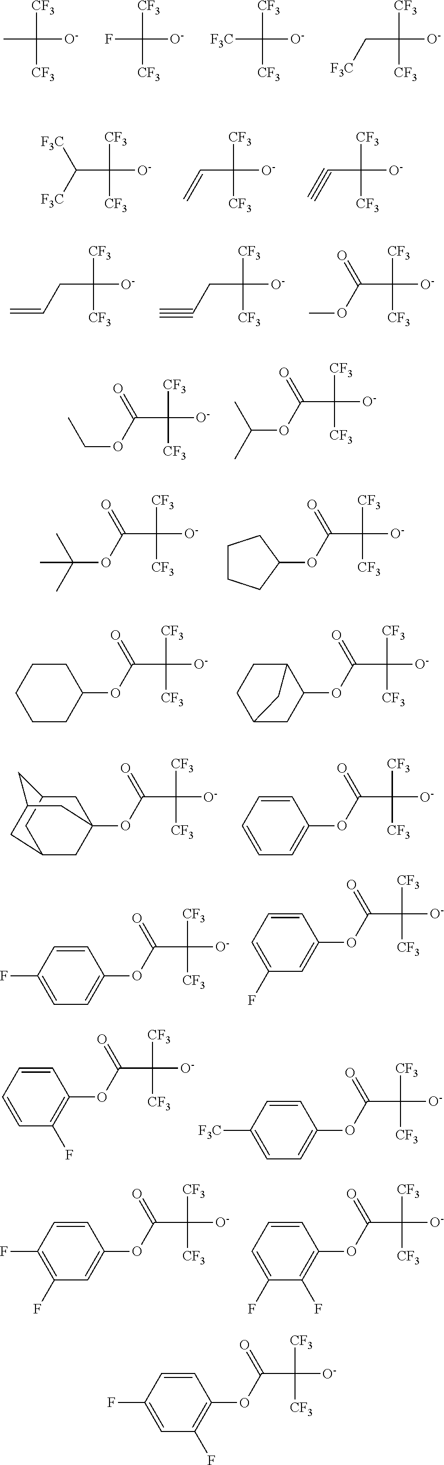

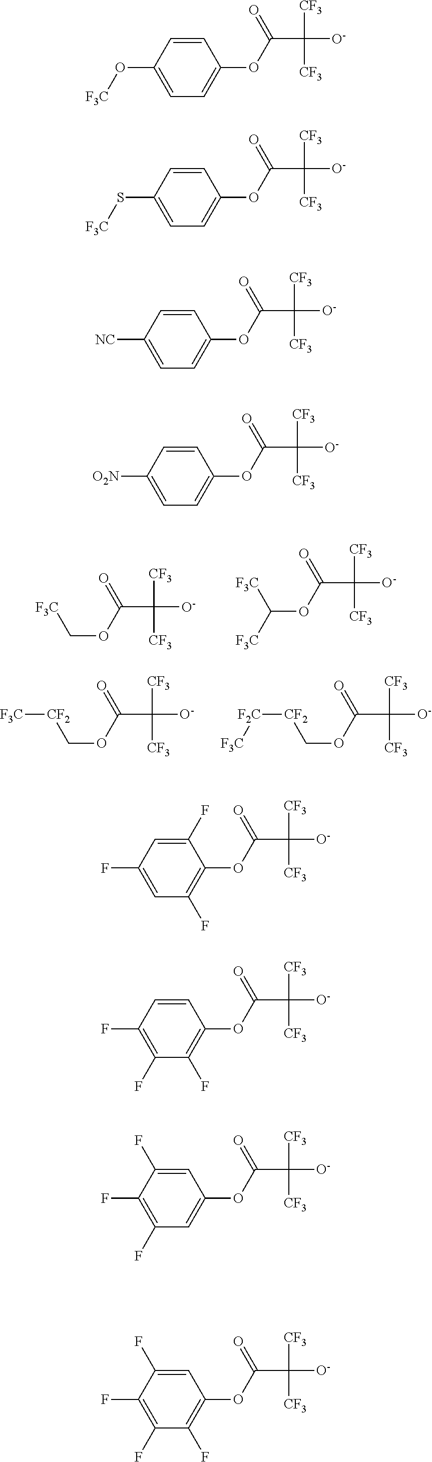

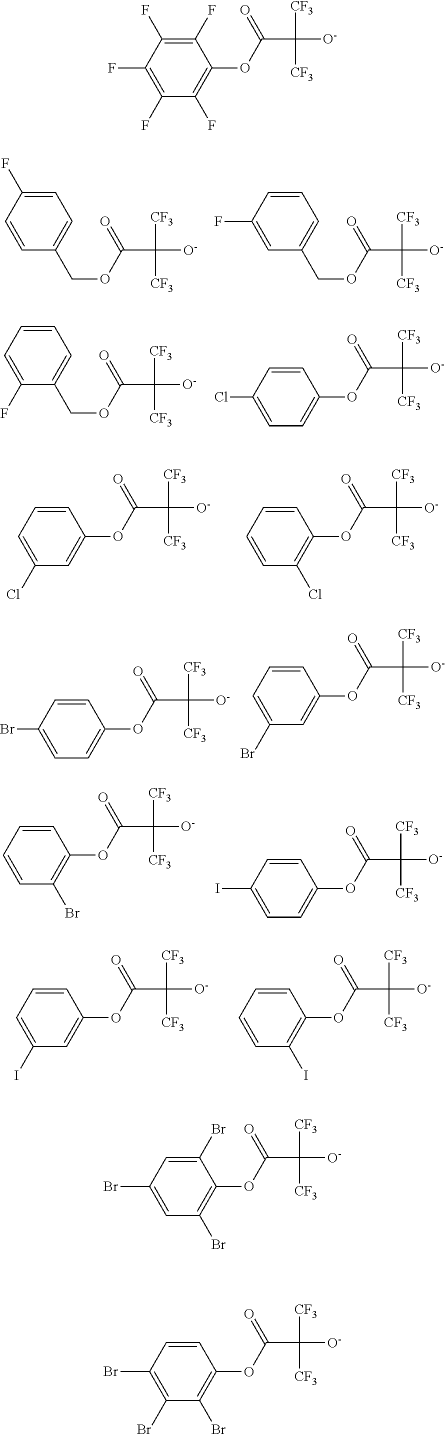

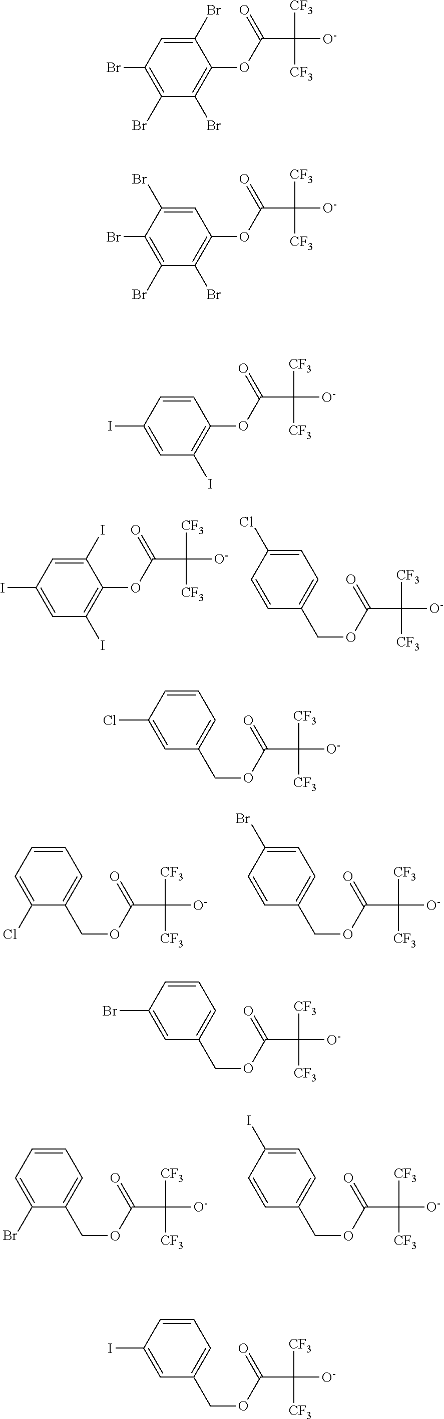

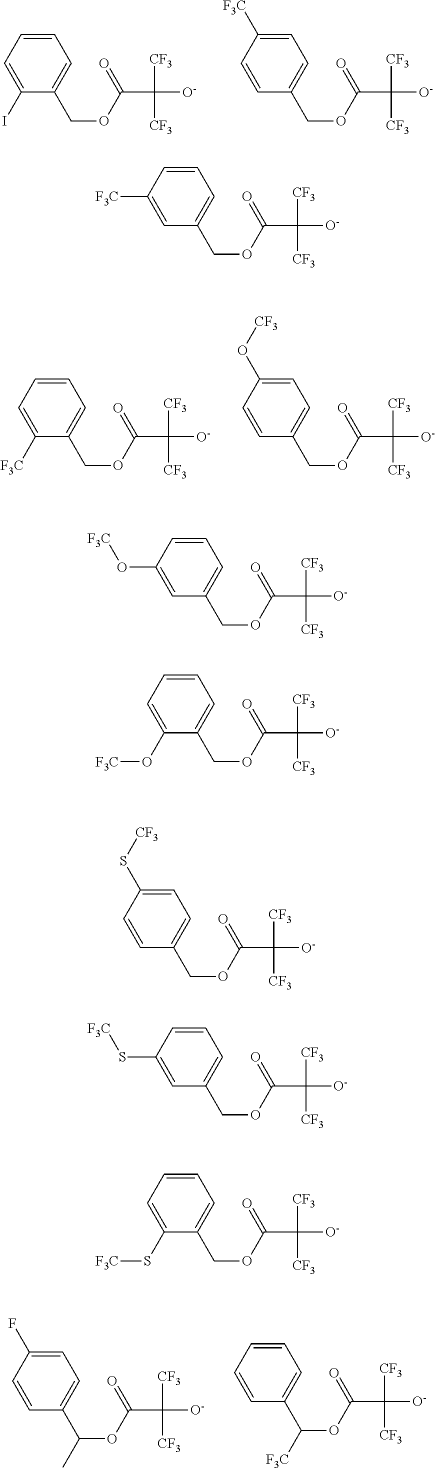

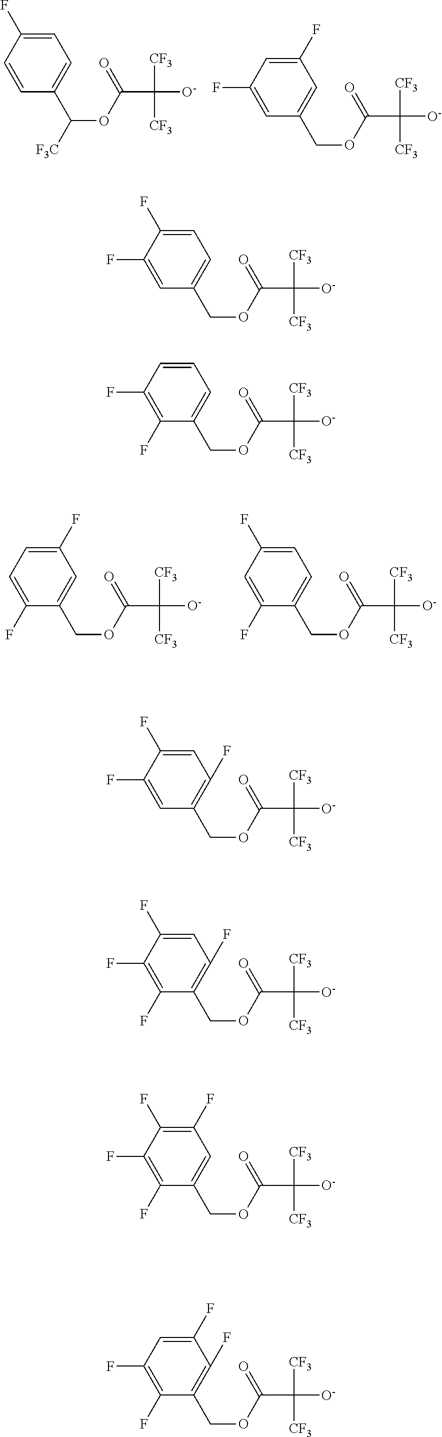

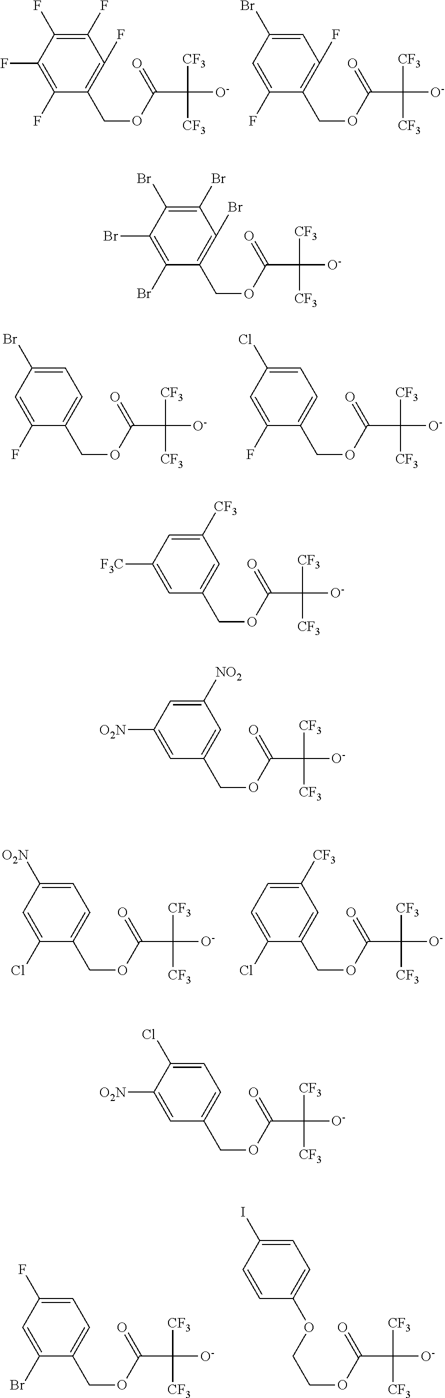

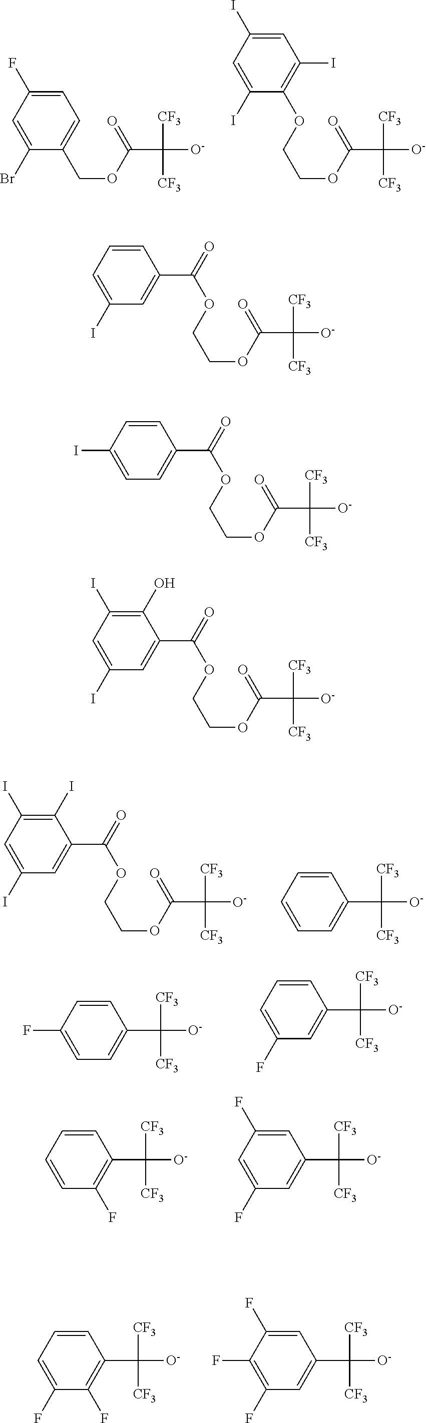

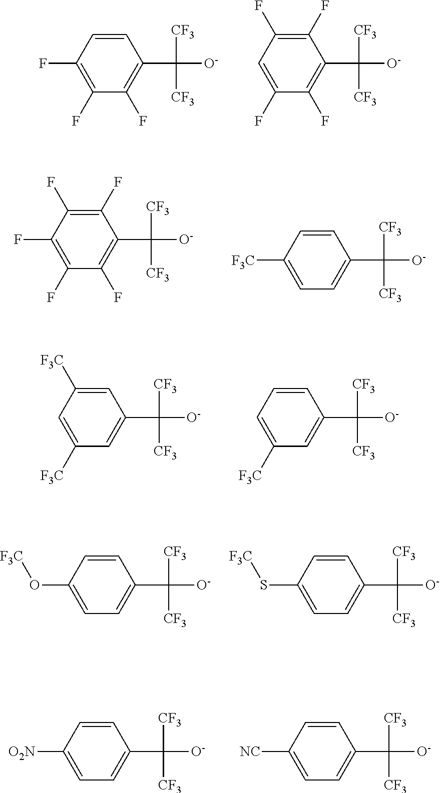

In formula (1), R² to R⁴ are each independently halogen or a C₁-C₂₀ hydrocarbyl group which may contain at least one atom selected from oxygen, sulfur, nitrogen and halogen.

Examples of the halogen represented by R² to R⁴ include fluorine, chlorine, bromine and iodine.

The C₁-C₂₀ hydrocarbyl group represented by R² to R⁴ may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, propenyl, butenyl and hexenyl; C₂-C₂₀ cyclic unsaturated aliphatic hydrocarbyl groups such as cyclohexenyl and norbornenyl; C₂-C₂₀ alkynyl groups such as ethynyl, propynyl and butynyl; C₆-C₂₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, and tert-butylnaphthyl; C₇-C₂₀ aralkyl groups such as benzyl and phenethyl; and combinations thereof.

In the hydrocarbyl group, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonic ester bond, carbonate bond, lactone ring, sultone ring, carboxylic anhydride (—C(═O)—O—C(═O)—) or haloalkyl moiety.

R² and R³ may bond together to form a ring with the sulfur atom to which they are attached. Preferred are rings of the structure shown below.

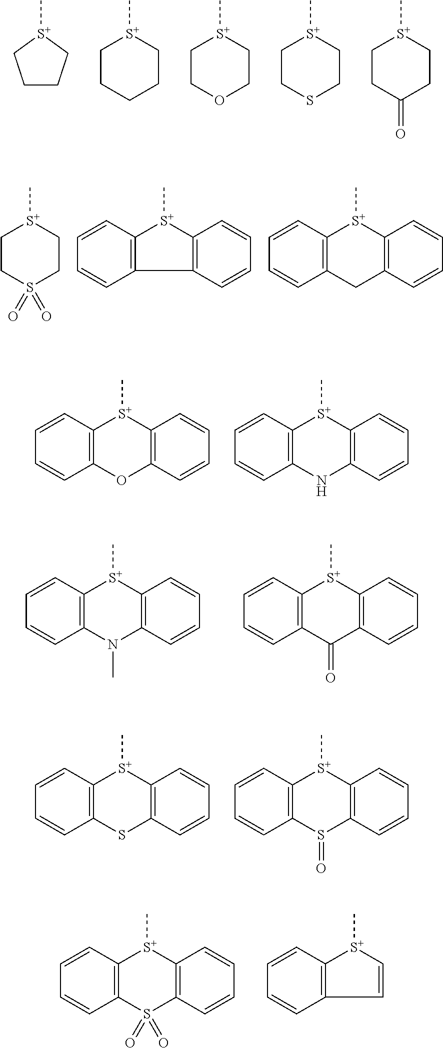

Herein, the broken line designates a point of attachment to R⁴.

As the cation in the sulfonium salt having formula (1), cations having the formulae (1-1) and (1-2) are preferred.

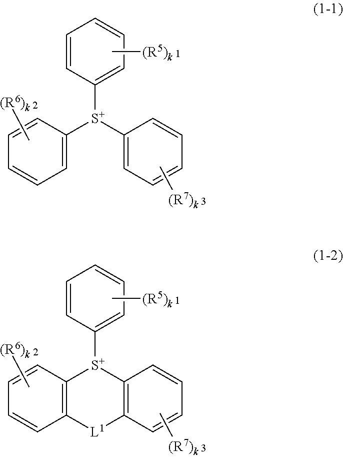

In formulae (1-1) and (1-2), R⁵, R⁶, and R⁷ are each independently halogen, hydroxy, nitro, cyano, carboxy, a C₁-C₁₄ hydrocarbyl group, C₁-C₁₄ hydrocarbyloxy group, C₂-C₁₄ hydrocarbylcarbonyl group, C₂-C₁₄ hydrocarbylcarbonyloxy group, C₂-C₁₄ hydrocarbyloxycarbonyl group, or C₁-C₁₄ hydrocarbylthio group.

Suitable halogen atoms include fluorine, chlorine, bromine and iodine. The C₁-C₁₄ hydrocarbyl group and hydrocarbyl moiety in the C₁-C₁₄ hydrocarbyloxy group, C₂-C₁₄ hydrocarbylcarbonyl group, C₂-C₁₄ hydrocarbylcarbonyloxy group, C₂-C₁₄ hydrocarbyloxycarbonyl group, and C₁-C₁₄ hydrocarbylthio group may be saturated or unsaturated and straight, branched or cyclic. Examples thereof include alkyl groups such as methyl, ethyl, propyl, isopropyl, n-butyl, sec-butyl, tert-butyl, n-pentyl, n-hexyl, n-octyl, 2-ethylhexyl, n-nonyl, and n-decyl; cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, tricyclo[5.2.1.0^{2,6}]decanyl, adamantyl and adamantylmethyl; alkenyl groups such as vinyl, allyl, propenyl, butenyl, and hexenyl; cyclic unsaturated aliphatic hydrocarbyl groups such as cyclohexenyl; aryl groups such as phenyl, naphthyl, thienyl, 4-hydroxyphenyl, 4-methoxyphenyl, 3-methoxyphenyl, 2-methoxyphenyl, 4-ethoxyphenyl, 4-tert-butoxyphenyl, 3-tert-butoxyphenyl, 2-methylphenyl, 3-methylphenyl, 4-methylphenyl, 4-ethylphenyl, 4-tert-butylphenyl, 4-n-butylphenyl, 2,4-dimethylphenyl, 2,4,6-triisopropylphenyl, methylnaphthyl, ethylnaphthyl, methoxynaphthyl, ethoxynaphthyl, n-propoxynaphthyl, n-butoxynaphthyl, dimethylnaphthyl, diethylnaphthyl, dimethoxynaphthyl, and diethoxynaphthyl; and aralkyl groups such as benzyl, 1-phenethyl and 2-phenethyl.

In the hydrocarbyl group, some or all of the hydrogen atoms may be substituted by a moiety containing a heteroatom such as oxygen, sulfur, nitrogen or halogen, so that the group may contain a hydroxy moiety, cyano moiety, fluorine, chlorine, bromine, iodine, or haloalkyl moiety. In the hydrocarbyl group, some constituent —CH₂— may be replaced by —O—, —C(═O)—, —S—, —S(═O)—, —S(═O)₂— or —N(R^{N1})—. R^{N1} is hydrogen or a C₁-C₁₀ hydrocarbyl group in which some or all of the hydrogen atoms may be substituted by a moiety containing a heteroatom such as oxygen, sulfur, nitrogen or halogen, so that the group may contain a hydroxy moiety, cyano moiety, fluorine, chlorine, bromine, iodine, or haloalkyl moiety, and some constituent —CH₂— may be replaced by —O—, —C(═O)— or —S(═O)₂—.

In formula (1-2), L¹ is a single bond, —CH₂—, —O—, —C(═O)—, —S—, —S(═O)—, —S(═O)₂— or —N(R^{N1})— wherein R^{N1} is as defined above.

In formulae (1-1) and (1-2), k¹, k² and k³ are each independently an integer of 0 to 5. When k¹ is 2 or more, groups R⁵ may be the same or different and two R⁵ may bond together to form a ring with the carbon atom on the benzene ring to which they are attached. When k² is 2 or more, groups R⁶ may be the same or different and two R⁶ may bond together to form a ring with the carbon atom on the benzene ring to which they are attached. When k³ is 2 or more, groups R⁷ may be the same or different and two R⁷ may bond together to form a ring with the carbon atom on the benzene ring to which they are attached.

Examples of the cation in the sulfonium salt having formula (1) are shown below, but not limited thereto.

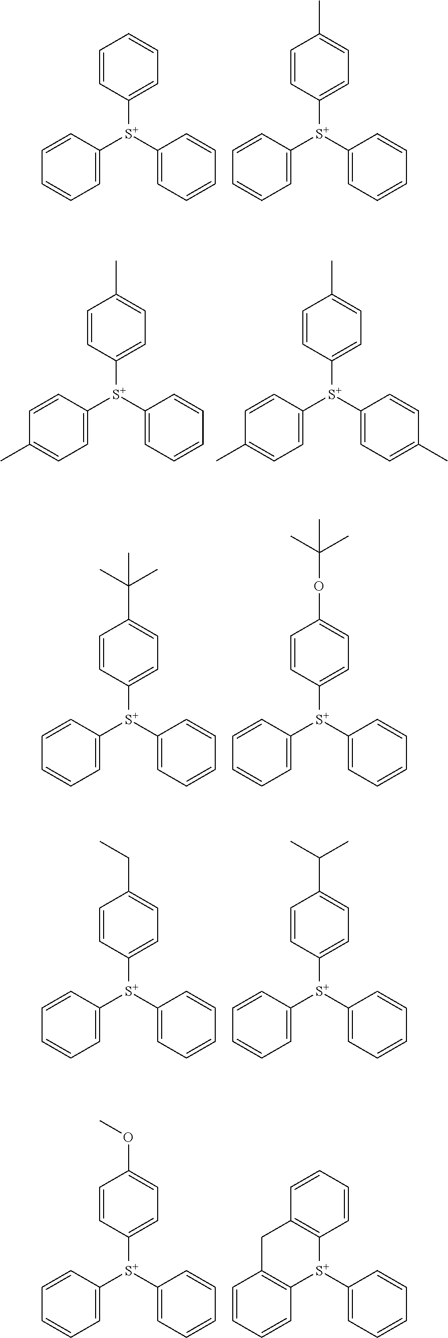

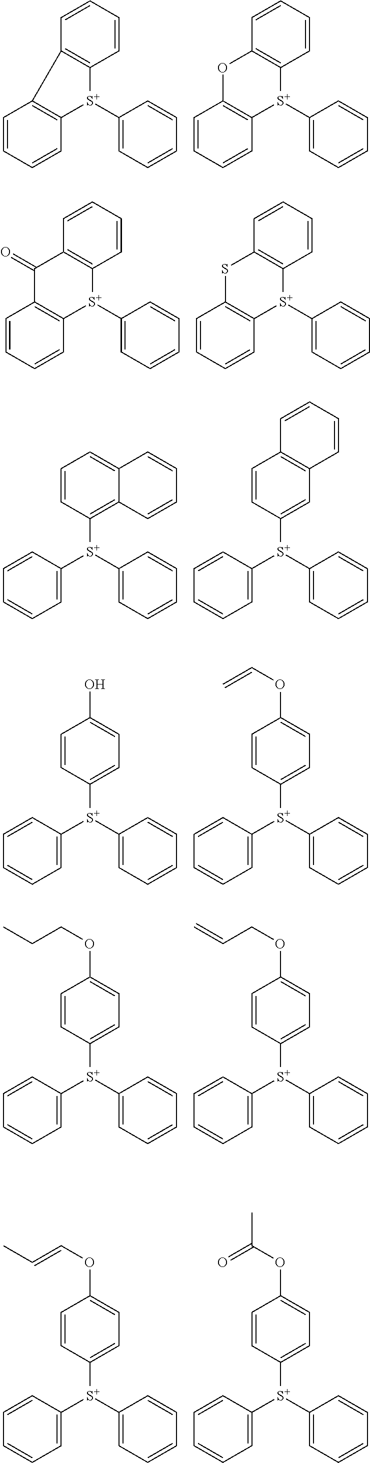

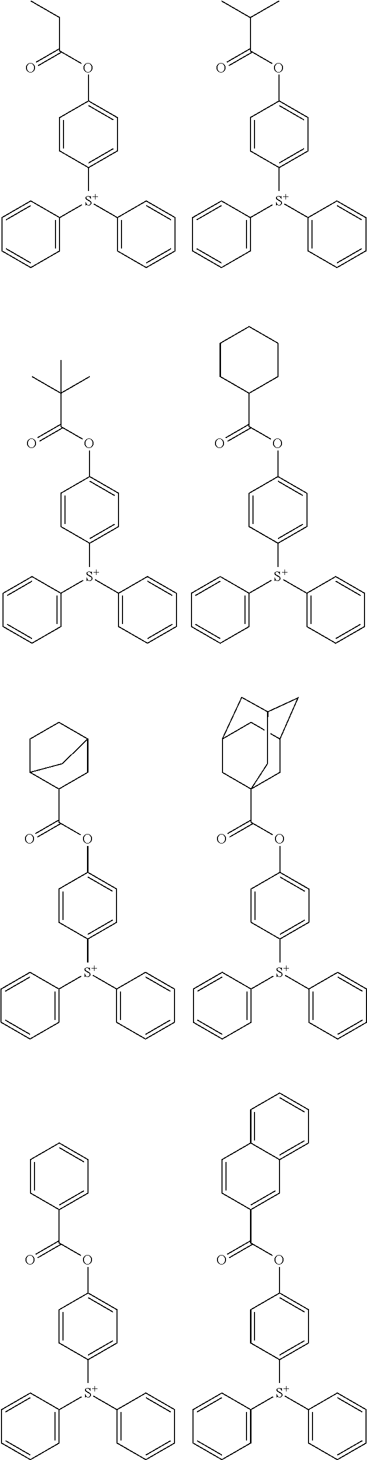

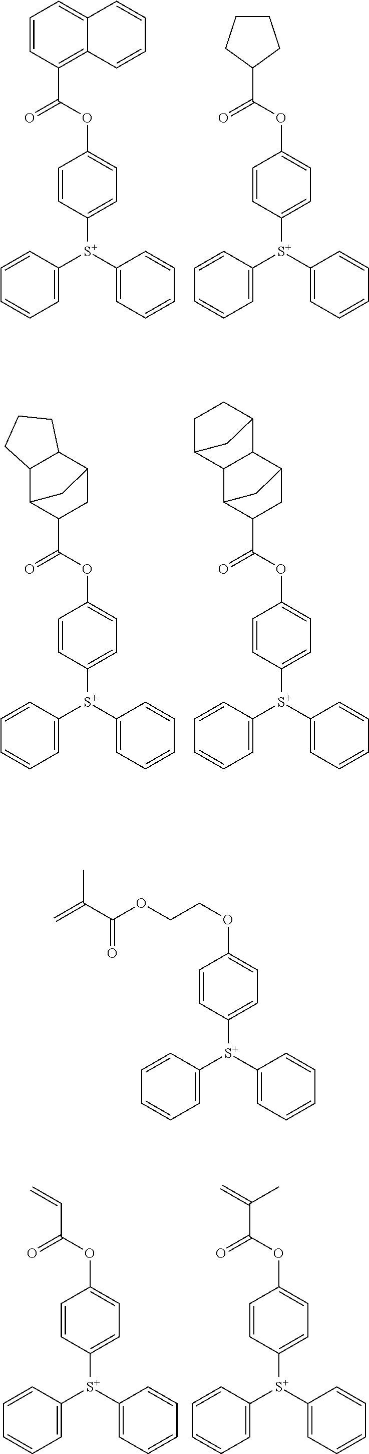

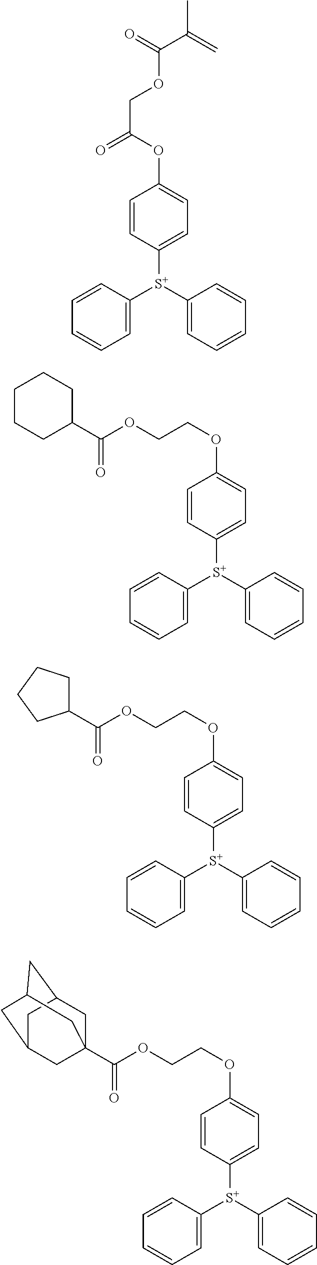 [SMILES: C=C(C)C(=O)OCC(=O)Oc1ccc([S+](c2ccccc2)c2ccccc2)cc1 ; O=C(OCCOc1ccc([S+](c2ccccc2)c2ccccc2)cc1)C1CCCCC1 ; O=C(OCCOc1ccc([S+](c2ccccc2)c2ccccc2)cc1)C1CCCC1 ; O=C(OCCOc1ccc([S+](c2ccccc2)c2ccccc2)cc1)C12CC3CC(CC(C3)C1)C2]

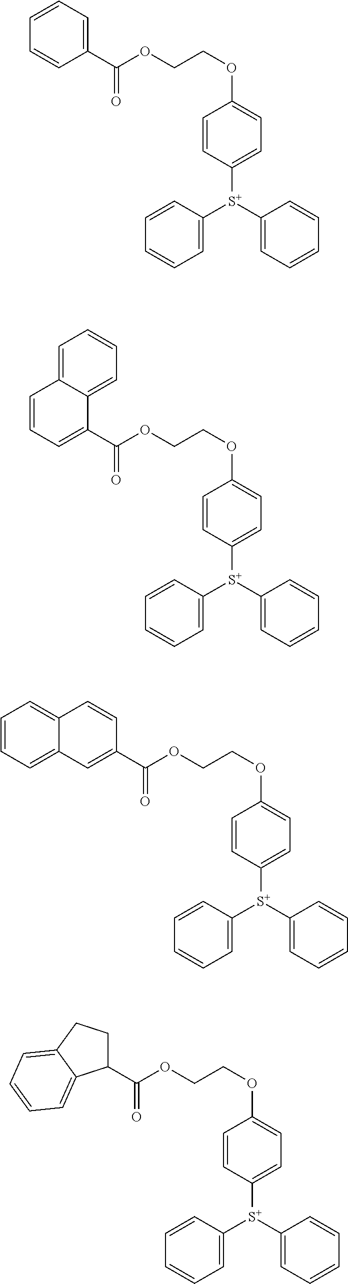 [SMILES: O=C(OCCOc1ccc([S+](c2ccccc2)c2ccccc2)cc1)c1ccccc1 ; O=C(OCCOc1ccc([S+](c2ccccc2)c2ccccc2)cc1)c1cccc2ccccc12 ; O=C(OCCOc1ccc([S+](c2ccccc2)c2ccccc2)cc1)c1ccc2ccccc2c1 ; O=C(OCCOc1ccc([S+](c2ccccc2)c2ccccc2)cc1)C1CCc2ccccc21]

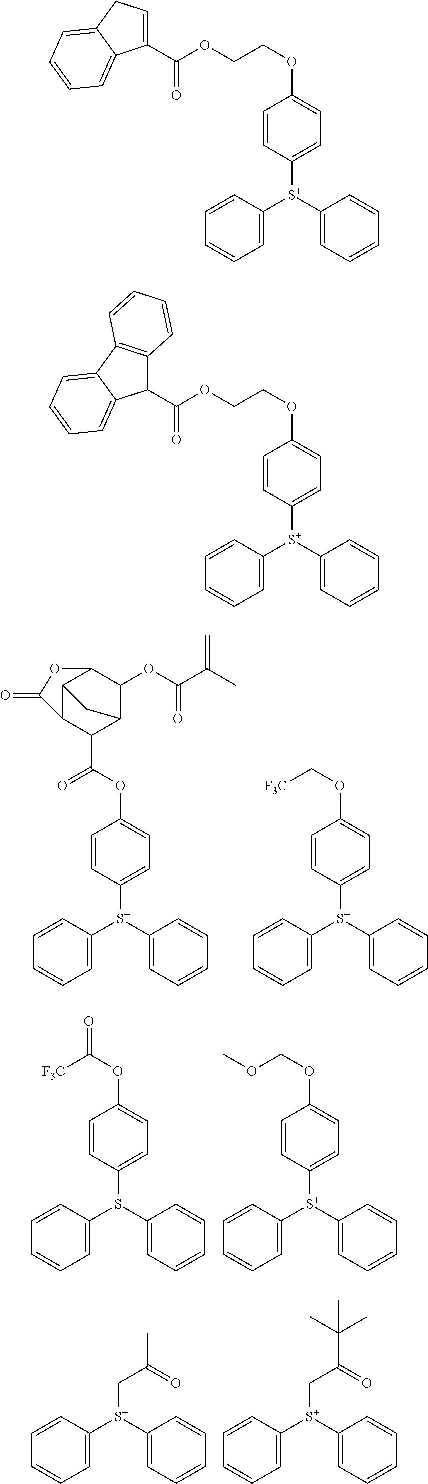

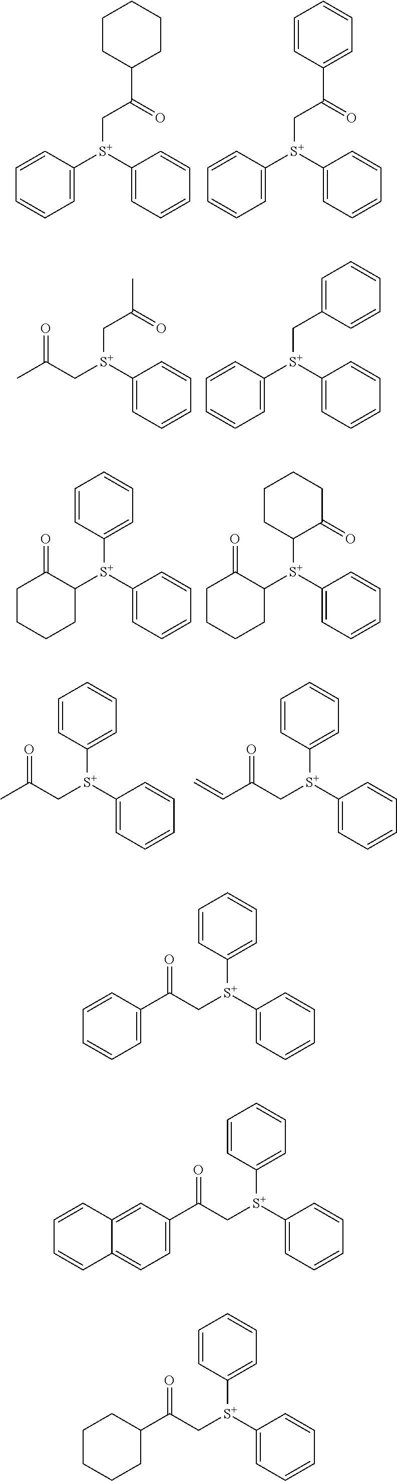

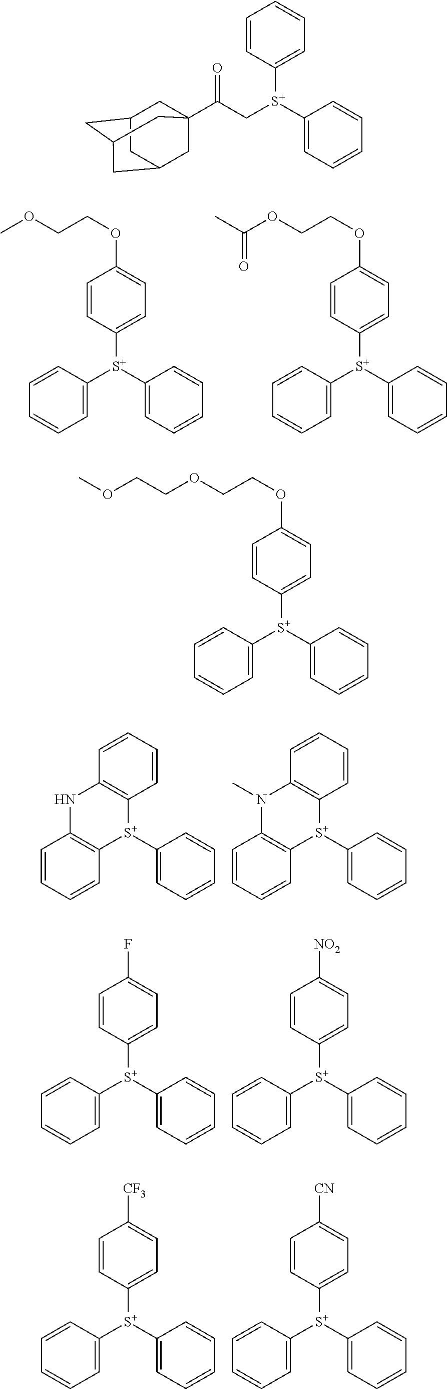

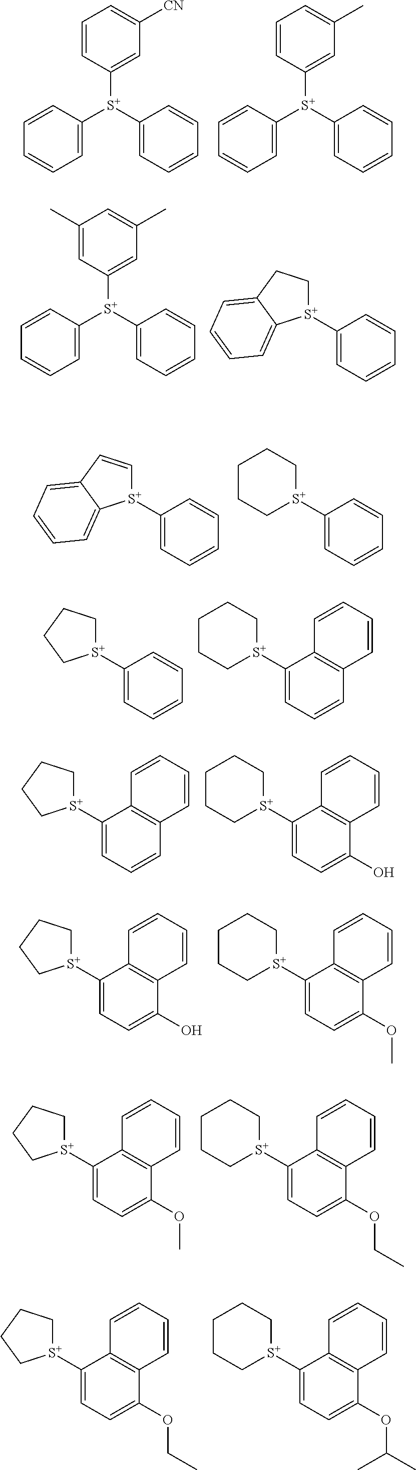

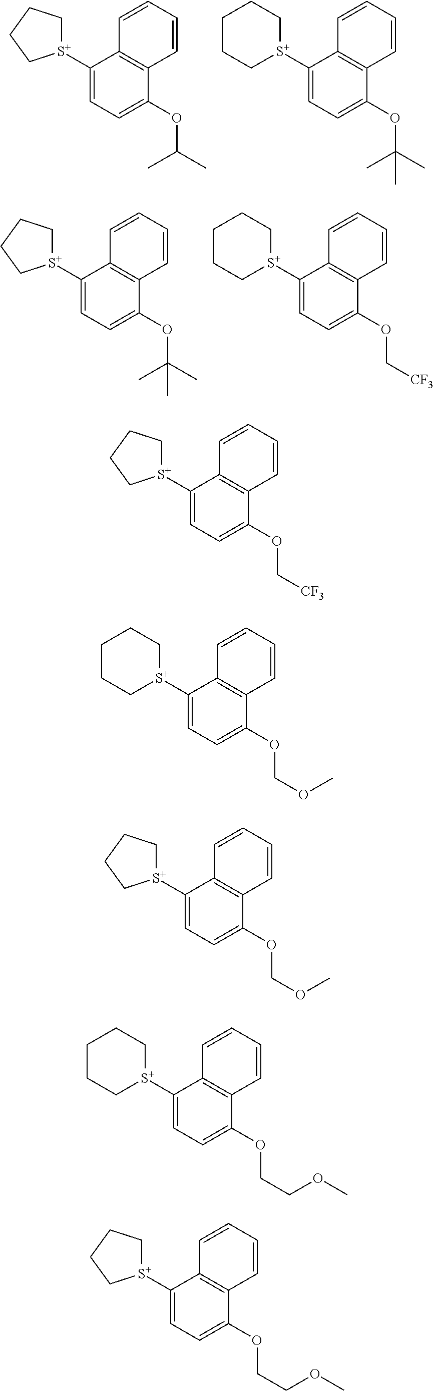

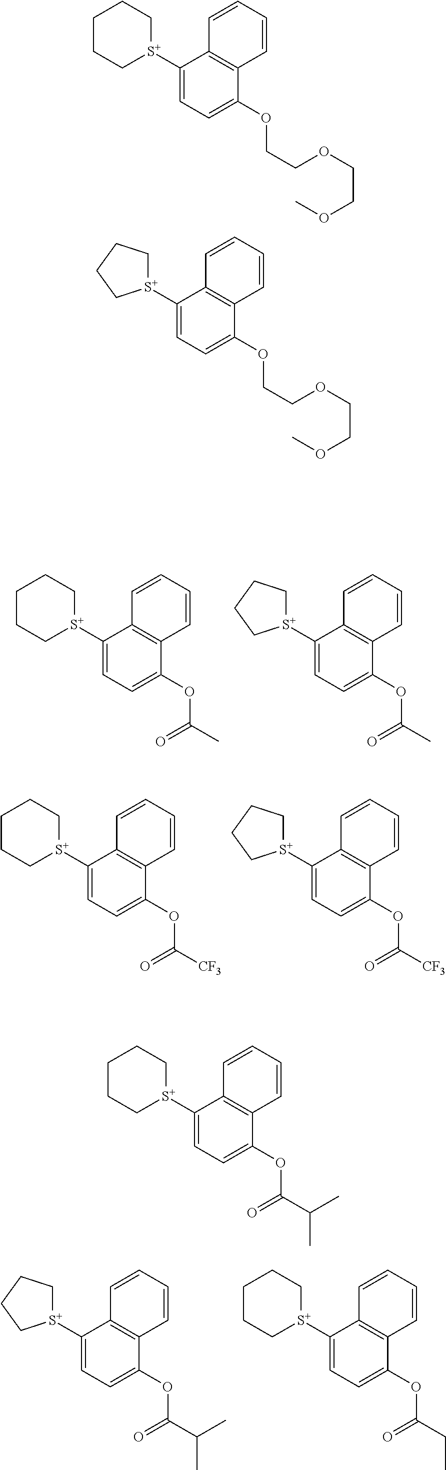

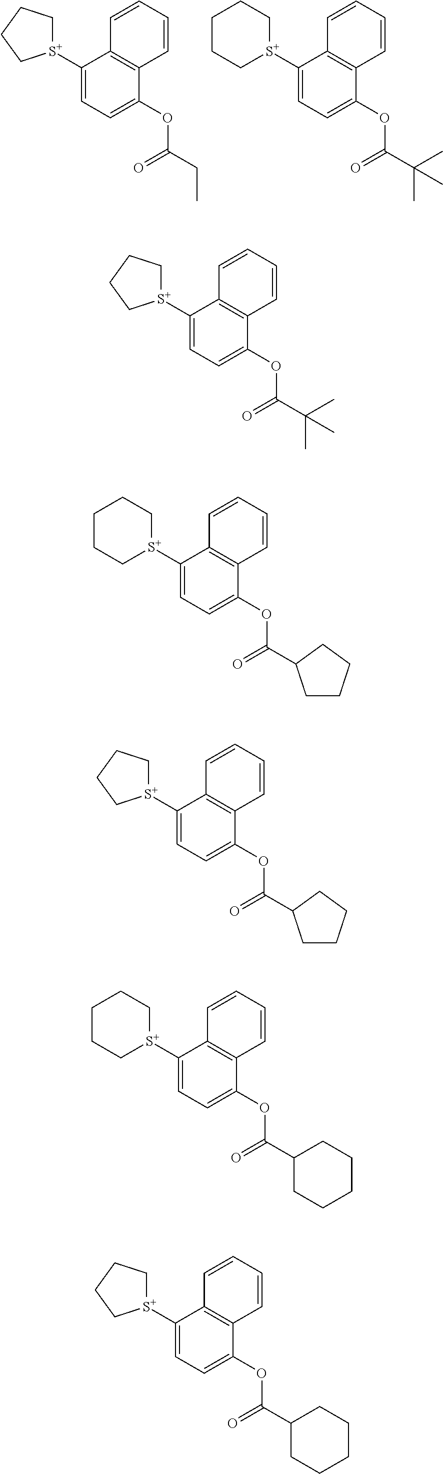

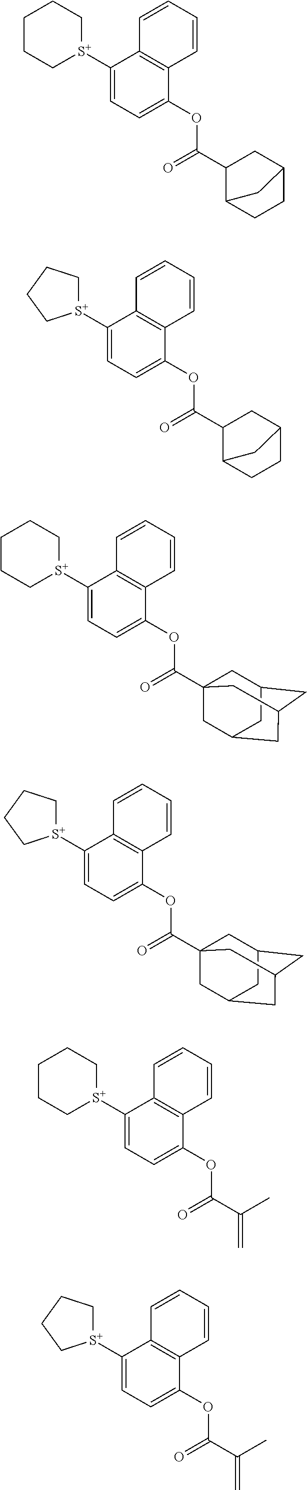 [SMILES: O=C(Oc1ccc([S+]2CCCCC2)c2ccccc12)C1CC2CCC1C2 ; O=C(Oc1ccc([S+]2CCCC2)c2ccccc12)C1CC2CCC1C2 ; O=C(Oc1ccc([S+]2CCCCC2)c2ccccc12)C12CC3CC(CC(C3)C1)C2 ; O=C(Oc1ccc([S+]2CCCC2)c2ccccc12)C12CC3CC(CC(C3)C1)C2 ; C=C(C)C(=O)Oc1ccc([S+]2CCCCC2)c2ccccc12 ; C=C(C)C(=O)Oc1ccc([S+]2CCCC2)c2ccccc12]

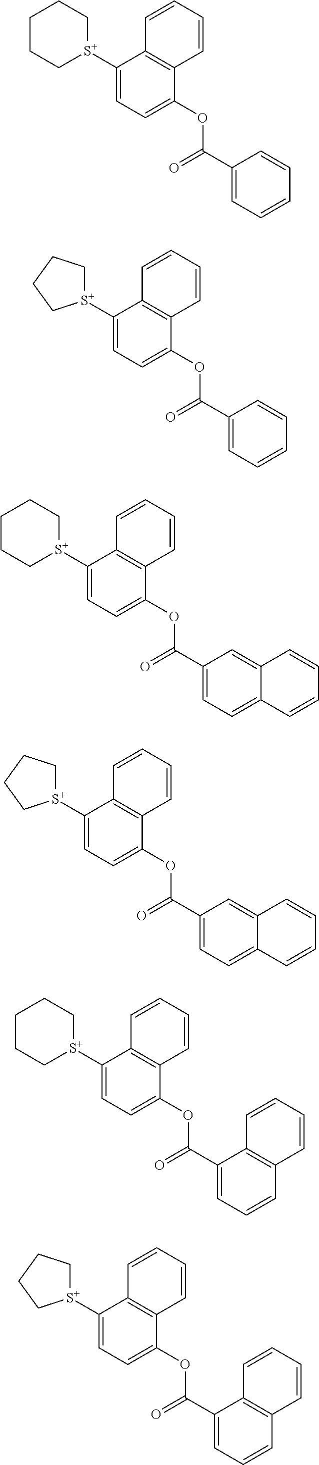 [SMILES: O=C(Oc1ccc([S+]2CCCCC2)c2ccccc12)c1ccccc1 ; O=C(Oc1ccc([S+]2CCCC2)c2ccccc12)c1ccccc1 ; O=C(Oc1ccc([S+]2CCCCC2)c2ccccc12)c1ccc2ccccc2c1 ; O=C(Oc1ccc([S+]2CCCC2)c2ccccc12)c1ccc2ccccc2c1 ; O=C(Oc1ccc([S+]2CCCCC2)c2ccccc12)c1cccc2ccccc12 ; O=C(Oc1ccc([S+]2CCCC2)c2ccccc12)c1cccc2ccccc12]

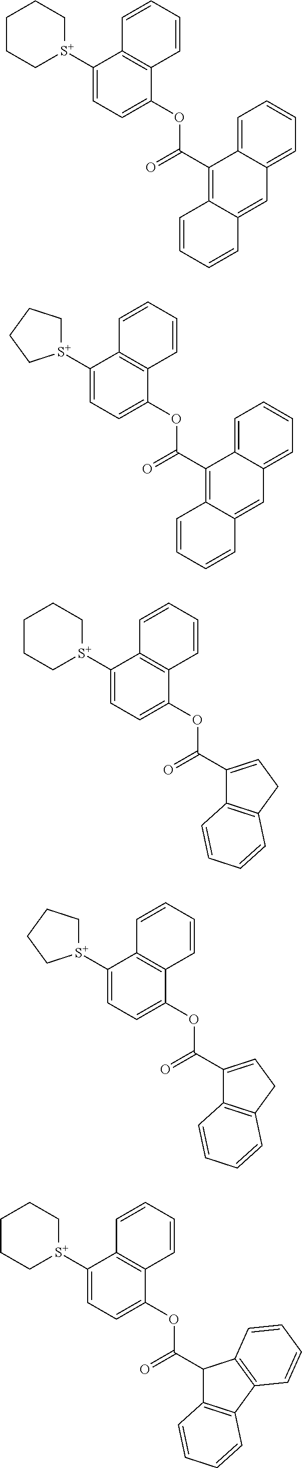 [SMILES: O=C(Oc1ccc([S+]2CCCCC2)c2ccccc12)c1c2ccccc2cc2ccccc12 ; O=C(Oc1ccc([S+]2CCCC2)c2ccccc12)c1c2ccccc2cc2ccccc12 ; O=C(Oc1ccc([S+]2CCCCC2)c2ccccc12)C1=CCc2ccccc21 ; O=C(Oc1ccc([S+]2CCCC2)c2ccccc12)C1=CCc2ccccc21 ; O=C(Oc1ccc([S+]2CCCCC2)c2ccccc12)C1c2ccccc2-c2ccccc21]

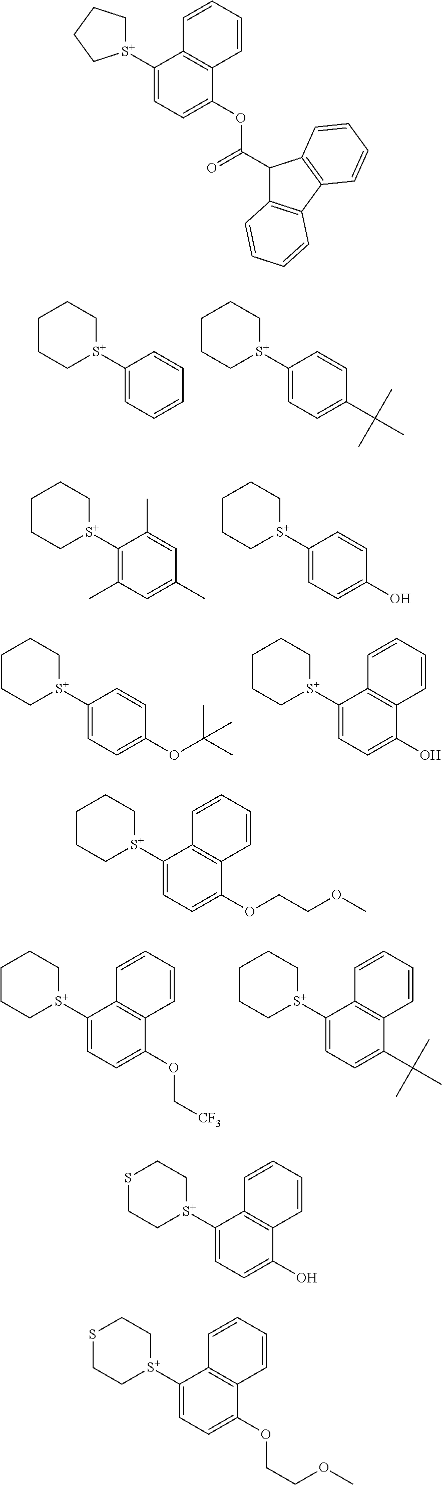

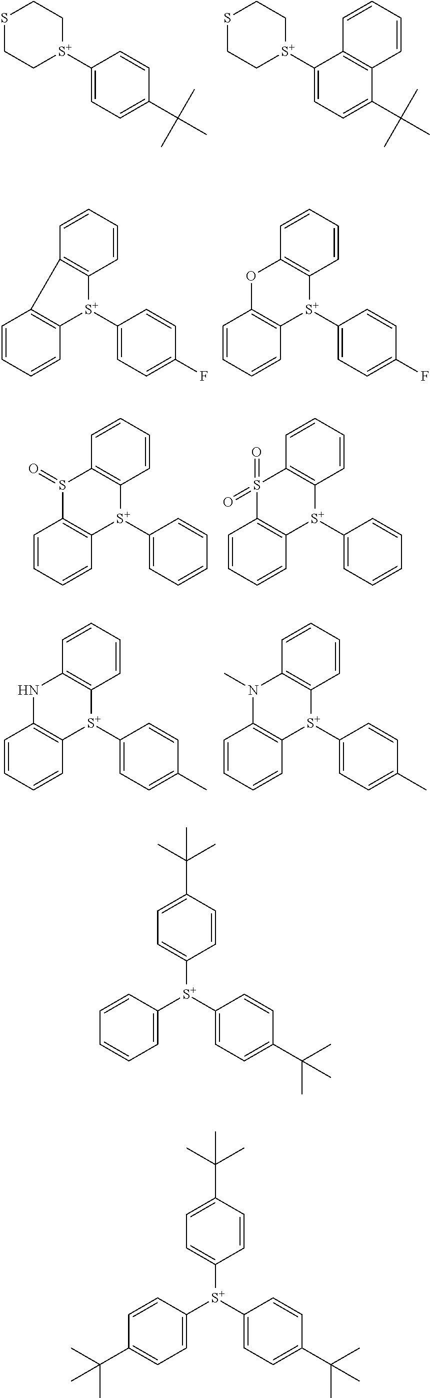

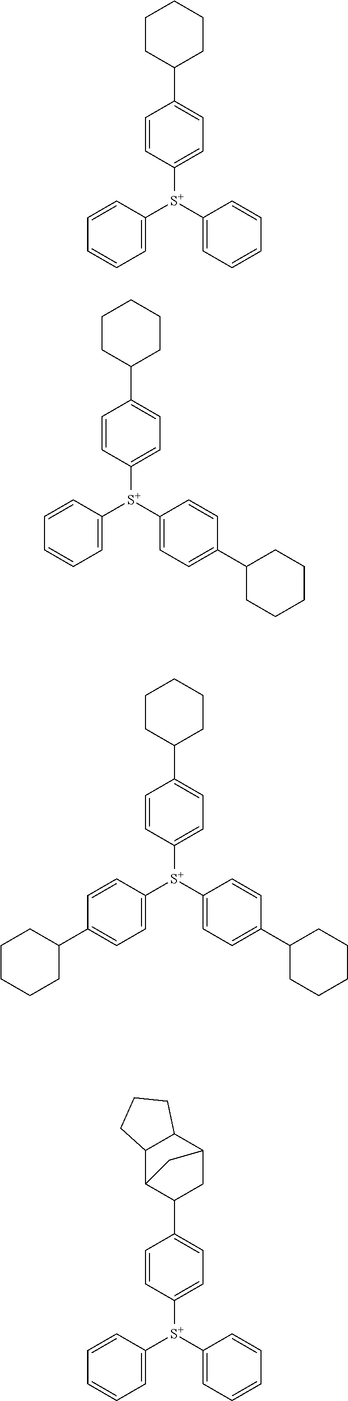 [SMILES: c1ccc([S+](c2ccccc2)c2ccc(C3CCCCC3)cc2)cc1 ; c1ccc([S+](c2ccc(C3CCCCC3)cc2)c2ccc(C3CCCCC3)cc2)cc1 ; c1cc([S+](c2ccc(C3CCCCC3)cc2)c2ccc(C3CCCCC3)cc2)ccc1C1CCCCC1 ; c1ccc([S+](c2ccccc2)c2ccc(C3CC4CC3C3CCCC43)cc2)cc1]

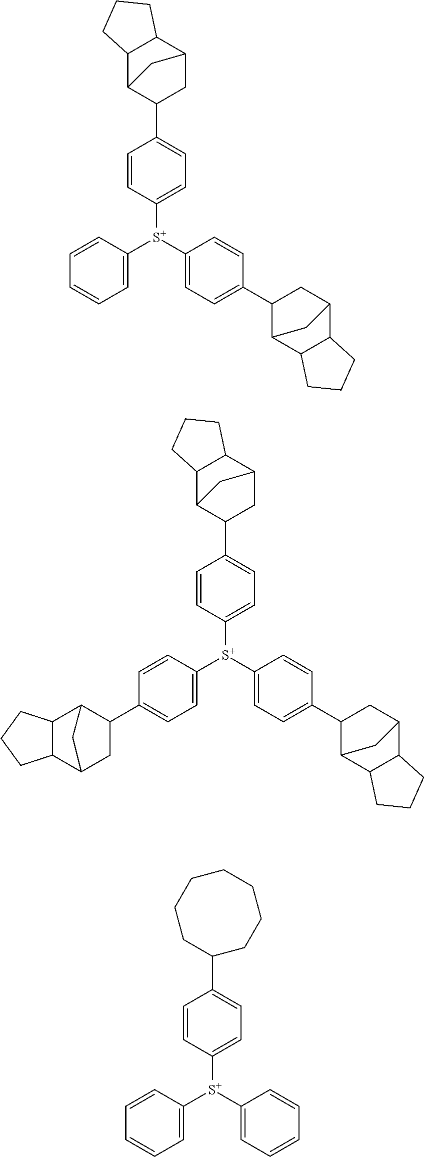 [SMILES: c1ccc([S+](c2ccc(C3CC4CC3C3CCCC43)cc2)c2ccc(C3CC4CC3C3CCCC43)cc2)cc1 ; c1cc([S+](c2ccc(C3CC4CC3C3CCCC43)cc2)c2ccc(C3CC4CC3C3CCCC43)cc2)ccc1C1CC2CC1C1CCCC21 ; c1ccc([S+](c2ccccc2)c2ccc(C3CCCCCCC3)cc2)cc1]

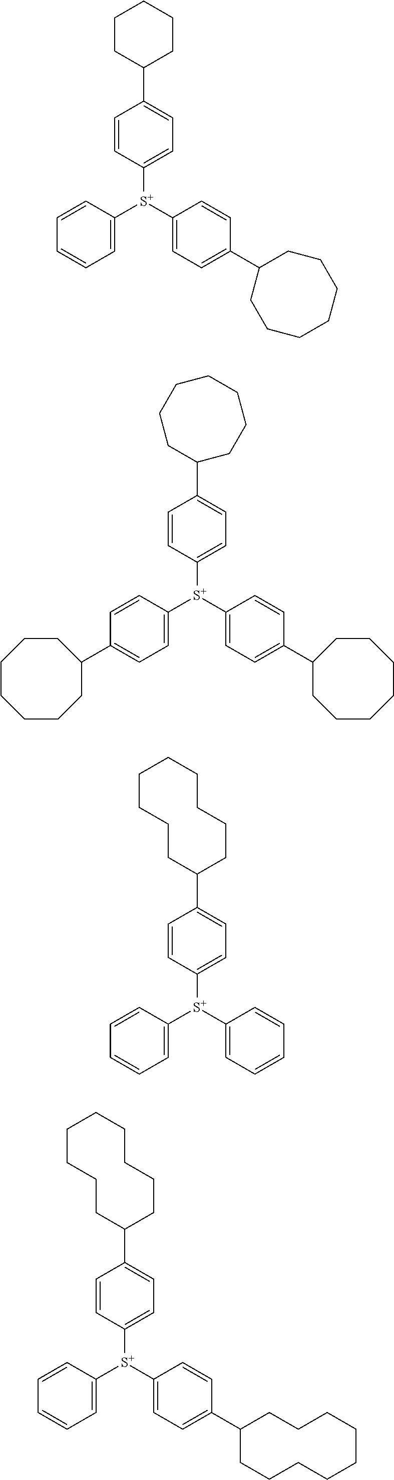 [SMILES: c1ccc([S+](c2ccc(C3CCCCCCC3)cc2)c2ccc(C3CCCCC3)cc2)cc1 ; c1cc([S+](c2ccc(C3CCCCCCC3)cc2)c2ccc(C3CCCCCCC3)cc2)ccc1C1CCCCCCC1 ; c1ccc([S+](c2ccccc2)c2ccc(C3CCCCCCCCC3)cc2)cc1 ; c1ccc([S+](c2ccc(C3CCCCCCCCC3)cc2)c2ccc(C3CCCCCCCCC3)cc2)cc1]

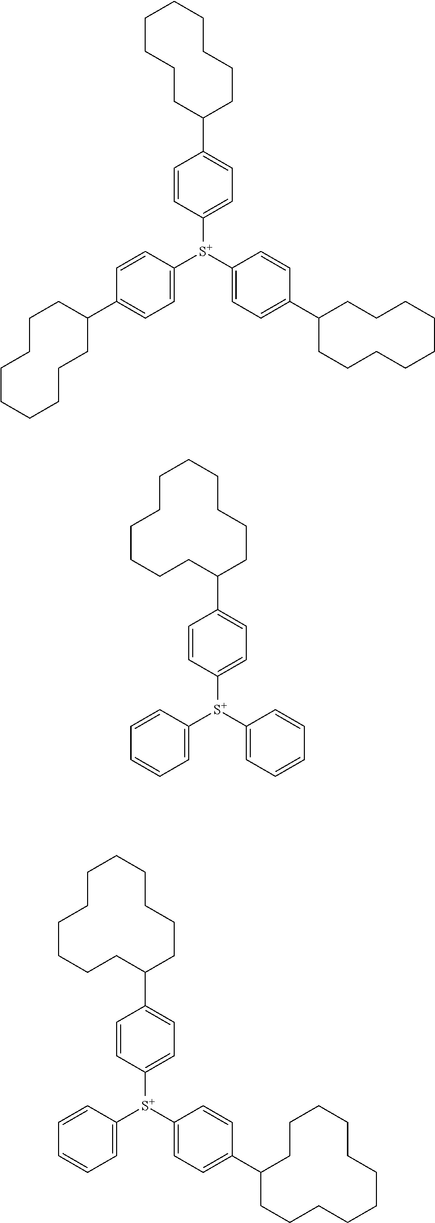 [SMILES: c1cc([S+](c2ccc(C3CCCCCCCCC3)cc2)c2ccc(C3CCCCCCCCC3)cc2)ccc1C1CCCCCCCCC1 ; c1ccc([S+](c2ccccc2)c2ccc(C3CCCCCCCCCCC3)cc2)cc1 ; c1ccc([S+](c2ccc(C3CCCCCCCCCCC3)cc2)c2ccc(C3CCCCCCCCCCC3)cc2)cc1]

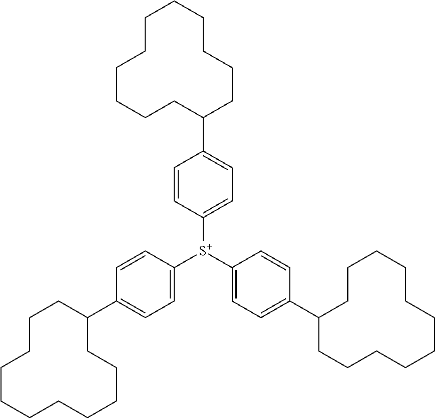 [SMILES: c1cc([S+](c2ccc(C3CCCCCCCCCCC3)cc2)c2ccc(C3CCCCCCCCCCC3)cc2)ccc1C1CCCCCCCCCCC1]

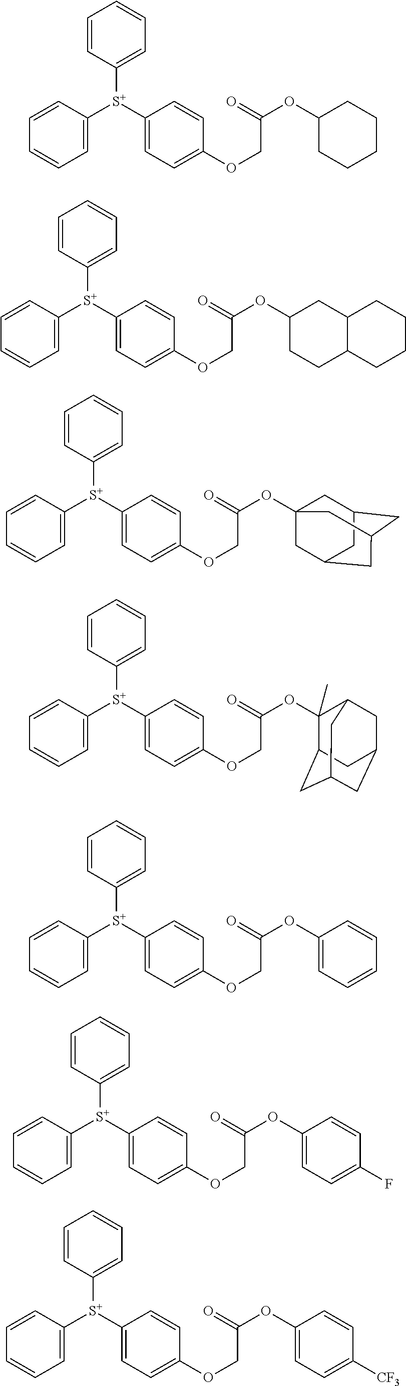

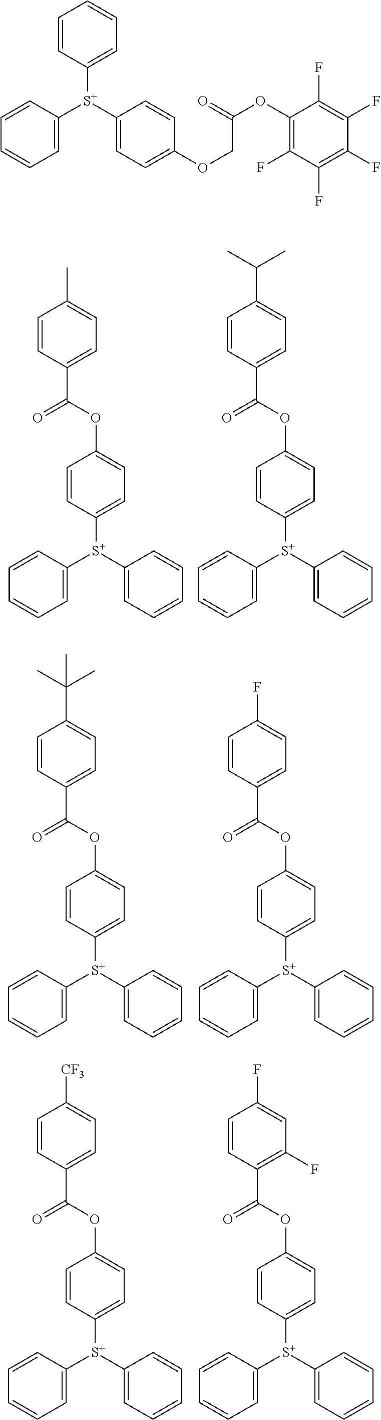

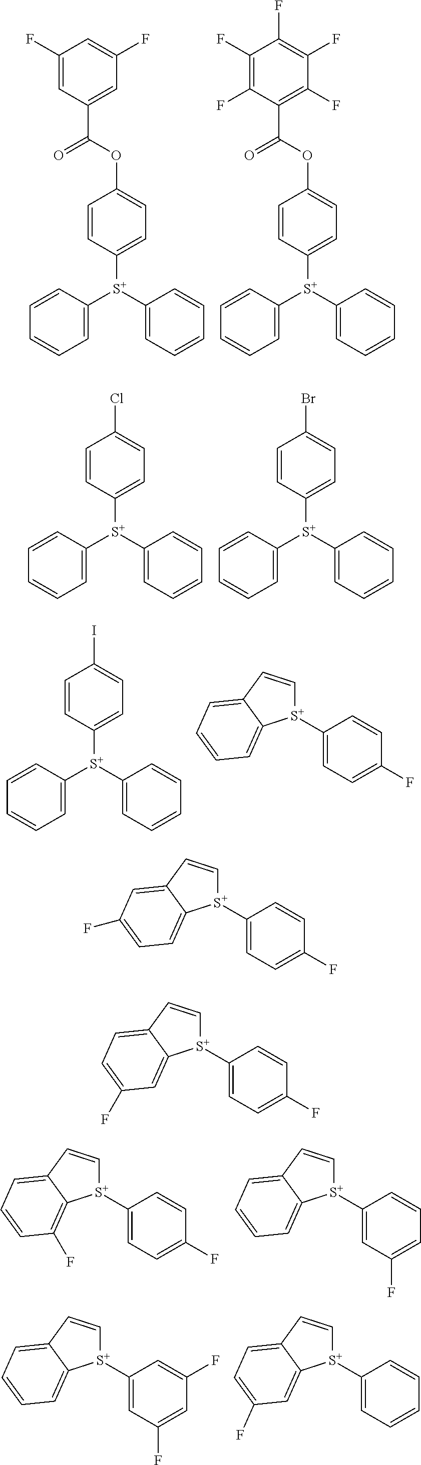

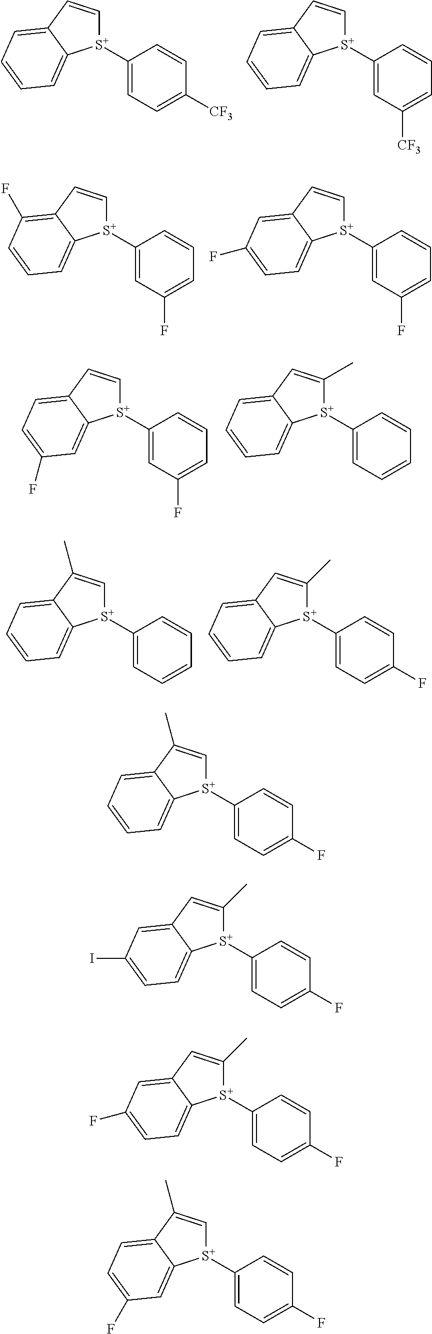

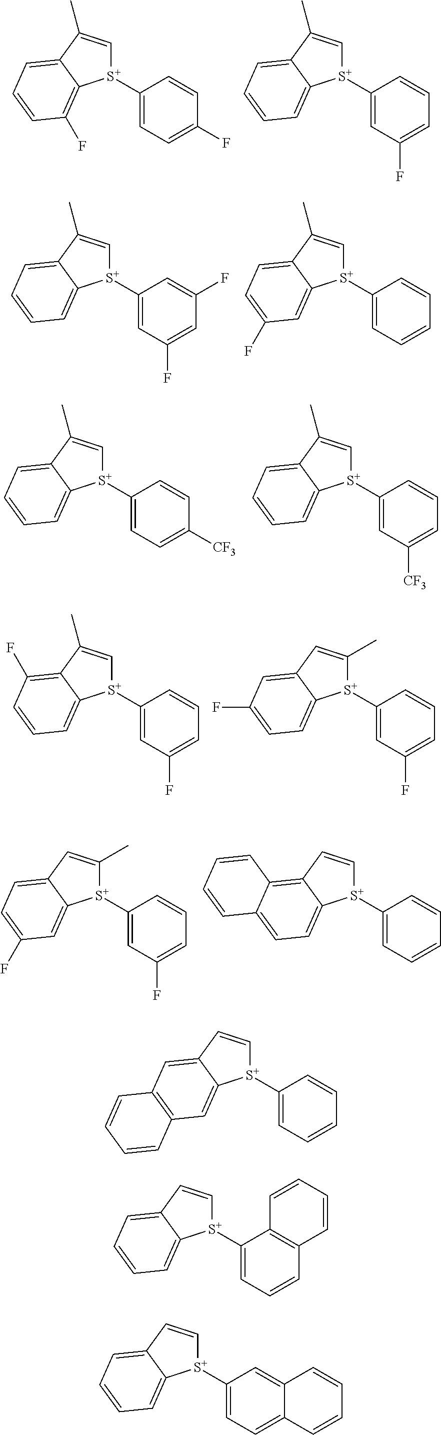

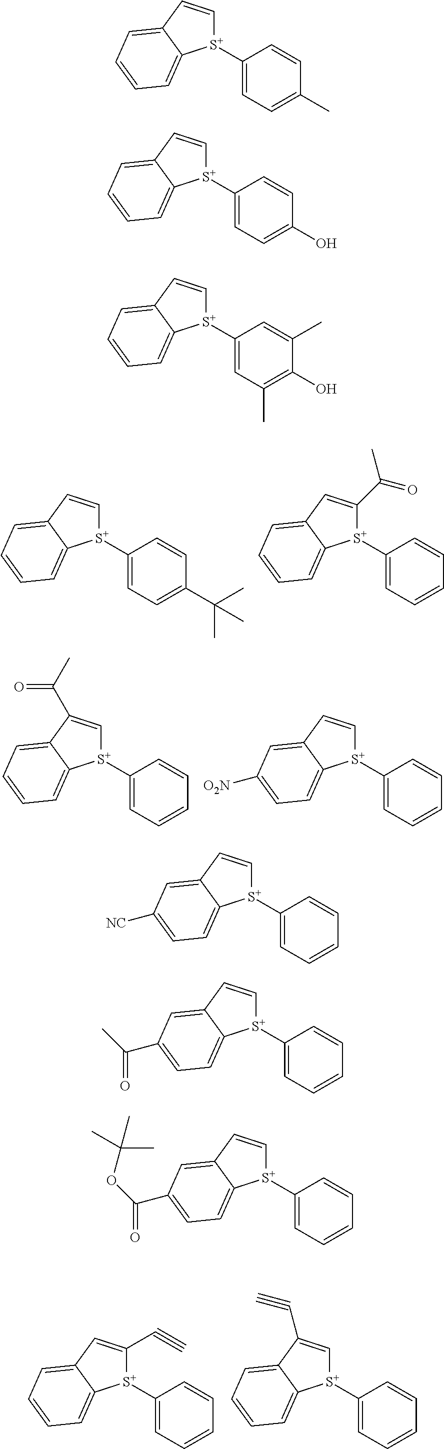

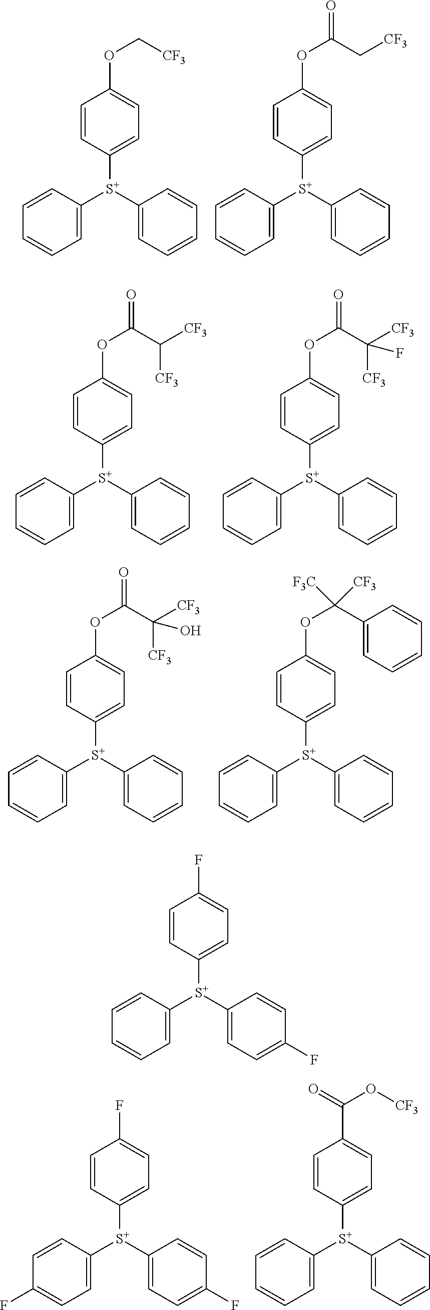

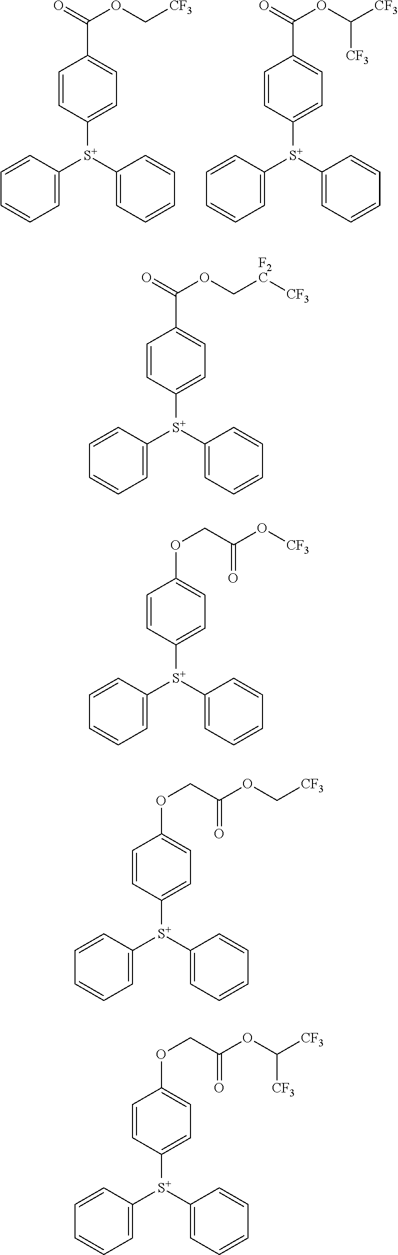 [SMILES: O=C(OCC(F)(F)F)c1ccc([S+](c2ccccc2)c2ccccc2)cc1 ; O=C(OC(C(F)(F)F)C(F)(F)F)c1ccc([S+](c2ccccc2)c2ccccc2)cc1 ; O=C(OCC(F)(F)C(F)(F)F)c1ccc([S+](c2ccccc2)c2ccccc2)cc1 ; O=C(COc1ccc([S+](c2ccccc2)c2ccccc2)cc1)OC(F)(F)F ; O=C(COc1ccc([S+](c2ccccc2)c2ccccc2)cc1)OCC(F)(F)F ; O=C(COc1ccc([S+](c2ccccc2)c2ccccc2)cc1)OC(C(F)(F)F)C(F)(F)F]

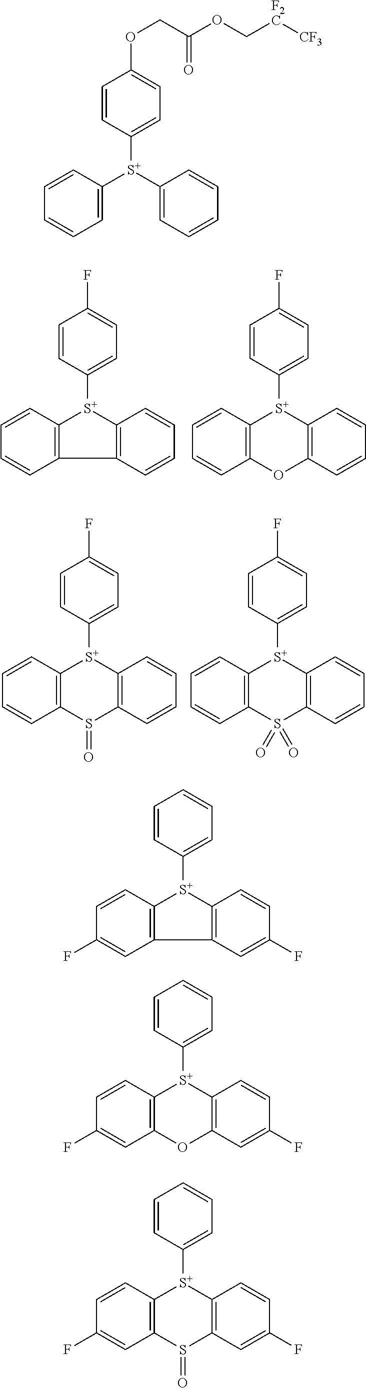

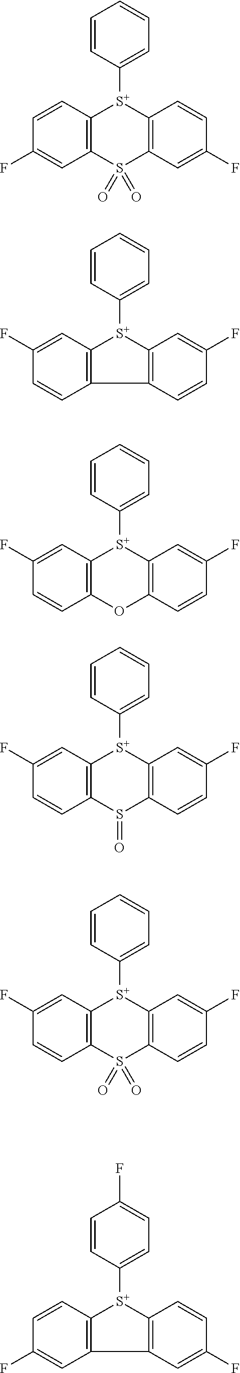 [SMILES: O=S1(=O)c2cc(F)ccc2[S+](c2ccccc2)c2ccc(F)cc21 ; Fc1ccc2c3ccc(F)cc3[s+](-c3ccccc3)c2c1 ; Fc1ccc2c(c1)[S+](c1ccccc1)c1cc(F)ccc1O2 ; O=S1c2ccc(F)cc2[S+](c2ccccc2)c2cc(F)ccc21 ; O=S1(=O)c2ccc(F)cc2[S+](c2ccccc2)c2cc(F)ccc21 ; Fc1ccc(-[s+]2c3ccc(F)cc3c3cc(F)ccc32)cc1]

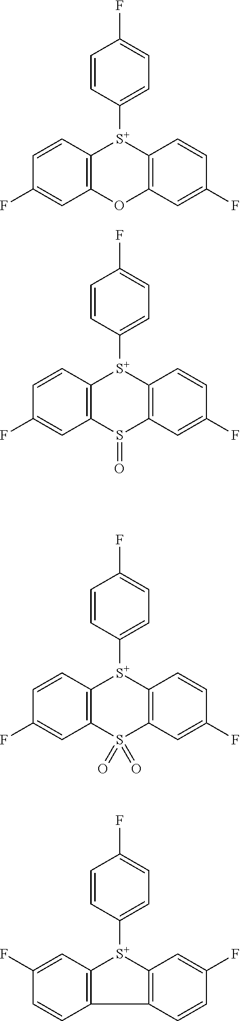 [SMILES: Fc1ccc([S+]2c3ccc(F)cc3Oc3cc(F)ccc32)cc1 ; O=S1c2cc(F)ccc2[S+](c2ccc(F)cc2)c2ccc(F)cc21 ; O=S1(=O)c2cc(F)ccc2[S+](c2ccc(F)cc2)c2ccc(F)cc21 ; Fc1ccc(-[s+]2c3cc(F)ccc3c3ccc(F)cc32)cc1]

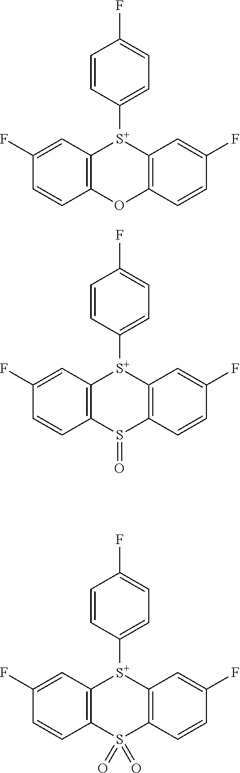 [SMILES: Fc1ccc([S+]2c3cc(F)ccc3Oc3ccc(F)cc32)cc1 ; O=S1c2ccc(F)cc2[S+](c2ccc(F)cc2)c2cc(F)ccc21 ; O=S1(=O)c2ccc(F)cc2[S+](c2ccc(F)cc2)c2cc(F)ccc21]

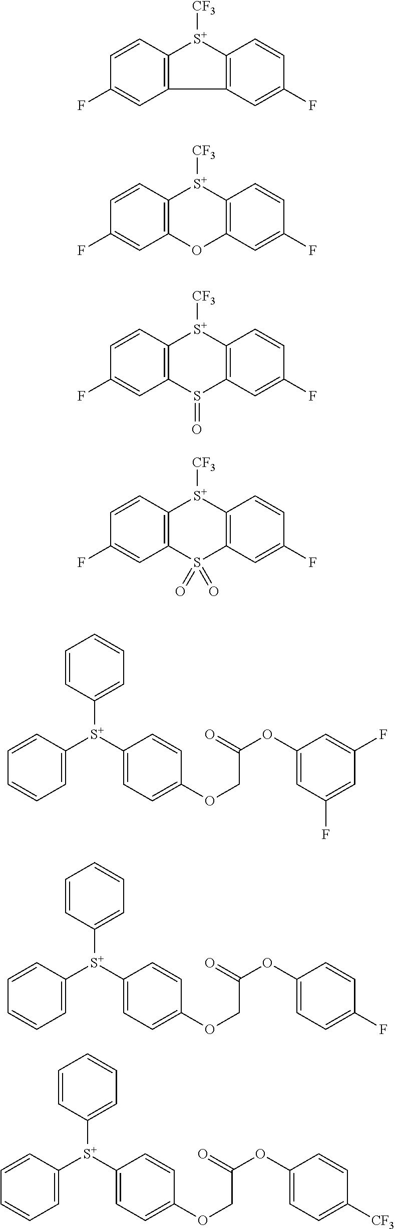

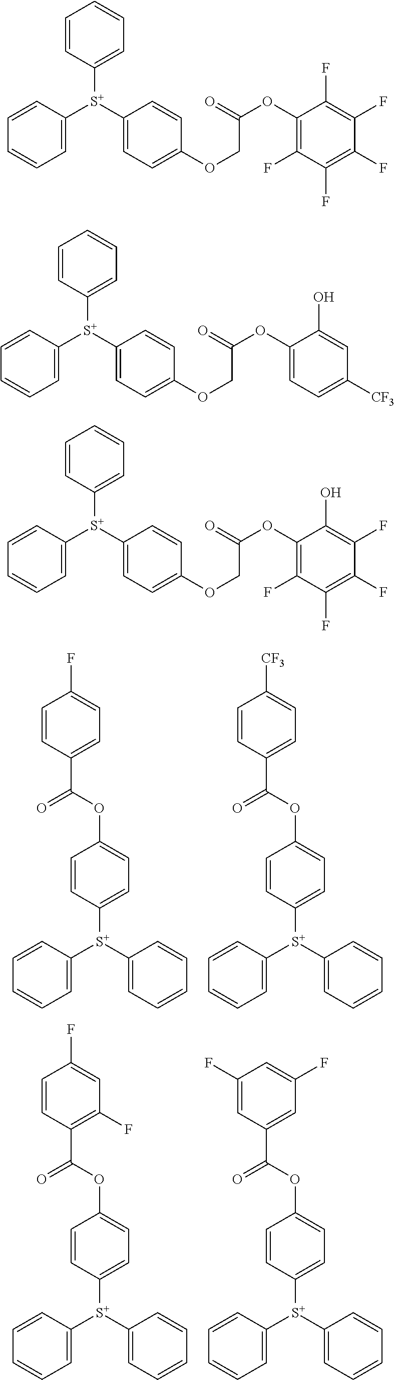

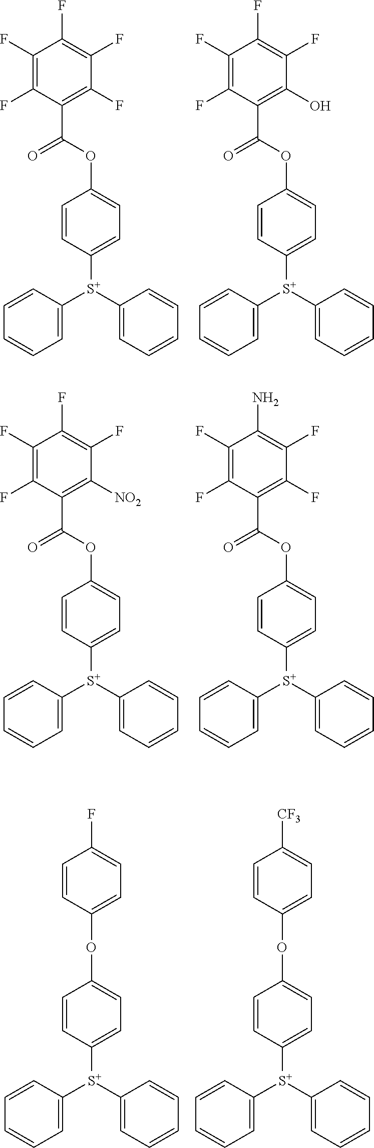 [SMILES: O=C(Oc1ccc([S+](c2ccccc2)c2ccccc2)cc1)c1c(F)c(F)c(F)c(F)c1F ; O=C(Oc1ccc([S+](c2ccccc2)c2ccccc2)cc1)c1c(O)c(F)c(F)c(F)c1F ; O=C(Oc1ccc([S+](c2ccccc2)c2ccccc2)cc1)c1c(F)c(F)c(F)c(F)c1[N+](=O)[O-] ; Nc1c(F)c(F)c(C(=O)Oc2ccc([S+](c3ccccc3)c3ccccc3)cc2)c(F)c1F ; Fc1ccc(Oc2ccc([S+](c3ccccc3)c3ccccc3)cc2)cc1 ; FC(F)(F)c1ccc(Oc2ccc([S+](c3ccccc3)c3ccccc3)cc2)cc1]

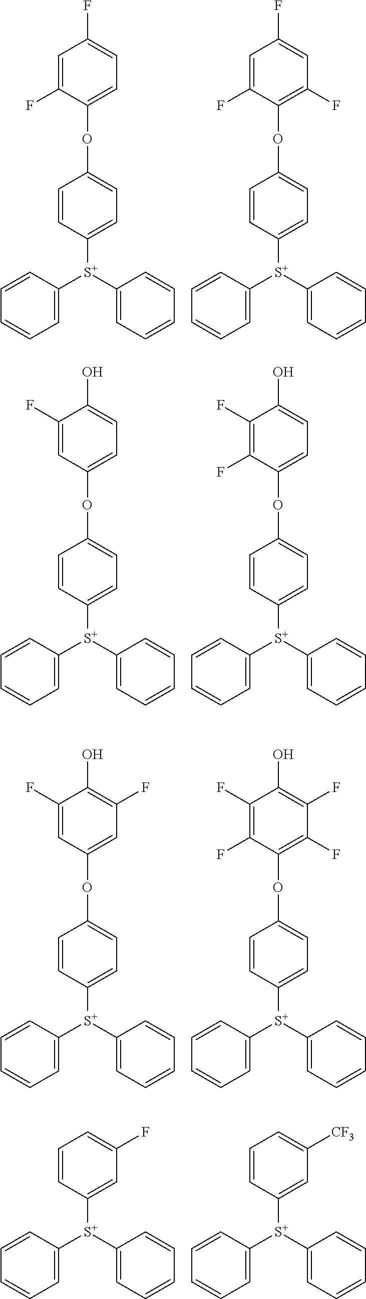

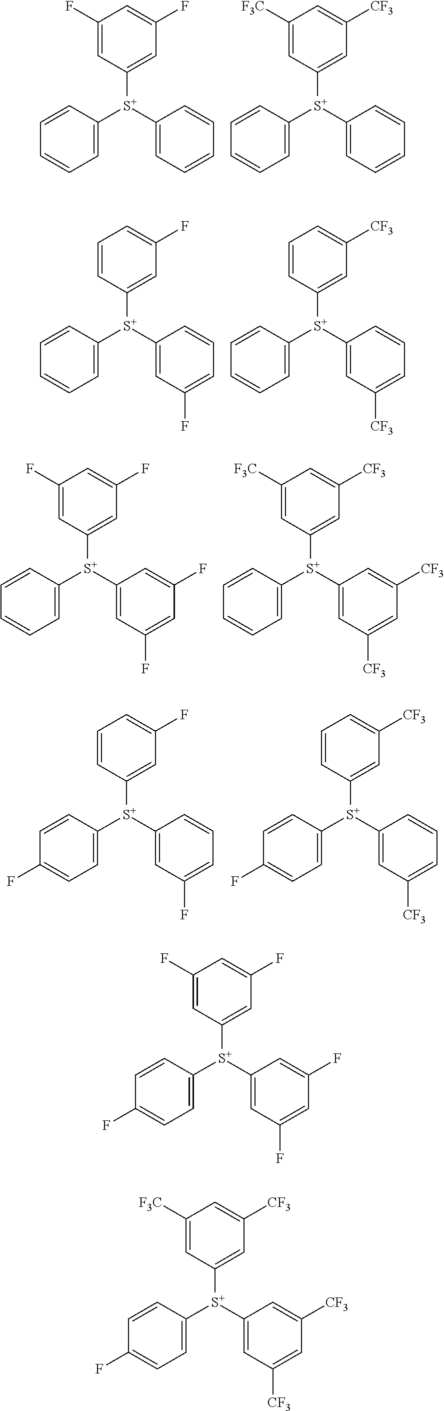

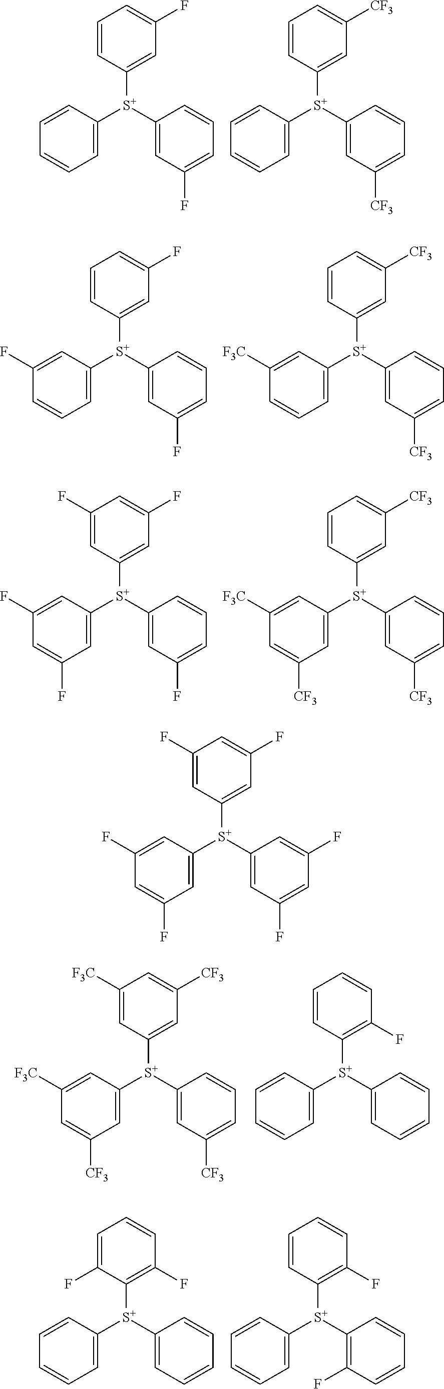

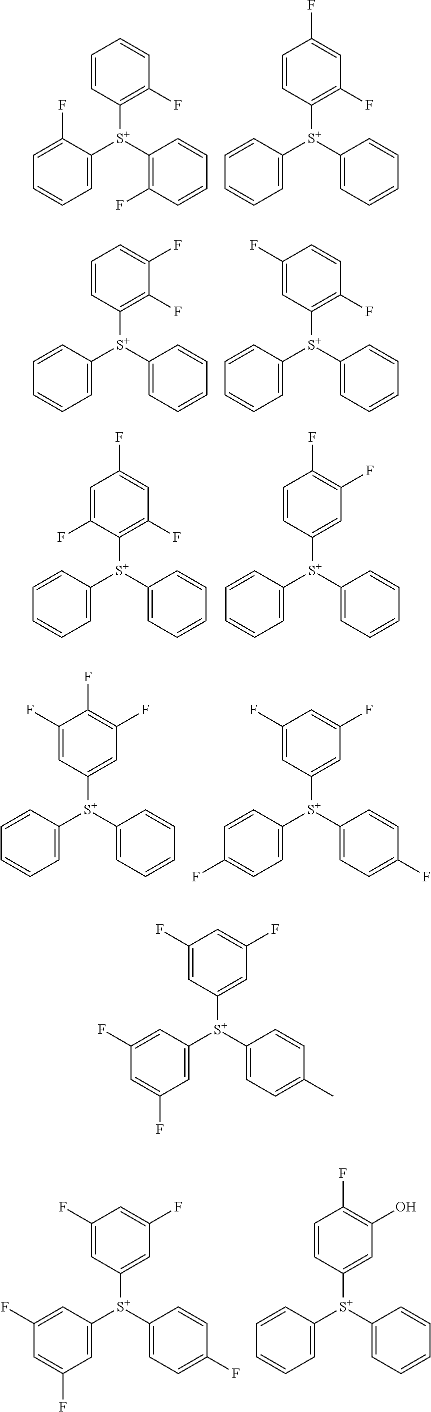

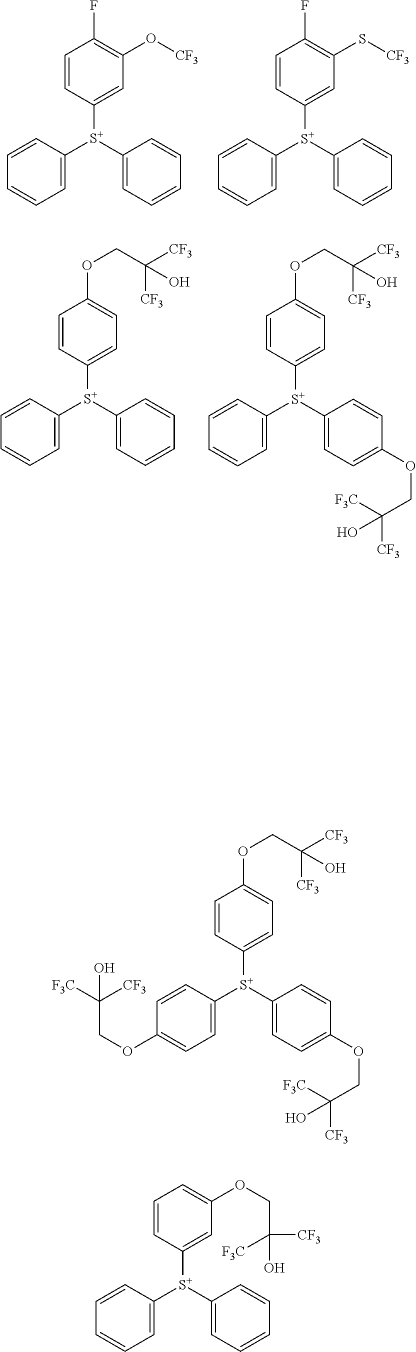 [SMILES: Fc1ccc([S+](c2ccccc2)c2ccccc2)cc1OC(F)(F)F ; Fc1ccc([S+](c2ccccc2)c2ccccc2)cc1SC(F)(F)F ; OC(COc1ccc([S+](c2ccccc2)c2ccccc2)cc1)(C(F)(F)F)C(F)(F)F ; OC(COc1ccc([S+](c2ccccc2)c2ccc(OCC(O)(C(F)(F)F)C(F)(F)F)cc2)cc1)(C(F)(F)F)C(F)(F)F ; OC(COc1ccc([S+](c2ccc(OCC(O)(C(F)(F)F)C(F)(F)F)cc2)c2ccc(OCC(O)(C(F)(F)F)C(F)(F)F)cc2)cc1)(C(F)(F)F)C(F)(F)F ; OC(COc1cccc([S+](c2ccccc2)c2ccccc2)c1)(C(F)(F)F)C(F)(F)F]

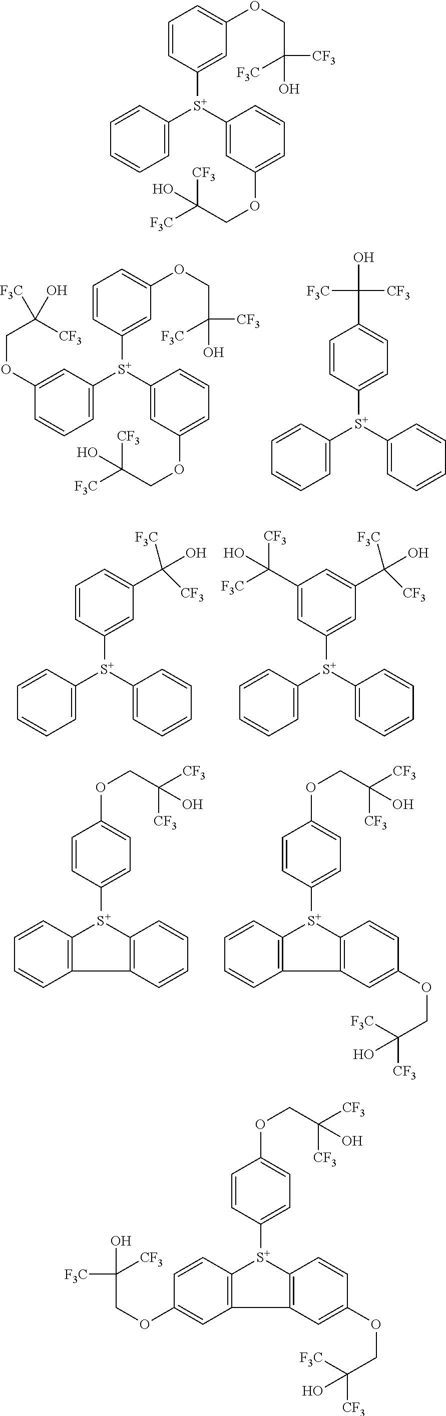

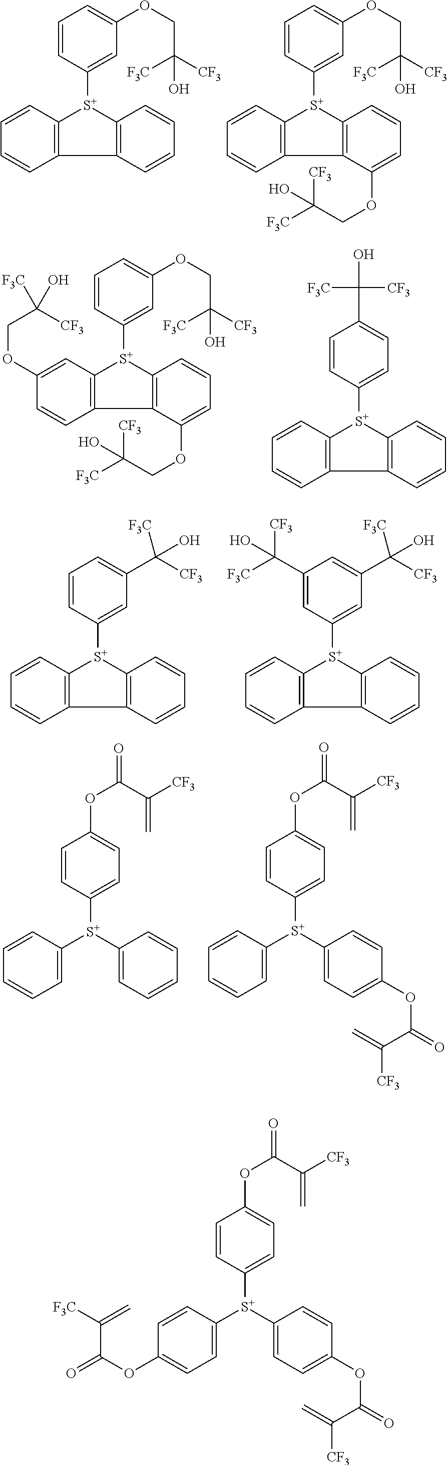

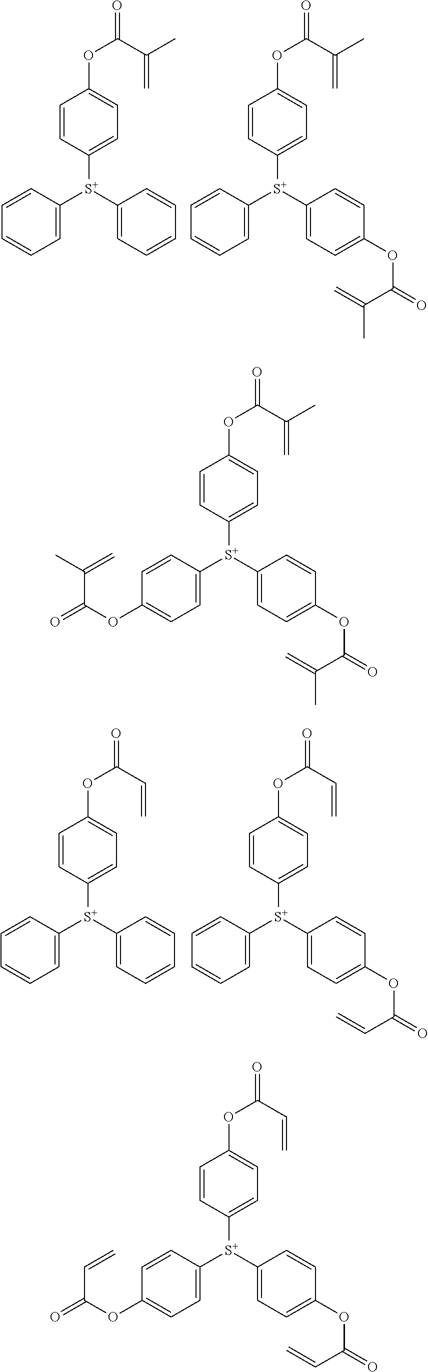 [SMILES: C=C(C)C(=O)Oc1ccc([S+](c2ccccc2)c2ccccc2)cc1 ; C=C(C)C(=O)Oc1ccc([S+](c2ccccc2)c2ccc(OC(=O)C(=C)C)cc2)cc1 ; C=C(C)C(=O)Oc1ccc([S+](c2ccc(OC(=O)C(=C)C)cc2)c2ccc(OC(=O)C(=C)C)cc2)cc1 ; C=CC(=O)Oc1ccc([S+](c2ccccc2)c2ccccc2)cc1 ; C=CC(=O)Oc1ccc([S+](c2ccccc2)c2ccc(OC(=O)C=C)cc2)cc1 ; C=CC(=O)Oc1ccc([S+](c2ccc(OC(=O)C=C)cc2)c2ccc(OC(=O)C=C)cc2)cc1]

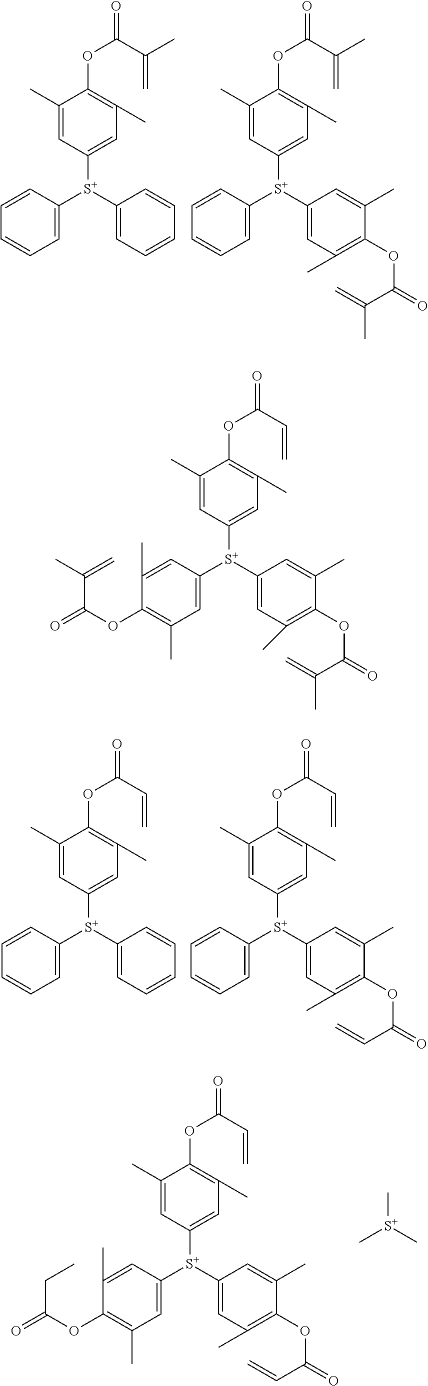

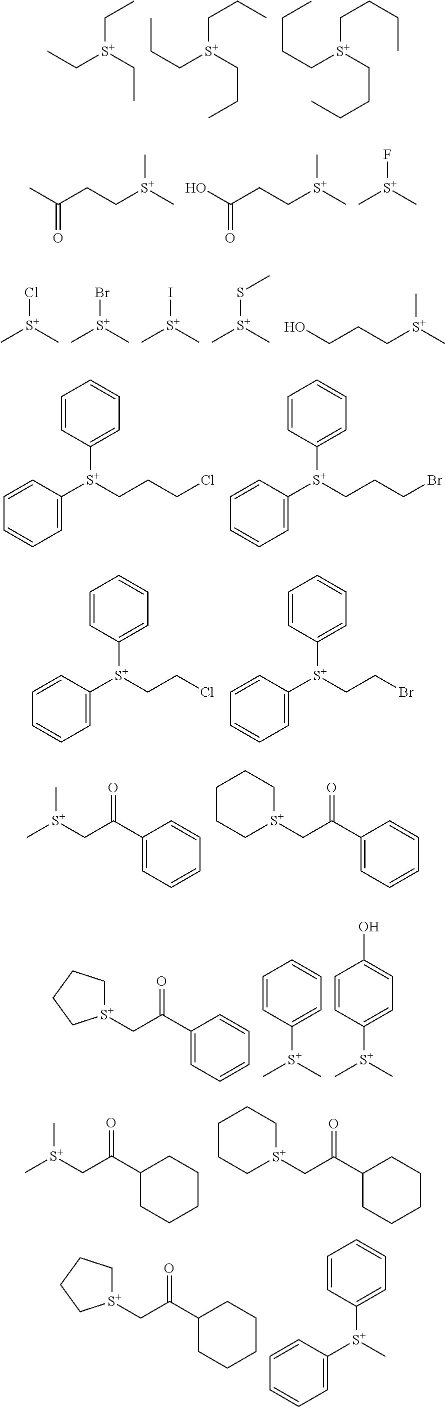

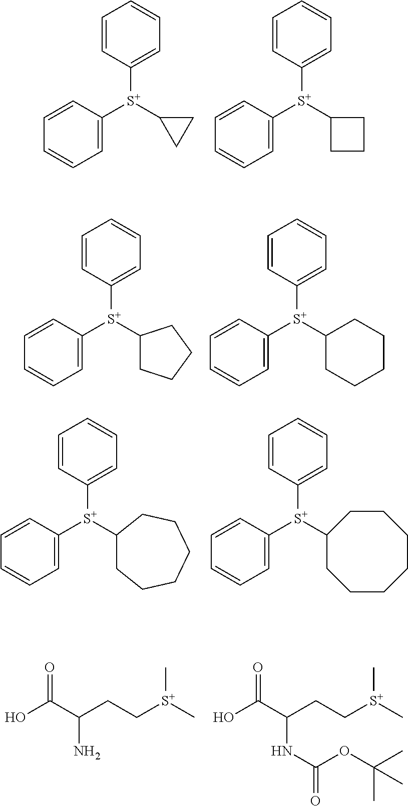

In the positive resist composition, the quencher (A) is preferably present in an amount of 0.01 to 30 parts by weight, more preferably 0.02 to 20 parts by weight per 100 parts by weight of the base polymer (C) to be described later.

### (B) Acid Generator

The acid generator as component (B) is a sulfonium salt consisting of a sulfonate anion having fluorine on the carbon atom at α- and/or β-position of the sulfo group (referred to as “fluorinated sulfonate anion,” hereinafter) and a sulfonium cation.

Preferably, the fluorinated sulfonate anion has the formula (2-1) or (2-2).

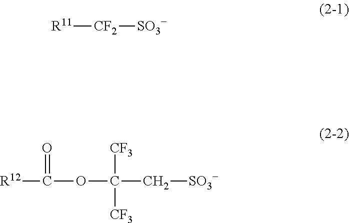

In formula (2-1), R¹¹ is fluorine or a C₁-C₂₀ hydrocarbyl group which may contain a heteroatom. The hydrocarbyl group may be saturated or unsaturated and straight, branched or cyclic. Examples thereof are as will be exemplified for the hydrocarbyl group R^{11A} in formula (2-1-1) below.

Preferred examples of the anion having formula (2-1) have the formula (2-1-1).

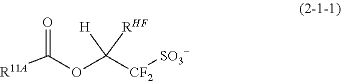

In formula (2-1-1), R^{11F} is hydrogen or trifluoromethyl, preferably trifluoromethyl. R^{11A} is a C₁-C₃₈ hydrocarbyl group which may contain a heteroatom. Suitable heteroatoms include oxygen, nitrogen, sulfur and halogen, with oxygen being preferred. Of the hydrocarbyl groups, those of 6 to 30 carbon atoms are preferred because a high resolution is available in fine pattern formation.

The hydrocarbyl group R^{11A} may be saturated or unsaturated and straight, branched or cyclic. Suitable hydrocarbyl groups include C₁-C₃₈ alkyl groups such as methyl, ethyl, propyl, isopropyl, butyl, isobutyl, sec-butyl, tert-butyl, pentyl, neopentyl, hexyl, heptyl, 2-ethylhexyl, nonyl, undecyl, tridecyl, pentadecyl, heptadecyl, icosanyl; C₃-C₃₈ cyclic saturated hydrocarbyl groups such as cyclopentyl, cyclohexyl, 1-adamantyl, 2-adamantyl, 1-adamantylmethyl, norbornyl, norbornylmethyl, tricyclodecanyl, tetracyclododecanyl, tetracyclododecanylmethyl, dicyclohexylmethyl; C₂-C₃₈ unsaturated aliphatic hydrocarbyl groups such as allyl, 3-cyclohexenyl, tetracyclododecenyl; C₆-C₃₈ aryl groups such as phenyl, 1-naphthyl, 2-naphthyl; C₇-C₃₈ aralkyl groups such as benzyl and diphenylmethyl; C₂₀-C₃₈ hydrocarbyl groups of steroid skeleton which may contain a heteroatom; and combinations thereof.

In the hydrocarbyl groups, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonic ester bond, carbonate bond, lactone ring, sultone ring, carboxylic anhydride (—C(═O)═O—C(═O)—) or haloalkyl moiety. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, and 3-oxocyclohexyl.

Examples of the anion having formula (2-1) are shown below, but not limited thereto.

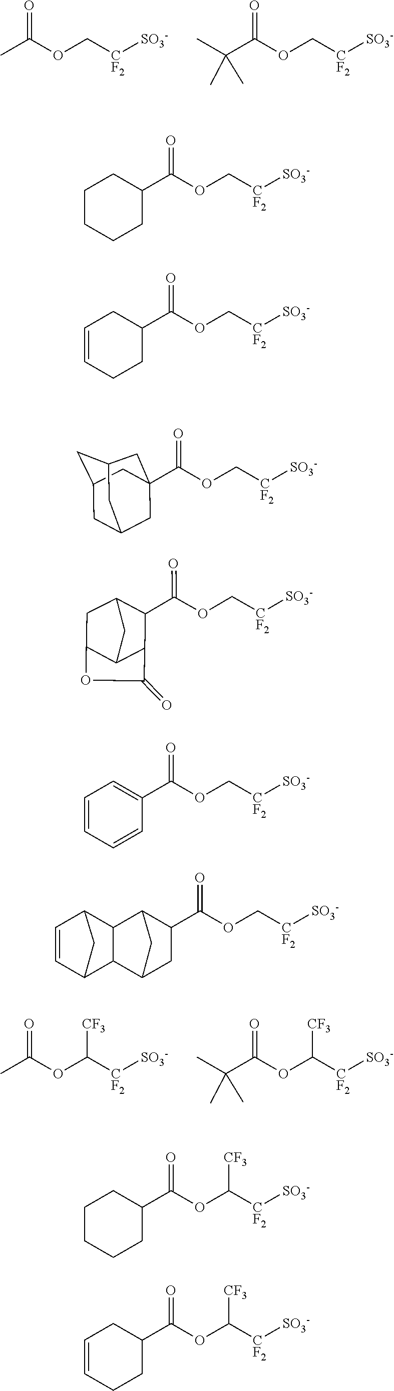

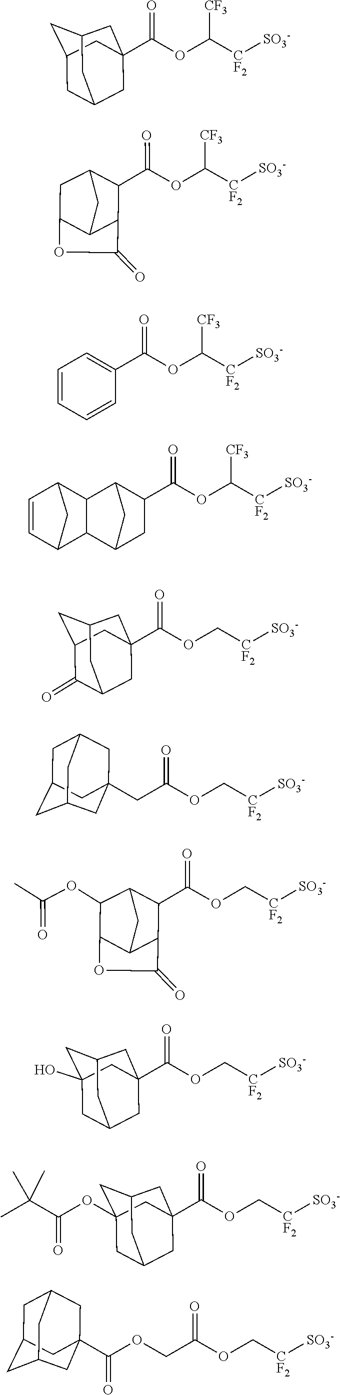

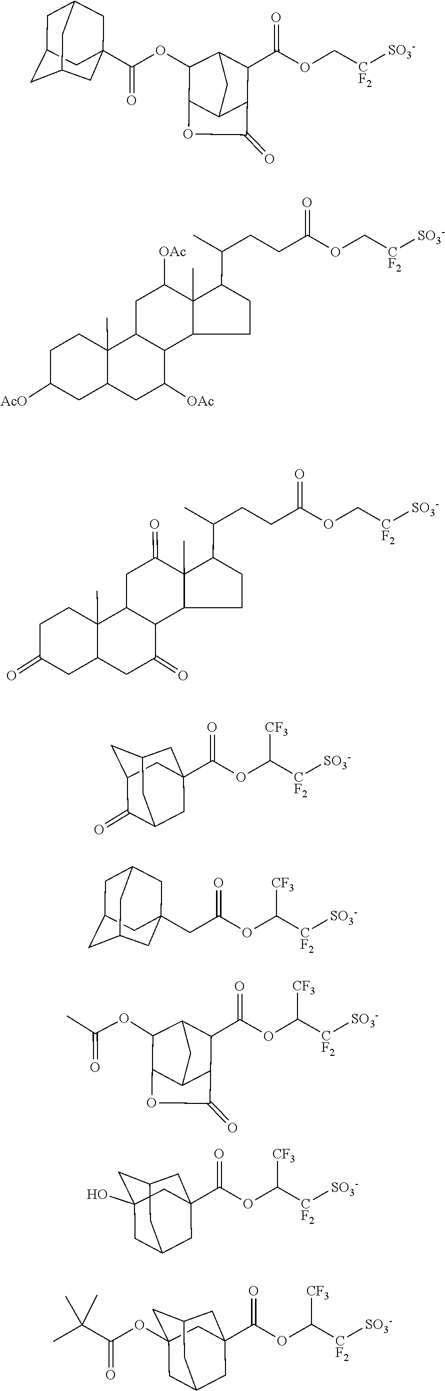

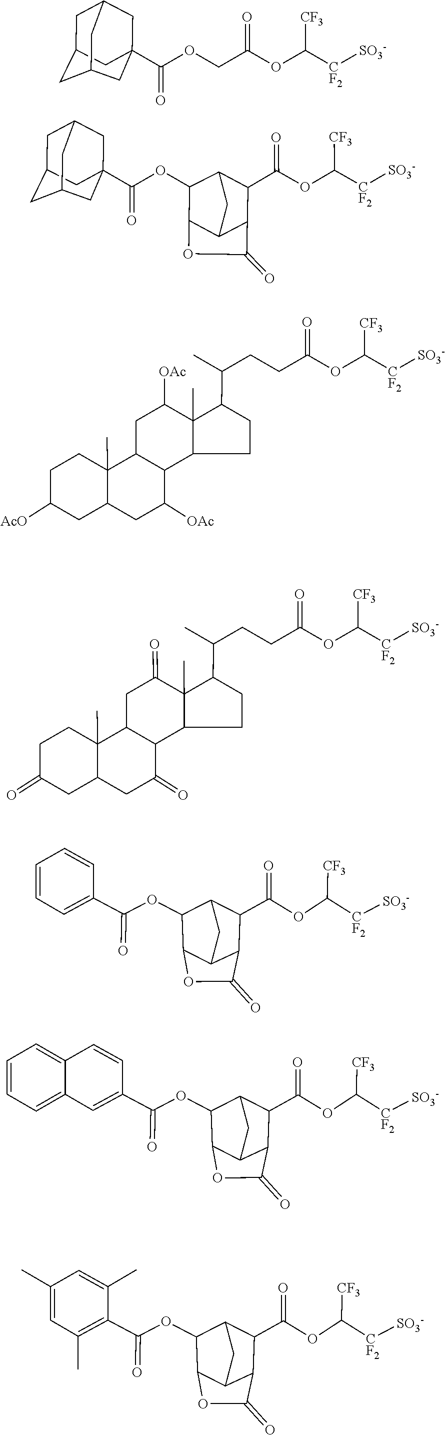

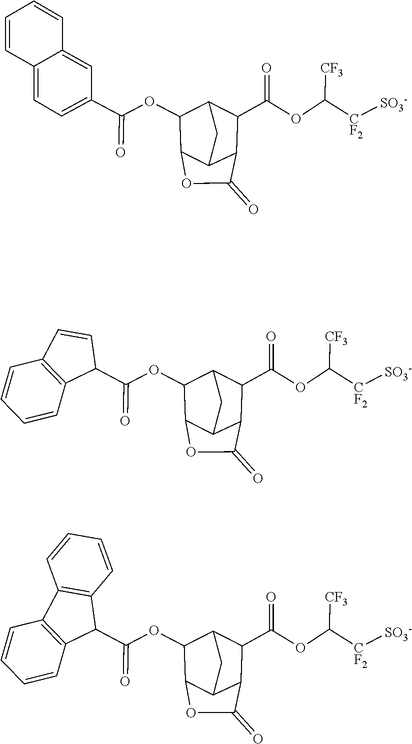

In formula (2-2), R¹² is a C₁-C₄₀ hydrocarbyl group which may contain a heteroatom. The hydrocarbyl group may be saturated or unsaturated and straight, branched or cyclic. Examples thereof are as exemplified above for the hydrocarbyl group R^{11A} in formula (2-1-1).

Examples of the anion having formula (2-2) are shown below, but not limited thereto.

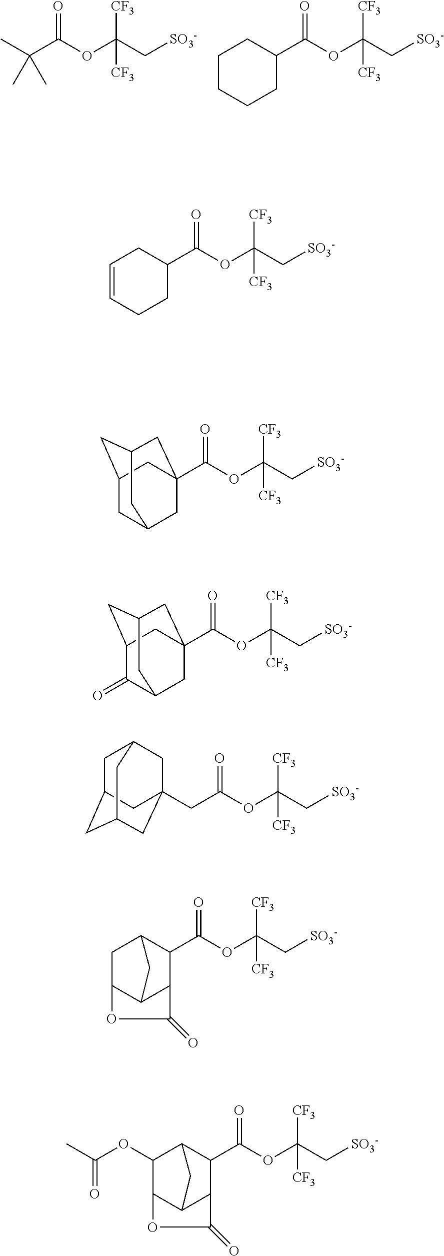

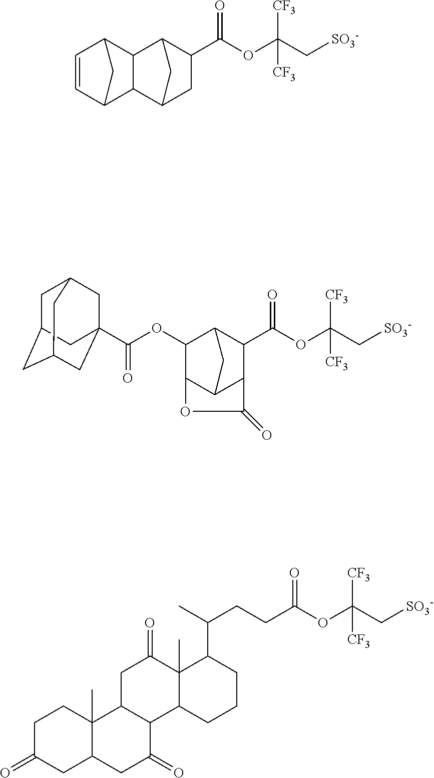

The compound having the anion of formula (2-2) has a sufficient acid strength to cleave acid labile groups in the base polymer because it is free of fluorine at α-position of the sulfo group, but has two trifluoromethyl groups at β-position. The compound is thus a useful acid generator.

Preferably the fluorinated sulfonate anion further contains iodine. Since iodine is highly absorptive to EUV, the containment of iodine in the anion increases the absorption of EUV upon exposure. Accordingly, the number of photons that the acid generator absorbs upon exposure increases, and the physical contrast is improved. The resulting resist composition has a higher sensitivity and contrast, further improved CDU and LWR, and a broader process window.

Specifically, the sulfonate anion containing iodine and having fluorine on the carbon atom at α- and/or β-position of the sulfo group is represented by the following formula (2-3), for example.

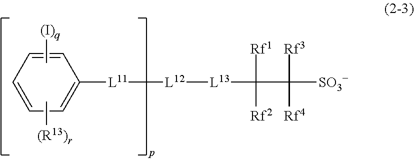

In formula (2-3), p is an integer of 1 to 3, q is an integer of 1 to 5, r is an integer of 0 to 3, and 1≤q+r≤5.

In formula (2-3), L¹¹ is a single bond, ether bond, ester bond, amide bond, imide bond, or C₁-C₆ saturated hydrocarbylene group in which some constituent —CH₂— may be replaced by an ester bond or ether bond. Notably, the constituent —CH₂— may be positioned at the end of the saturated hydrocarbylene group.

The C₁-C₆ saturated hydrocarbylene group L¹ may be straight, branched or cyclic. Examples thereof include C₁-C₆ alkanediyl groups such as methanediyl, ethane-1,1-diyl, ethane-1,2-diyl, propane-1,3-diyl, butane-1,4-diyl, pentane-1,5-diyl, and hexane-1,6-diyl; C₃-C₆ cyclic saturated hydrocarbylene groups such as cyclopropanediyl, cyclobutanediyl, cyclopentanediyl, and cyclohexanediyl; and combinations thereof.

In formula (2-3), L¹² is a single bond or a C₁-C₂₀ hydrocarbylene group in case of p=1, and a C₁-C₂₀ (p+1)-valent hydrocarbon group in case of p=2 or 3; the hydrocarbylene group and (p+1)-valent hydrocarbon group may contain at least one atom selected from oxygen, sulfur and nitrogen.

The C₁-C₂₀ hydrocarbylene group L¹² may be saturated or unsaturated and straight, branched or cyclic. Examples thereof include C₁-C₂₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, and dodecane-1,12-diyl; C₃-C₂₀ cyclic saturated hydrocarbylene groups such as cyclopentanediyl, cyclohexanediyl, norbornanediyl and adamantanediyl; C₂-C₂₀ unsaturated aliphatic hydrocarbylene groups such as vinylene and propene-1,3-diyl; C₆-C₂₀ arylene groups such as phenylene and naphthylene; and combinations thereof The C₁-C₂₀ (p+1)-valent hydrocarbon group L¹² may be saturated or unsaturated and straight, branched or cyclic. Examples thereof include groups obtained by removing one or two hydrogen atoms from the above-described examples of the C₁-C₂₀ hydrocarbylene group.

In formula (2-3), L¹³ is a single bond, ether bond or ester bond.

In formula (2-3), R¹³ is a hydroxy group, carboxy group, fluorine, chlorine, bromine or amino group, or a C₁-C₂₀ hydrocarbyl group, C₁-C₂₀ hydrocarbyloxy group, C₂-C₂₀ hydrocarbylcarbonyl group, C₂-C₂₀ hydrocarbyloxycarbonyl group, C₂-C₂₀ hydrocarbylcarbonyloxy group, or C₁-C₂₀ hydrocarbylsulfonyloxy group which may contain fluorine, chlorine, bromine, hydroxy, amino or ether bond, or —N(R^{13A})(R^{13B}), —N(R^{13C})—C(═O)—R^{13D}, or —N(R^{13C})—C(═O)—O—R^{13D}. R^{13A} and R^{13B} are each independently hydrogen or a C₁-C₆ saturated hydrocarbyl group. R^{13C} is hydrogen or a C₁-C₆ saturated hydrocarbyl group in which some or all of the hydrogen atoms may be substituted by halogen, hydroxy, C₁-C₆ saturated hydrocarbyloxy, C₂-C₆ saturated hydrocarbylcarbonyl or C₂-C₆ saturated hydrocarbylcarbonyloxy moiety. R^{13D} is a C₁-C₁₆ aliphatic hydrocarbyl, C₆-C₁₂ aryl or C₇-C₁₅ aralkyl group, in which some or all of the hydrogen atoms may be substituted by halogen, hydroxy, C₁-C₆ saturated hydrocarbyloxy, C₂-C₆ saturated hydrocarbylcarbonyl or C₂-C₆ saturated hydrocarbylcarbonyloxy moiety. When p and/or r is 2 or more, groups R¹³ may be the same or different.

The C₁-C₂₀ hydrocarbyl group, and hydrocarbyl moiety in the C₁-C₂₀ hydrocarbyloxy group, C₂-C₂₀ hydrocarbylcarbonyl group, C₂-C₂₀ hydrocarbyloxycarbonyl group, C₂-C₂₀ hydrocarbylcarbonyloxy group or C₁-C₂₀ hydrocarbylsulfonyloxy group, represented by R¹³, may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, adamantyl; C₂-C₂₀ alkenyl groups such as vinyl, propenyl, butenyl and hexenyl; C₃-C₂₀ cyclic unsaturated aliphatic hydrocarbyl groups such as cyclohexenyl and norbornenyl; C₂-C₂₀ alkynyl groups such as ethynyl, propynyl and butynyl; C₆-C₂₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl; C₇-C₂₀ aralkyl groups such as benzyl and phenethyl; and combinations thereof.

The C₁-C₆ saturated hydrocarbyl groups represented by R^{13A}, R^{13B} and R^{13C} may be straight, branched or cyclic. Examples thereof include C₁-C₆ alkyl groups such as methyl, ethyl, n-propyl, isopropyl, n-butyl, isobutyl, sec-butyl, tert-butyl, n-pentyl and n-hexyl; and C₃-C₆ cyclic saturated hydrocarbyl groups such as cyclopropyl, cyclobutyl, cyclopentyl and cyclohexyl. Examples of the saturated hydrocarbyl moiety in the C₁-C₆ saturated hydrocarbyloxy group represented by R^{13C} are as exemplified above for the saturated hydrocarbyl group. Examples of the saturated hydrocarbyl moiety in the C₂-C₆ saturated hydrocarbylcarbonyl group and C₂-C₆ saturated hydrocarbylcarbonyloxy group represented by R^{13C} are as exemplified above for the C₁-C₆ saturated hydrocarbyl group, but of 1 to 5 carbon atoms.

The aliphatic hydrocarbyl group represented by R^{13D} may be saturated or unsaturated and straight, branched or cyclic. Examples thereof include C₁-C₁₆ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, and pentadecyl; C₃-C₁₆ cyclic saturated hydrocarbyl groups such as cyclopropyl, cyclopentyl, cyclohexyl, cyclopropylmethyl, 4-methylcyclohexyl, cyclohexylmethyl, norbornyl, adamantyl; C₂-C₁₆ alkenyl groups such as vinyl, propenyl, butenyl and hexenyl; C₂-C₁₆ alkynyl groups such as ethynyl, propynyl and butynyl; C₃-C₁₆ cyclic unsaturated aliphatic hydrocarbyl groups such as cyclohexenyl and norbornenyl; and combinations thereof. Examples of the C₆-C₁₂ aryl group R^{13D} include phenyl and naphthyl. Examples of the C₇-C₁₅ aralkyl group R^{13D} include benzyl and phenethyl. Of the groups represented by R^{13D}, examples of the hydrocarbyl moiety in the C₁-C₆ saturated hydrocarbyloxy group are as exemplified above for the C₁-C₆ saturated hydrocarbyl group represented by R^{13A}, R^{13B} and R^{13C}; examples of the hydrocarbyl moiety in the C₂-C₆ saturated hydrocarbylcarbonyl group or C₂-C₆ saturated hydrocarbylcarbonyloxy group are as exemplified above for the C₁-C₆ saturated hydrocarbyl group, but of 1 to 5 carbon atoms.

In formula (2-3), Rf¹ to Rf⁴ are each independently hydrogen, fluorine or trifluoromethyl, at least one of Rf¹ to Rf⁴ being fluorine or trifluoromethyl. Also Rf¹ and Rf², taken together, may form a carbonyl group. The total number of fluorine atoms in Rf¹ to Rf⁴ is preferably at least 2, more preferably at least 3.

Examples of the anion having formula (2-3) are shown below, but not limited thereto.

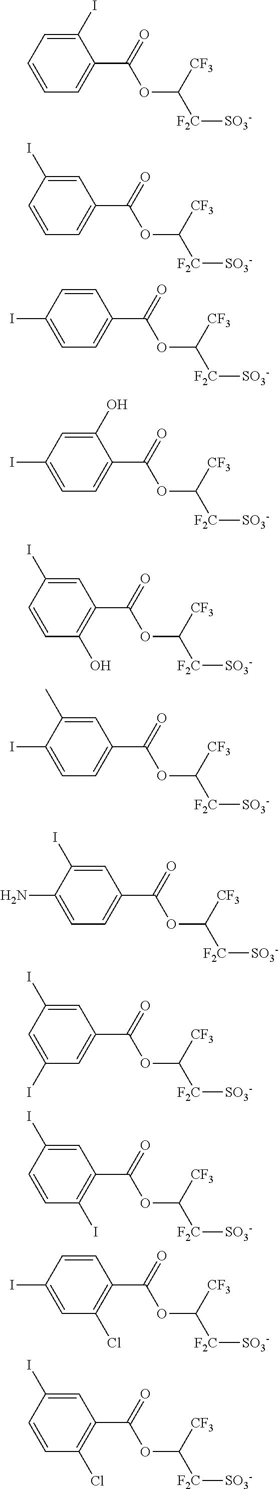

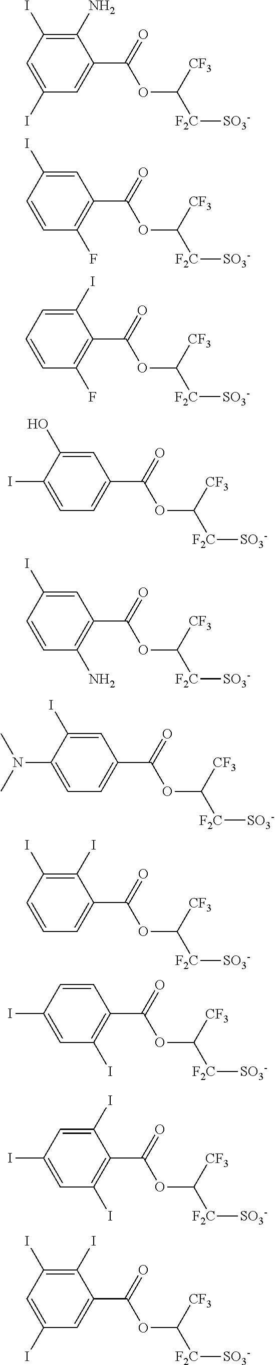

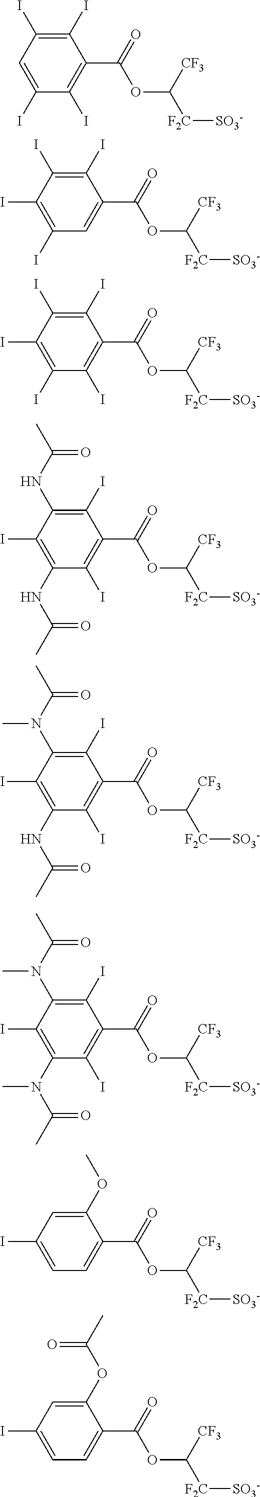

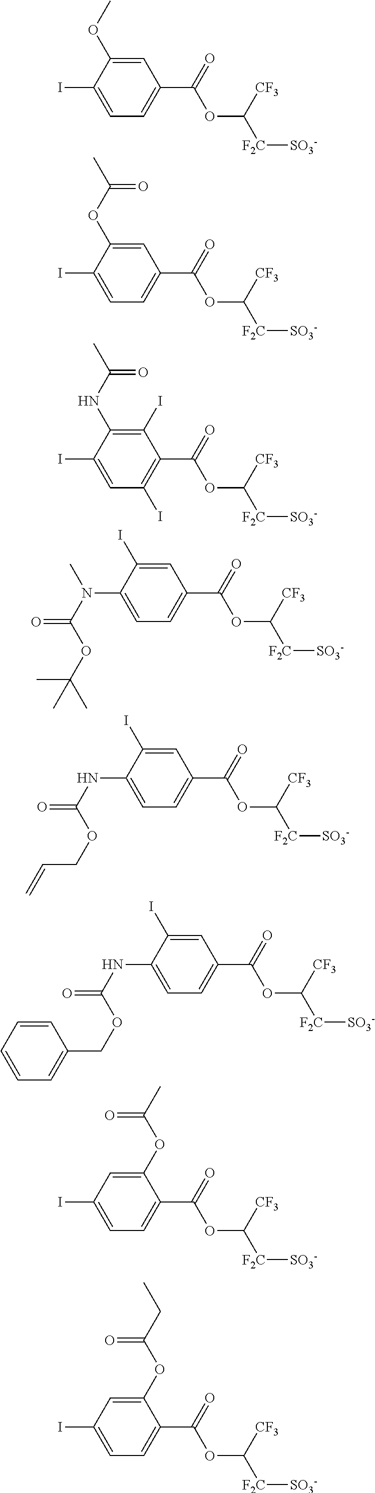

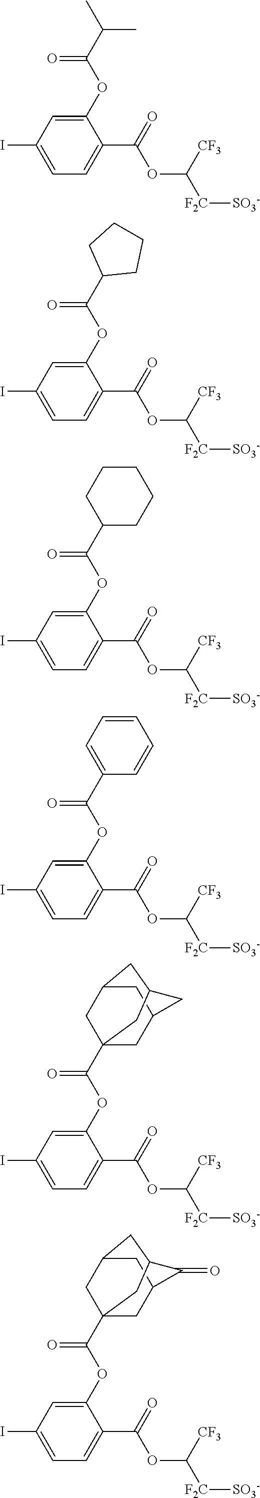

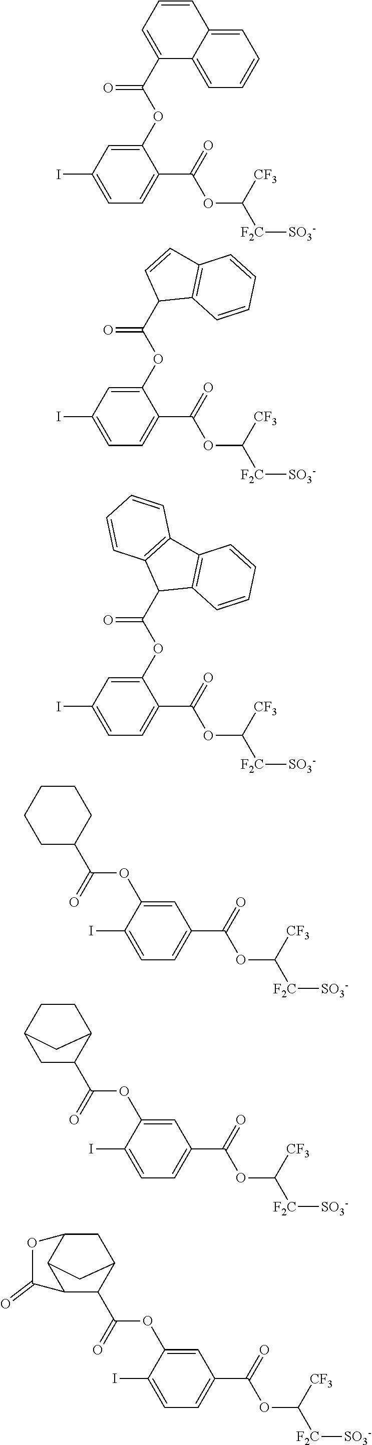

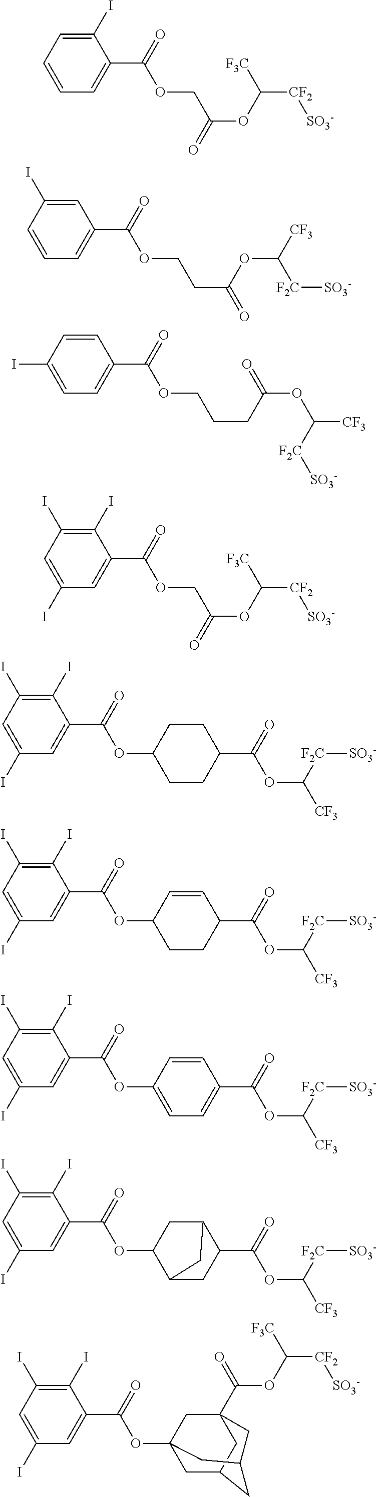

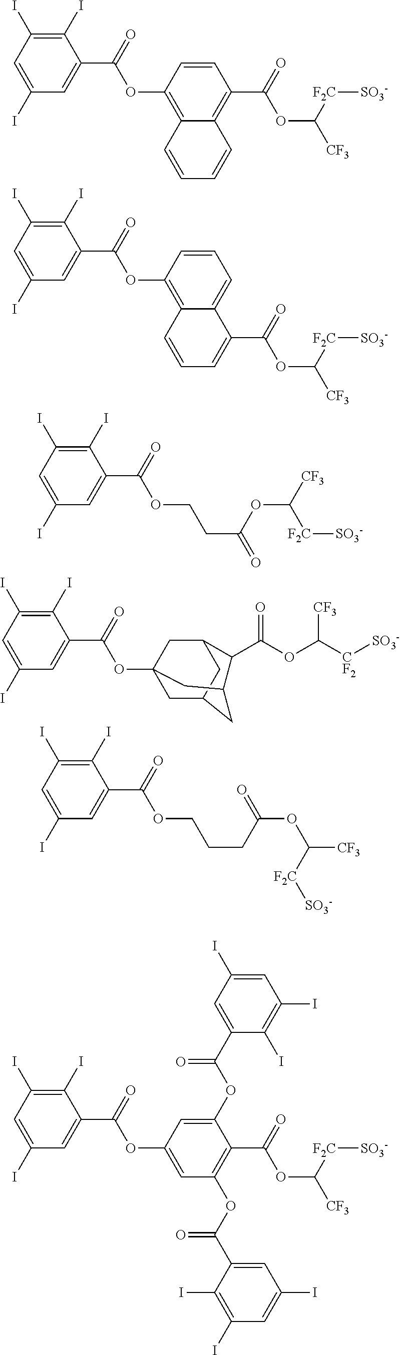

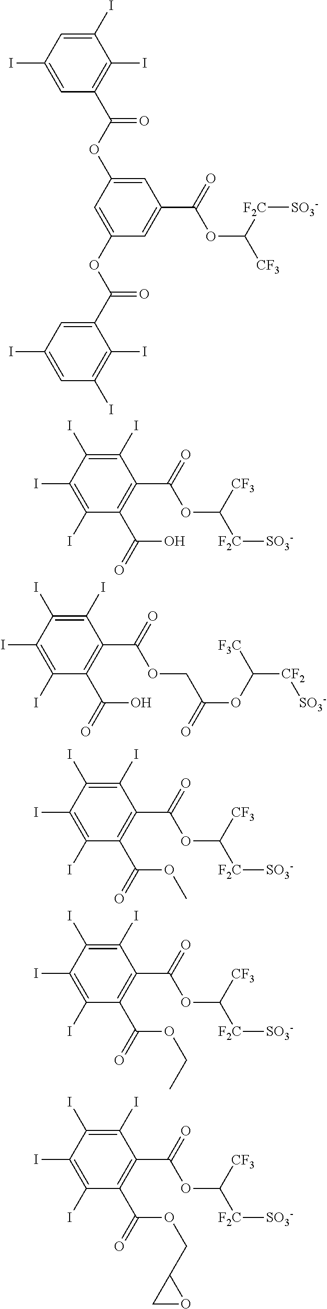

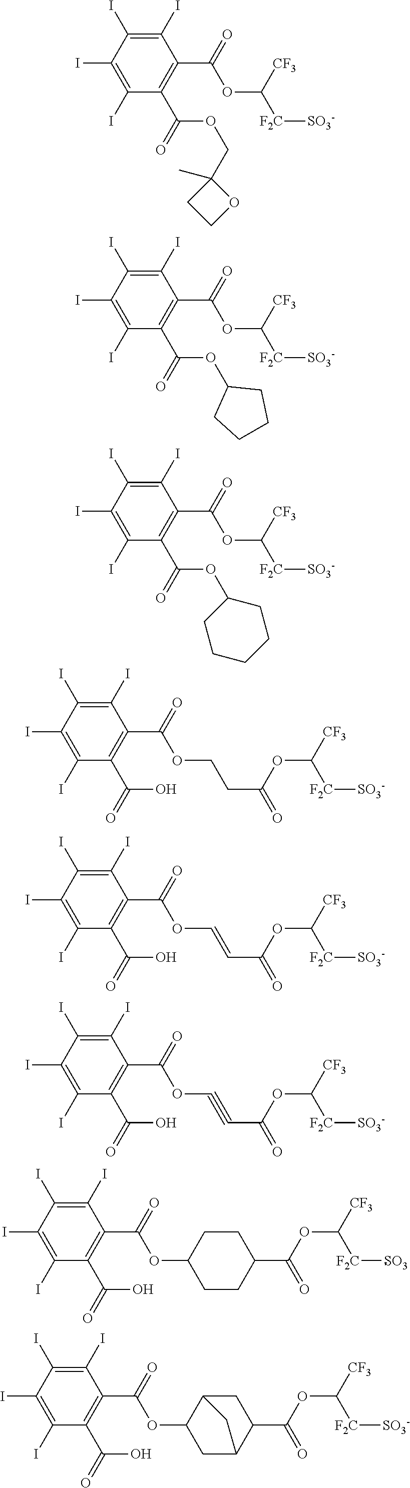

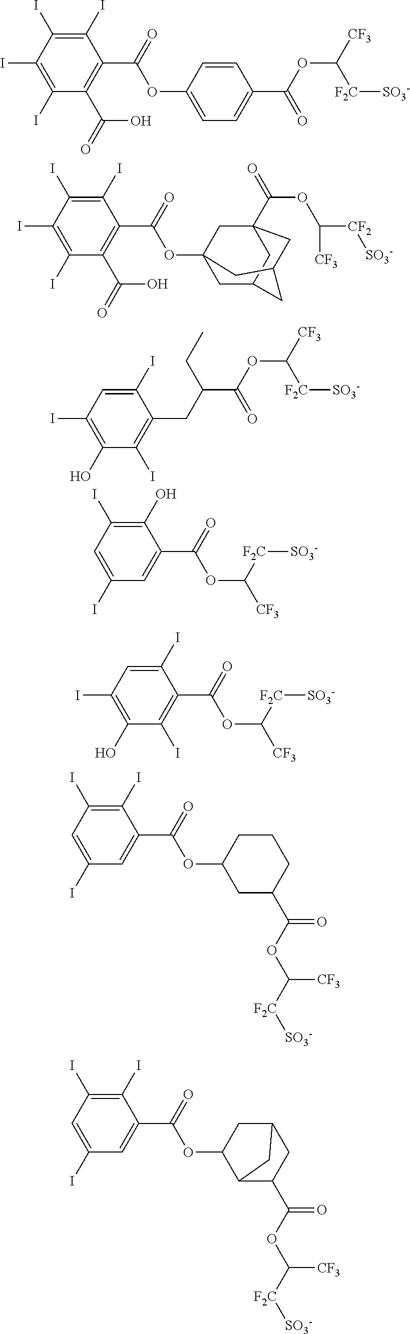

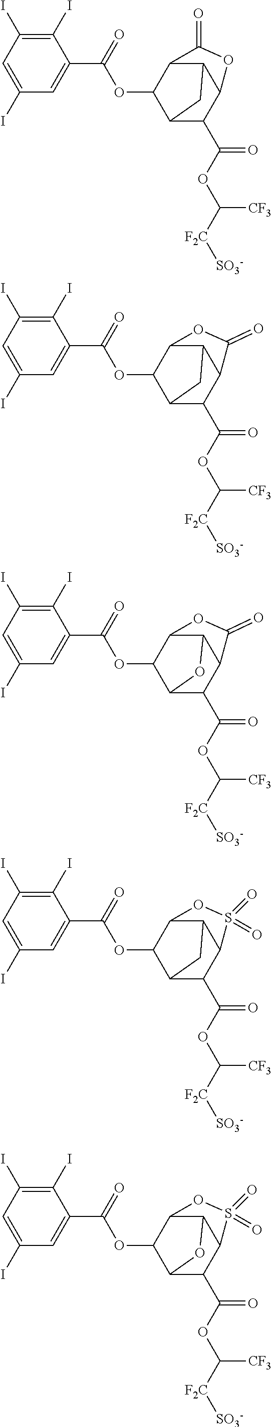

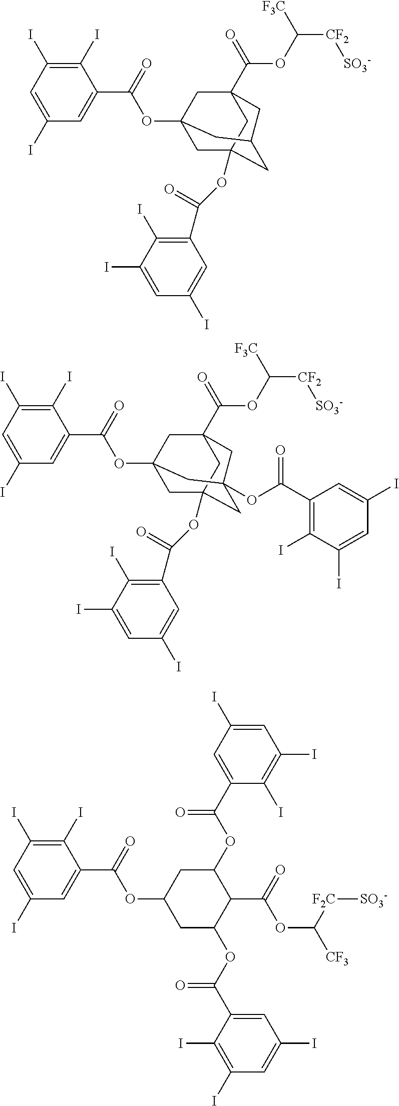

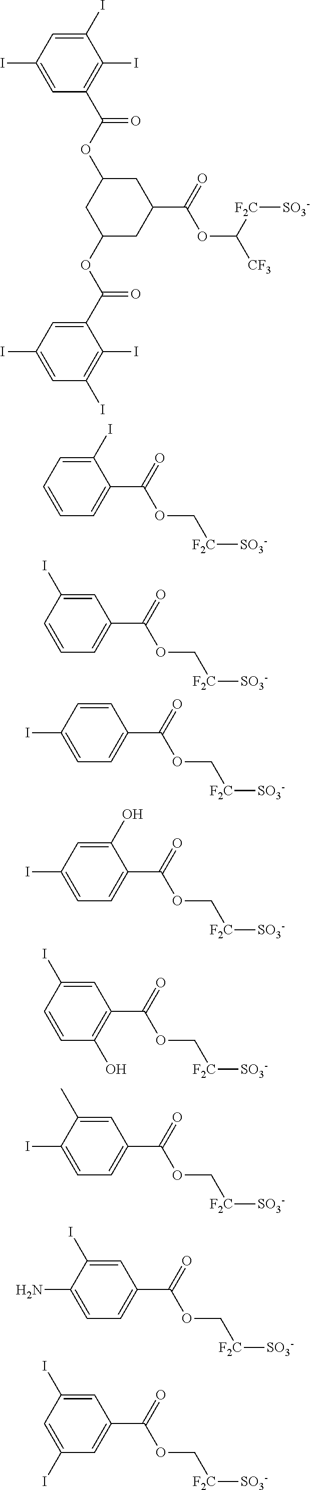

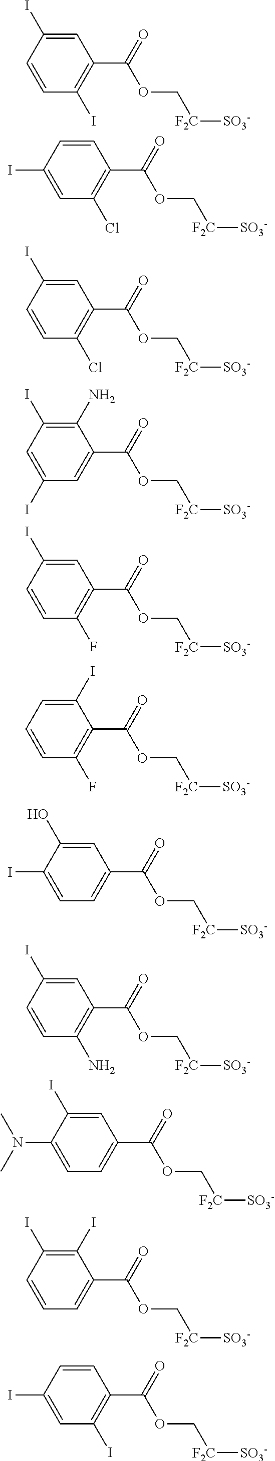

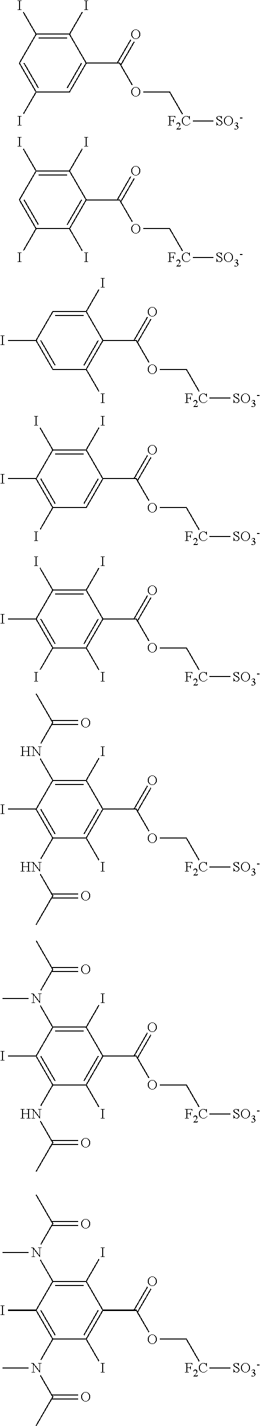

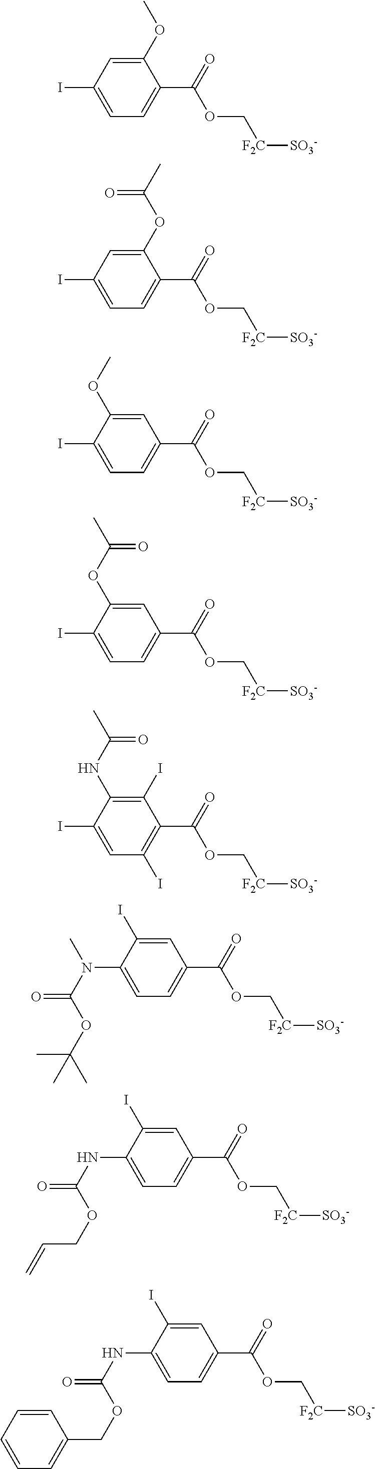

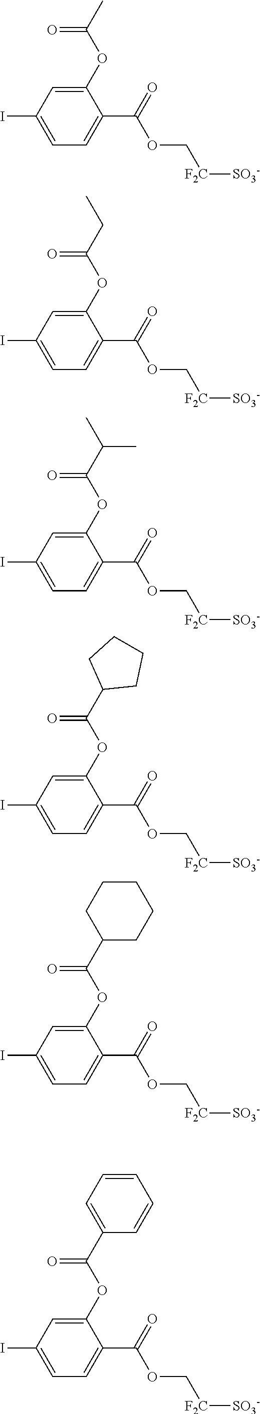

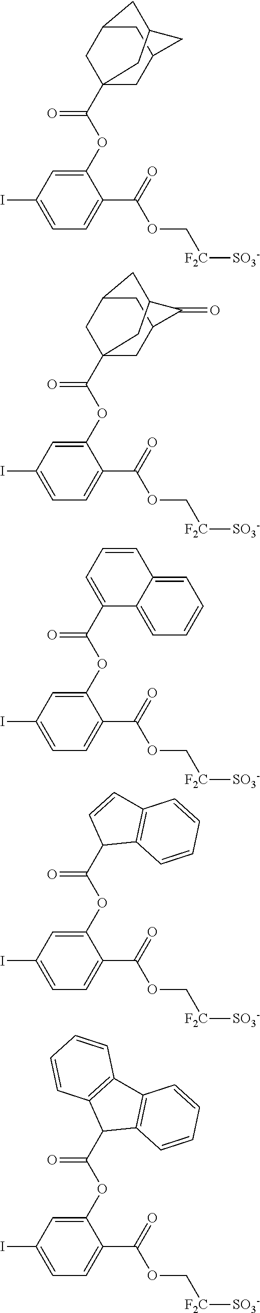

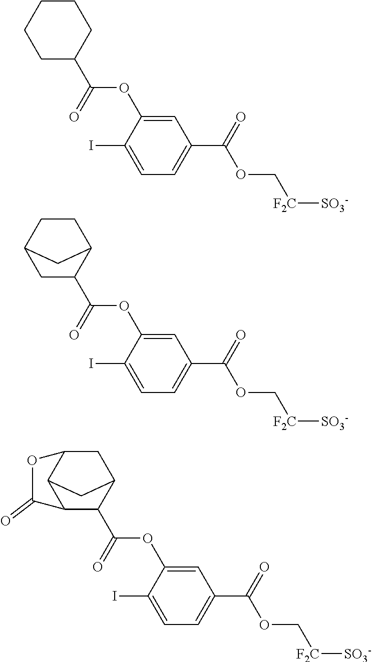

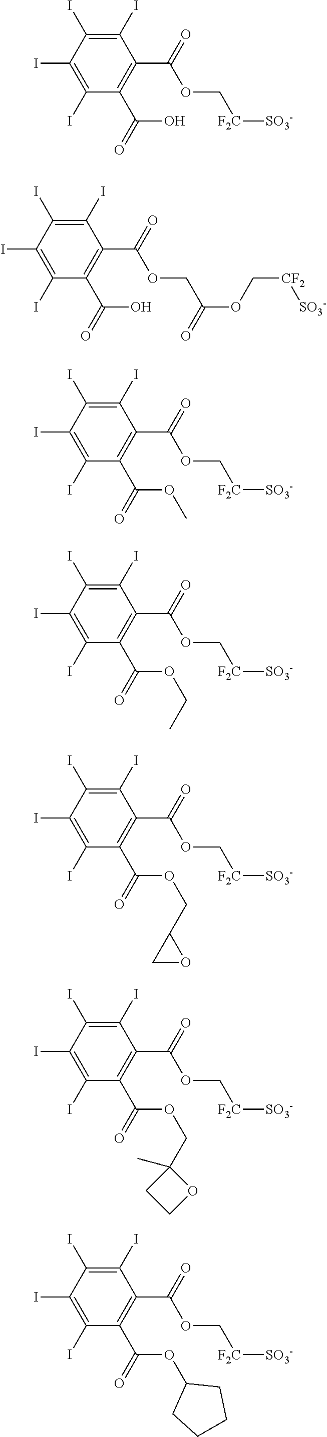

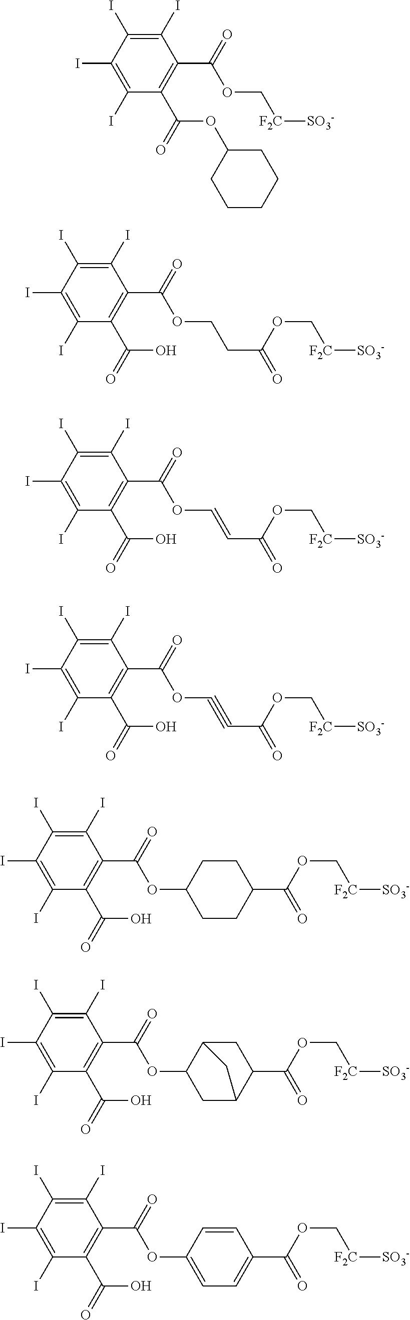

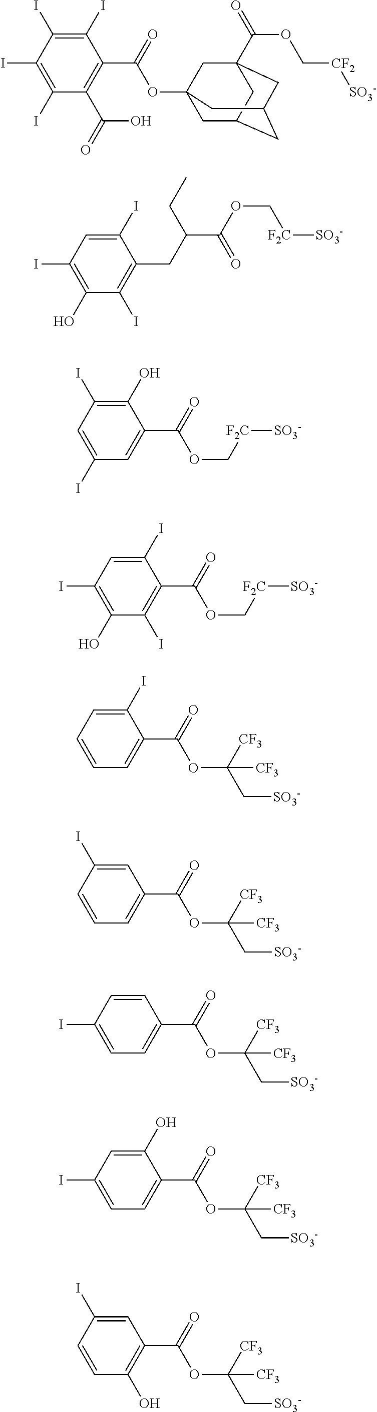

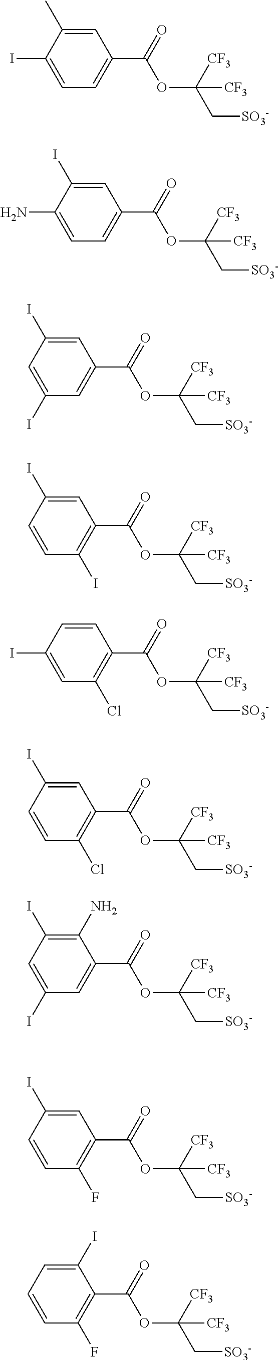

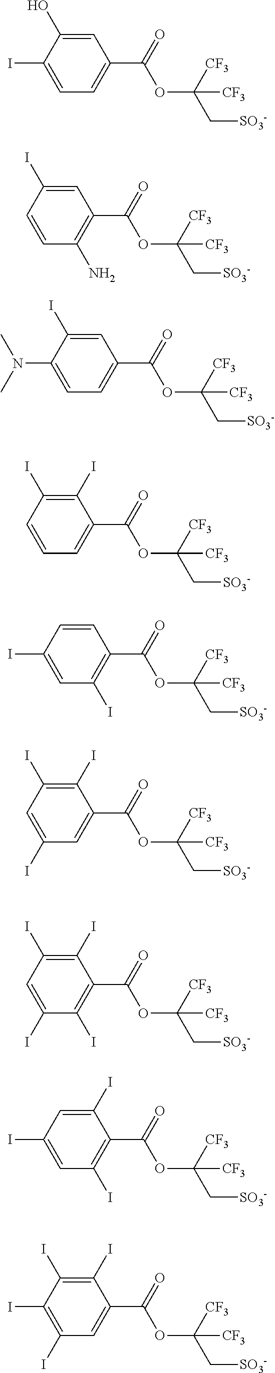

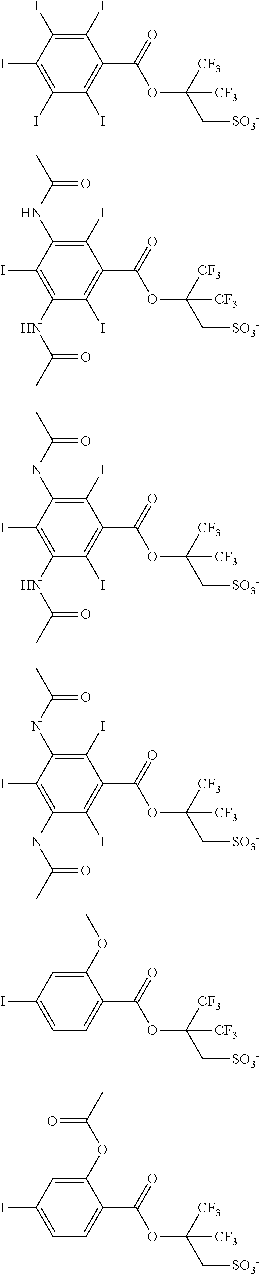

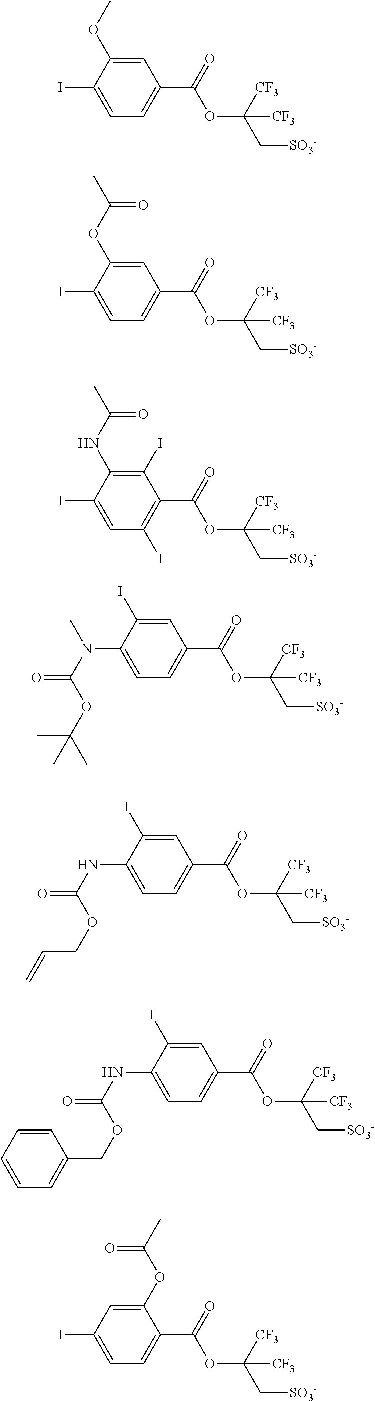

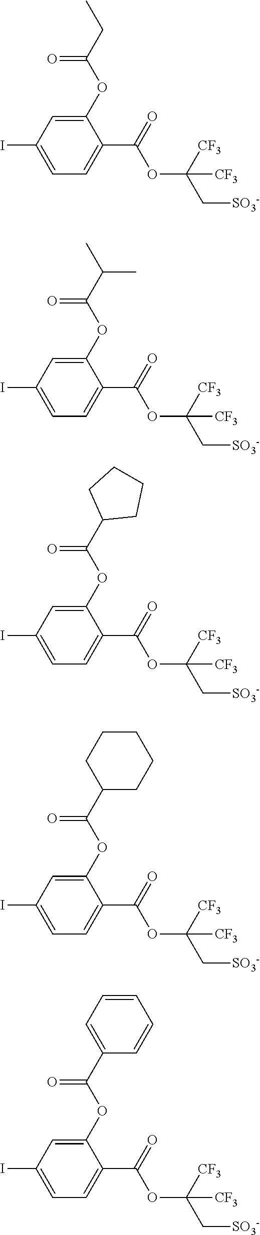

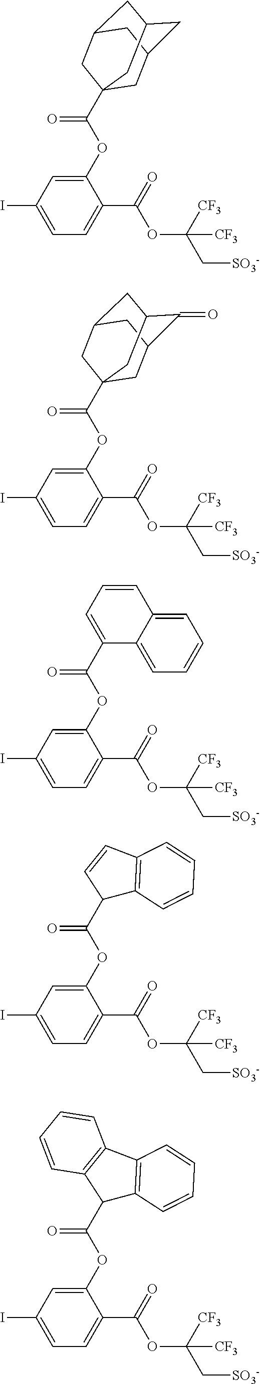

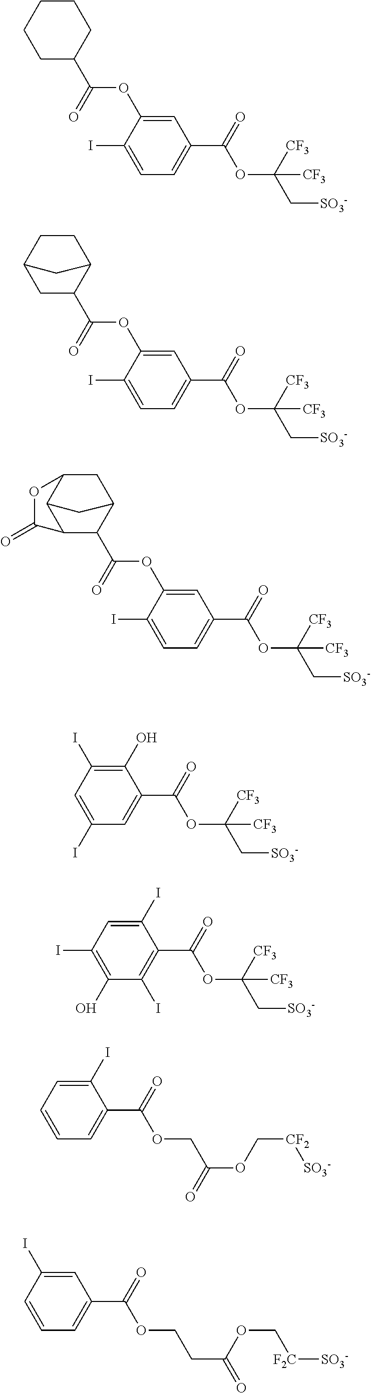

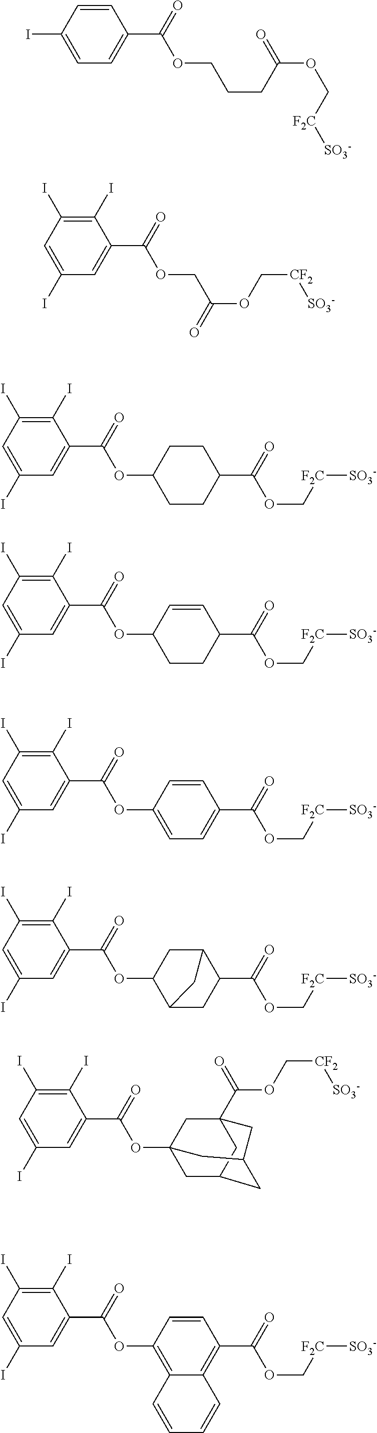

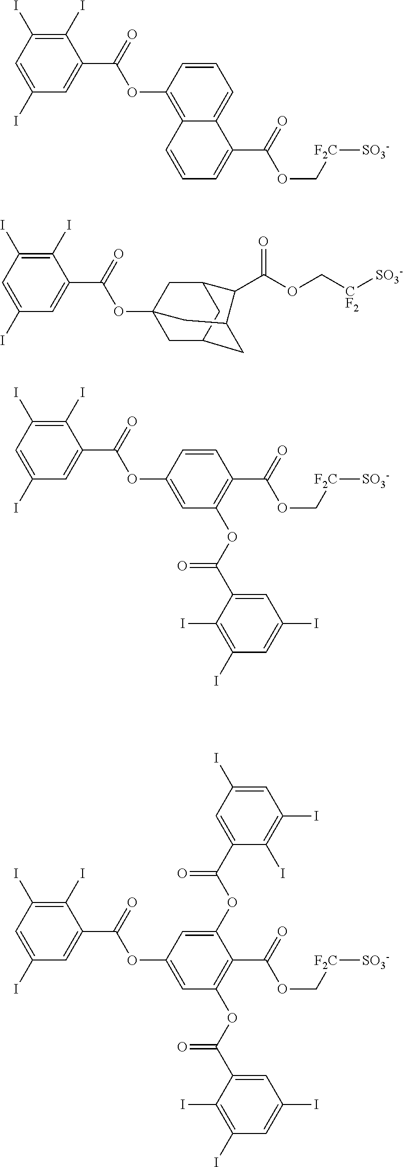

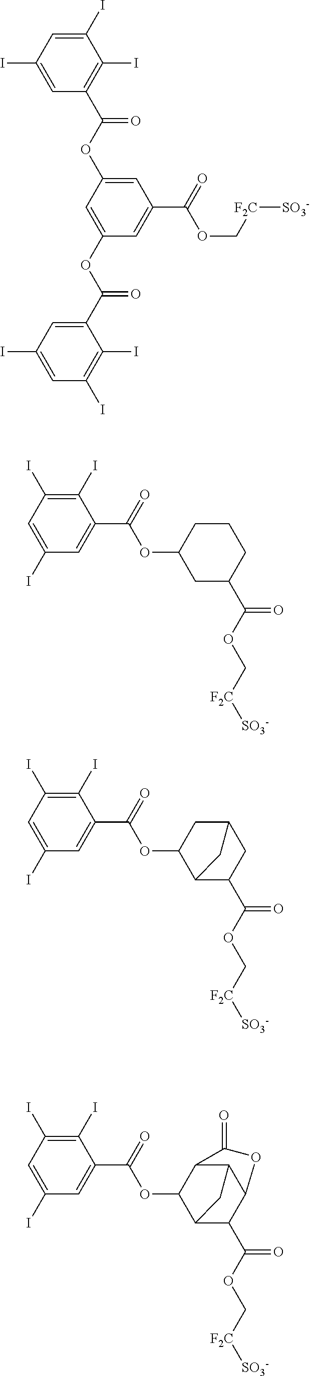

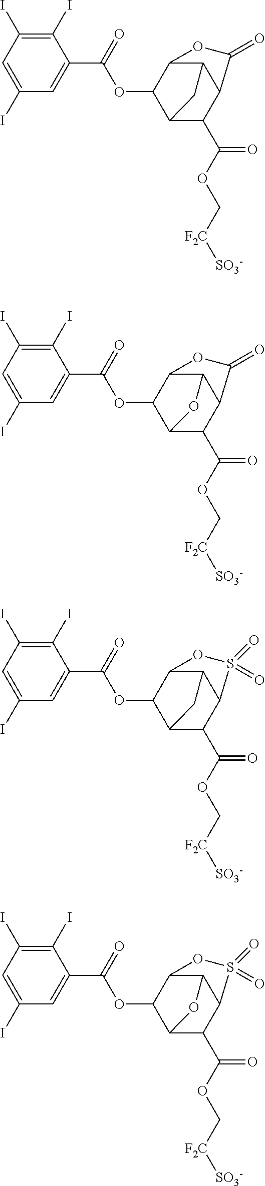

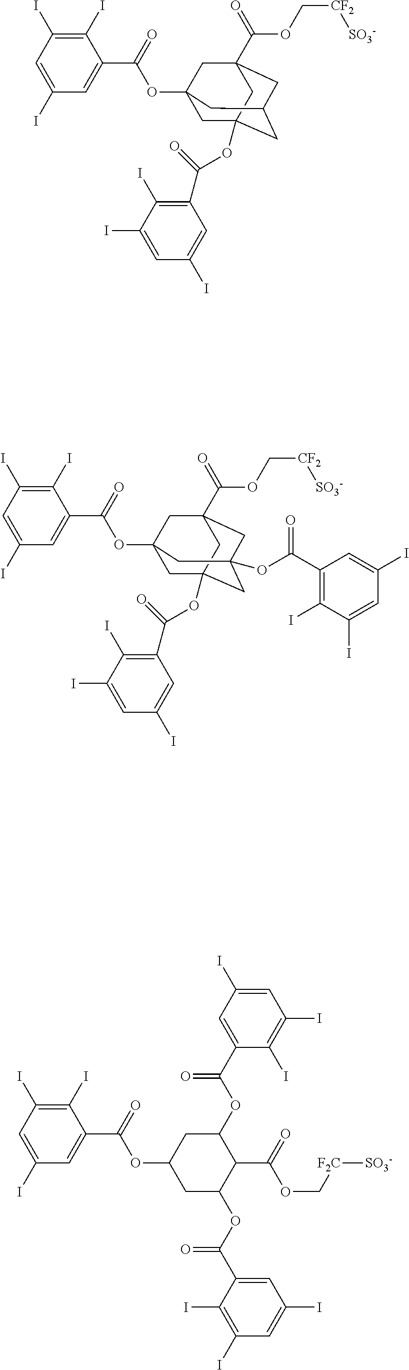

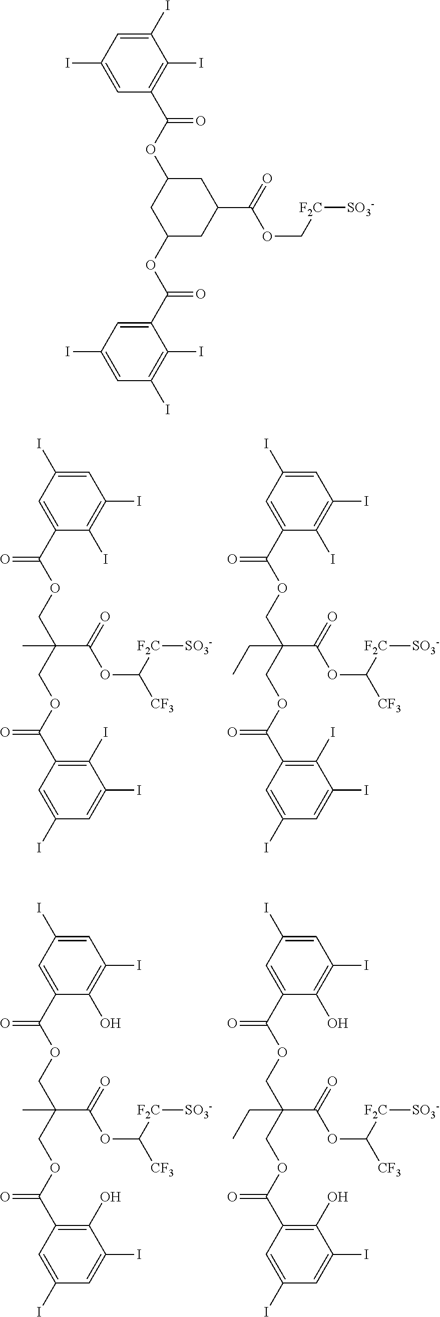

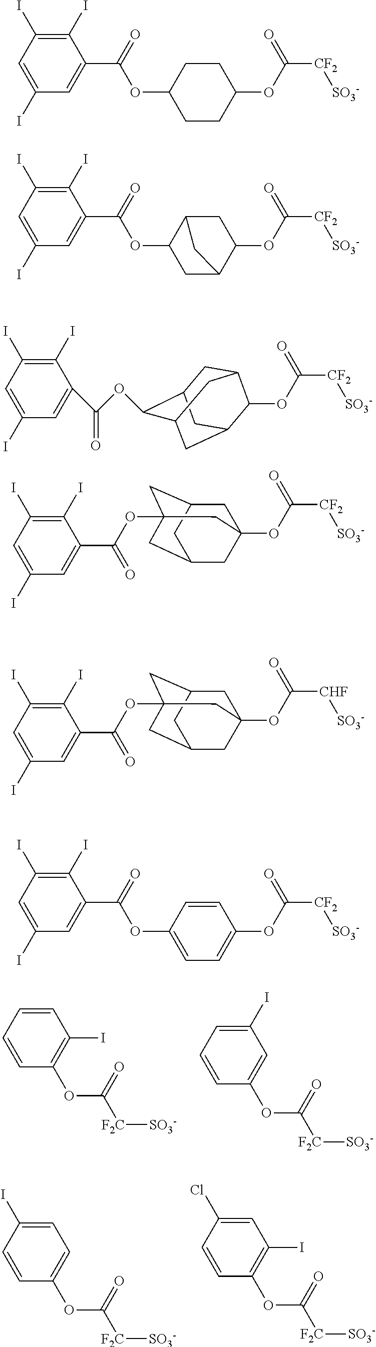

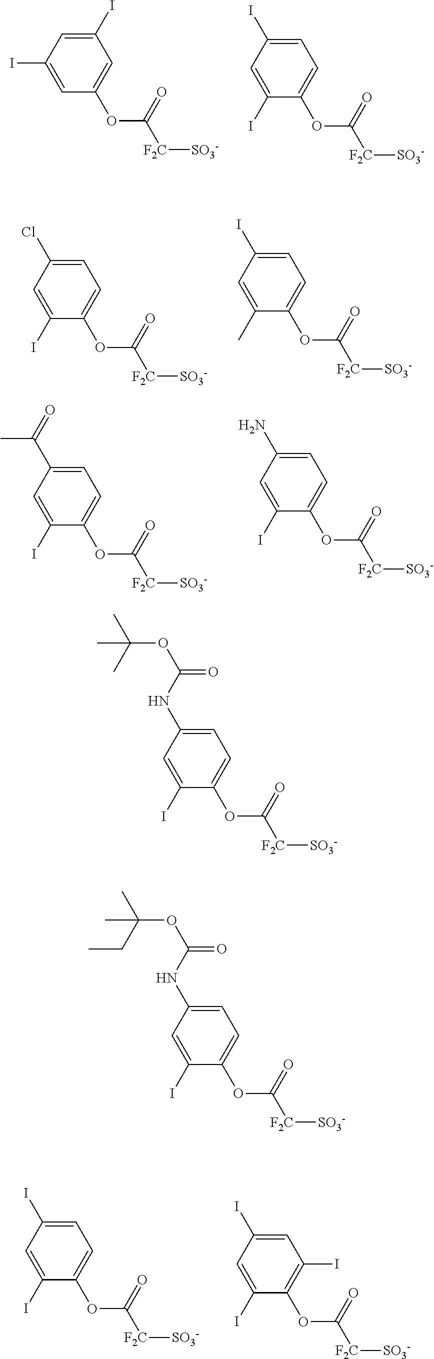

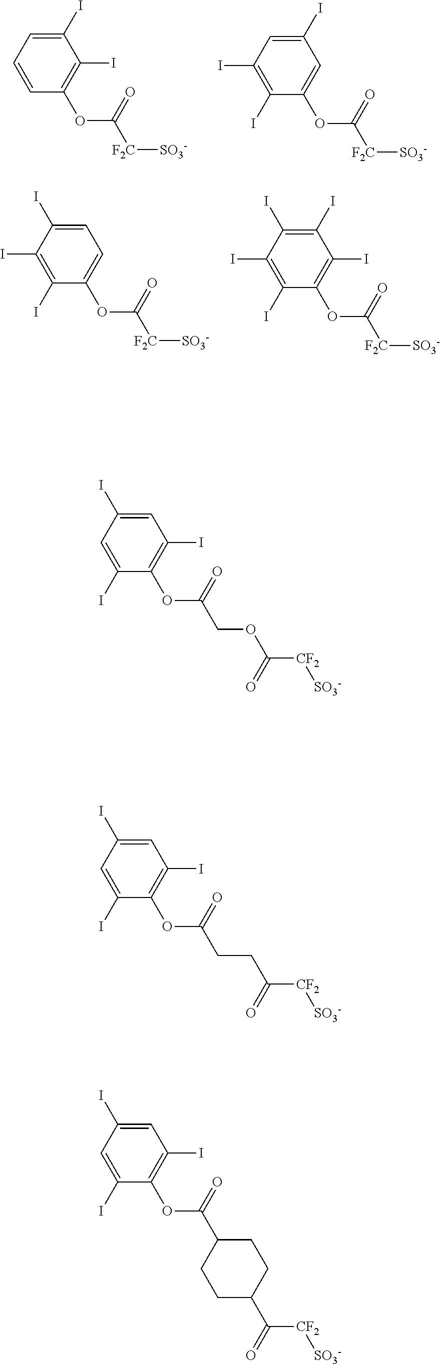

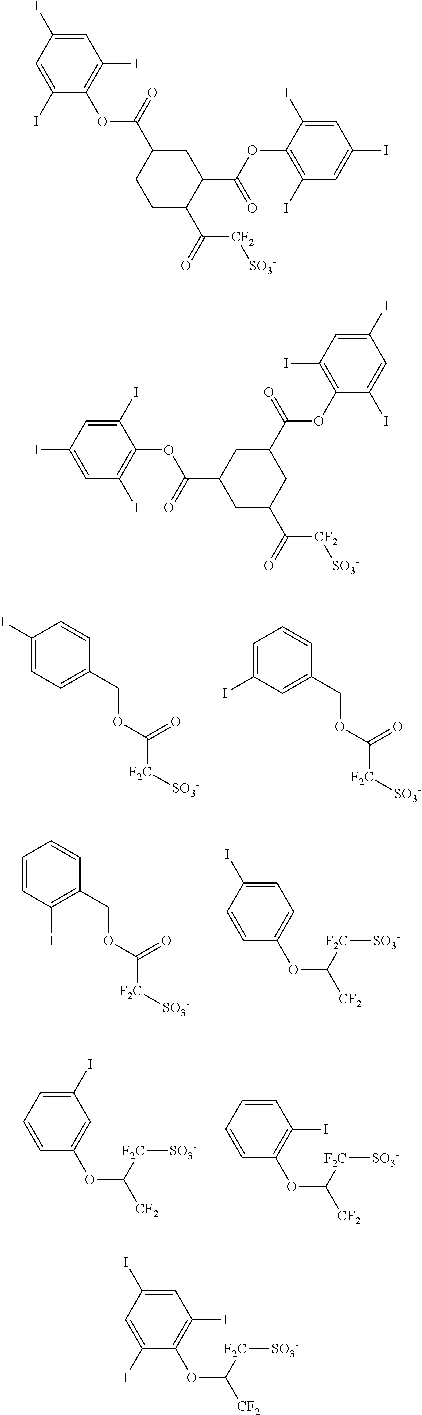

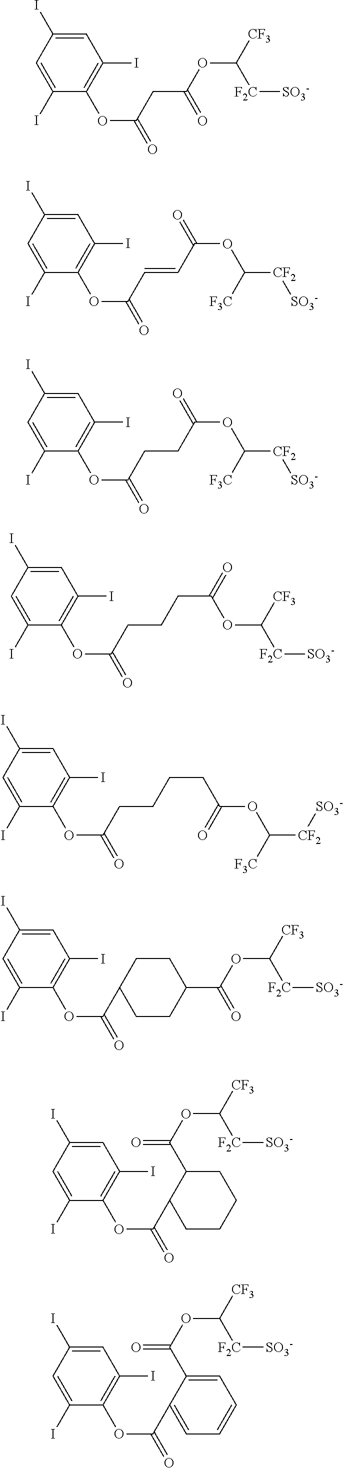

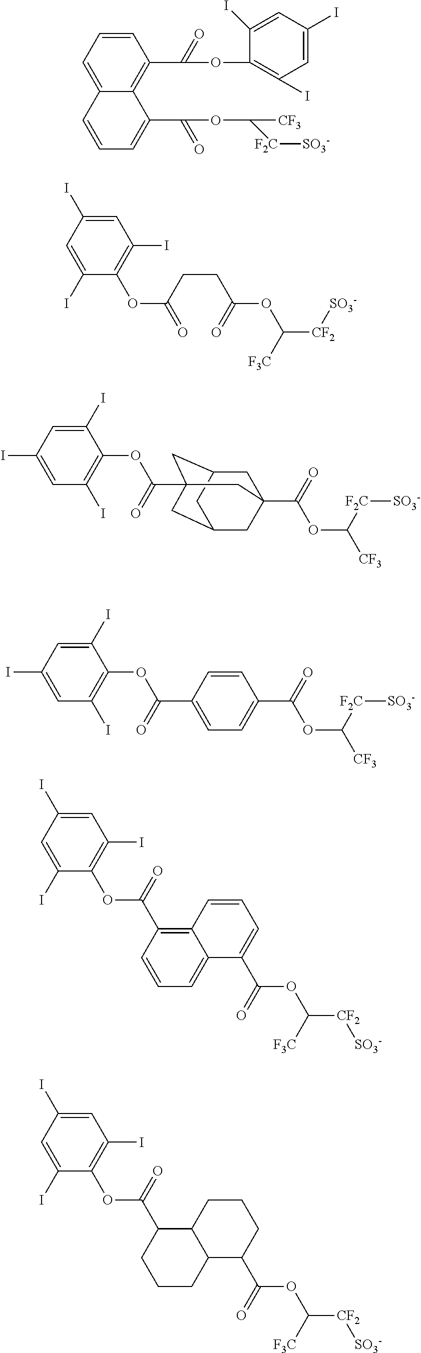

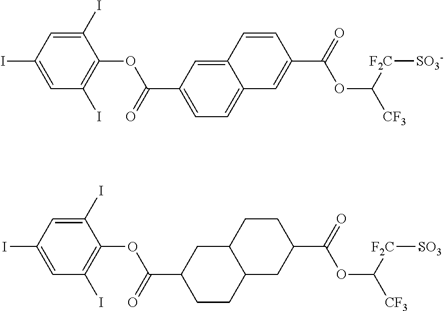

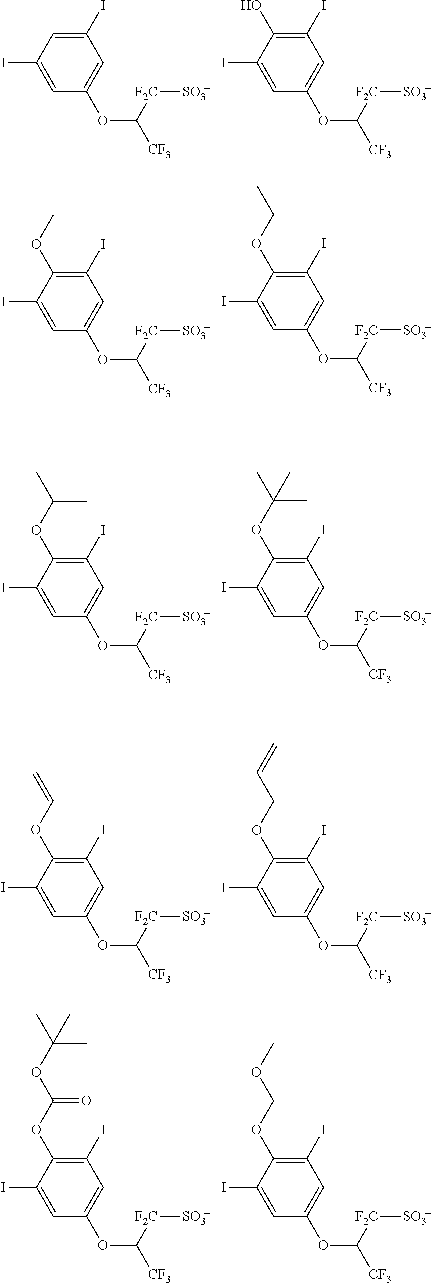

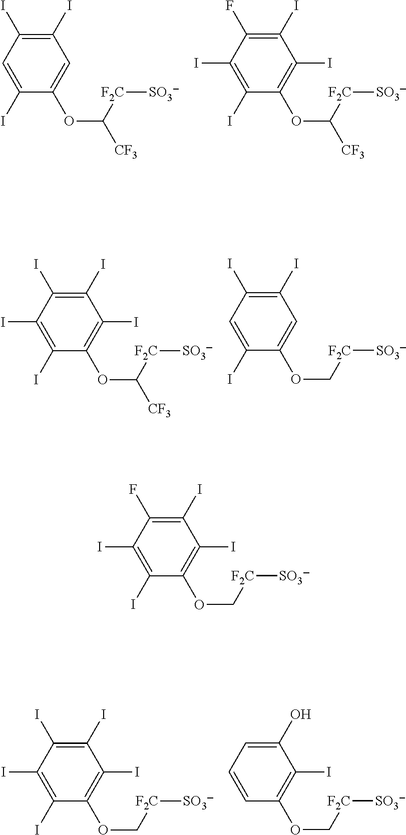

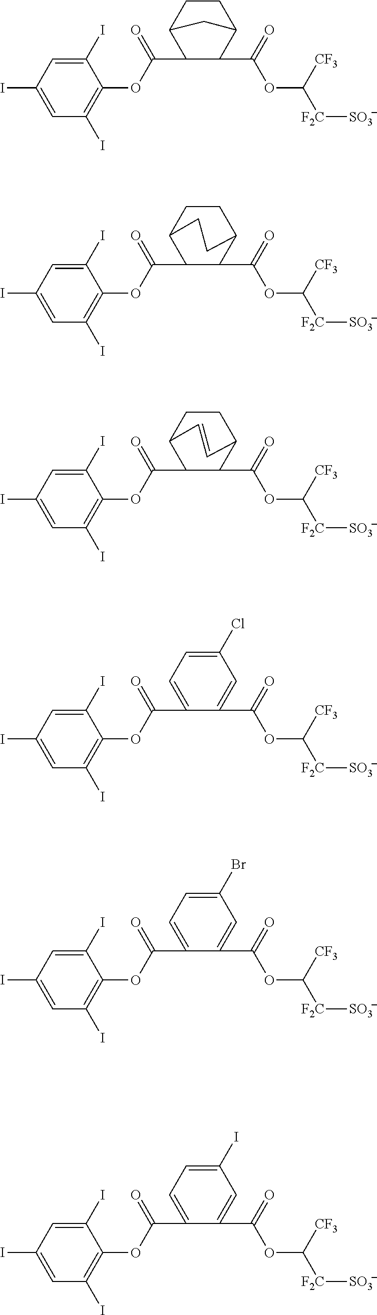

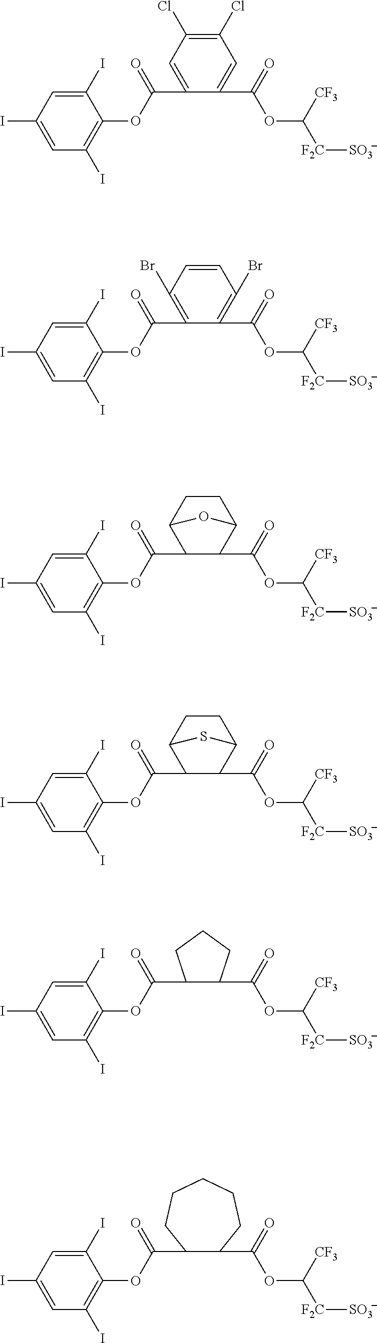

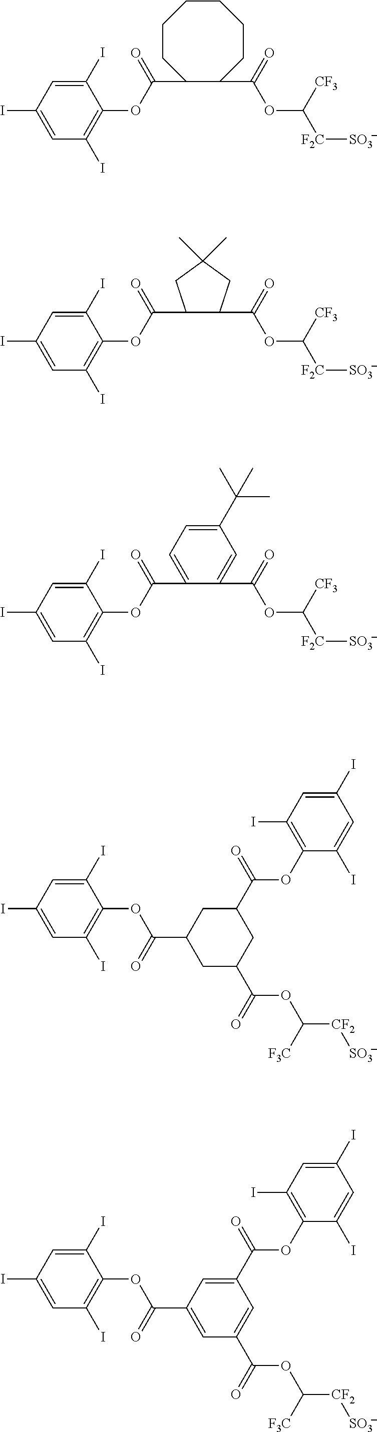

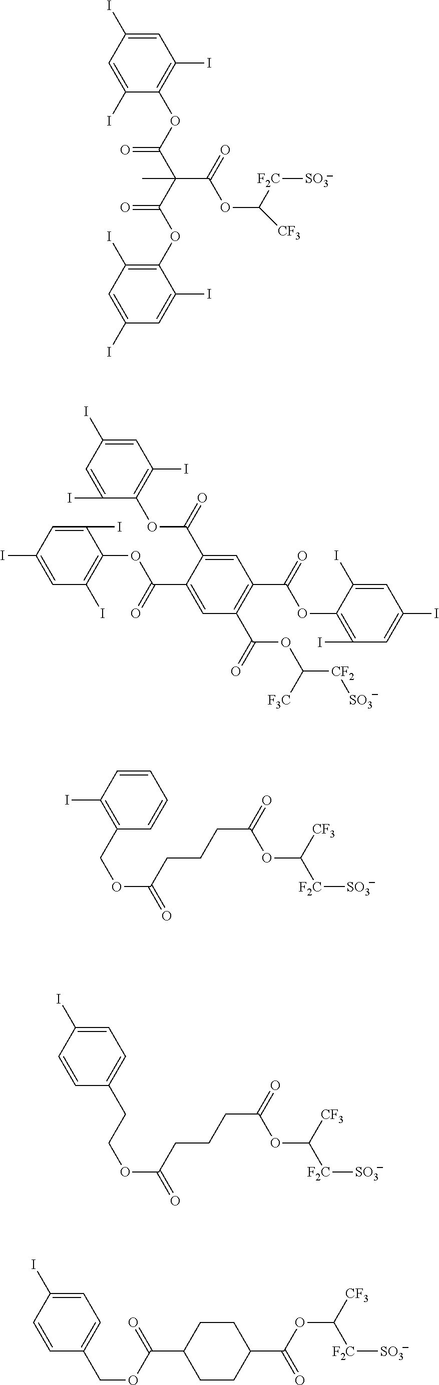

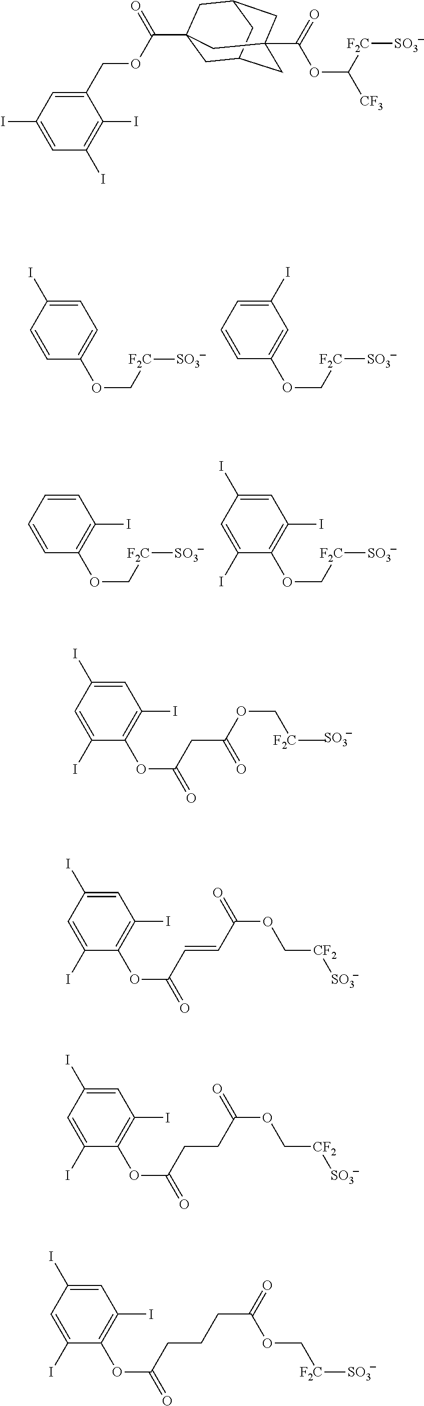

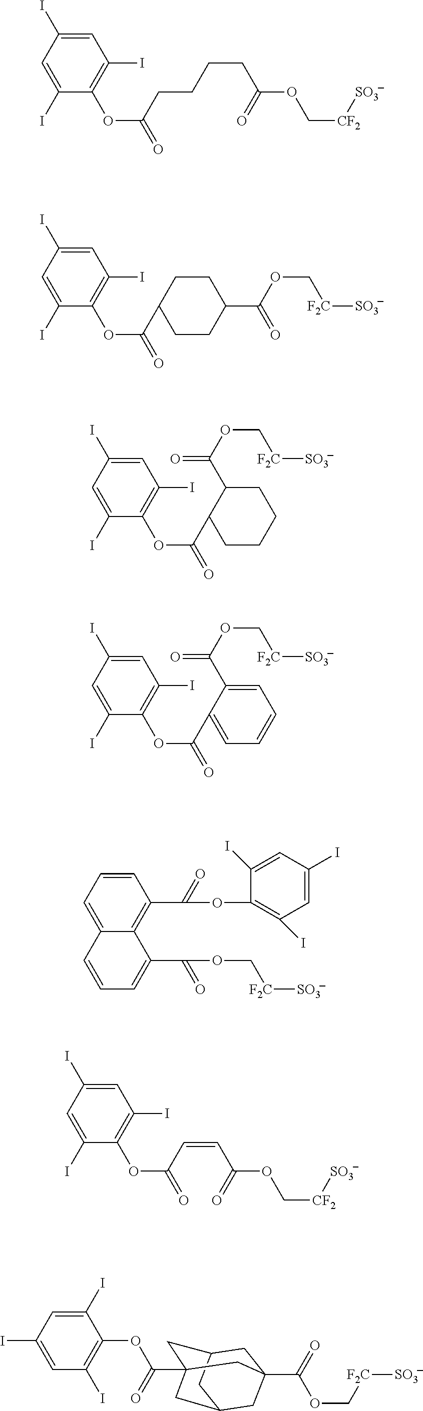

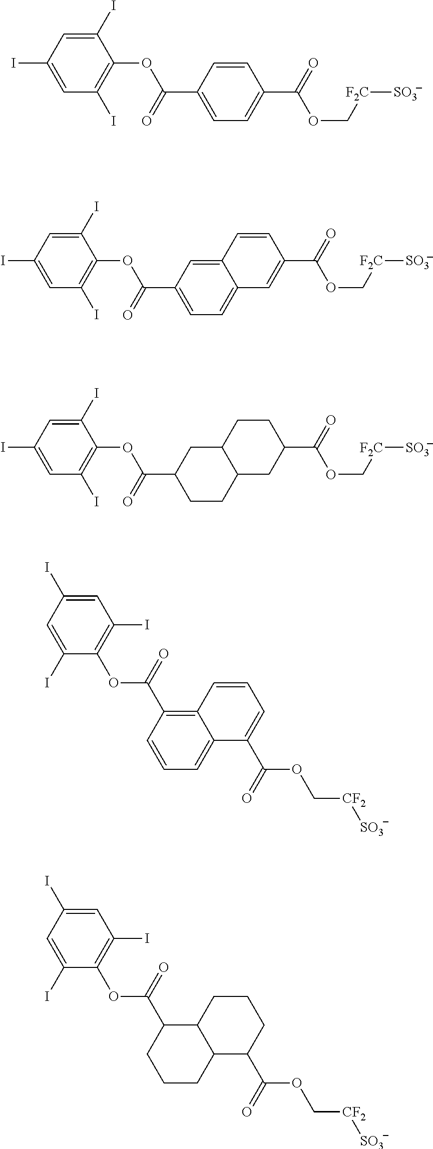

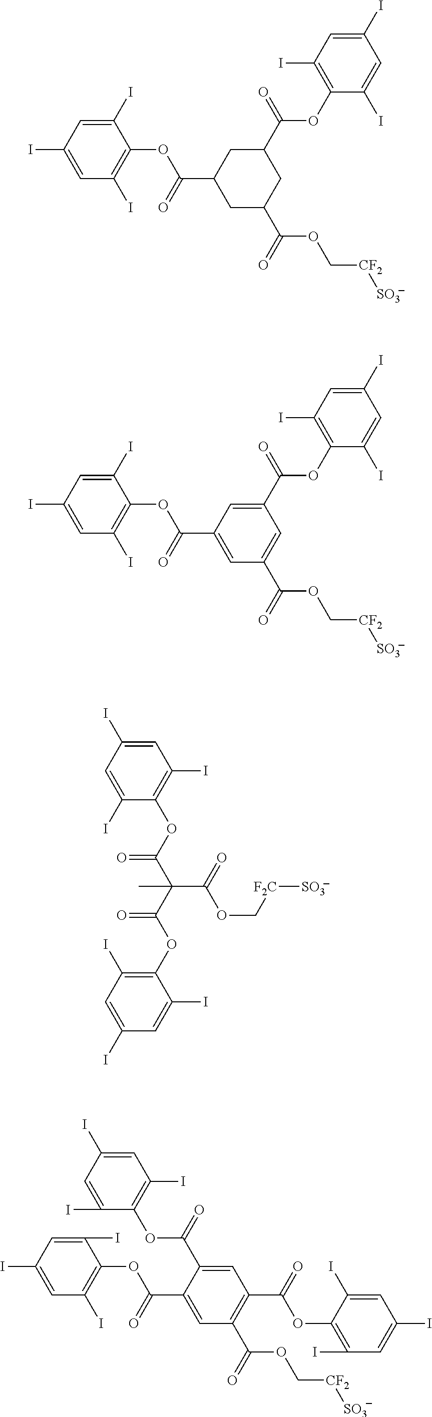

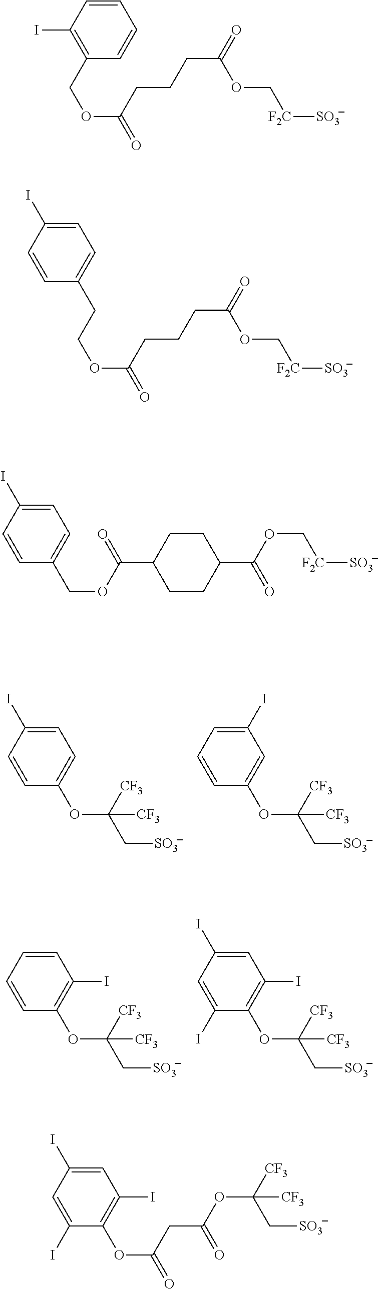

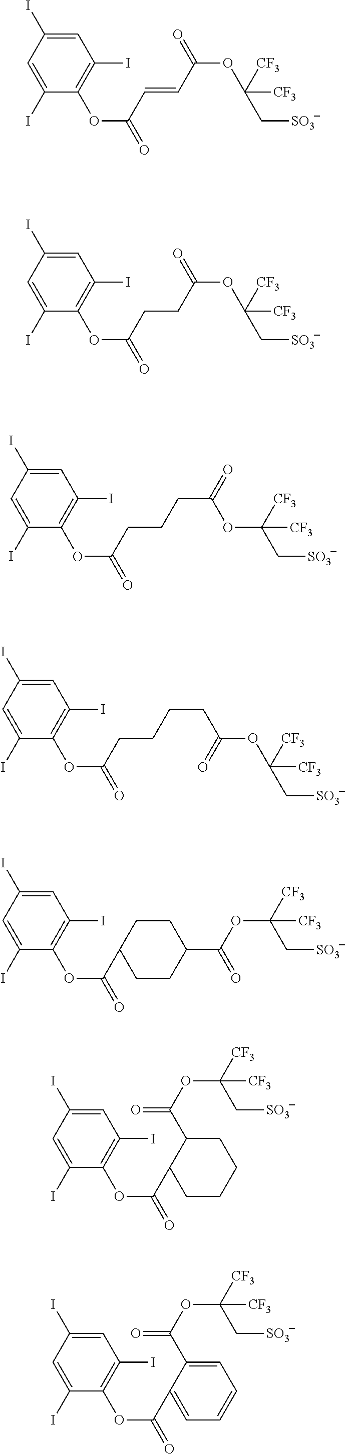

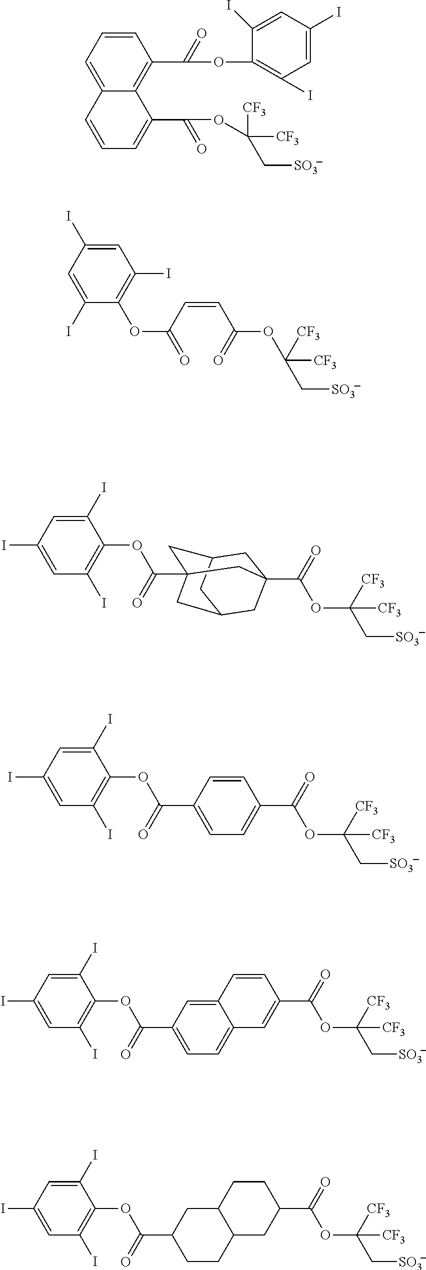

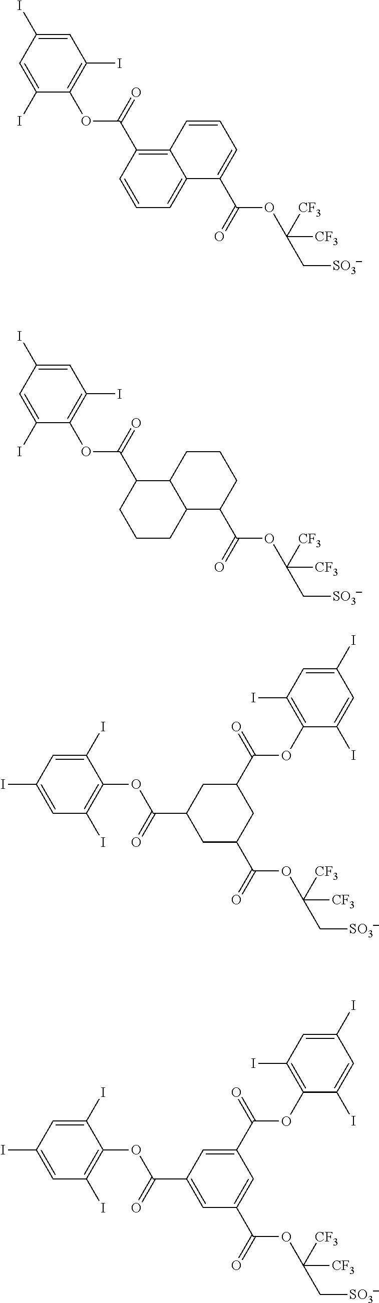

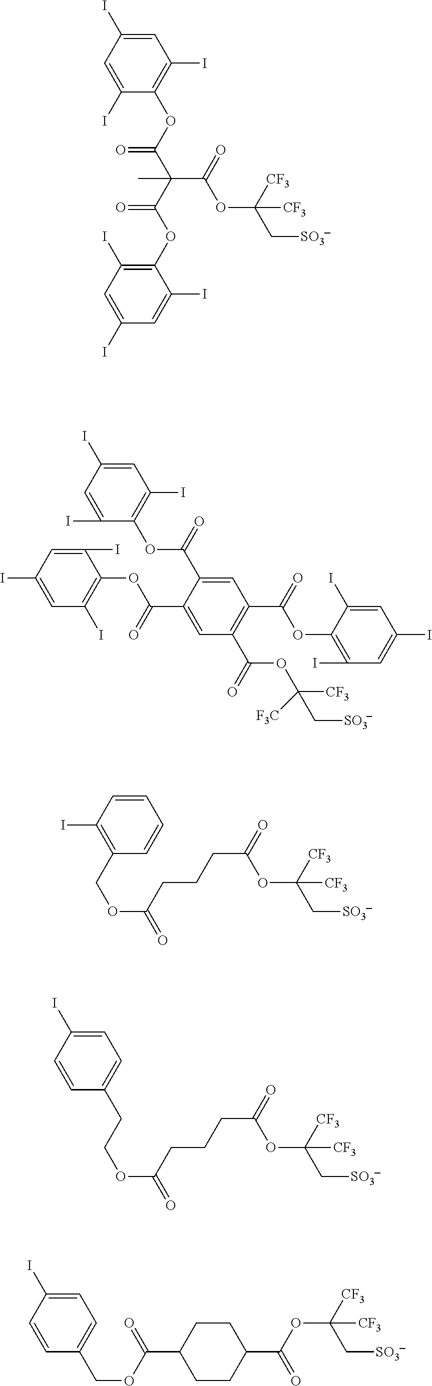

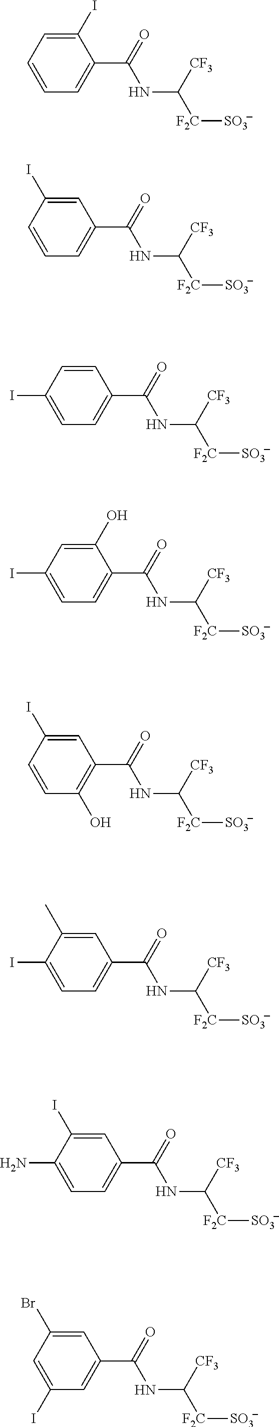

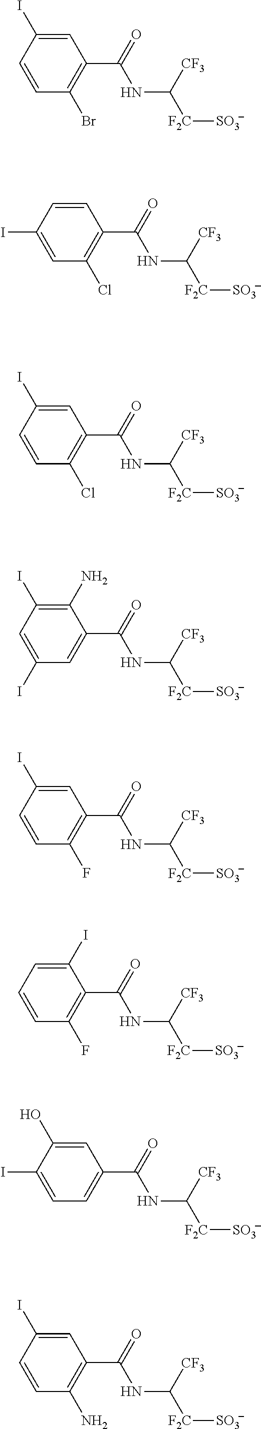

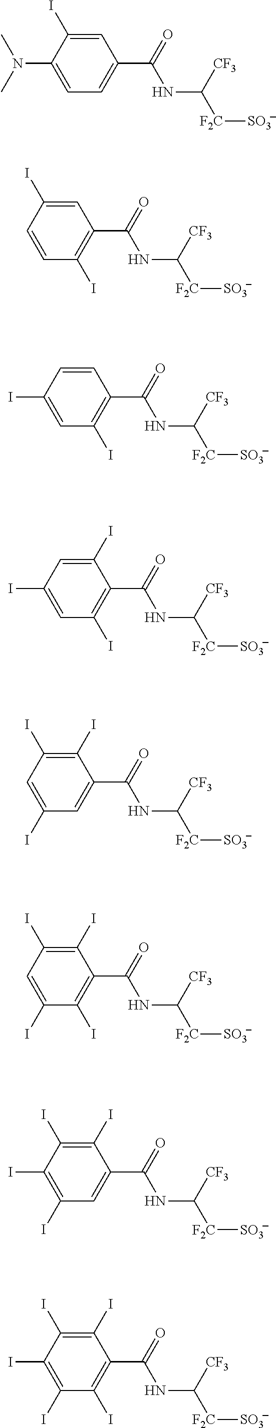

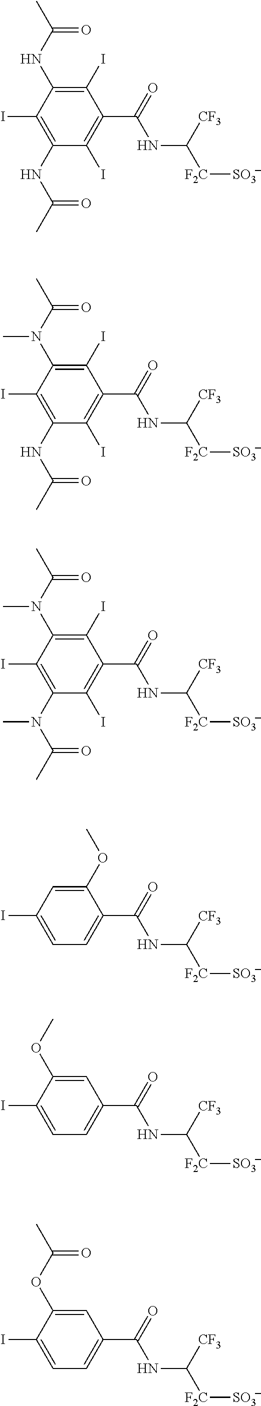

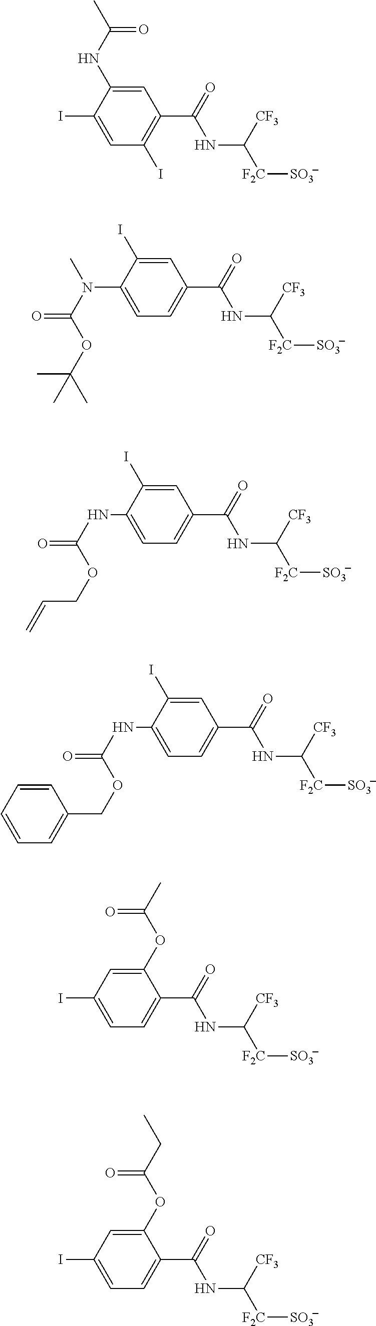

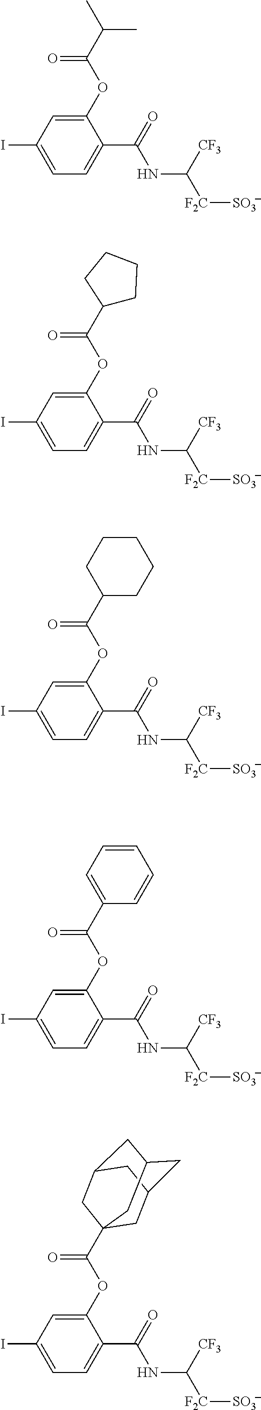

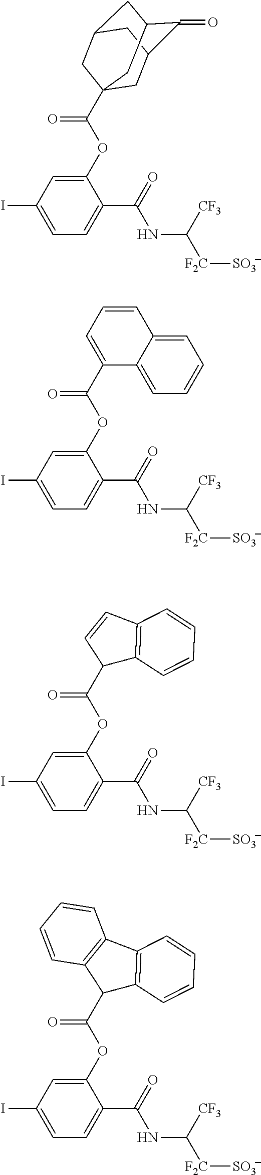

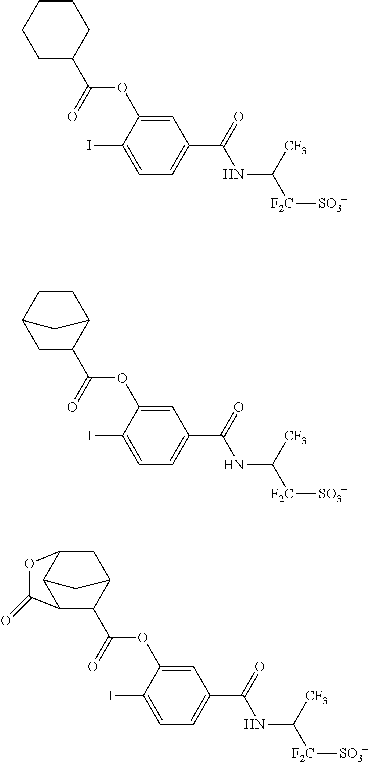

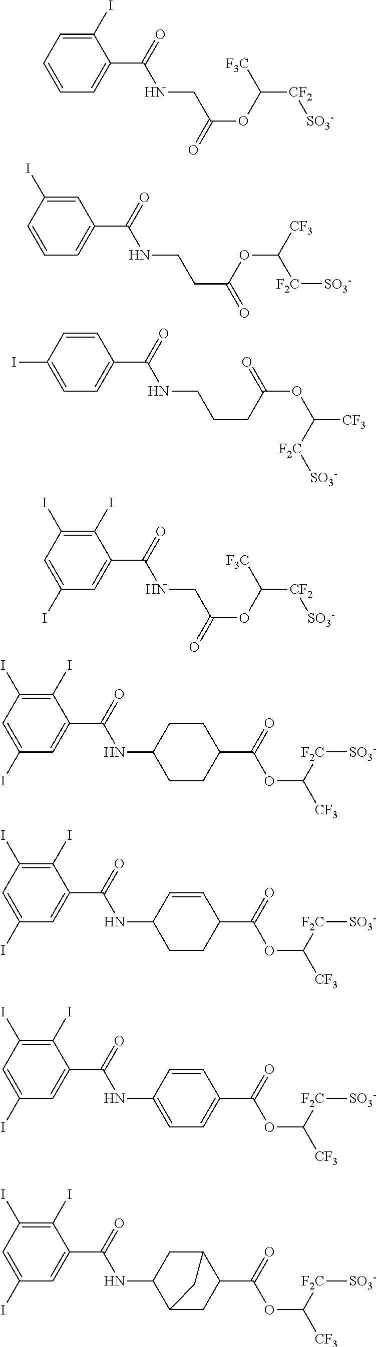

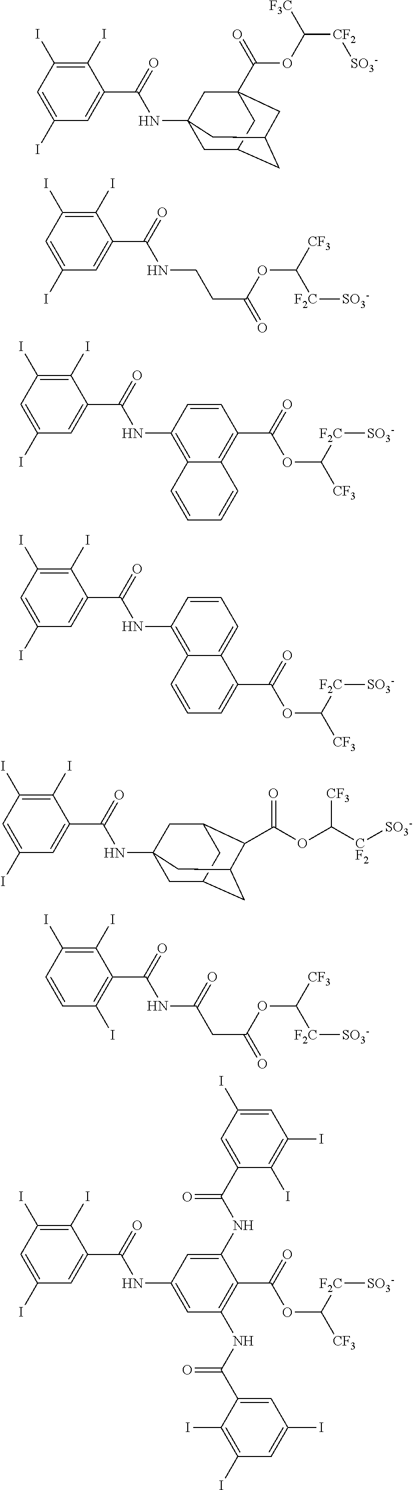

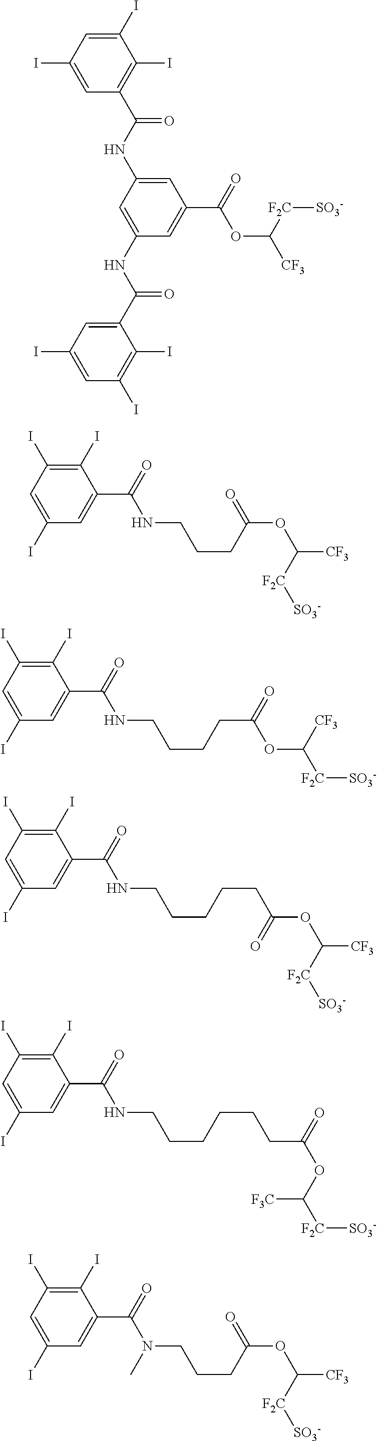

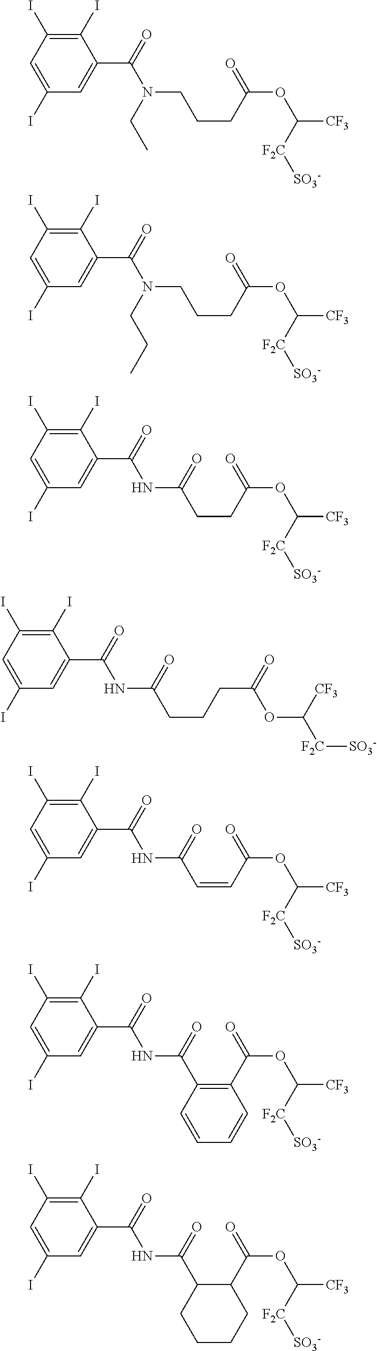

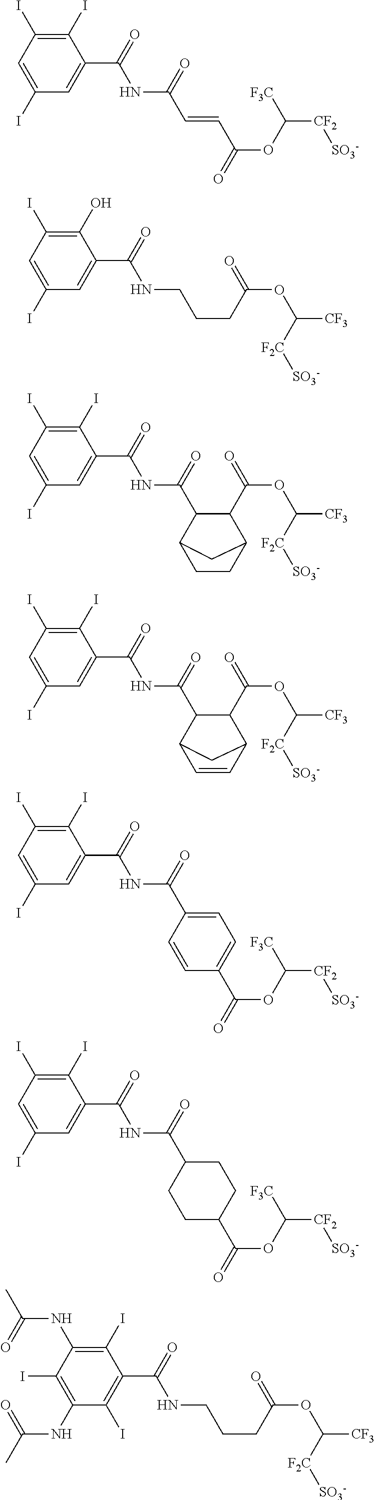

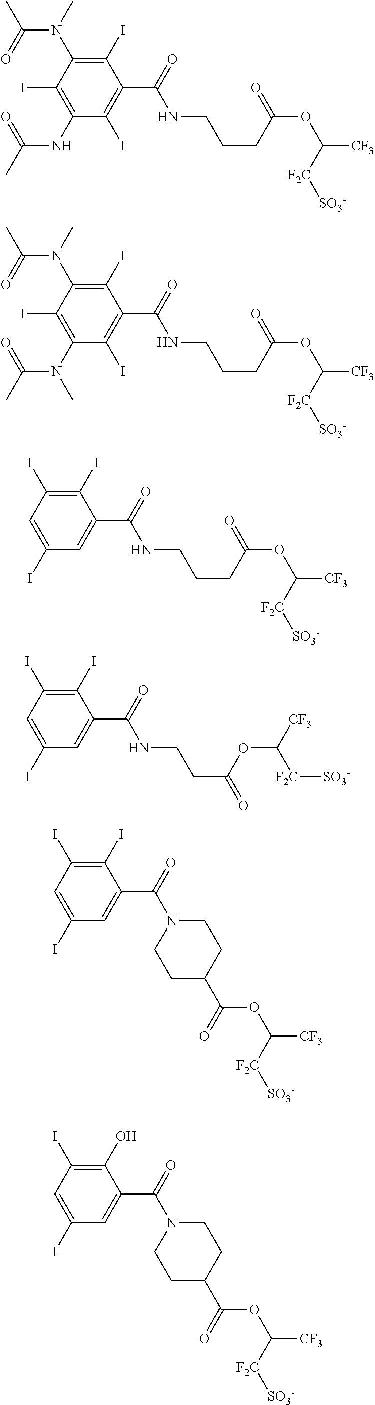

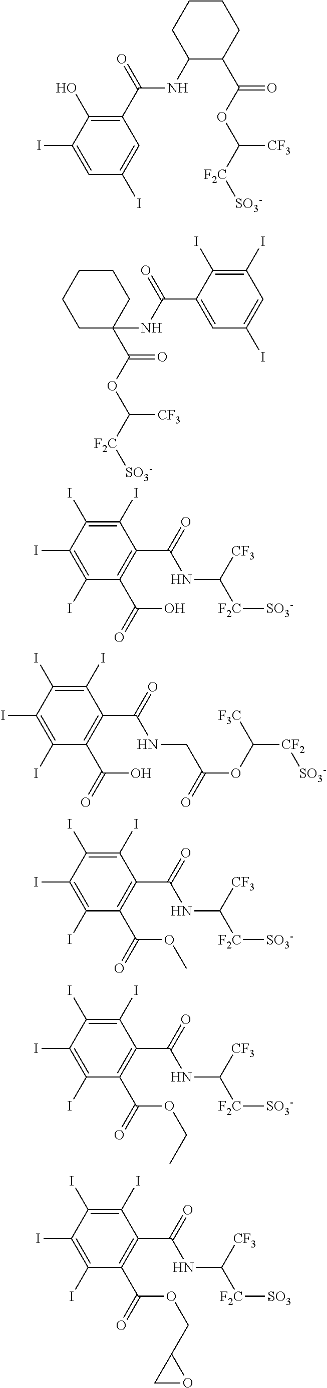

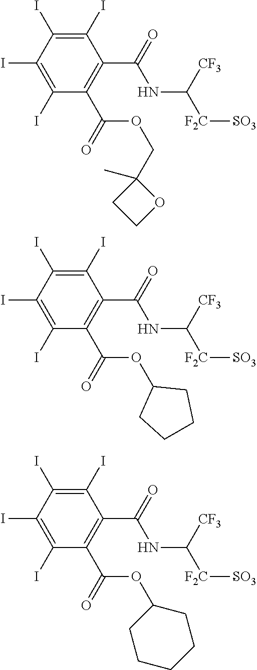

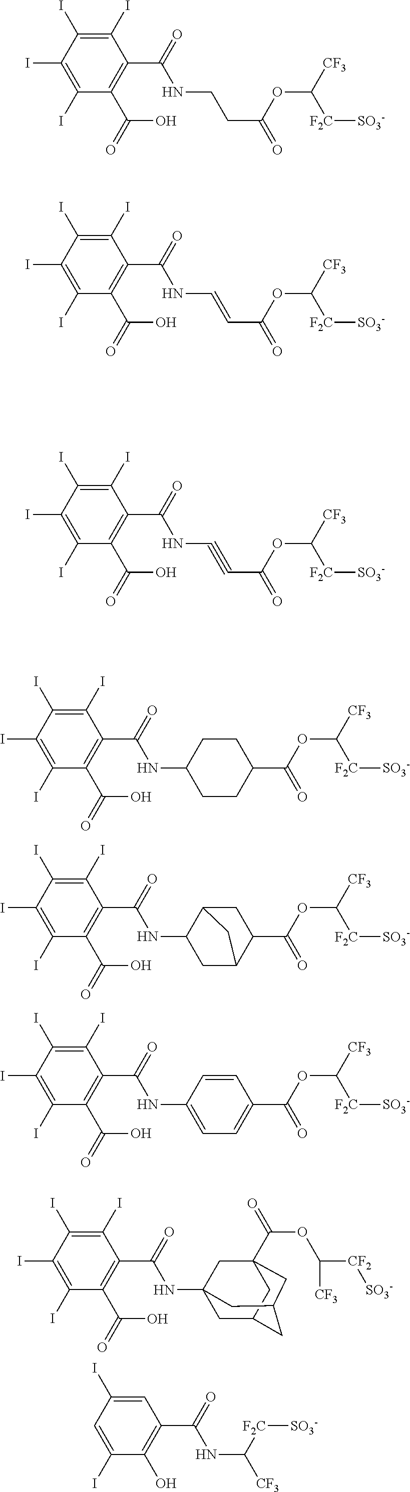

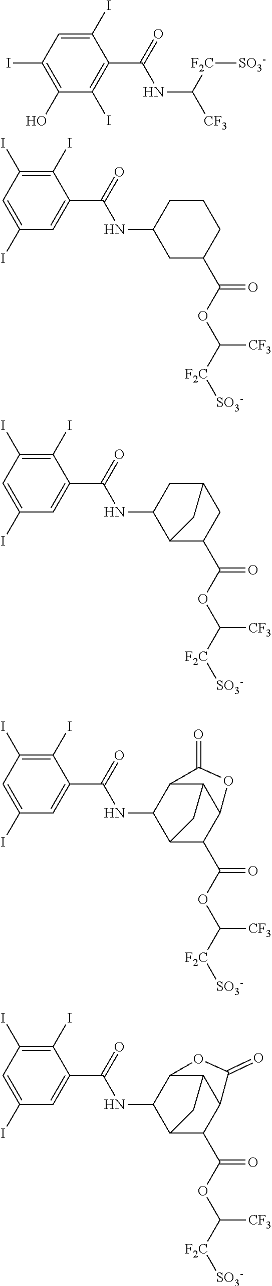

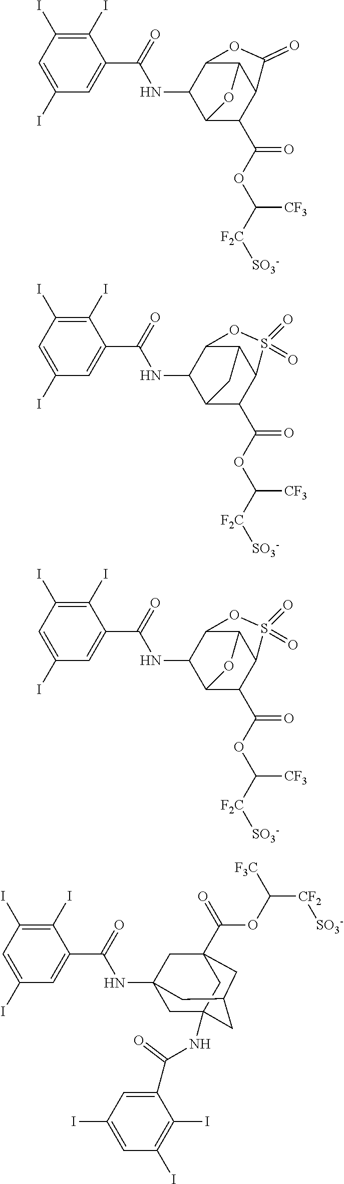

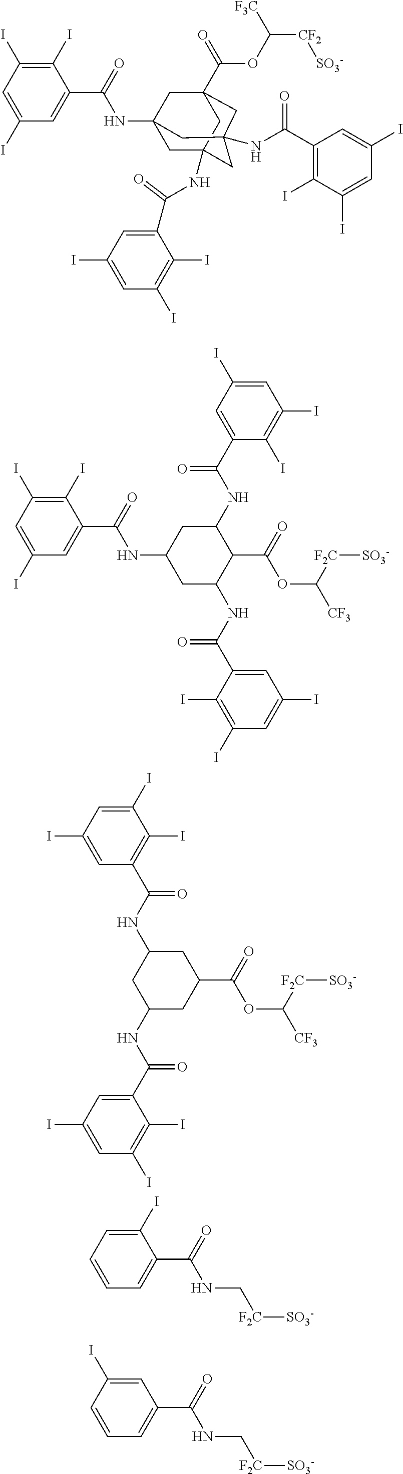

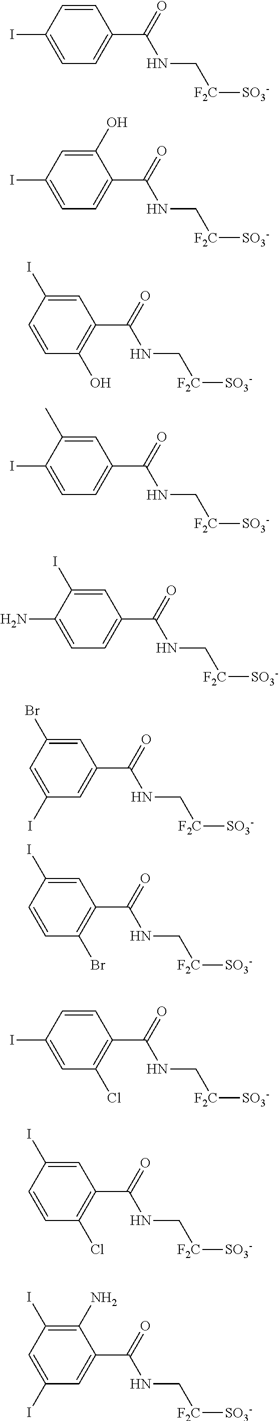

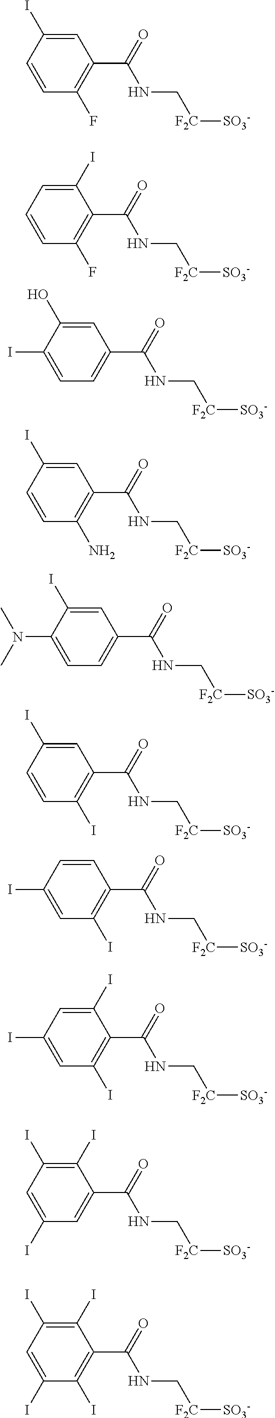

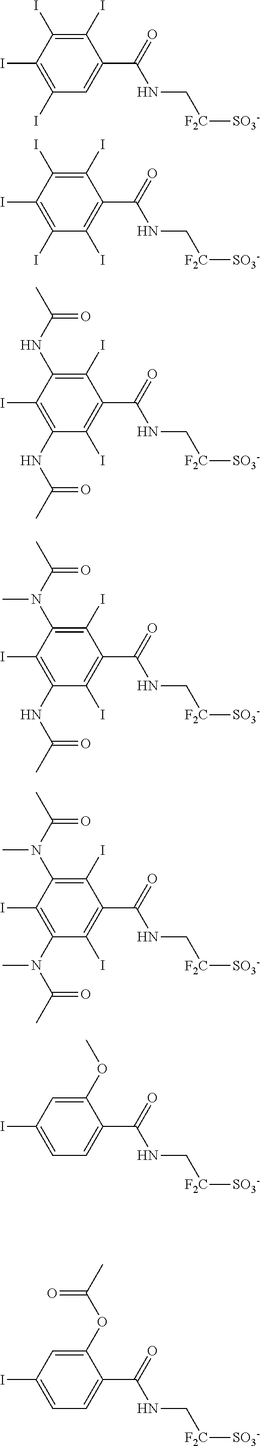

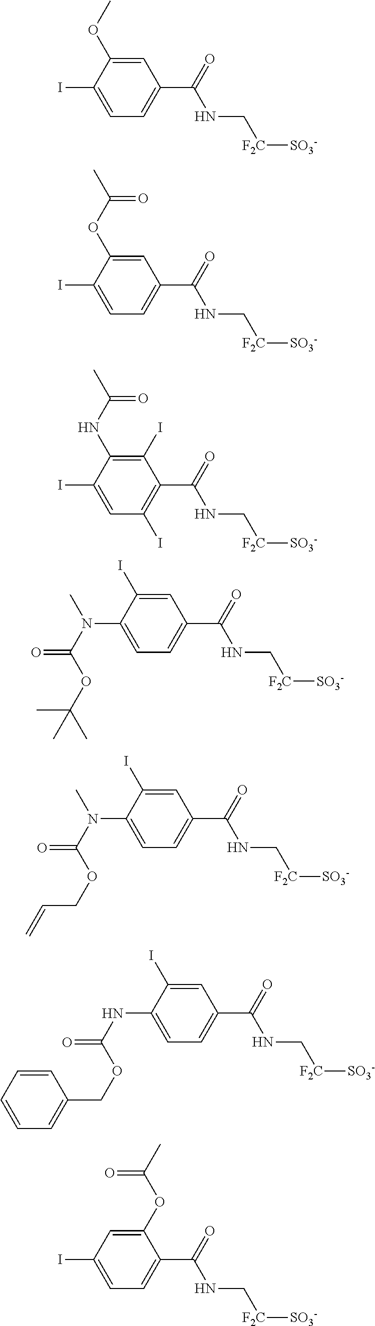

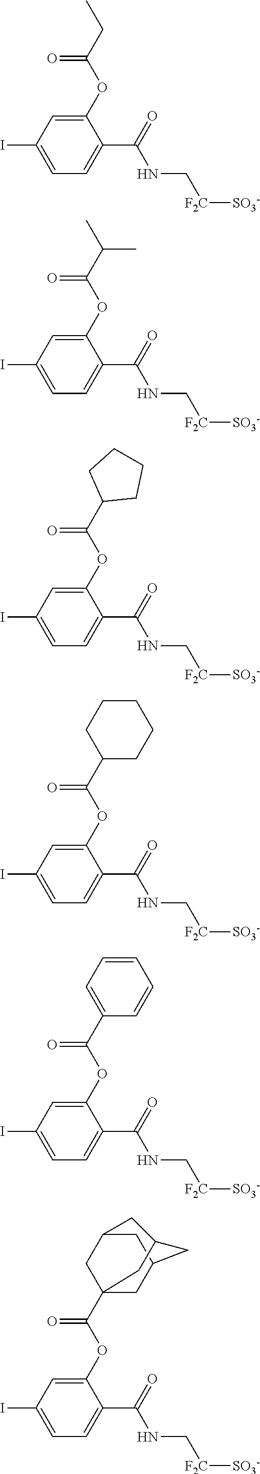

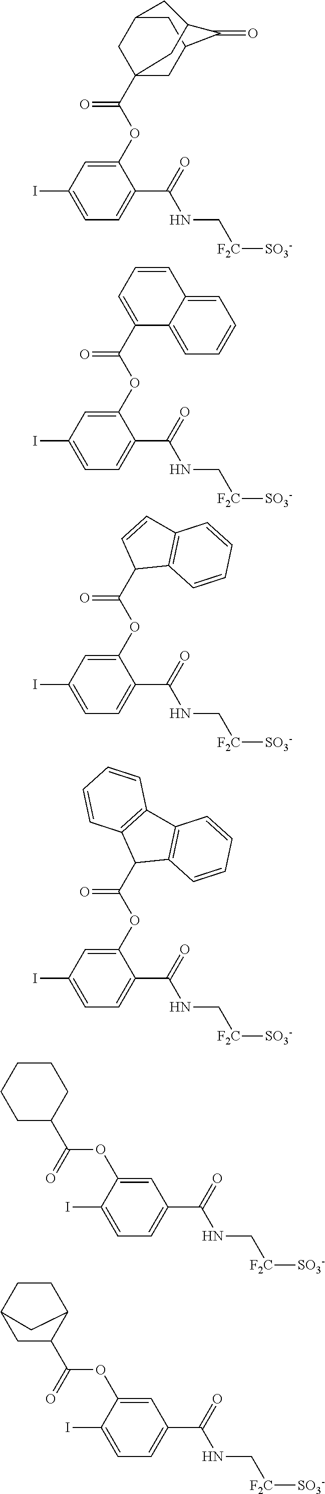

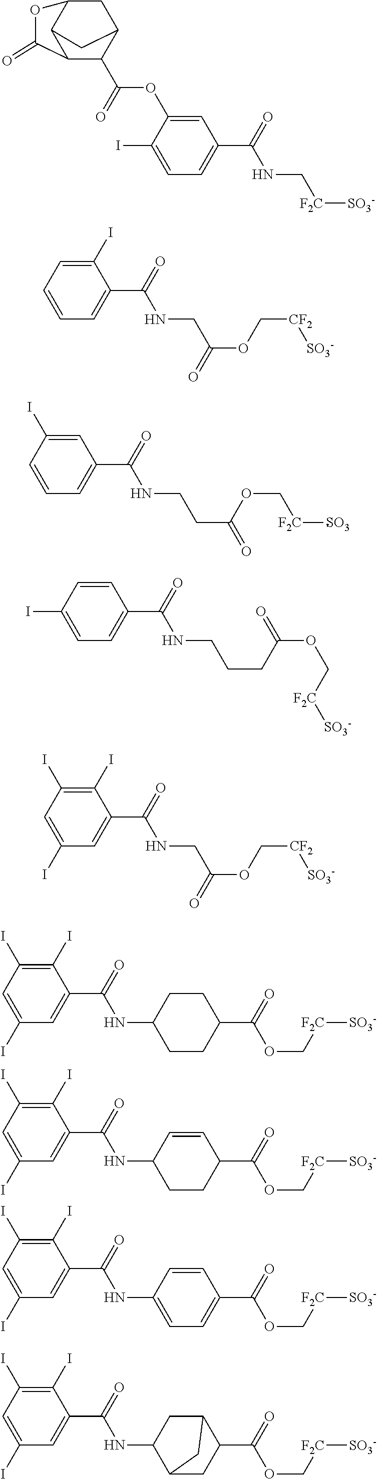

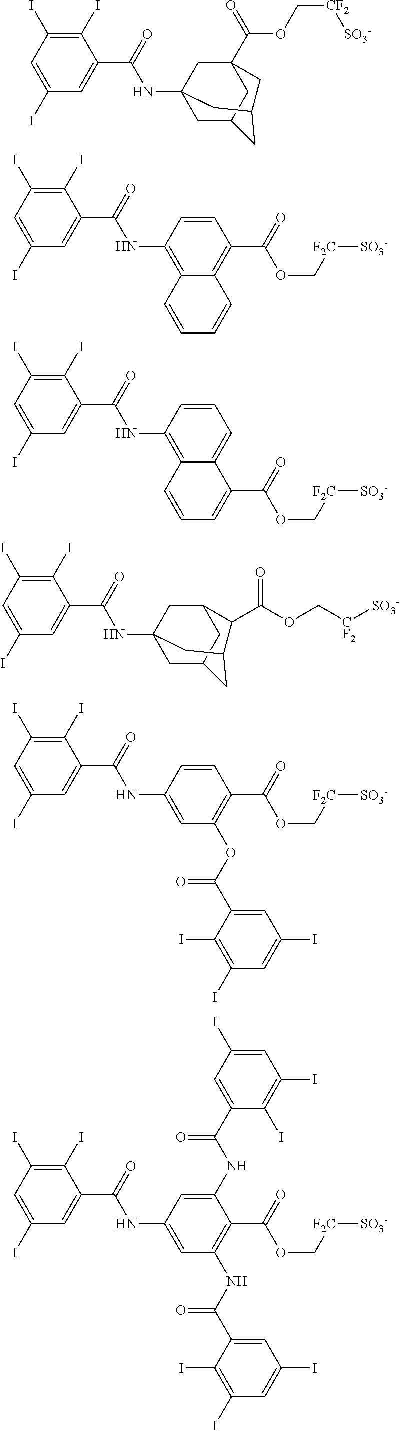

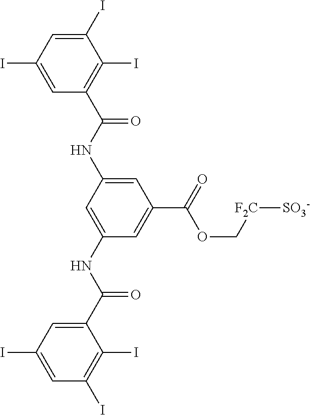

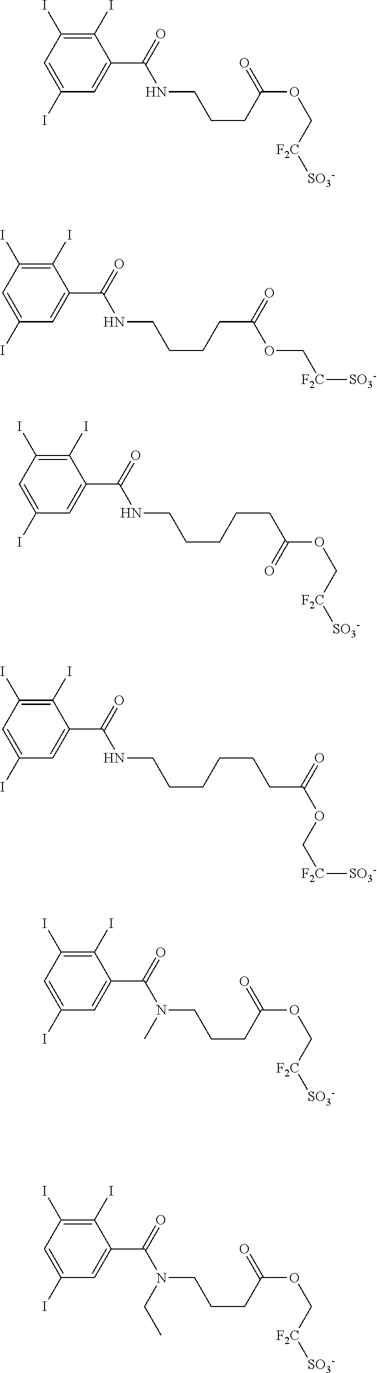

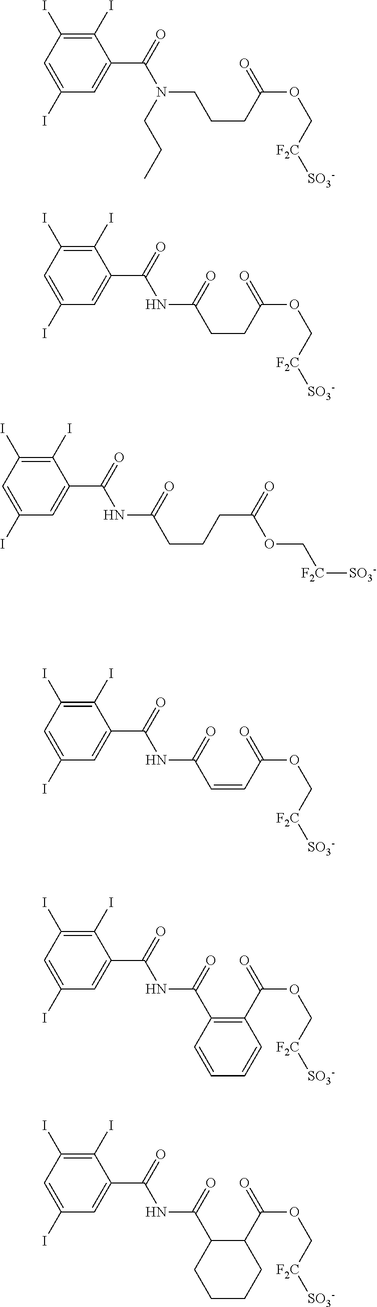

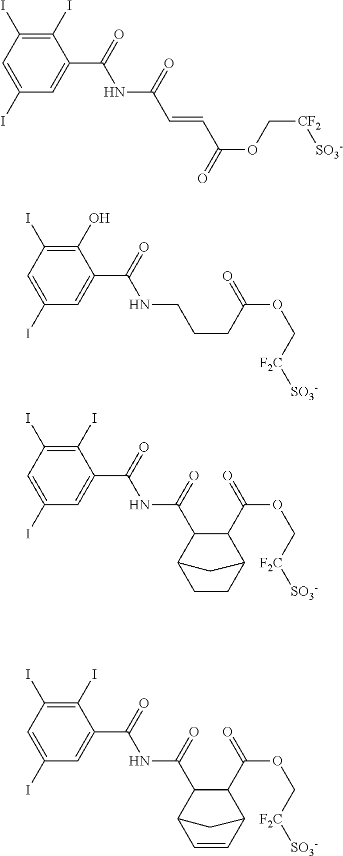

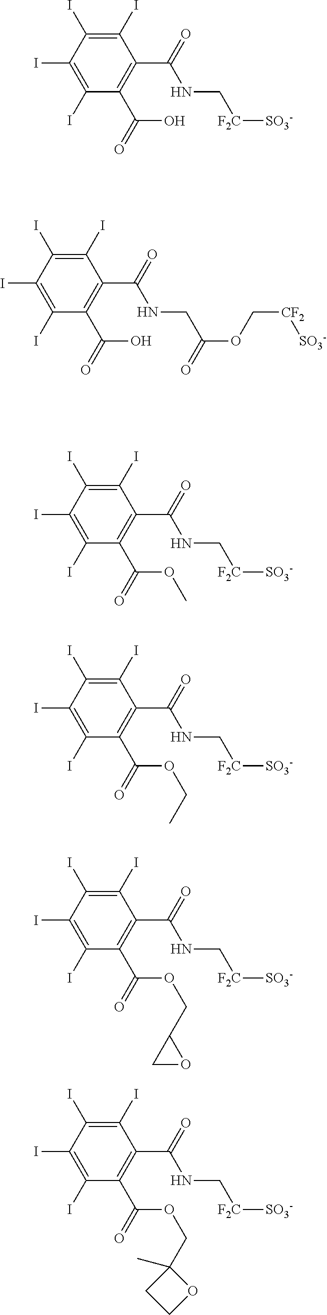

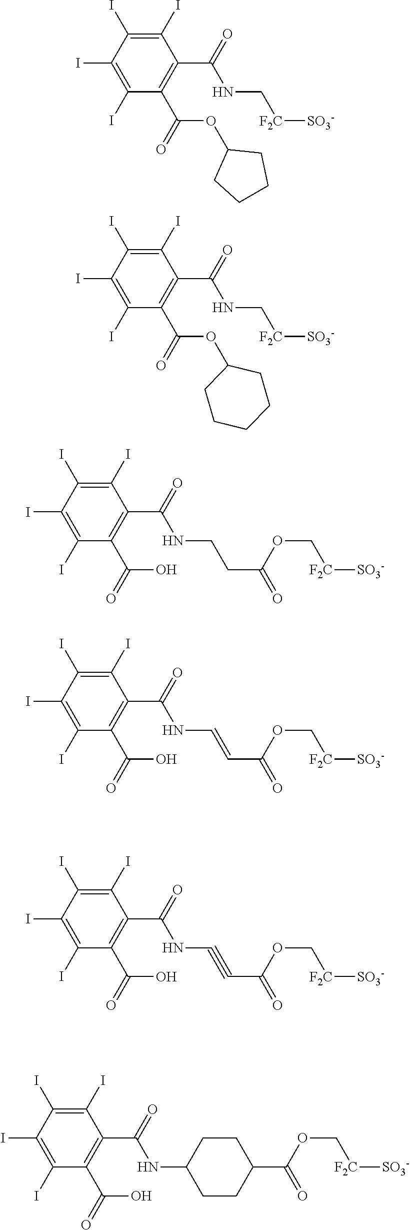

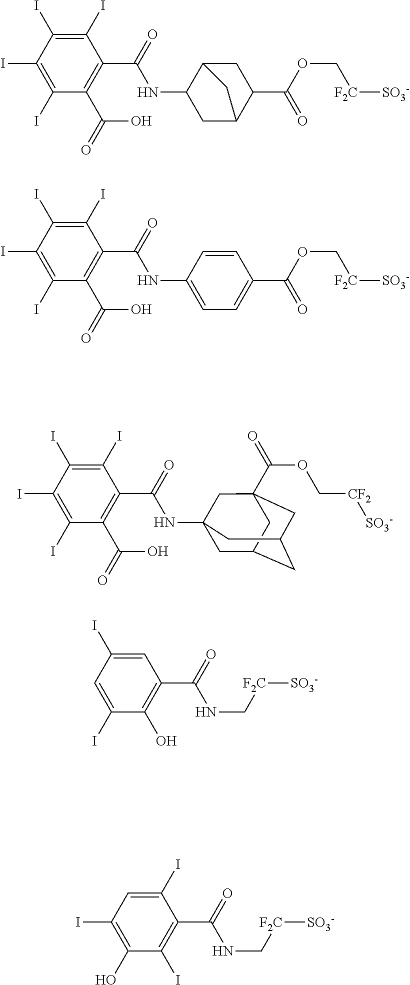

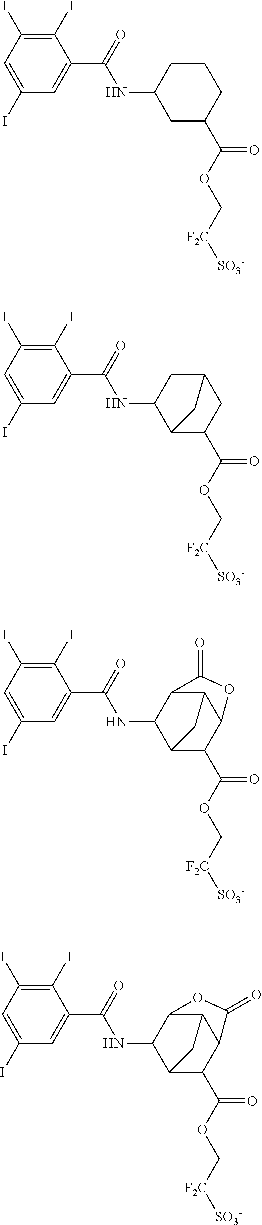

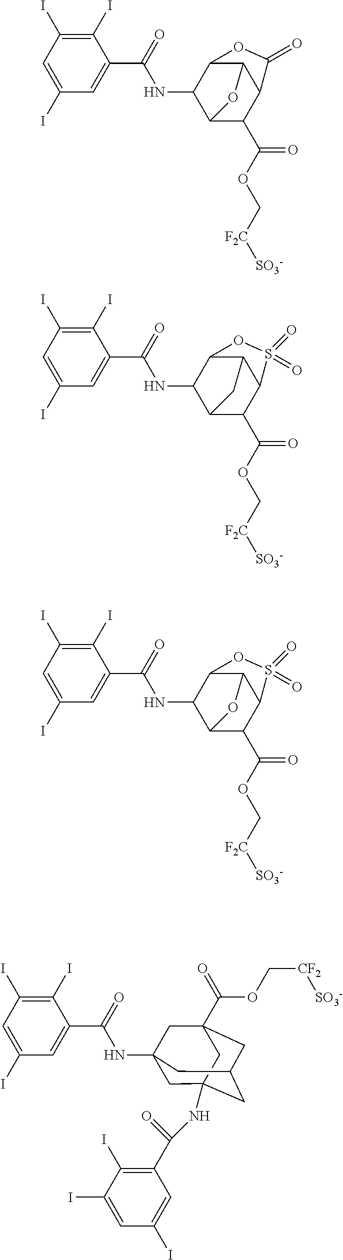

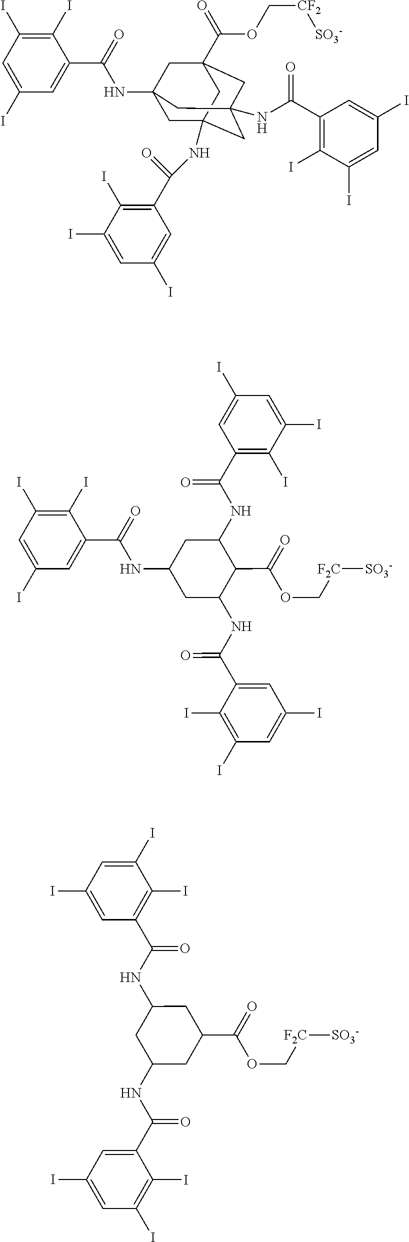

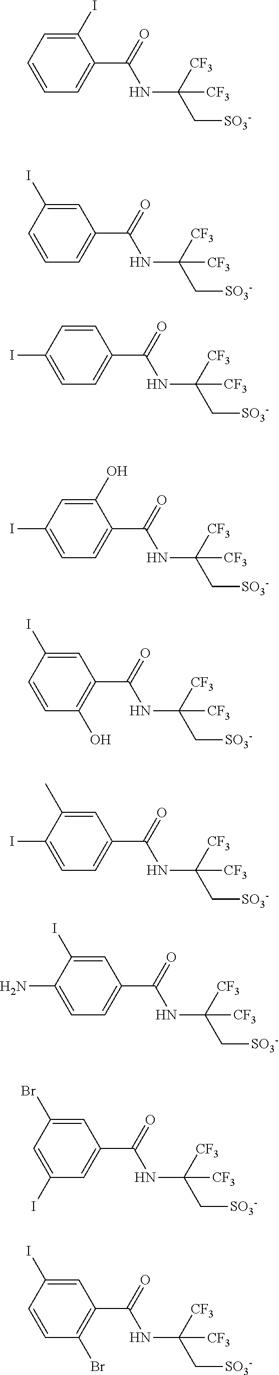

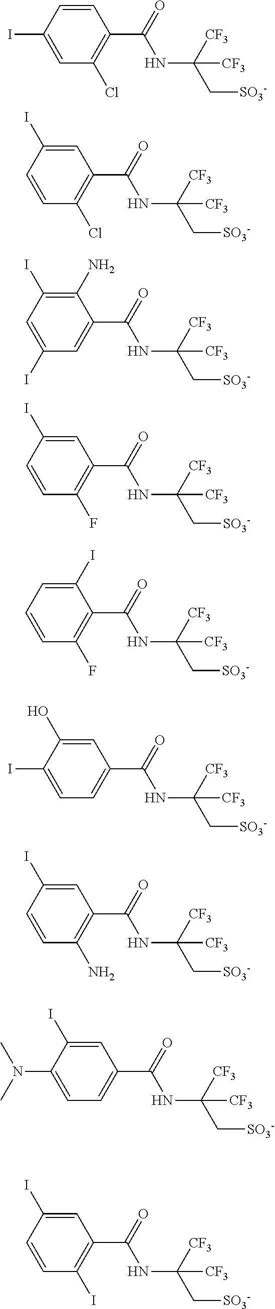

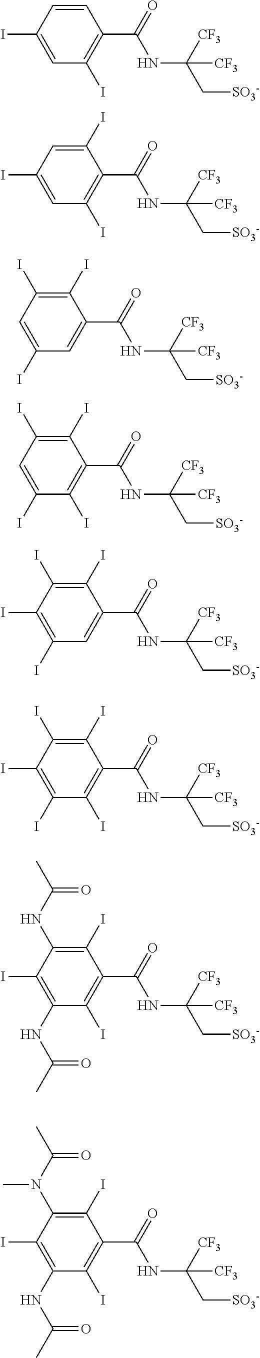

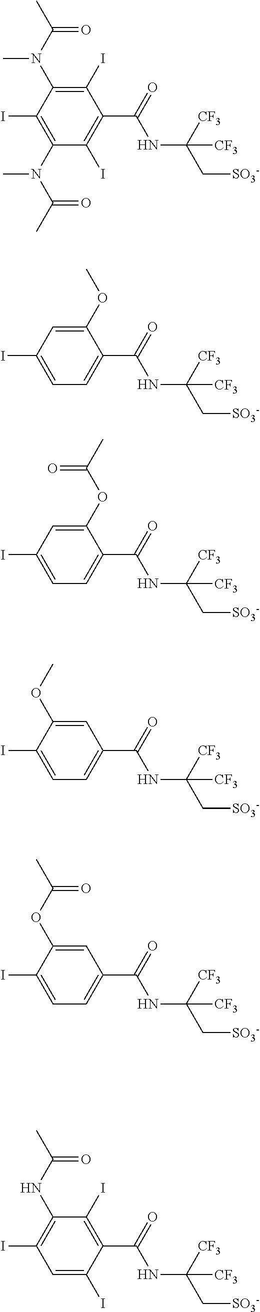

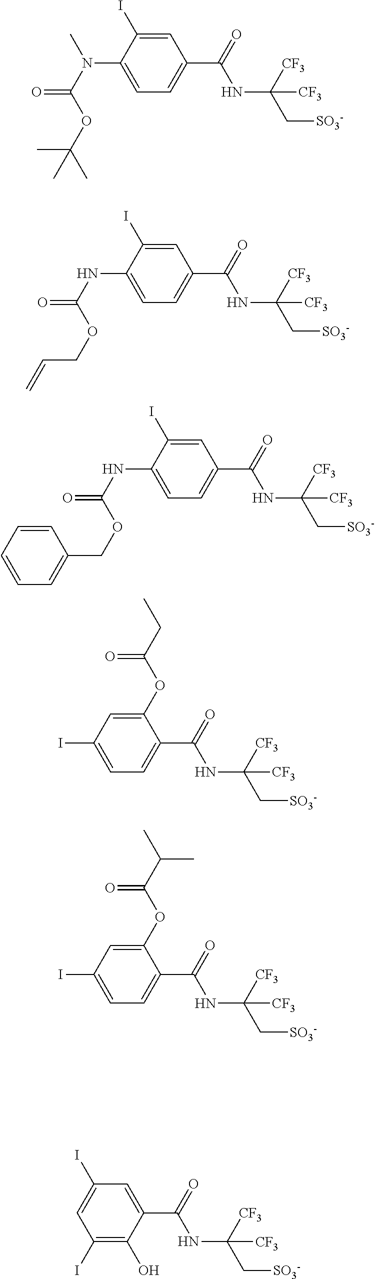

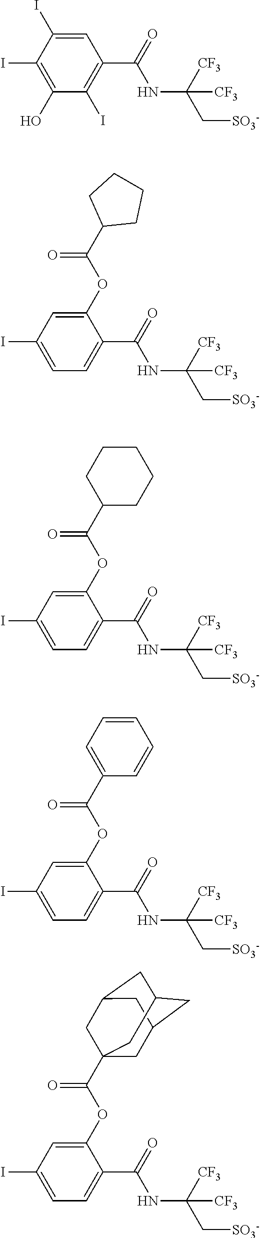

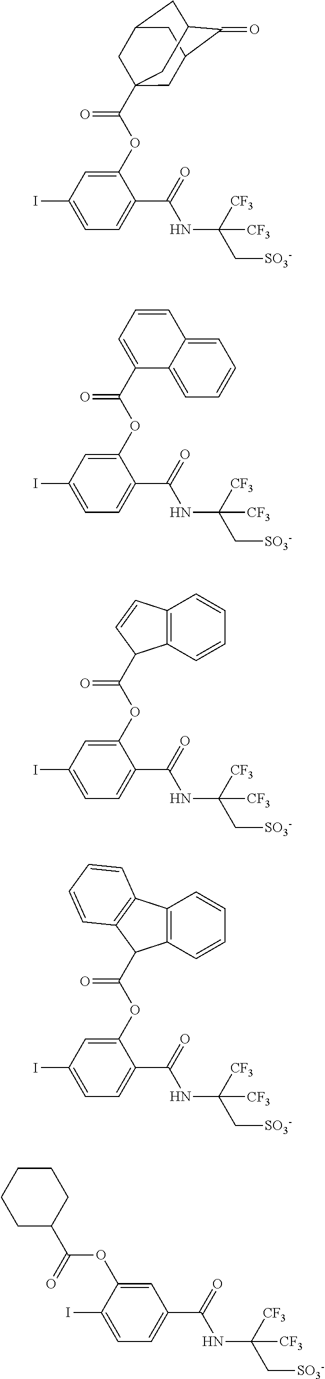

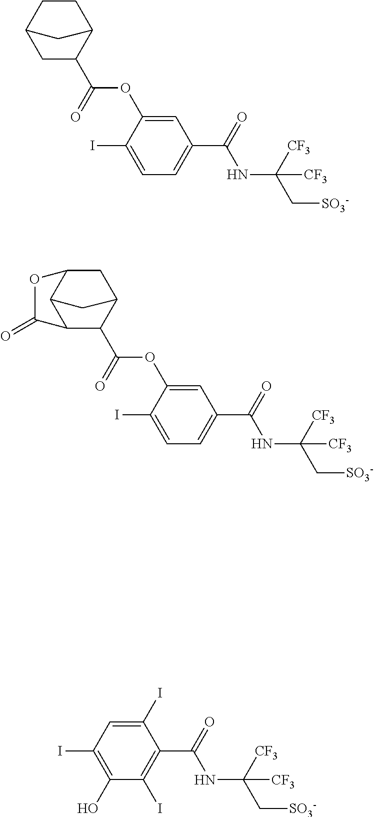

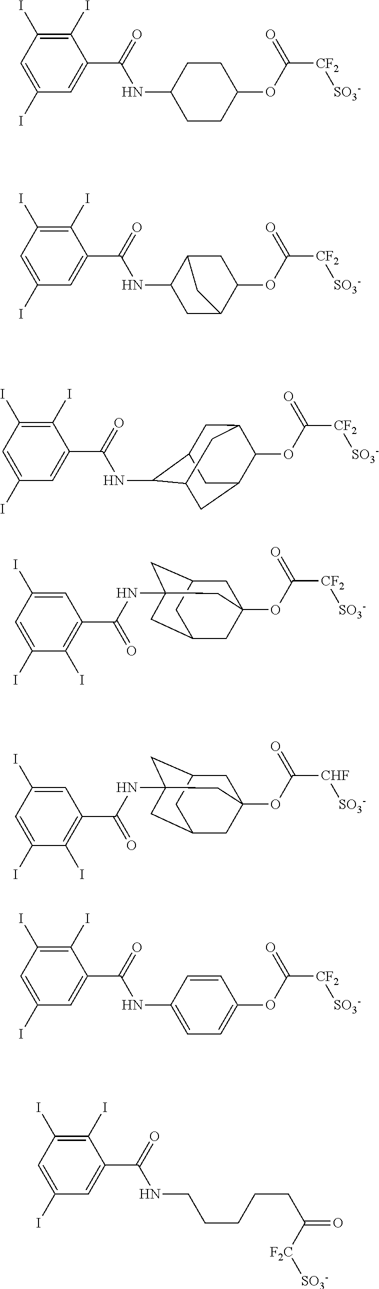

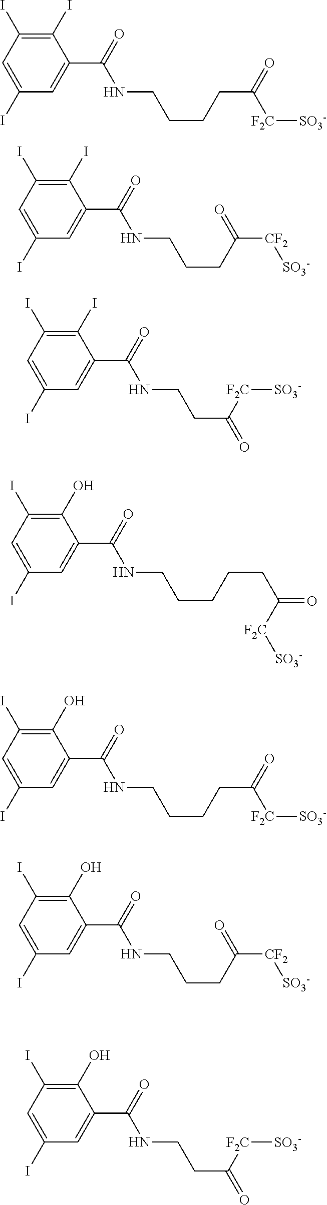

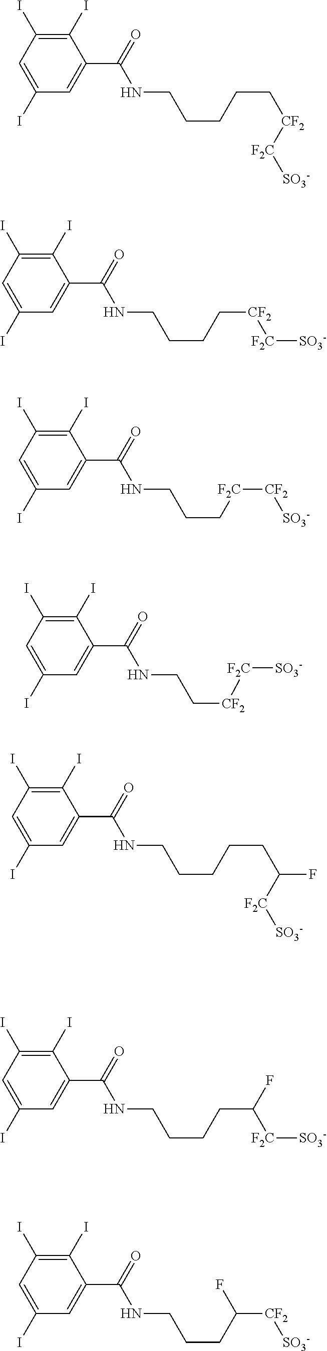

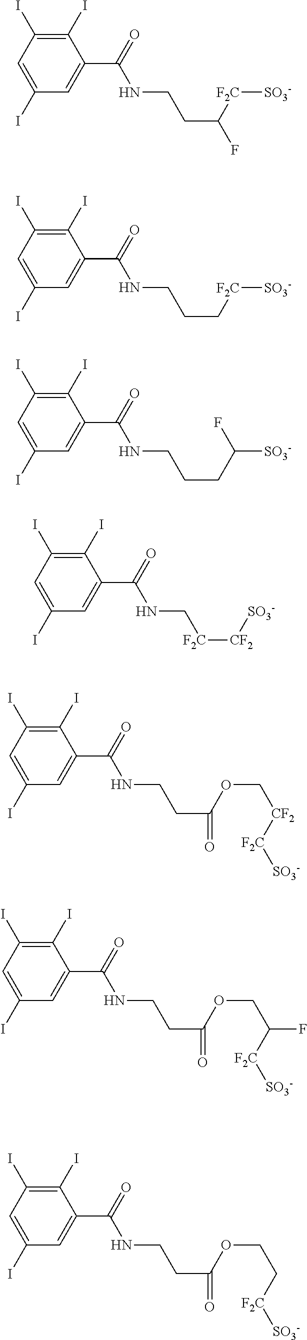

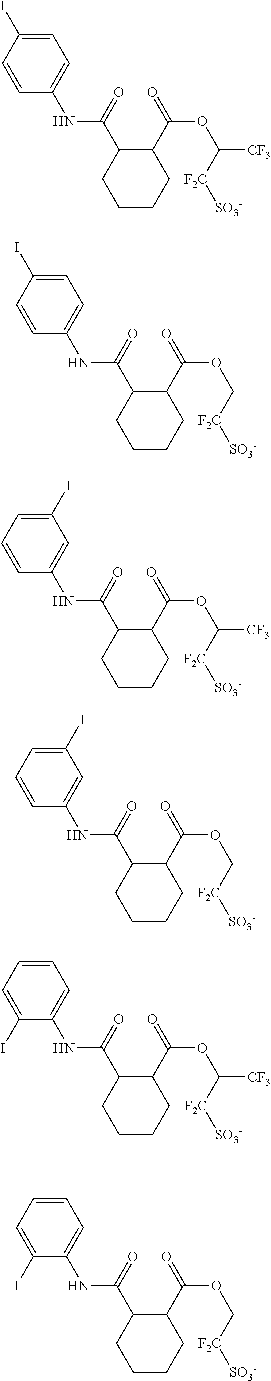

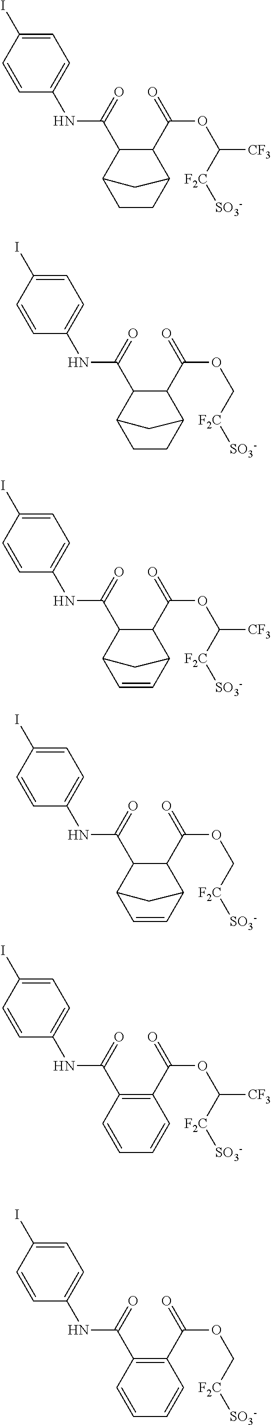

The sulfonium cation in the sulfonium salt as component (B) preferably has the formula (2-4).

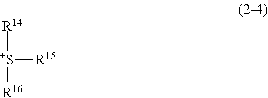

In formula (2-4), R¹⁴ to R¹⁶ are each independently halogen exclusive of fluorine or a C₁-C₂₀ hydrocarbyl group which may contain at least one element selected from oxygen, sulfur, nitrogen, and halogen exclusive of fluorine. R¹⁴ and R¹⁵ may bond together to form a ring with the sulfur atom to which they are attached. Examples of the groups R¹⁴ to R¹⁶ are as exemplified for R² to R⁴ in formula (1), with the proviso that fluorinated groups are excluded.

Of the sulfonium cations having formula (2-4), those cations having the following formulae (2-4-1) and (2-4-2) are more preferred.

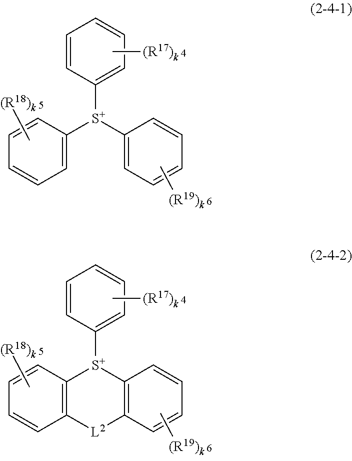

In formulae (2-4-1) and (2-4-2), R¹⁷, R¹⁸ and R¹⁹ are each independently halogen, hydroxy, nitro, cyano, carboxy, a C₁-C₁₄ hydrocarbyl group, C₁-C₁₄ hydrocarbyloxy group, C₂-C₁₄ hydrocarbylcarbonyl group, C₂-C₁₄ hydrocarbylcarbonyloxy group, C₂-C₁₄ hydrocarbyloxycarbonyl group, or C₁-C₁₄ hydrocarbylthio group.

In the hydrocarbyl group, some or all of the hydrogen atoms may be substituted by a moiety containing a heteroatom such as oxygen, sulfur, nitrogen or halogen, so that the group may contain a hydroxy moiety, cyano moiety, fluorine, chlorine, bromine, iodine, or haloalkyl moiety. In the hydrocarbyl group, some constituent —CH₂— may be replaced by —O—, —C(═O)—, —S—, —S(═O)—, —S(═O)₂— or —N(R^{N2})—. R^{N2} is hydrogen or a C₁-C₁₀ hydrocarbyl group in which some hydrogen may be substituted by a moiety containing a heteroatom such as oxygen, sulfur, nitrogen or halogen, so that the group may contain a hydroxy moiety, cyano moiety, fluorine, chlorine, bromine, iodine, or haloalkyl moiety, and some constituent —CH₂— may be replaced by —O—, —C(═O)— or —S(═O)₂—.

In formula (2-4-2), L² is a single bond, —CH₂—, —O—, —C(═O)—, —S—, —S(═O)—, —S(═O)₂— or —N(R^{N2})— wherein R^{N2} is as defined above.

In formulae (2-4-1) and (2-4-2), k⁴, k⁵ and k⁶ are each independently an integer of 0 to 5. When k⁴ is 2 or more, groups R^{1′} may be the same or different and two R^{1′} may bond together to form a ring with the carbon atom on the benzene ring to which they are attached. When k⁵ is 2 or more, groups R¹⁸ may be the same or different and two R¹⁸ may bond together to form a ring with the carbon atom on the benzene ring to which they are attached. When k⁶ is 2 or more, groups R¹⁹ may be the same or different and two R¹⁹ may bond together to form a ring with the carbon atom on the benzene ring to which they are attached.

Examples of the sulfonium cation having formula (2-4) are as exemplified above for the cation in the sulfonium salt having formula (1), exclusive of fluorinated groups.

In the positive resist composition, the acid generator (B) is preferably present in an amount of 0.1 to 100 parts by weight, more preferably 1 to 50 parts by weight per 100 parts by weight of the base polymer (C) to be described below.

### (C) Base Polymer

Component (C) is a base polymer comprising repeat units of at least one type selected from repeat units (a1) having a carboxy group whose hydrogen is substituted by an acid labile group and repeat units (a2) having a phenolic hydroxy group whose hydrogen is substituted by an acid labile group.

In a preferred embodiment, the repeat unit (a1) has the formula (a1) and the repeat unit (a2) has the formula (a2).

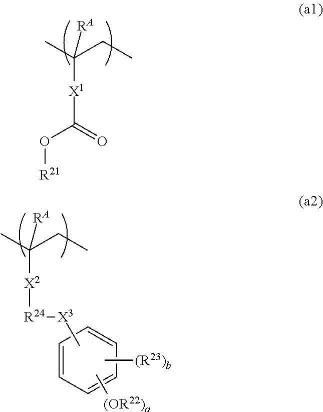

In formulae (a1) and (a2), R^{A} is each independently hydrogen or methyl. X¹ is a single bond, phenylene group, naphthylene group, or a C₁-C₁₂ linking group containing at least one moiety selected from an ether bond, ester bond and lactone ring. X² is a single bond, ester bond or amide bond. X³ is a single bond, ether bond or ester bond. R²¹ and R²² are each independently an acid labile group. R²³ is fluorine, trifluoromethyl, cyano or a C₁-C₆ saturated hydrocarbyl group. R²⁴ is a single bond or a C₁-C₆ alkanediyl group in which some carbon may be replaced by an ether bond or ester bond. The subscript “a” is 1 or 2, b is an integer of 0 to 4, and 1≤a+b≤5.

Examples of the monomer from which repeat units (a1) are derived are shown below, but not limited thereto. Herein R^{A} and R^{A2} are as defined above.

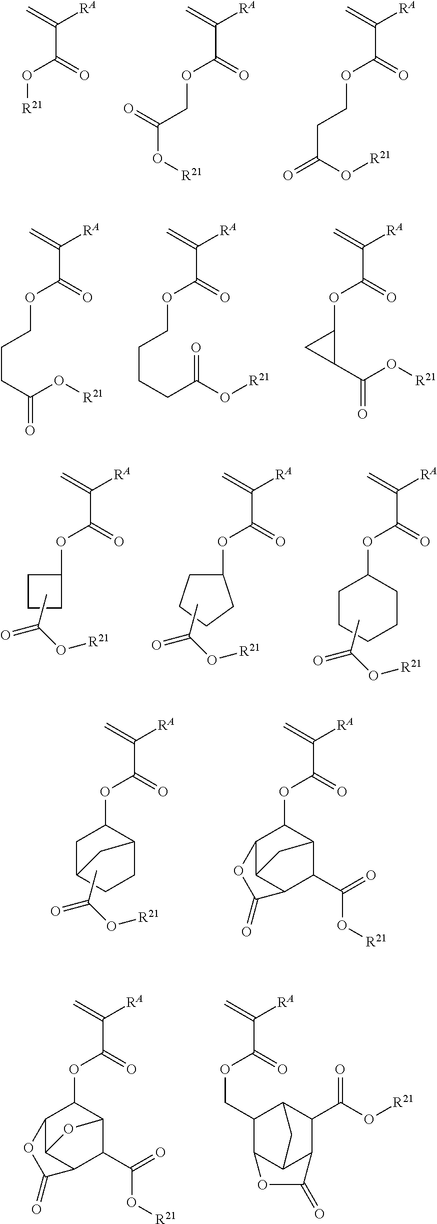

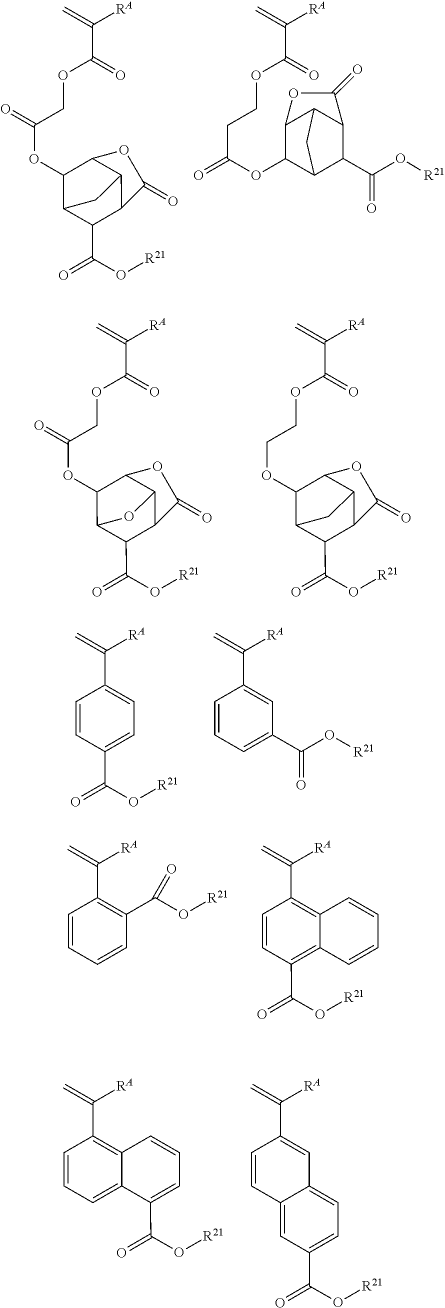

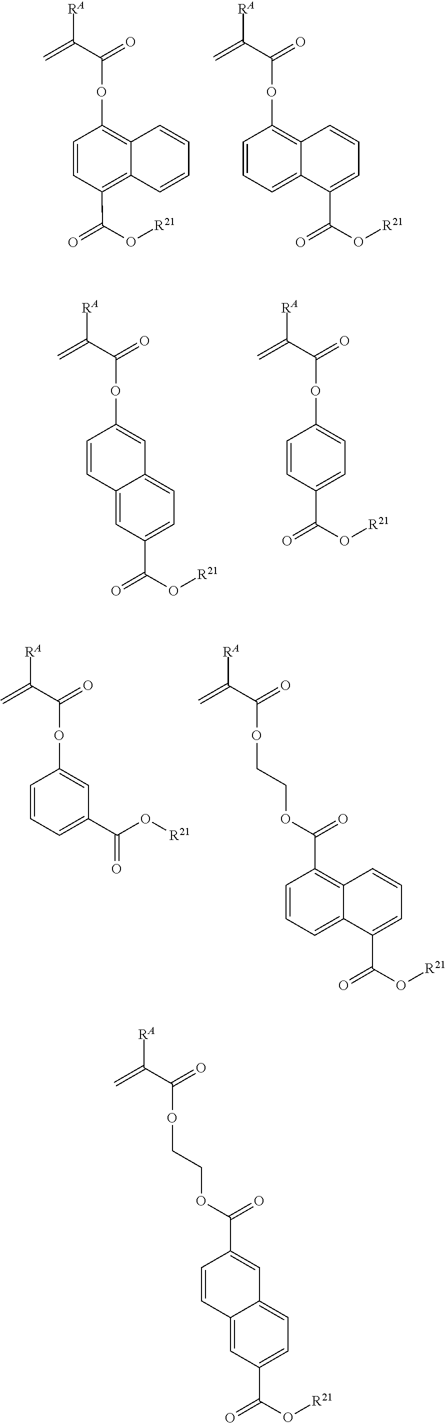

Examples of the monomer from which repeat units (a2) are derived are shown below, but not limited thereto. Herein R^{A} and R²² are as defined above.

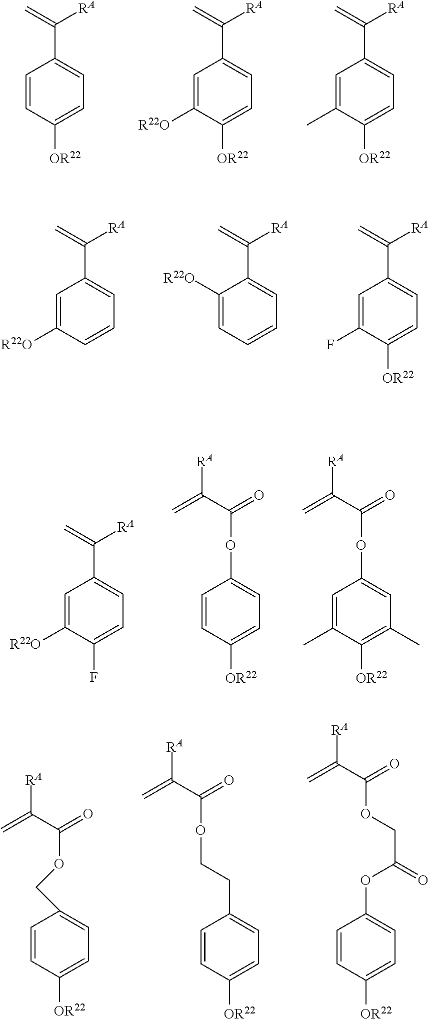

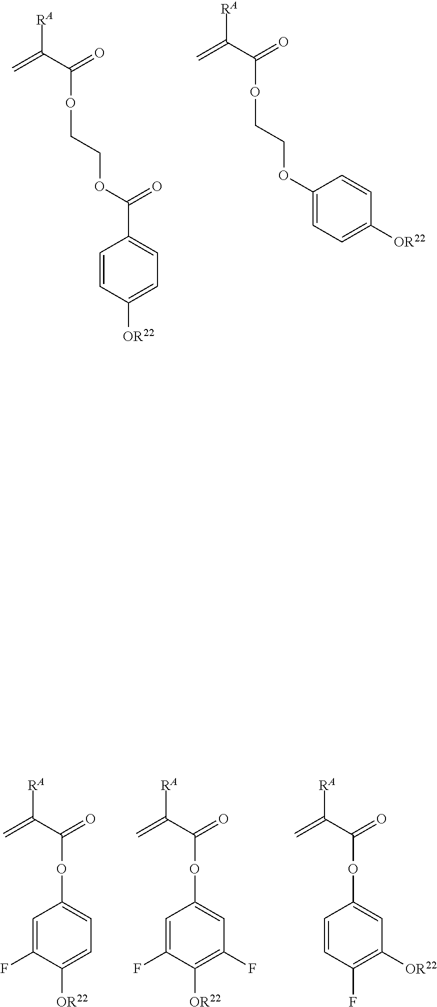

The acid labile groups represented by R²¹ and R²² may be selected from a variety of such groups, for example, those groups having the following formulae (AL-1) to (AL-3).

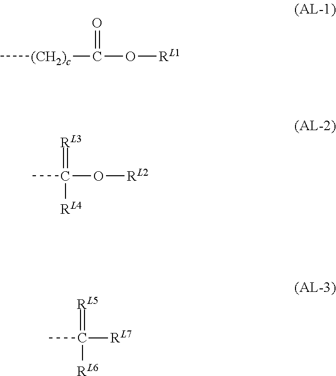

In formulae (AL-1), c is an integer of 0 to 6. R^{L1} is a C₄-C₄₀, preferably C₄-C₁₅ tertiary hydrocarbyl group, a trihydrocarbylsilyl group in which each hydrocarbyl moiety is a C₁-C₆ saturated hydrocarbyl moiety, a C₄-C₂₀ saturated hydrocarbyl group containing a carbonyl moiety, ether bond or ester bond, or a group having formula (AL-3).

The tertiary hydrocarbyl group R^{L1} may be saturated or unsaturated and branched or cyclic. Examples thereof include tert-butyl, tert-pentyl, 1,1-diethylpropyl, 1-ethylcyclopentyl, 1-butylcyclopentyl, 1-ethylcyclohexyl, 1-butylcyclohexyl, 1-ethyl-2-cyclopentenyl, 1-ethyl-2-cyclohexenyl, and 2-methyl-2-adamantyl. Suitable trihydrocarbylsilyl groups include trimethylsilyl, triethylsilyl, and dimethyl-tert-butylsilyl. The saturated hydrocarbyl group containing a carbonyl moiety, ether bond or ester bond may be straight, branched or cyclic, preferably cyclic, and examples thereof include 3-oxocyclohexyl, 4-methyl-2-oxooxan-4-yl, 5-methyl-2-oxooxolan-5-yl, 2-tetrahydropyranyl, and 2-tetrahydrofuranyl.

Examples of the acid labile group having formula (AL-1) include tert-butoxycarbonyl, tert-butoxycarbonylmethyl, tert-pentyloxycarbonyl, tert-pentyloxycarbonylmethyl, 1,1-diethylpropyloxycarbonyl, 1,1-diethylpropyloxycarbonylmethyl, 1-ethylcyclopentyloxycarbonyl, 1-ethylcyclopentyloxycarbonylmethyl, 1-ethyl-2-cyclopentenyloxycarbonyl, 1-ethyl-2-cyclopentenyloxycarbonylmethyl, 1-ethoxyethoxycarbonylmethyl, 2-tetrahydropyranyloxycarbonylmethyl, and 2-tetrahydrofuranyloxycarbonylmethyl.

Other examples of the acid labile group having formula (AL-1) include groups having the formulae (AL-1)-1 to (AL-1)-10.

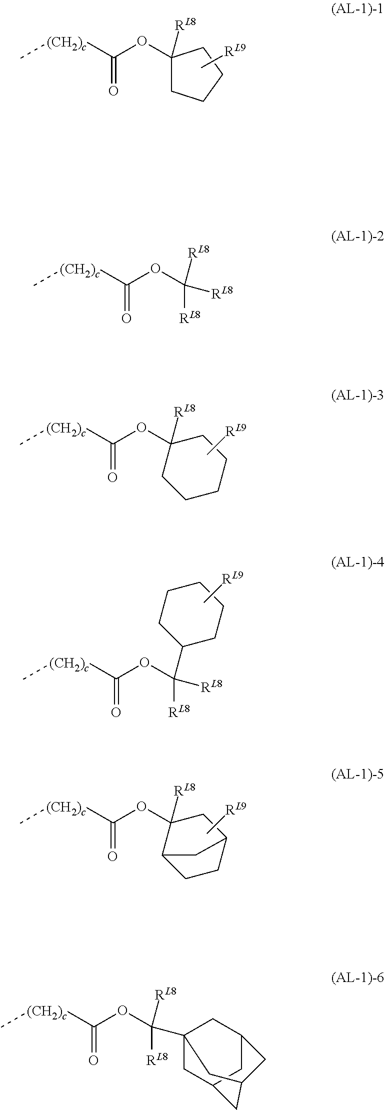

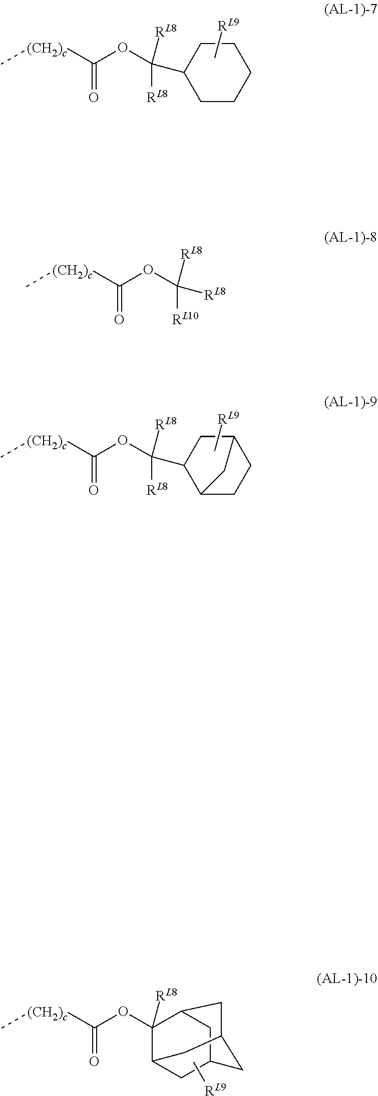

In formulae (AL-1)-1 to (AL-1)-10, c is as defined above. R^{L8} is each independently a C₁-C₁₀ saturated hydrocarbyl group or C₆-C₂₀ aryl group. R^{L9} is hydrogen or a C₁-C₁₀ saturated hydrocarbyl group. R^{L10} is a C₂-C₁₀ saturated hydrocarbyl group or C₆-C₂₀ aryl group. The saturated hydrocarbyl group may be straight, branched or cyclic. In formula (AL-2), R^{L2} and R^{L3} are each independently hydrogen or a C₁-C₁₈, preferably C₁-C₁₀ saturated hydrocarbyl group. The saturated hydrocarbyl group may be straight, branched or cyclic and examples thereof include methyl, ethyl, propyl, isopropyl, n-butyl, sec-butyl, tert-butyl, cyclopentyl, cyclohexyl, 2-ethylhexyl and n-octyl.

In formula (AL-2), R^{L4} is a C₁-C₁₈, preferably C₁-C₁₀ hydrocarbyl group which may contain a heteroatom. The hydrocarbyl group may be saturated or unsaturated and straight, branched or cyclic and typical examples thereof include C₁-C₁₈ saturated hydrocarbyl groups, in which some hydrogen may be substituted by hydroxy, alkoxy, oxo, amino or alkylamino. Examples of the substituted saturated hydrocarbyl group are shown below.

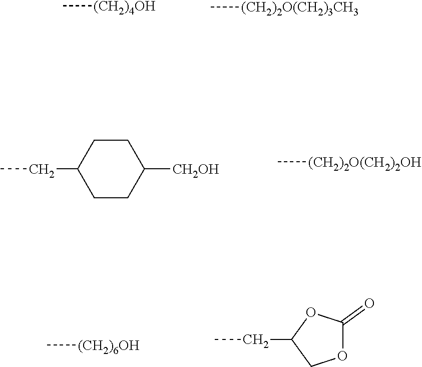

A pair of R^{L2} and R^{L3}, R^{L2} and R^{L4}, or R^{L3} and R^{L4} may bond together to form a ring with the carbon atom or carbon and oxygen atoms to which they are attached. R^{L2} and R^{L3}, R^{L2} and R^{L4}, and R^{L3} and R^{L4} taken together to form a ring are each independently a C₁-C₁₈, preferably C₁-C₁₀ alkanediyl group. The ring thus formed is preferably of 3 to 10, more preferably 4 to 10 carbon atoms.

Of the acid labile groups having formula (AL-2), suitable straight or branched groups include those having formulae (AL-2)-1 to (AL-2)-69, but are not limited thereto.

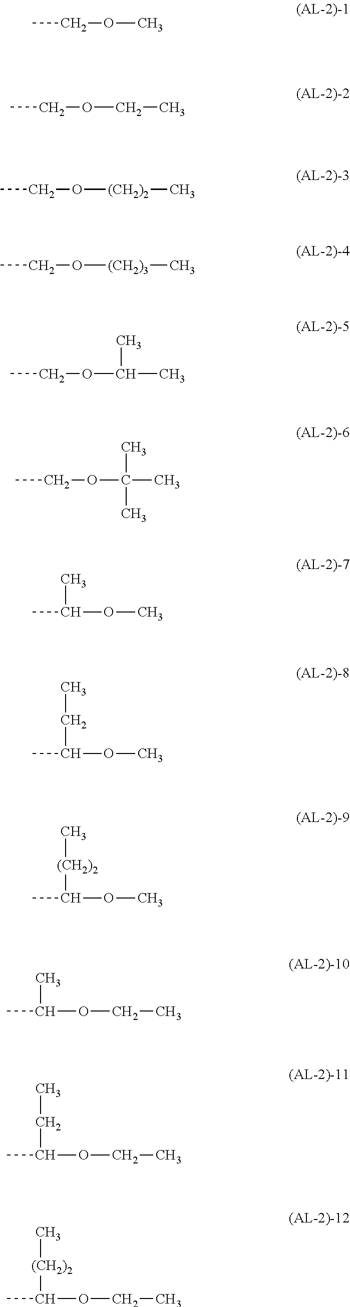

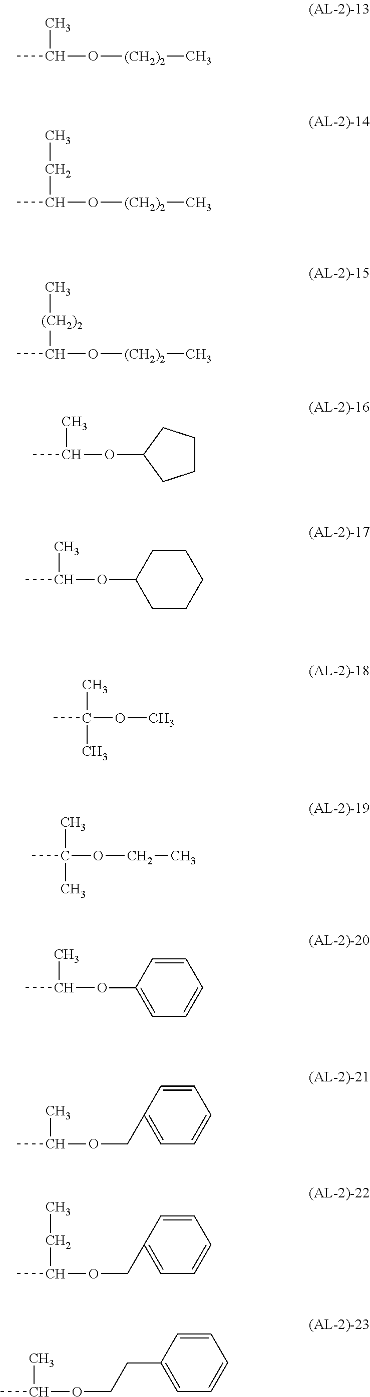

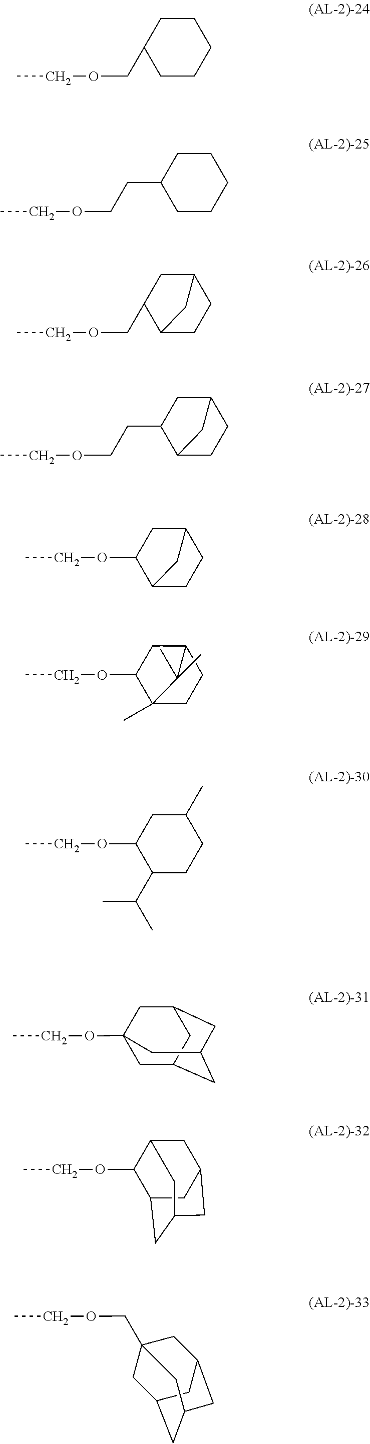

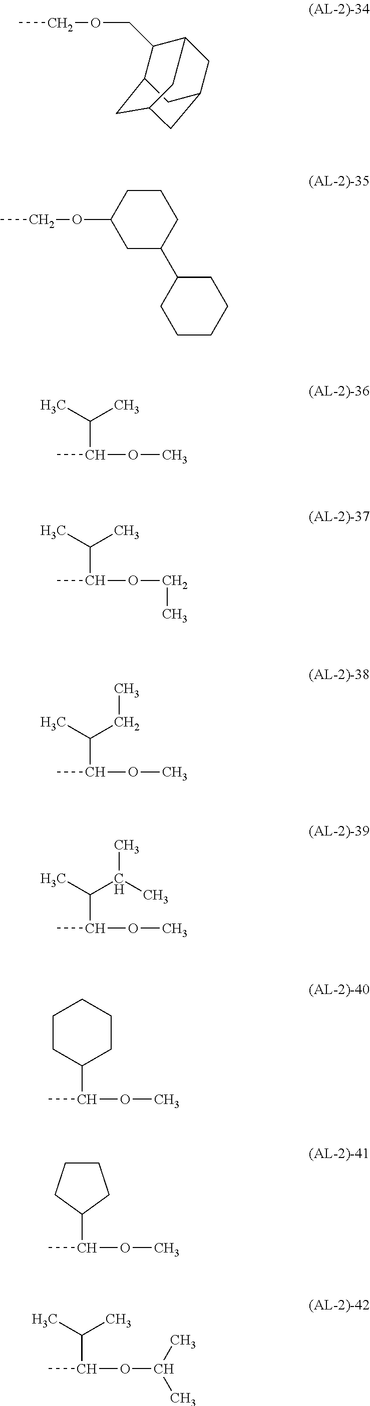

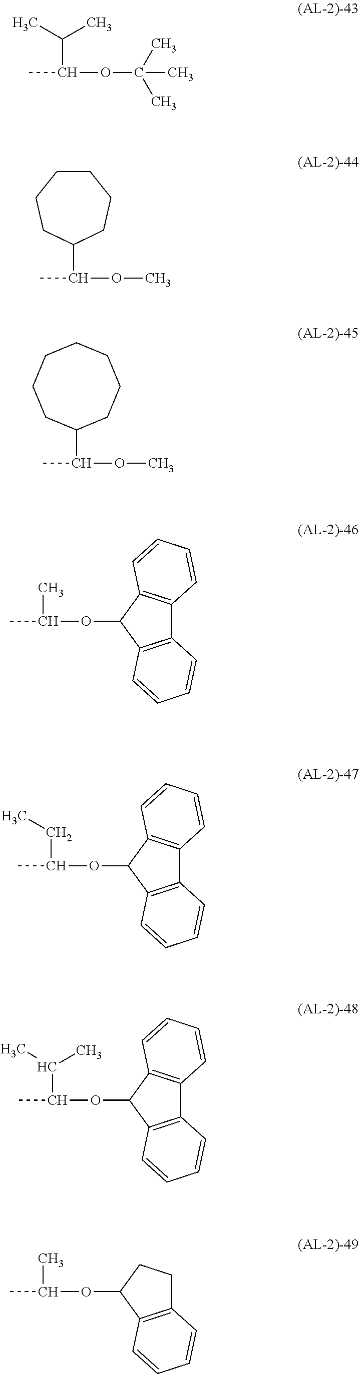

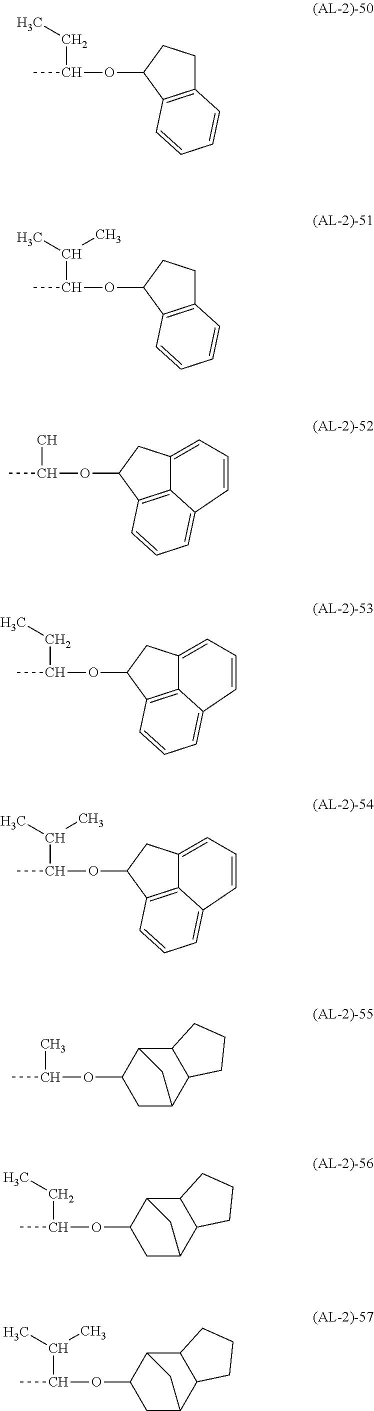

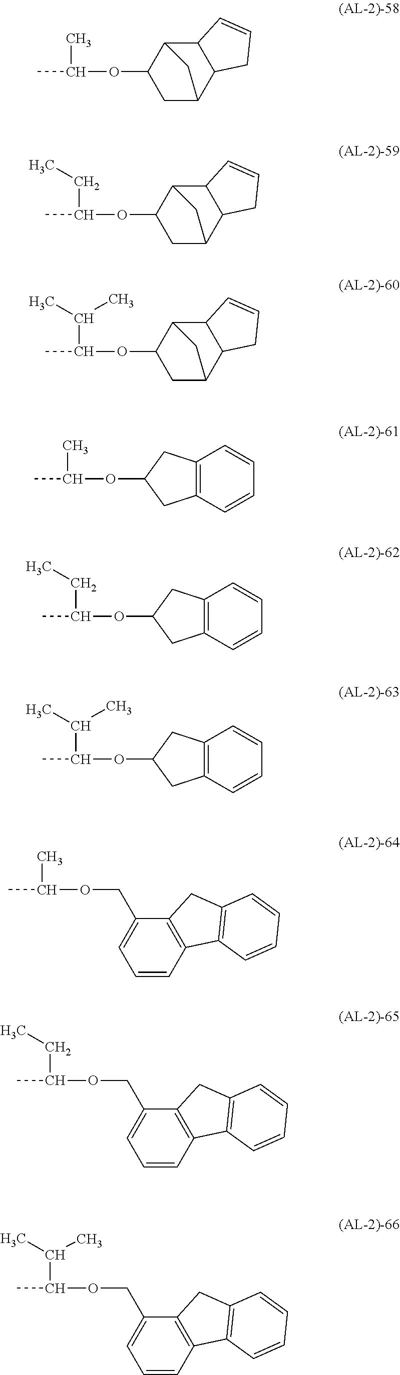

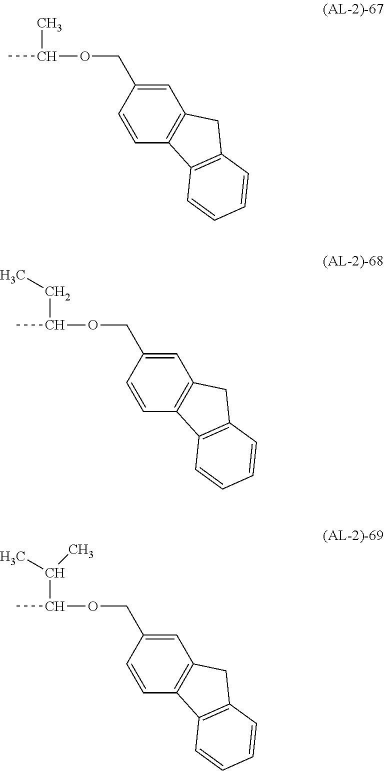

Of the acid labile groups having formula (AL-2), suitable cyclic groups include tetrahydrofuran-2-yl, 2-methyltetrahydrofuran-2-yl, tetrahydropyran-2-yl, and 2-methyltetrahydropyran-2-yl.

Also included are acid labile groups having the following formulae (AL-2a) and (AL-2b). The base polymer may be crosslinked within the molecule or between molecules with these acid labile groups.

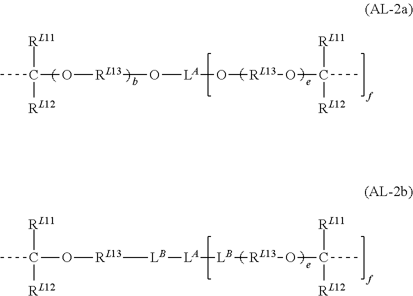

In formulae (AL-2a) and (AL-2b), R^{L11} and R^{L12} are each independently hydrogen or a C₁-C₈ saturated hydrocarbyl group which may be straight, branched or cyclic. Also, R^{L11} and R^{L12} may bond together to form a ring with the carbon atom to which they are attached, and in this case, R^{L11} and R^{L12} are each independently a C₁-C₈ alkanediyl group. R^{L13} is each independently a C₁-C₁₀ saturated hydrocarbylene group which may be straight, branched or cyclic. The subscripts d and e are each independently an integer of 0 to 10, preferably 0 to 5, and f is an integer of 1 to 7, preferably 1 to 3.

In formulae (AL-2a) and (AL-2b), L^{A} is a (f+1)-valent C₁-C₅₀ aliphatic saturated hydrocarbon group, (f+1)-valent C₃-C₅₀ alicyclic saturated hydrocarbon group, (f+1)-valent C₆-C₅₀ aromatic hydrocarbon group or (f+1)-valent C₃-C₅₀ heterocyclic group. In these groups, some constituent —CH₂— may be replaced by a heteroatom-containing moiety, or some carbon-bonded hydrogen may be substituted by a hydroxy, carboxy, acyl moiety or fluorine. L^{A} is preferably a C₁-C₂₀ saturated hydrocarbon group such as saturated hydrocarbylene, trivalent saturated hydrocarbon or tetravalent saturated hydrocarbon group, or C₆-C₃₀ arylene group. The saturated hydrocarbon group may be straight, branched or cyclic. L^{B} is —C(═O)—O—, —NH—C(═O)—O— or —NH—C(═O)—NH—.

Examples of the crosslinking acetal groups having formulae (AL-2a) and (AL-2b) include groups having the formulae (AL-2)-70 to (AL-2)-77.

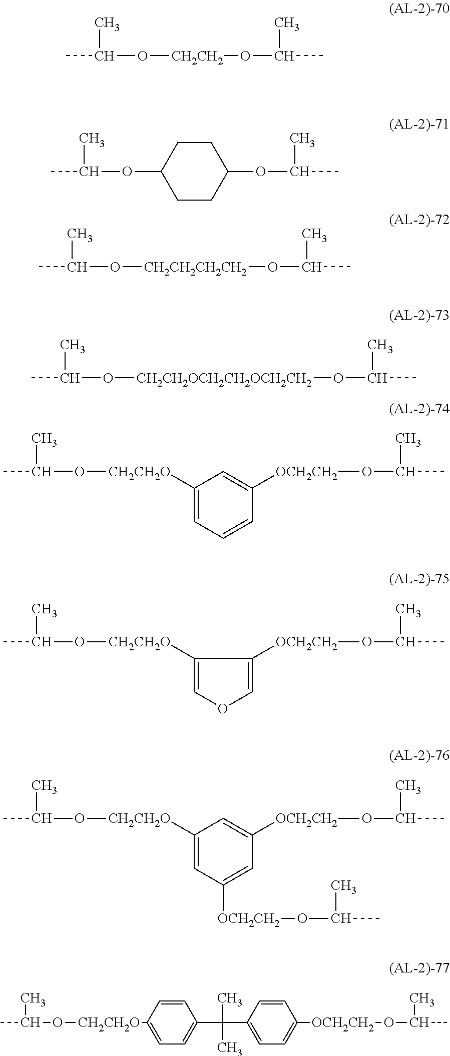

In formula (AL-3), R^{L5}, R^{L6} and R^{L7} are each independently a C₁-C₂₀ hydrocarbyl group which may contain a heteroatom such as oxygen, sulfur, nitrogen or fluorine. The hydrocarbyl group may be saturated or unsaturated and straight, branched or cyclic. Examples thereof include C₁-C₂₀ alkyl groups, C₃-C₂₀ cyclic saturated hydrocarbyl groups, C₂-C₂₀ alkenyl groups, C₃-C₂₀ cyclic unsaturated aliphatic hydrocarbyl groups, and C₆-C₁₀ aryl groups. A pair of R^{L5} and R^{L6}, R^{L5} and R^{L7}, or R^{L6} and R^{L7} may bond together to form a C₃-C₂₀ aliphatic ring with the carbon atom to which they are attached.

Examples of the group having formula (AL-3) include tert-butyl, 1,1-diethylpropyl, 1-ethylnorbornyl, 1-methyl cyclopentyl, 1-ethylcyclopentyl, 1-isopropylcyclopentyl, 1-methylcyclohexyl, 2-(2-methyl)adamantyl, 2-(2-ethyl)adamantyl, and tert-pentyl.

Examples of the group having formula (AL-3) also include groups having the formulae (AL-3)-1 to (AL-3)-19.

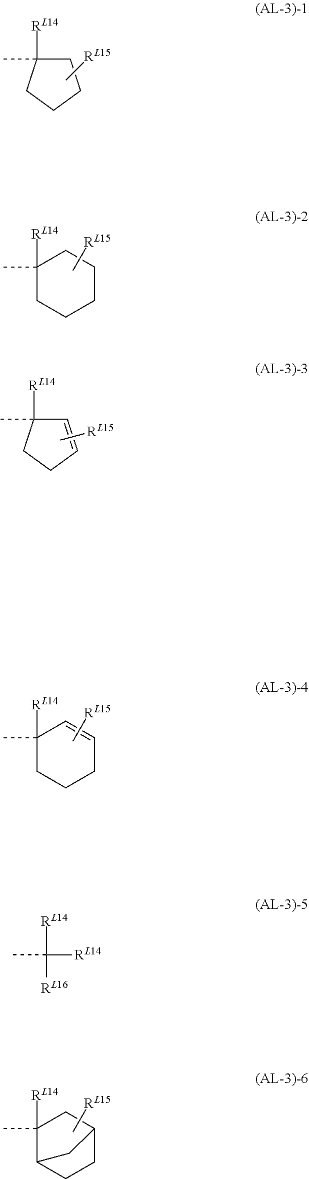

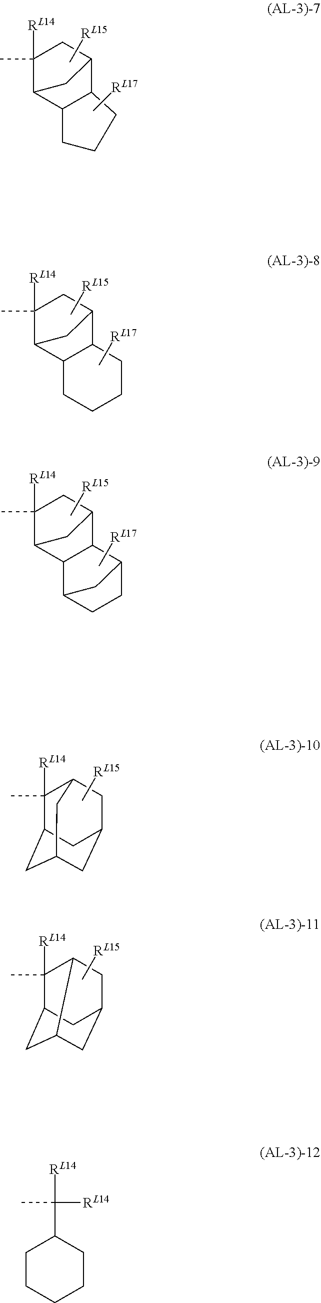

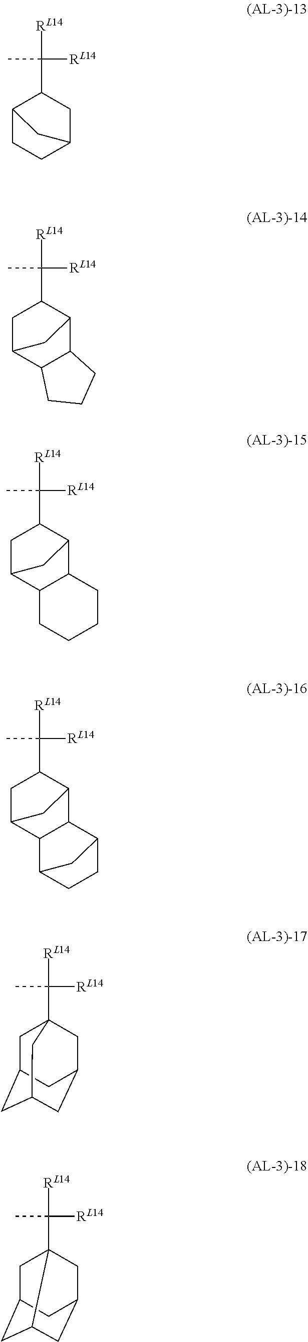

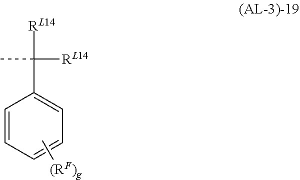

In formulae (AL-3)-1 to (AL-3)-19, R^{L14} is each independently a C₁-C₈ saturated hydrocarbyl group or C₆-C₂₀ aryl group. R^{L15} and R^{L17} are each independently hydrogen or a C₁-C₂₀ saturated hydrocarbyl group. R^{L16} is a C₆-C₂₀ aryl group. The saturated hydrocarbyl group may be straight, branched or cyclic. Typical of the aryl group is phenyl. R^{F} is fluorine or trifluoromethyl, and g is an integer of 1 to 5.

Other examples of the group having formula (AL-3) include groups having the formulae (AL-3)-20 and (AL-3)-21. The base polymer may be crosslinked within the molecule or between molecules with these acid labile groups.

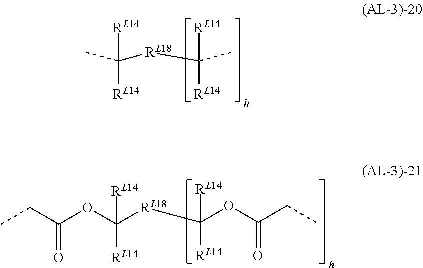

In formulae (AL-3)-20 and (AL-3)-21, R^{L14} is as defined above. R^{L18} is a C₁-C₂₀ (h+1)-valent saturated hydrocarbylene group or C₆-C₂₀ (h+1)-valent arylene group, which may contain a heteroatom such as oxygen, sulfur or nitrogen. The saturated hydrocarbylene group may be straight, branched or cyclic. The subscript h is an integer of 1 to 3

Examples of the monomer from which repeat units containing an acid labile group of formula (AL-3) are derived include (meth)acrylates having an exo-form structure represented by the formula (AL-3)-22.

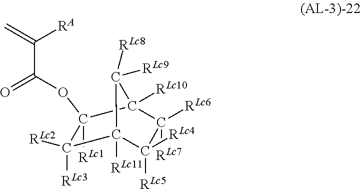

In formula (AL-3)-22, R^{A} is as defined above. R^{Lc1} is a C₁-C₈ saturated hydrocarbyl group or an optionally substituted C₆-C₂₀ aryl group; the saturated hydrocarbyl group may be straight, branched or cyclic. R^{Lc2} to R^{Lc111} are each independently hydrogen or a C₁-C₁₅ hydrocarbyl group which may contain a heteroatom; oxygen is a typical heteroatom. Suitable hydrocarbyl groups include C₁-C₁₅ alkyl groups and C₆-C₁₅ aryl groups. Alternatively, a pair of R^{Lc2} and R^{Lc3}, R^{Lc4} and R^{Lc6}, R^{Lc4} and R^{Lc7}, R^{Lc5} and R^{Lc7}, R^{Lc5} and R^{Lc11}, R^{Lc6} and R^{Lc10}, R^{Lc8} and R^{Lc9}, or R^{Lc9} and R^{Lc10}, taken together, may form a ring with the carbon atom to which they are attached, and in this event, the ring-forming group is a C₁-C₁₅ hydrocarbylene group which may contain a heteroatom. Also, a pair of R^{Lc2} and R^{Lc11}, R^{Lc8} and R^{Lc11}, or R^{Lc4} and R^{Lc6} which are attached to vicinal carbon atoms may bond together directly to form a double bond. The formula also represents an enantiomer.

Examples of the monomer having formula (AL-3)-22 are described in U.S. Pat. No. 6,448,420 (JP-A 2000-327633). Illustrative non-limiting examples of suitable monomers are given below. R^{A} is as defined above.

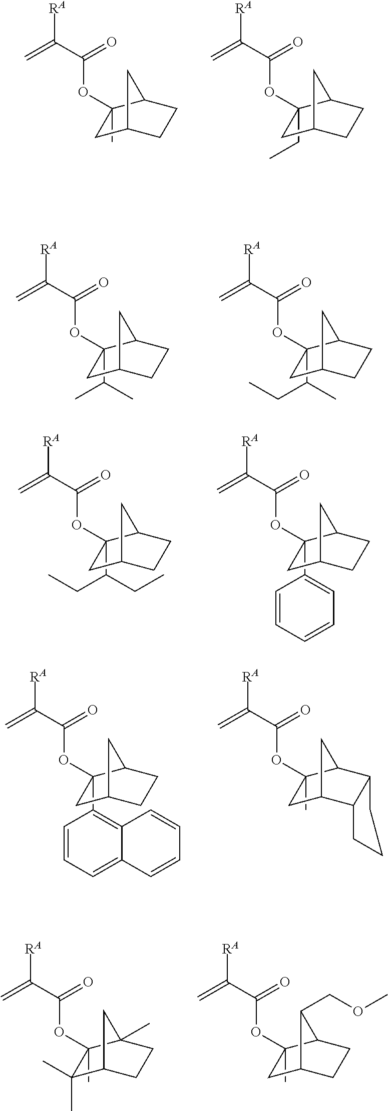

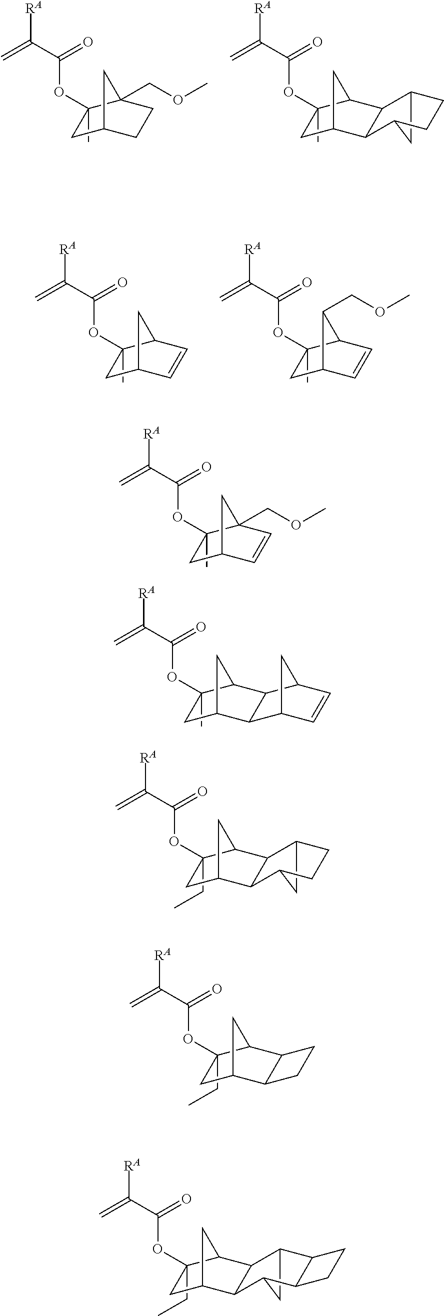

Examples of the monomer from which the repeat units having an acid labile group of formula (AL-3) are derived include (meth)acrylates having a furandiyl, tetrahydrofurandiyl or oxanorbornanediyl group as represented by the following formula (AL-3)-23.

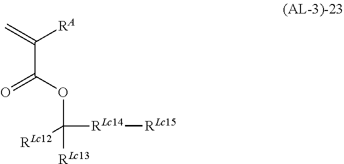

In formula (AL-3)-23, R^{A} is as defined above. R^{Lc12} and R^{Lc13} are each independently a C₁-C₁₀ hydrocarbyl group, or R^{Lc12} and R^{Lc13}, taken together, may form an aliphatic ring with the carbon atom to which they are attached. R^{Lc14} is furandiyl, tetrahydrofurandiyl or oxanorbornanediyl. R^{Lc15} is hydrogen or a C₁-C₁₀ hydrocarbyl group which may contain a heteroatom. The hydrocarbyl group may be straight, branched or cyclic, and is typically a C₁-C₁₀ saturated hydrocarbyl group.

Examples of the monomer having formula (AL-3)-23 are shown below, but not limited thereto. Herein R^{A} is as defined above.

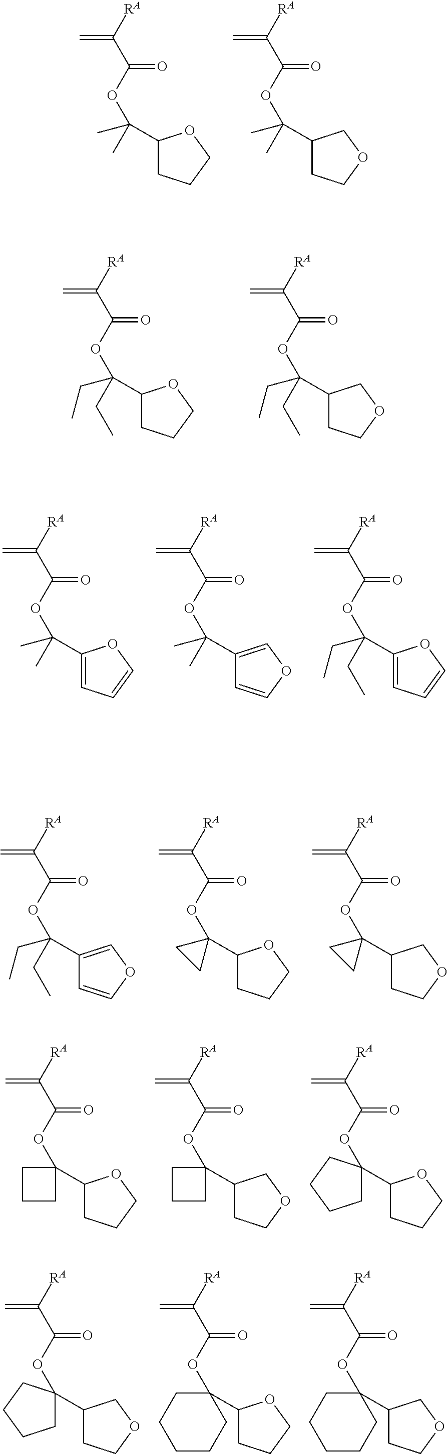

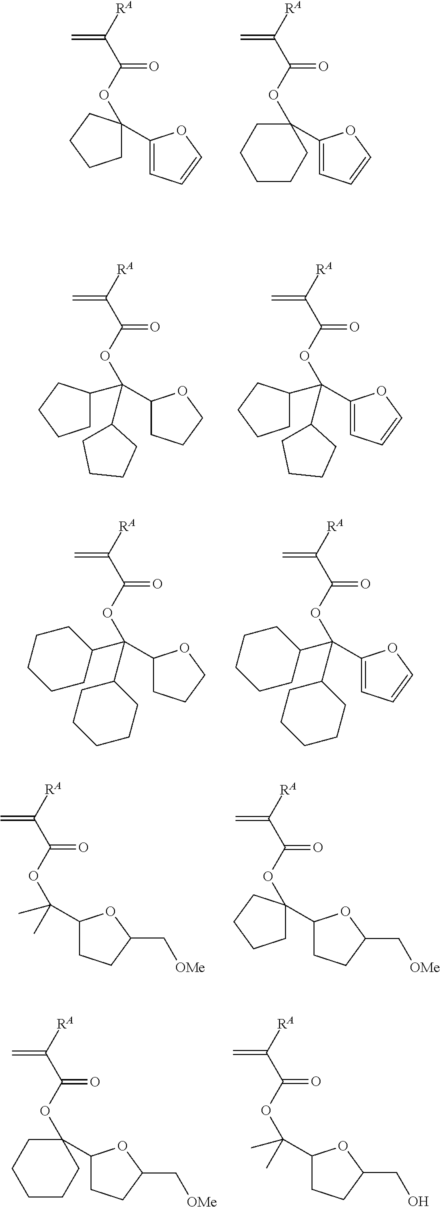

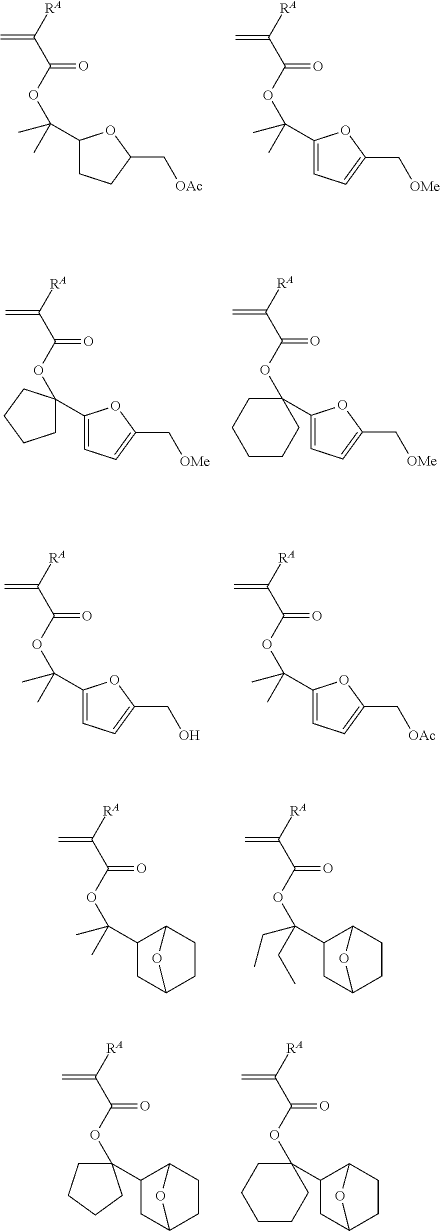

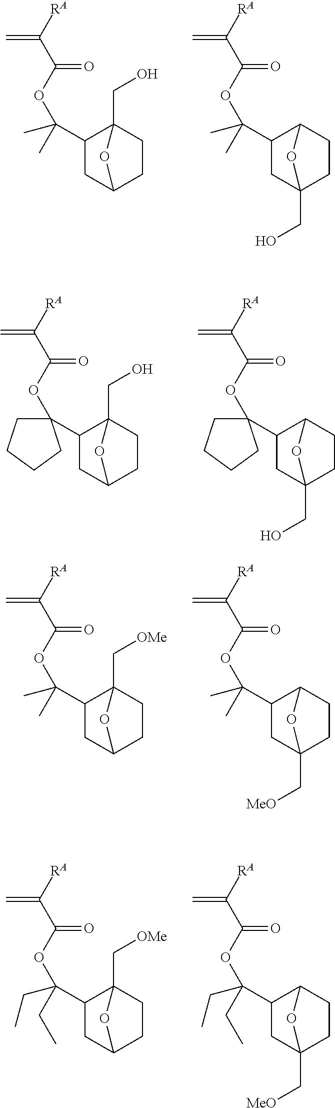

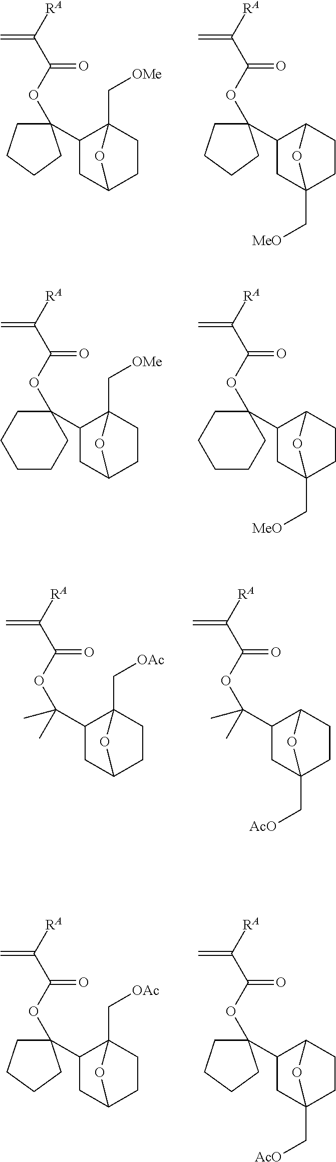

The base polymer may further include repeat units (b) having an adhesive group. The adhesive group is selected from hydroxy, carboxy, lactone ring, carbonate bond, thiocarbonate bond, carbonyl, cyclic acetal, ether bond, ester bond, sulfonic ester bond, cyano, amide bond, —O—C(═O)—S—, and —O—C(═O)—NH—.

Examples of the monomer from which repeat units (b) are derived are given below, but not limited thereto. Herein R^{A} is as defined above.

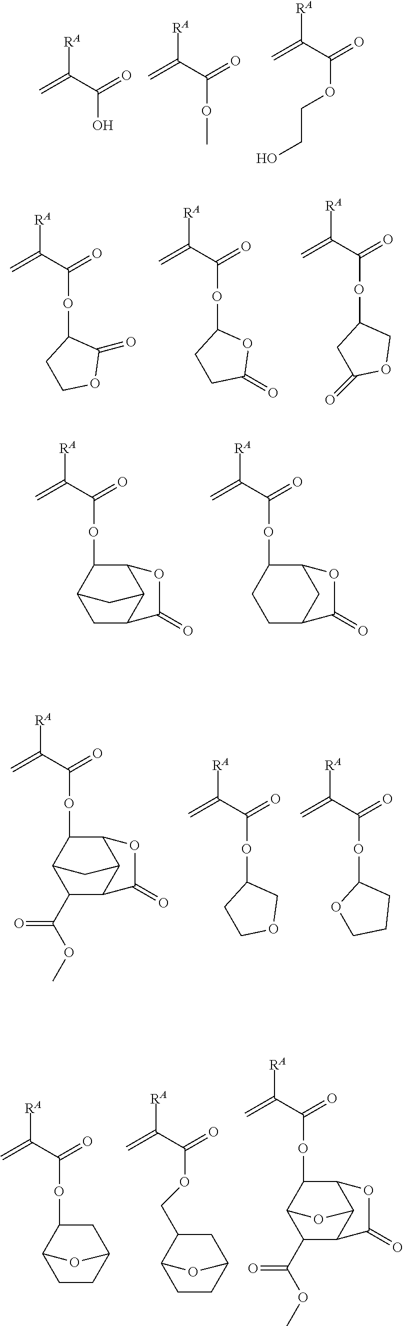

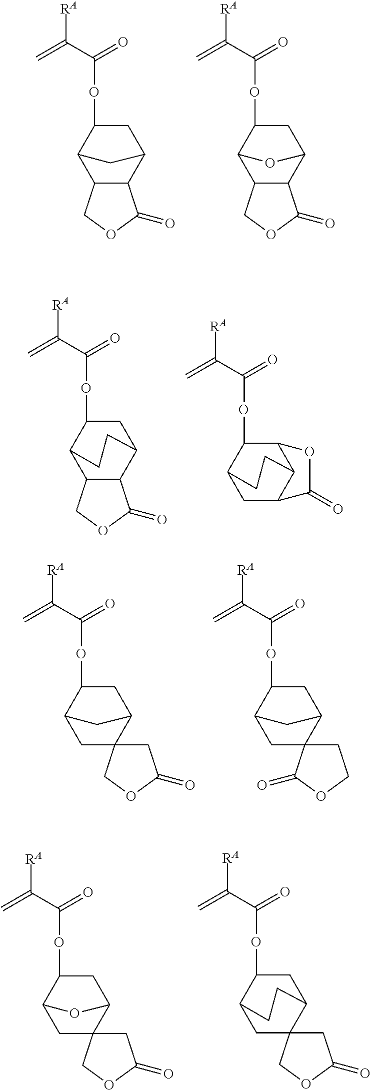

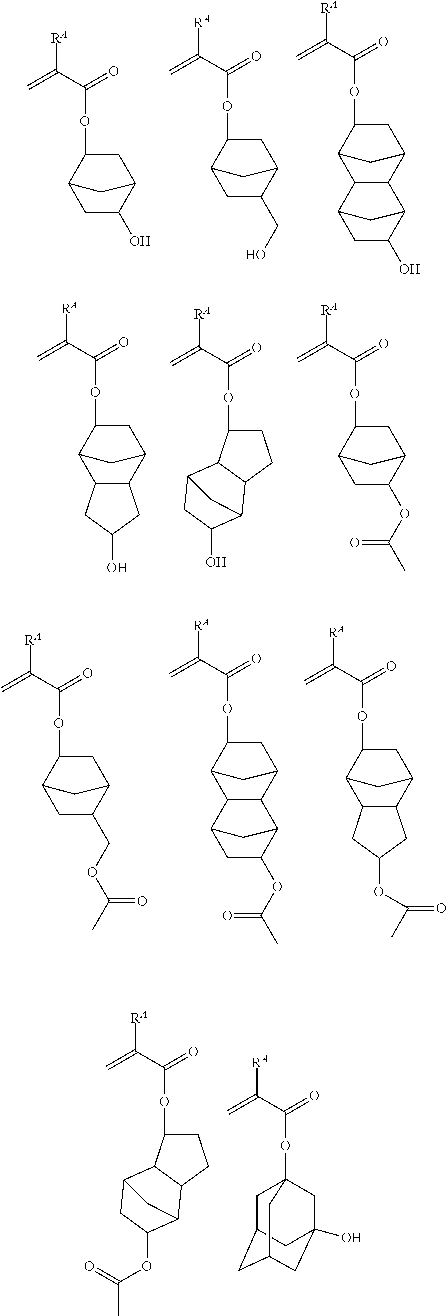

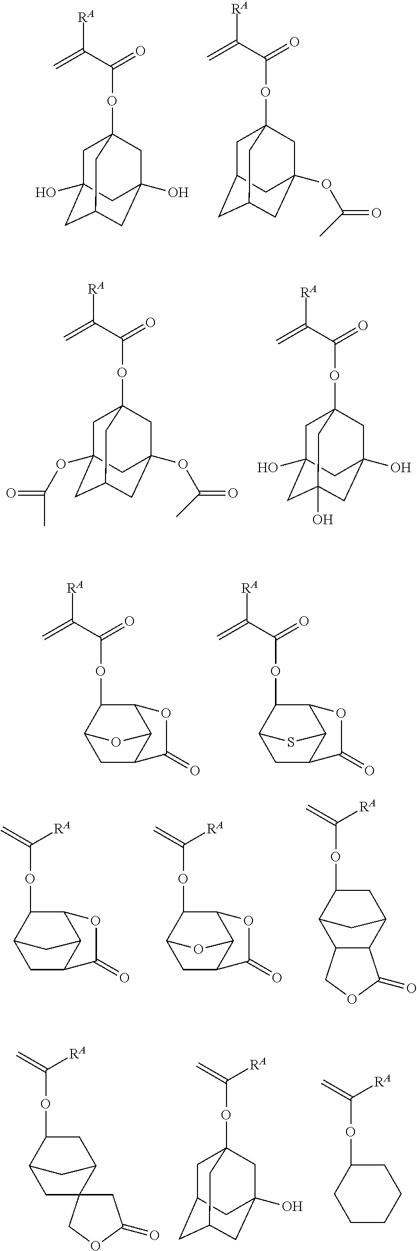

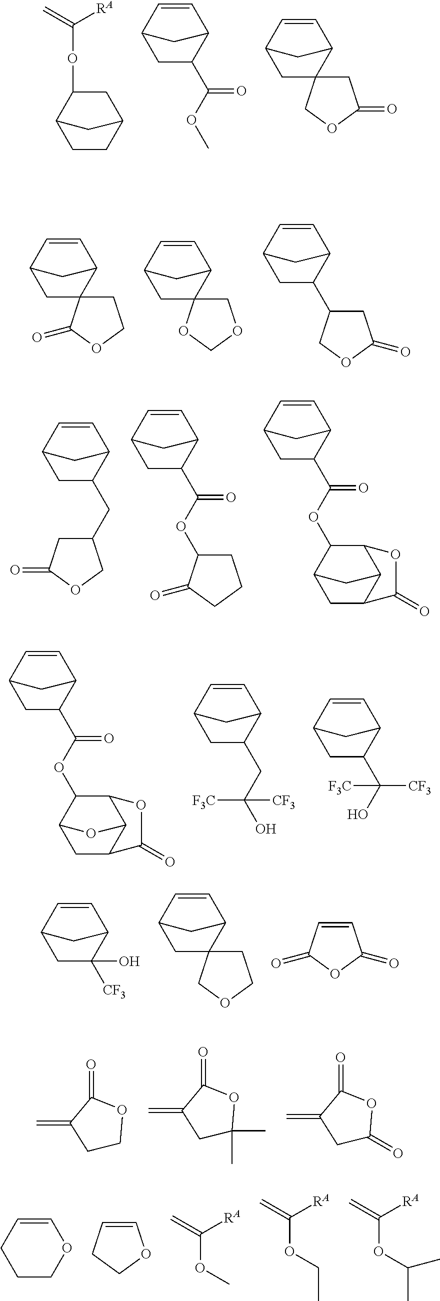

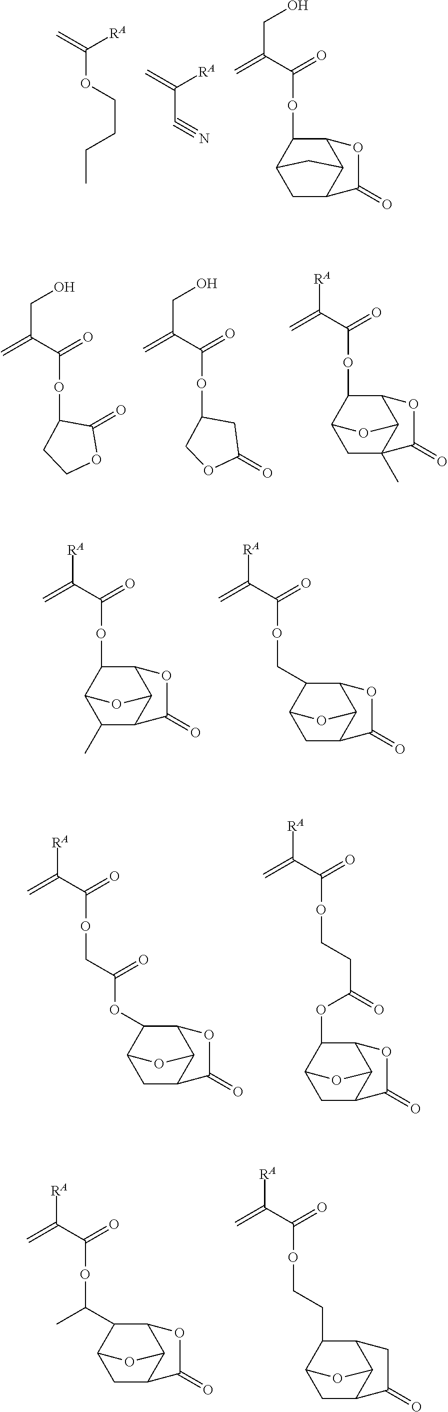

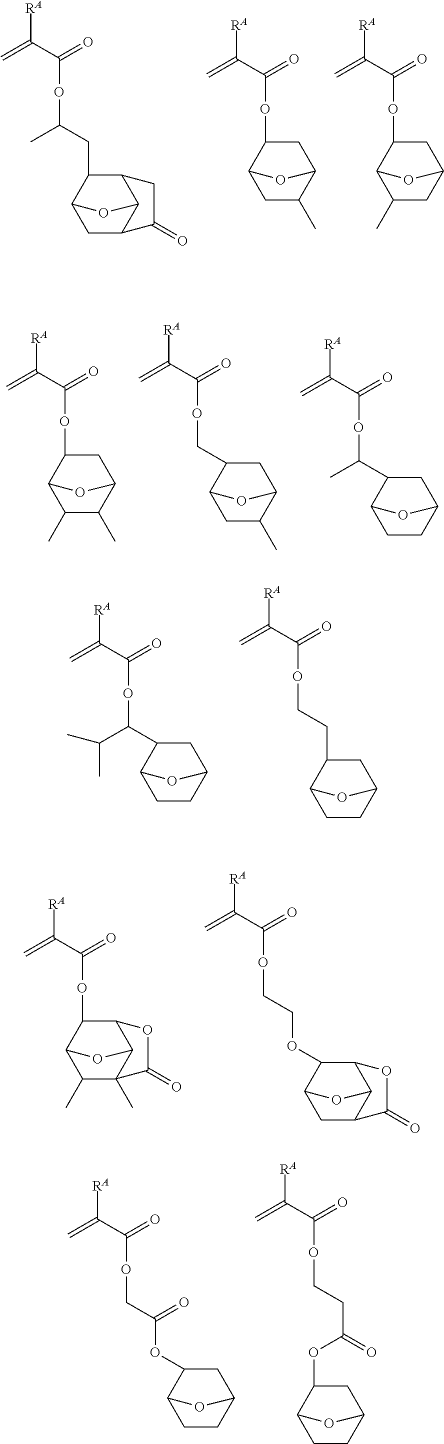

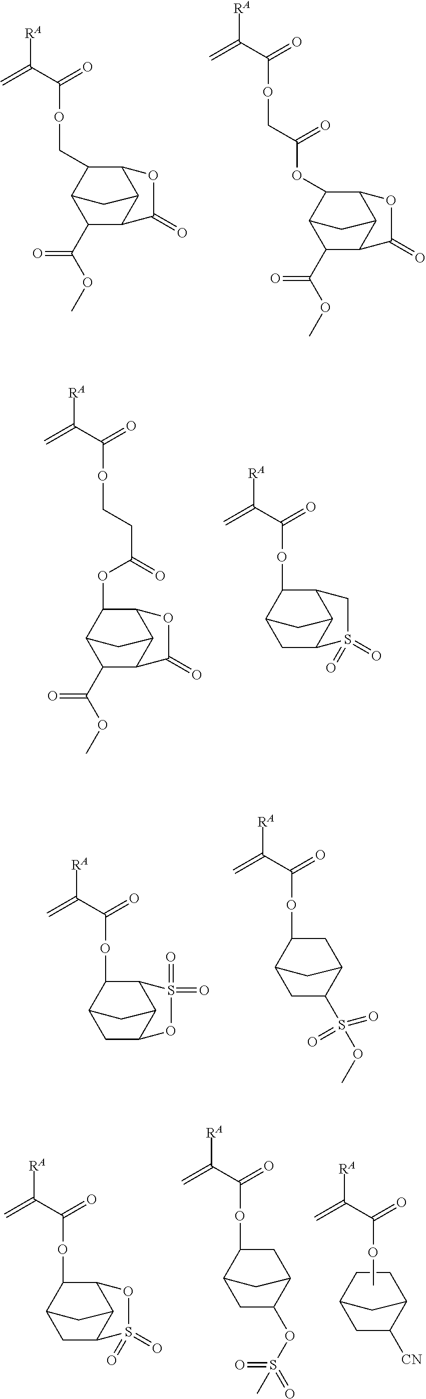

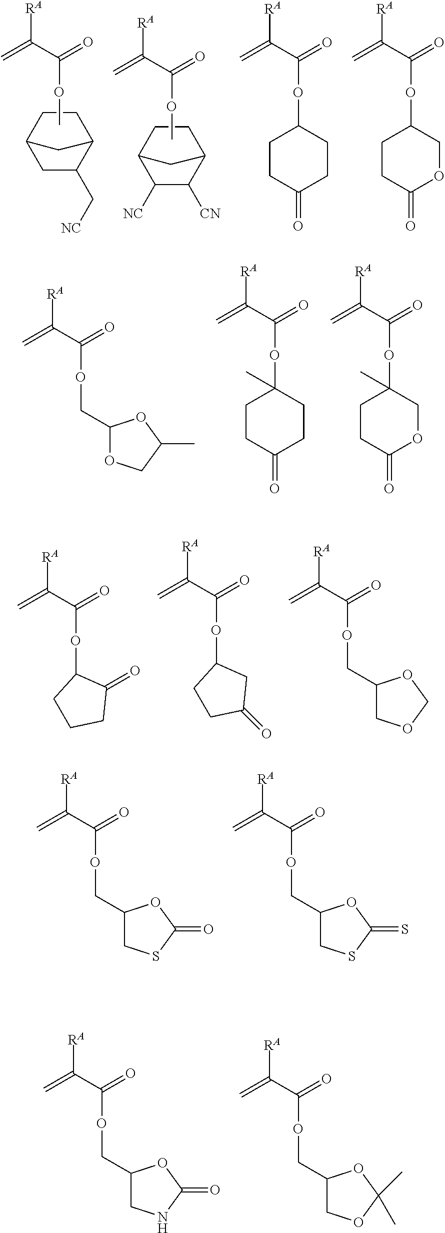

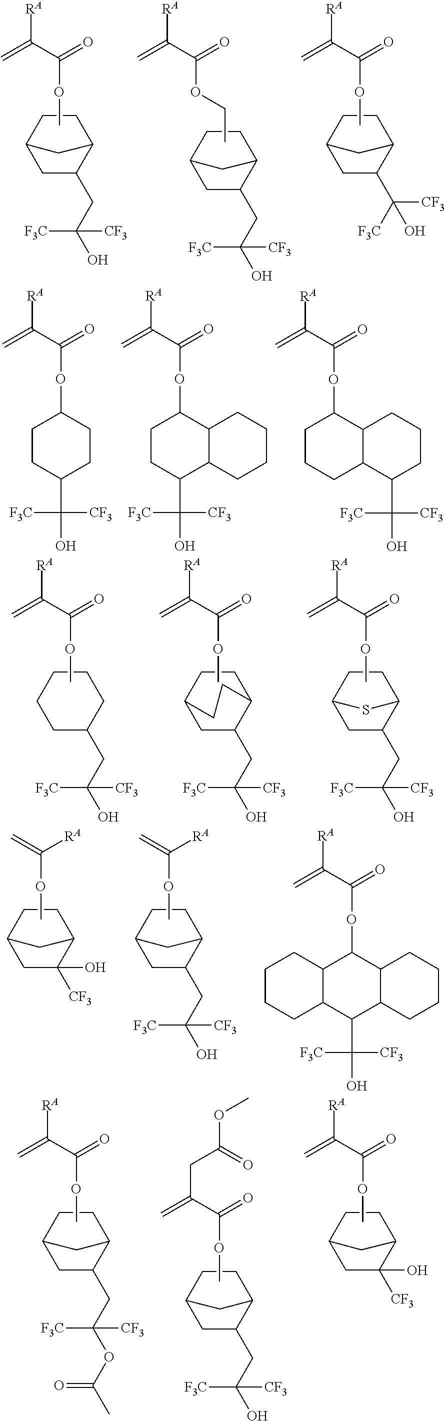

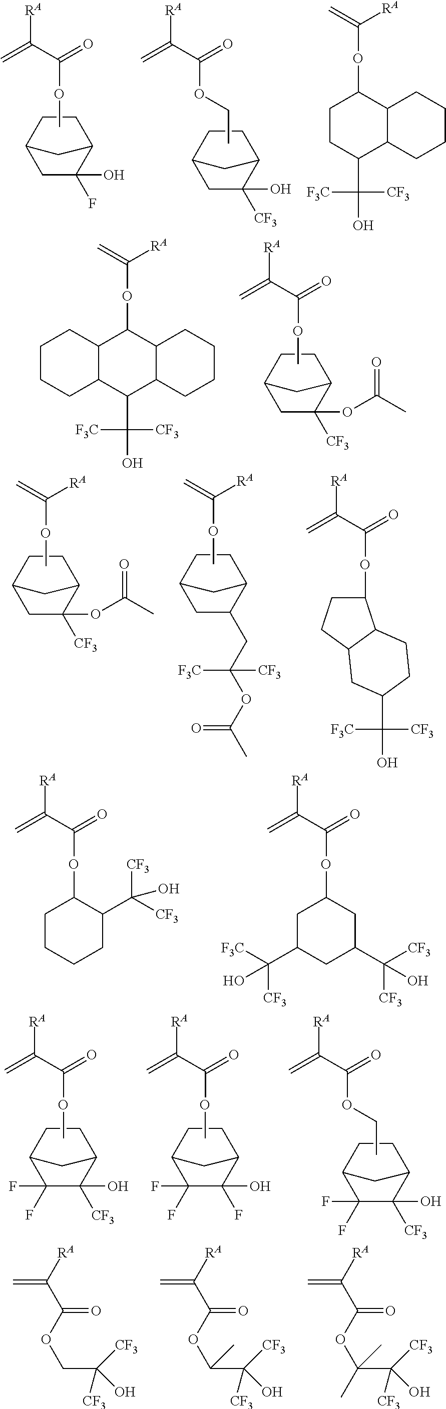

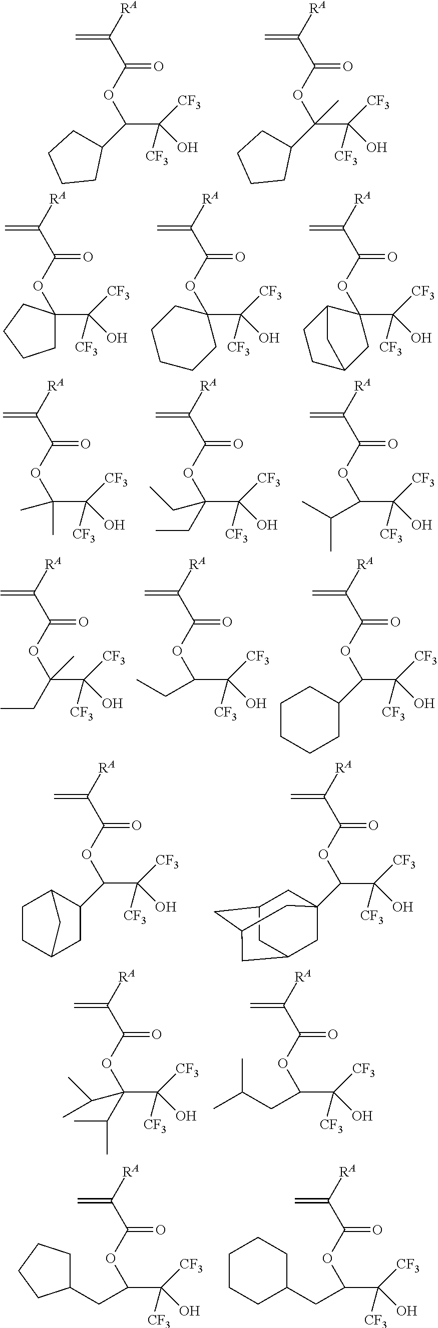

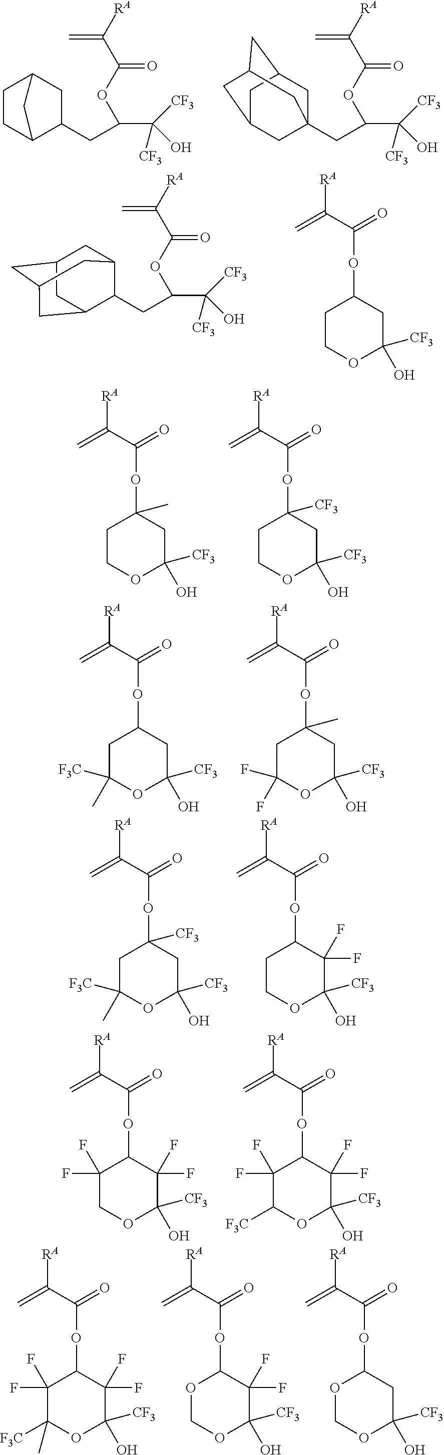

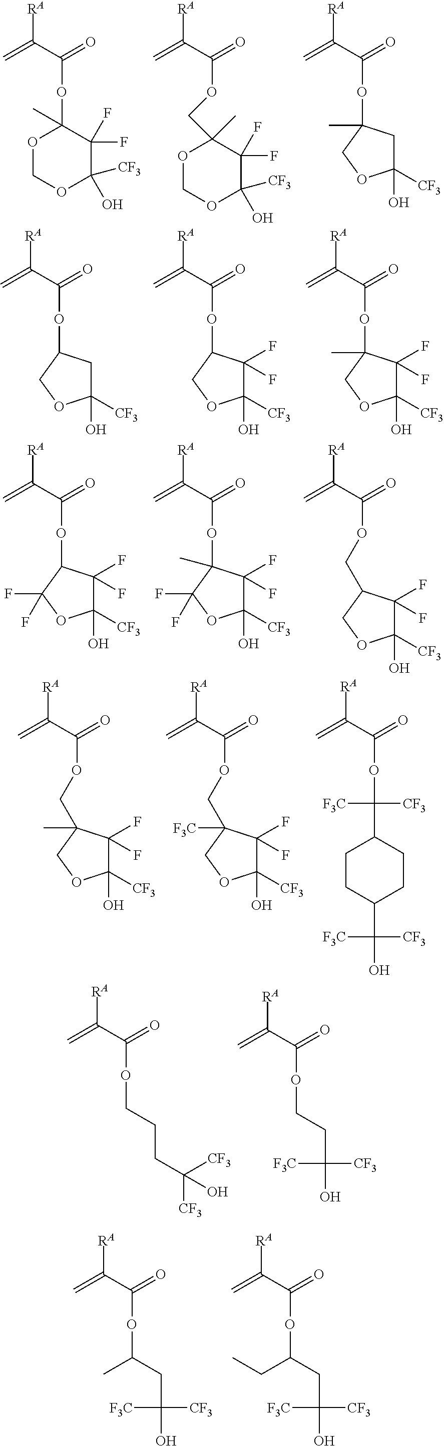

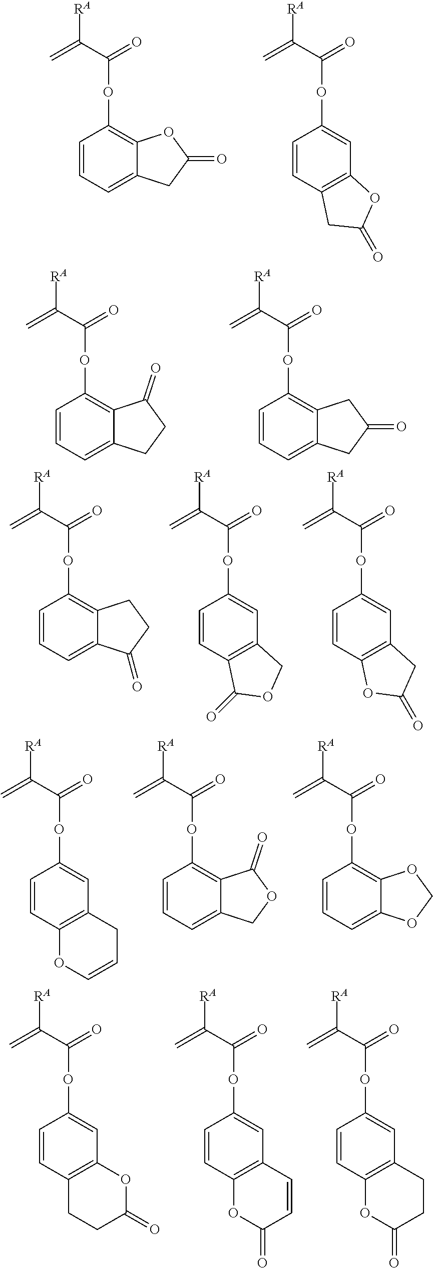

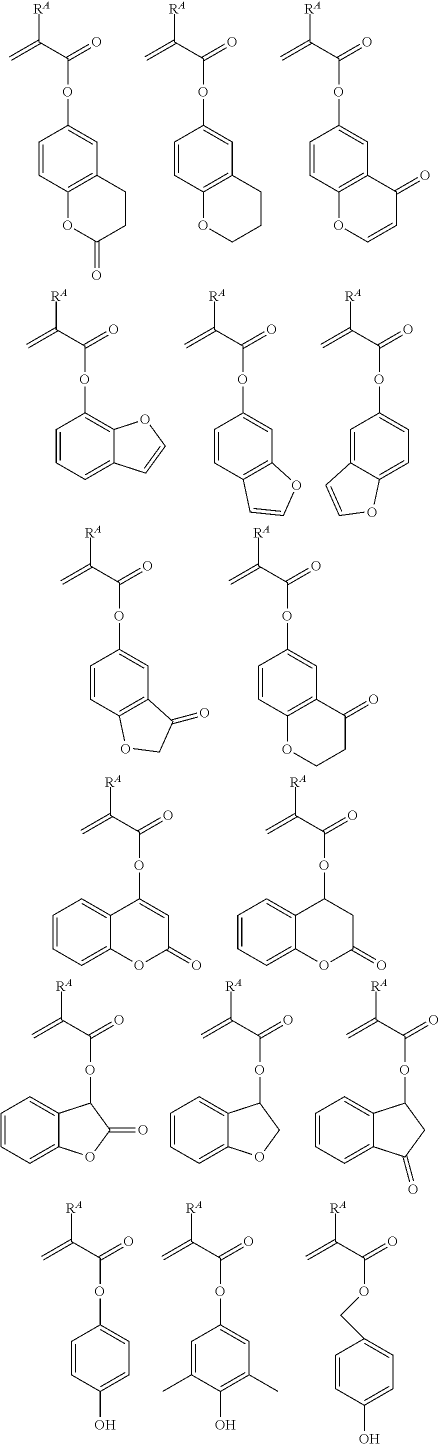

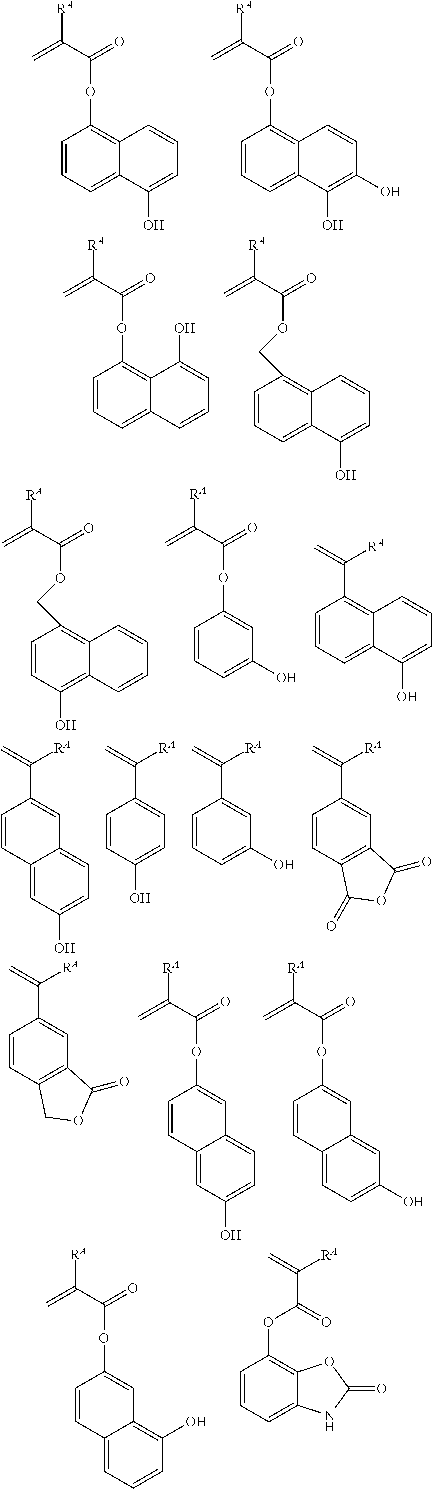

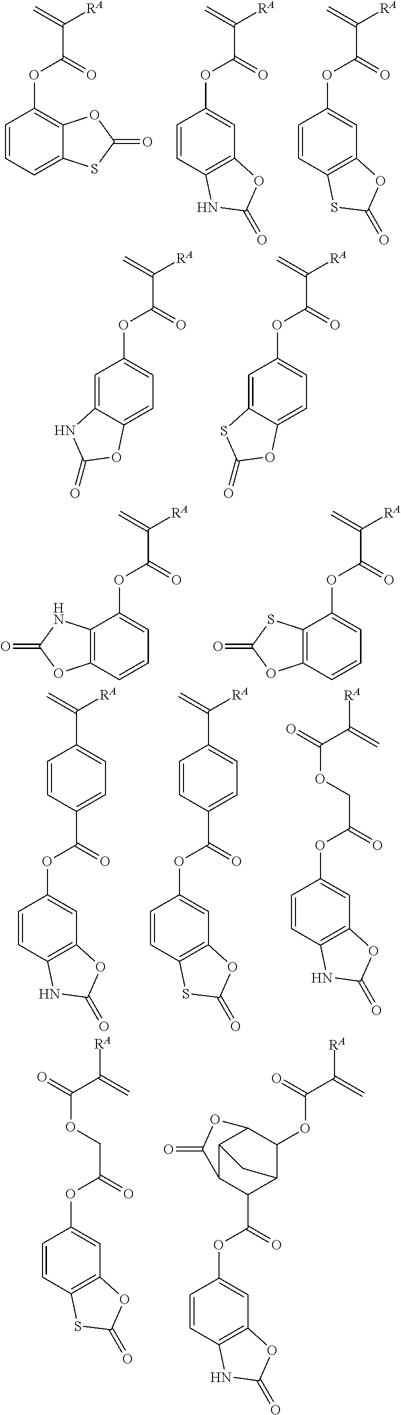

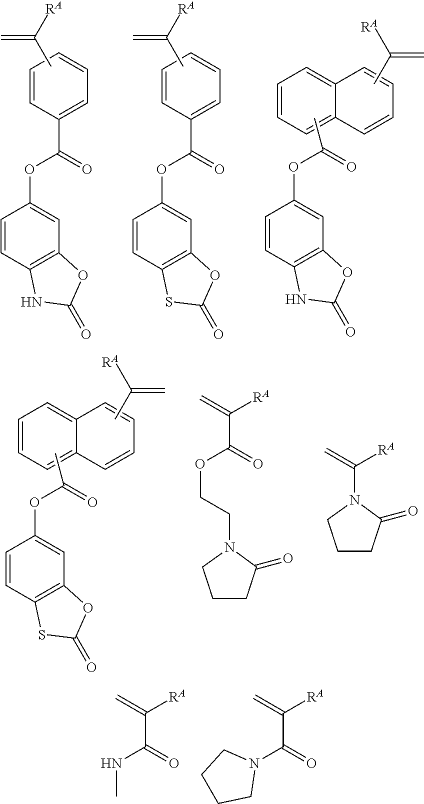

The base polymer may further include repeat units (c) of at least one type selected from repeat units having the following formulae (c1), (c2) and (c3). These units are simply referred to as repeat units (c1), (c2) and (c3), which may be used alone or in combination of two or more types.

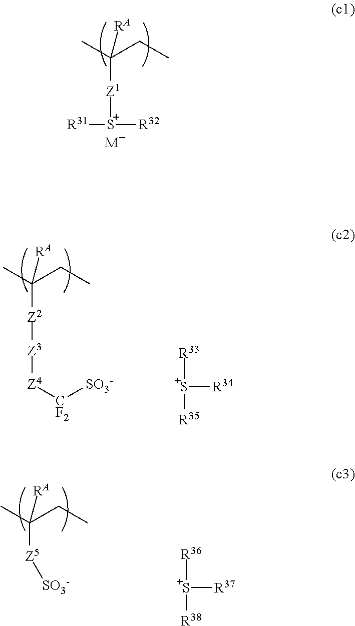

In formulae (c1) to (c3), R^{A} is each independently hydrogen or methyl. Z¹ is a single bond, or a C₁-C₆ aliphatic hydrocarbylene group, phenylene group, naphthylene group, or a C₇-C₁₈ group obtained by combining the foregoing, or —O—Z″—, —C(═O)—O—Z″— or —C(═O)—NH—Z¹¹—, wherein Z¹¹ is a C₁-C₆ aliphatic hydrocarbylene group, phenylene group, naphthylene group, or a C₇-C₁₈ group obtained by combining the foregoing, which may contain a carbonyl moiety, ester bond, ether bond or hydroxy moiety. Z² is a single bond or ester bond. Z³ is a single bond, —Z³¹—C(═O)—O—, —Z³¹—O—, or —Z³¹—O—C(═O)—, wherein Z³¹ is a C₁-C₁₂ hydrocarbylene group, phenylene group or a C₇-C₁₈ group obtained by combining the foregoing, which may contain a carbonyl moiety, ester bond, ether bond, bromine or iodine. Z⁴ is methylene, 2,2,2-trifluoro-1,1-ethanediyl or carbonyl. Z⁵ is a single bond, methylene, ethylene, phenylene, fluorinated phenylene, trifluoromethyl-substituted phenylene group, —O—Z⁵¹—, —C(═O)—O—Z⁵¹— or —C(═O)—NH—Z⁵¹—, wherein Z⁵¹ is a C₁-C₆ aliphatic hydrocarbylene group, phenylene group, fluorinated phenylene group, or trifluoromethyl-substituted phenylene group, which may contain a carbonyl moiety, ester bond, ether bond or hydroxy moiety.

In formulae (c1) to (c3), R³¹ to R³⁸ are each independently halogen or a C₁-C₂₀ hydrocarbyl group which may contain a heteroatom. The hydrocarbyl group may be saturated or unsaturated and straight, branched or cyclic. Examples thereof are as exemplified above for the hydrocarbyl groups R² to R⁴ in formula (1).

In formula (c1), M⁻ is a non-nucleophilic counter ion. Examples of the non-nucleophilic counter ion include halide ions such as chloride and bromide ions; fluoroalkylsulfonate ions such as triflate, 1,1,1-trifluoroethanesulfonate, and nonafluorobutanesulfonate; arylsulfonate ions such as tosylate, benzenesulfonate, 4-fluorobenzenesulfonate, and 1,2,3,4,5-pentafluorobenzenesulfonate; alkylsulfonate ions such as mesylate and butanesulfonate; imide ions such as bis(trifluoromethylsulfonyl)imide, bis(perfluoroethylsulfonyl)imide and bis(perfluorobutylsulfonyl)imide; methide ions such as tris(trifluoromethylsulfonyl)methide and tris(perfluoroethylsulfonyl)methide.

Also included are sulfonate ions having fluorine substituted at α-position as represented by the formula (c1-1) and sulfonate ions having fluorine substituted at α-position and trifluoromethyl at β-position as represented by the formula (c1-2).

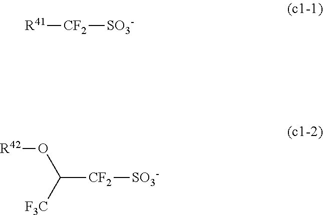

In formula (c1-1), R⁴¹ is hydrogen, or a C₁-C₂₀ hydrocarbyl group which may contain an ether bond, ester bond, carbonyl moiety, lactone ring, or fluorine atom. The hydrocarbyl group may be saturated or unsaturated and straight, branched or cyclic and examples thereof are as exemplified above for the hydrocarbyl group R^{11A} in formula (2-1-1).

In formula (c1-2), R⁴² is hydrogen, or a C₁-C₃₀ hydrocarbyl group or C₂-C₃₀ hydrocarbylcarbonyl group which may contain an ether bond, ester bond, carbonyl moiety or lactone ring. The hydrocarbyl group and hydrocarbyl moiety in the hydrocarbylcarbonyl group may be saturated or unsaturated and straight, branched or cyclic and examples thereof are as exemplified above for the hydrocarbyl group R^{11A} in formula (2-1-1).

Examples of the cation in the monomer from which repeat unit (c1) is derived are shown below, but not limited thereto. R^{A} is as defined above.

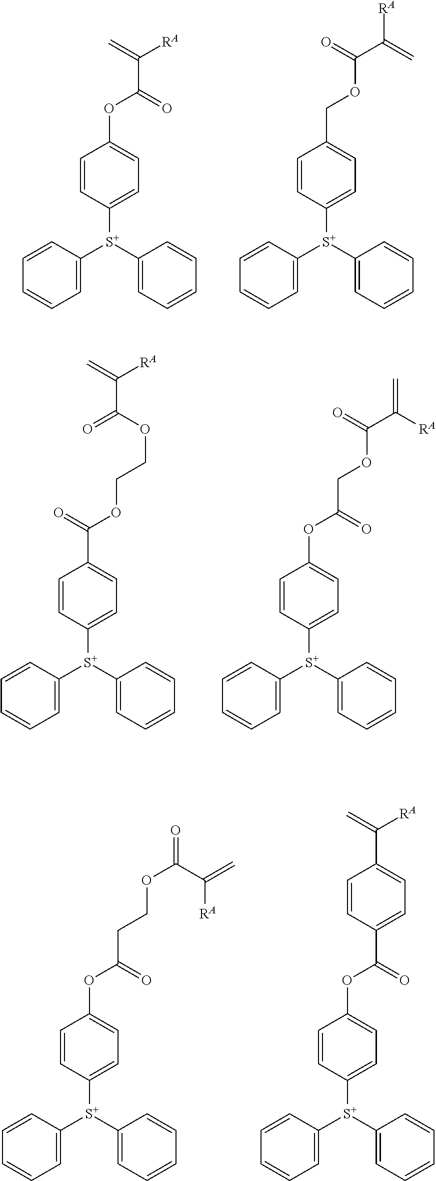

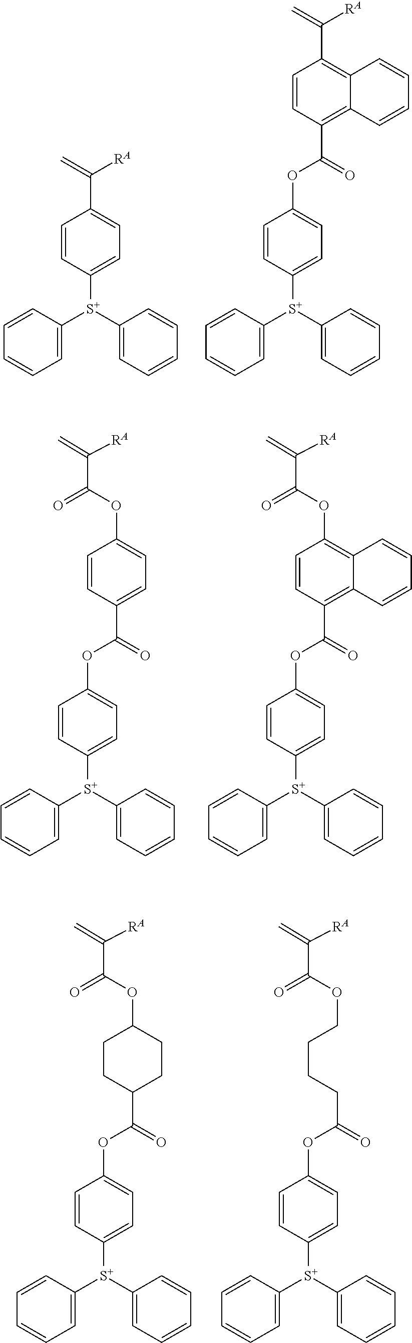

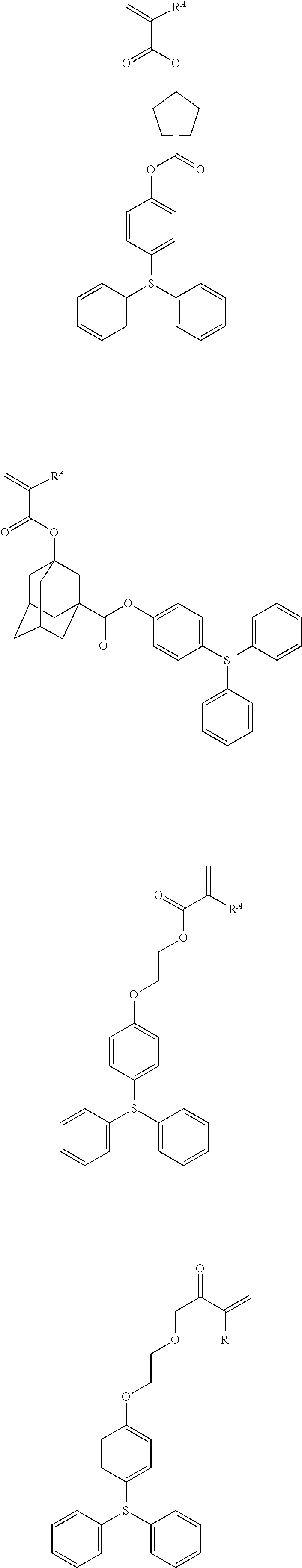

Examples of the cation in the monomer from which repeat unit (c2) or (c3) is derived are as exemplified above for the cation in the sulfonium salt having formula (1).

Examples of the anion in the monomer from which repeat unit (c2) is derived are shown below, but not limited thereto. R^{A} is as defined above.

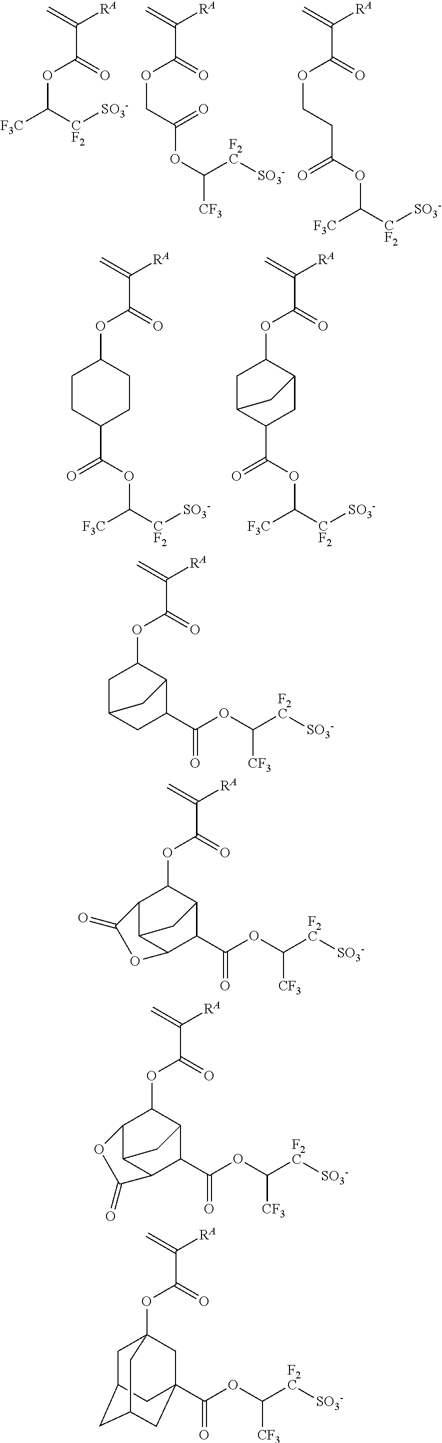

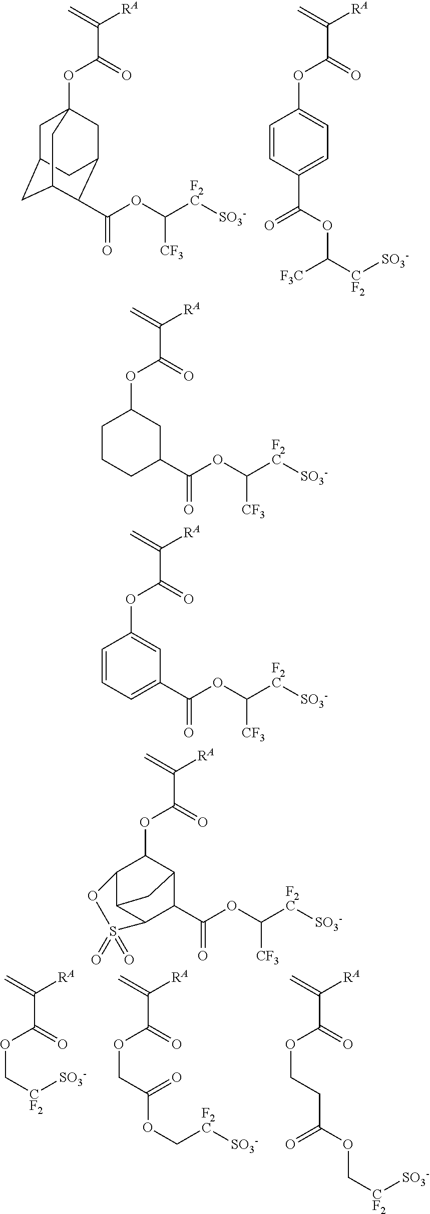

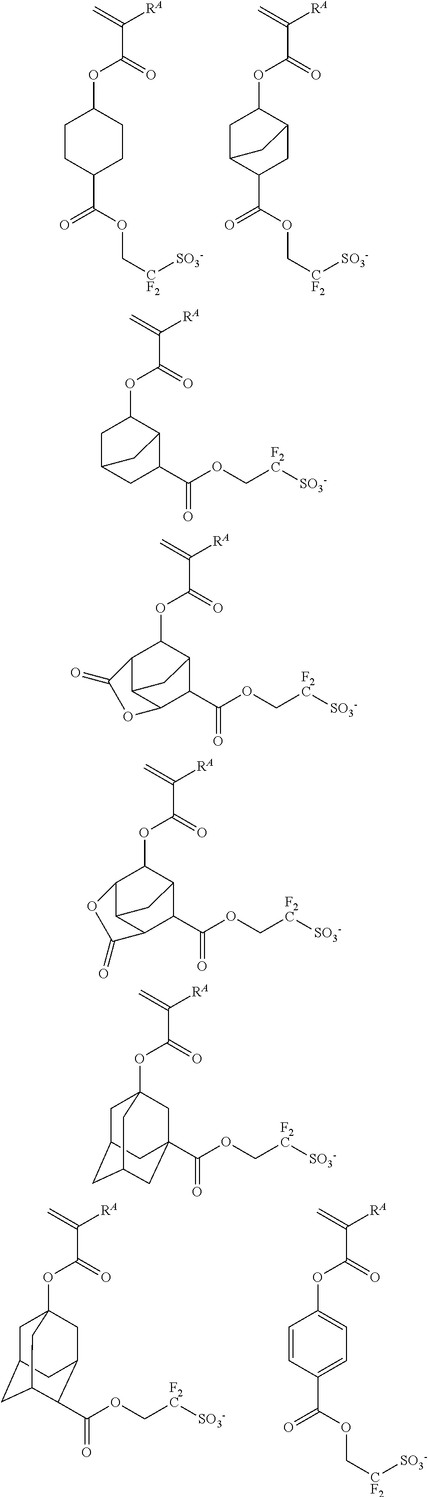

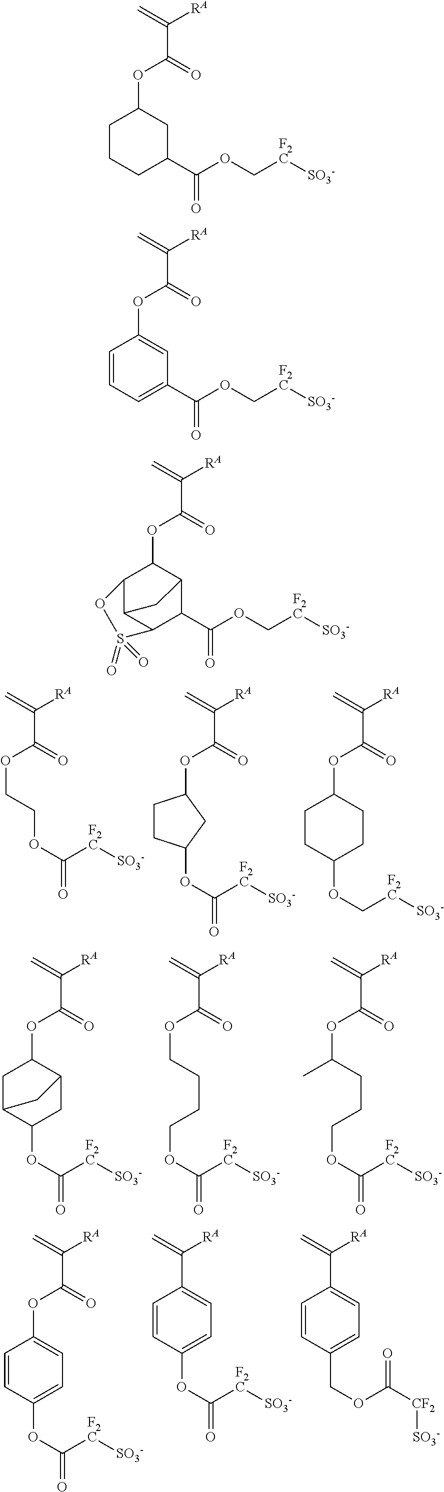

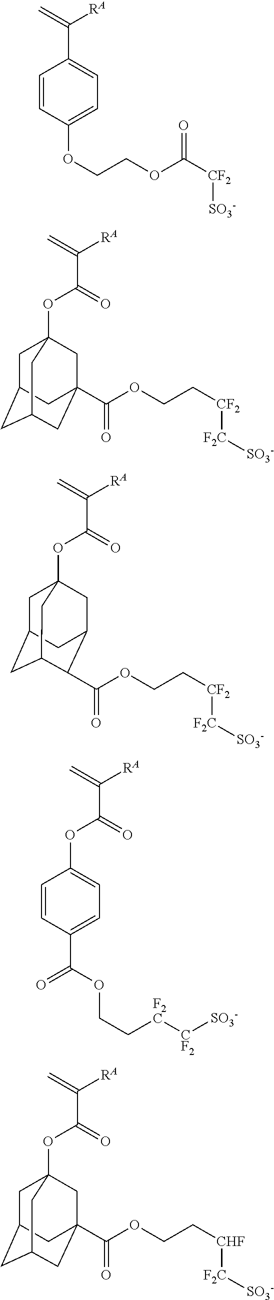

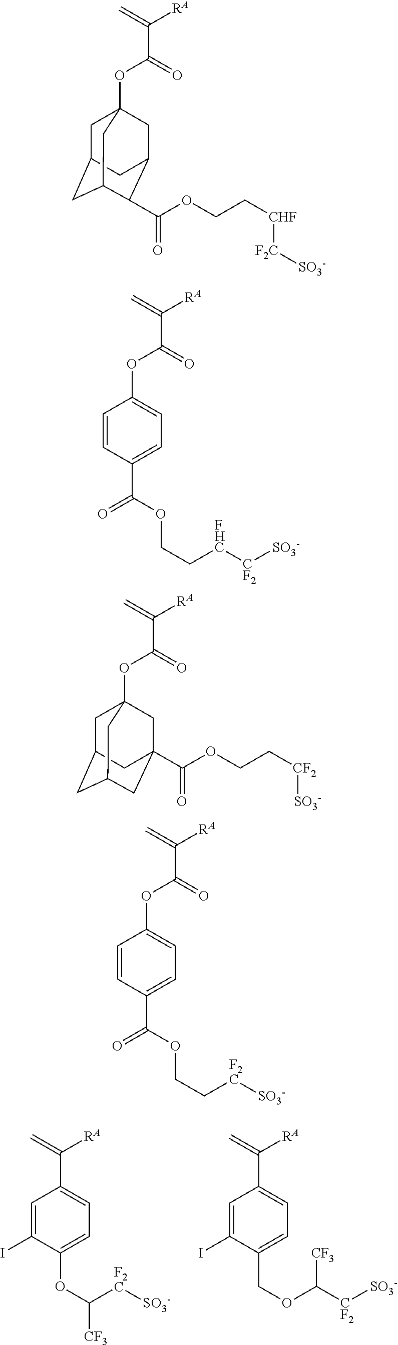

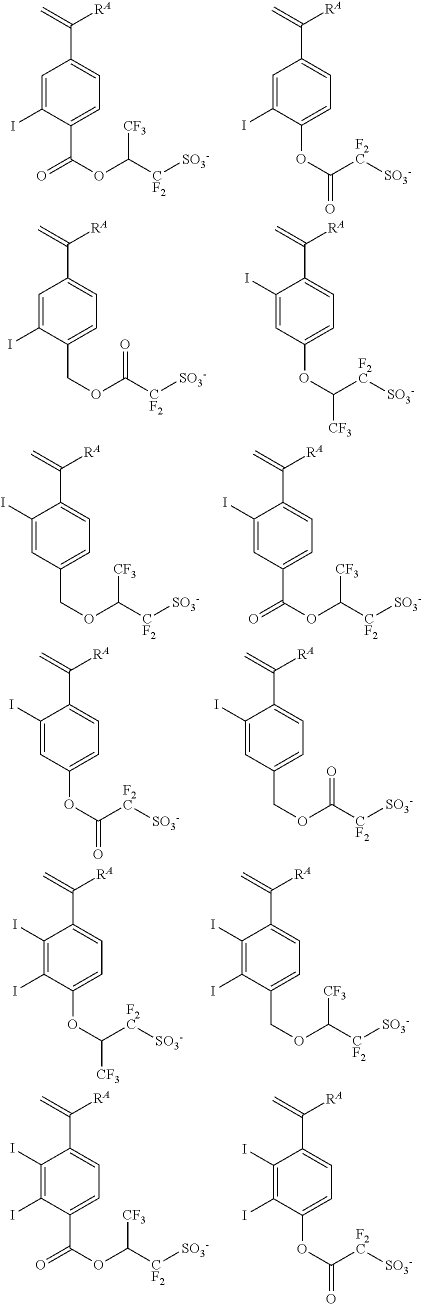

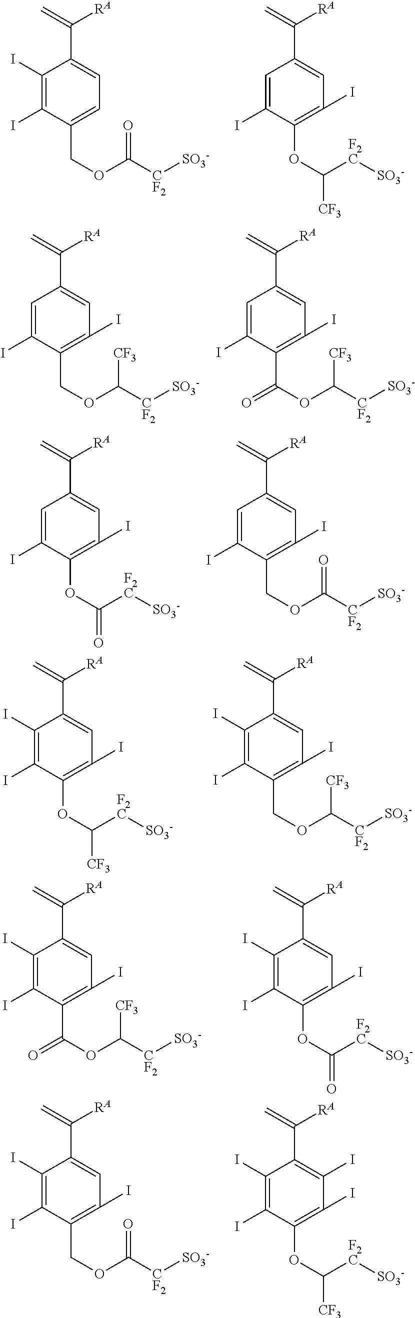

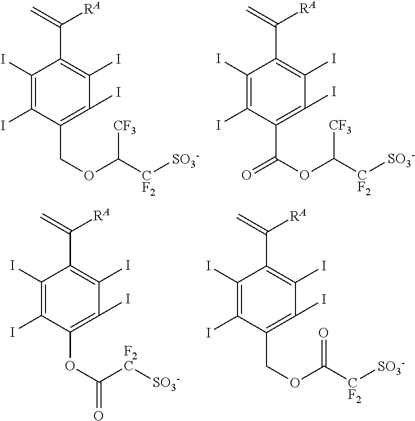

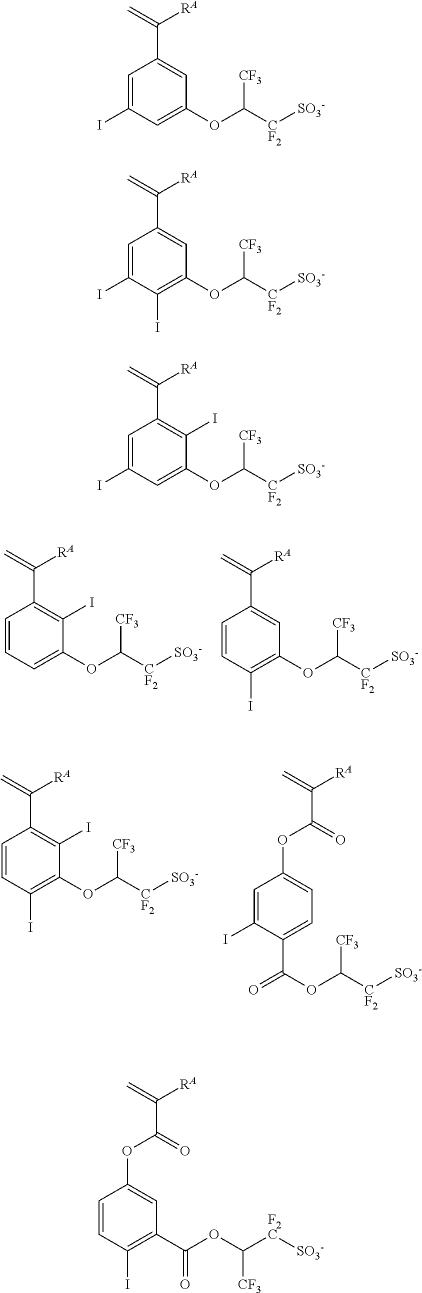

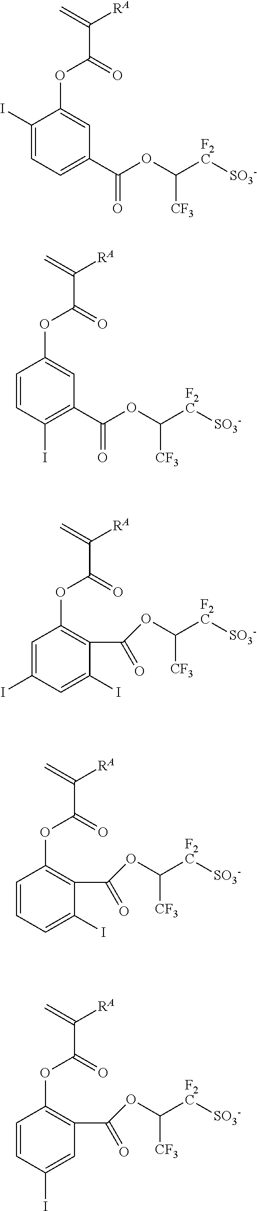

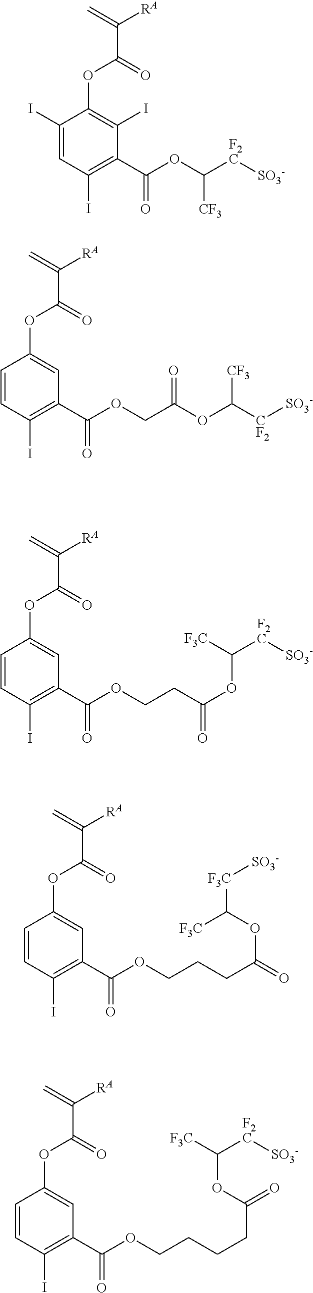

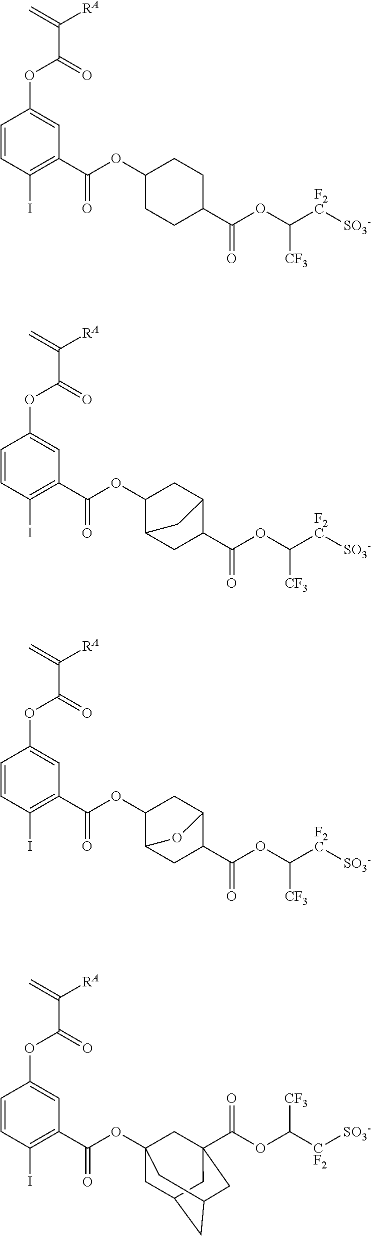

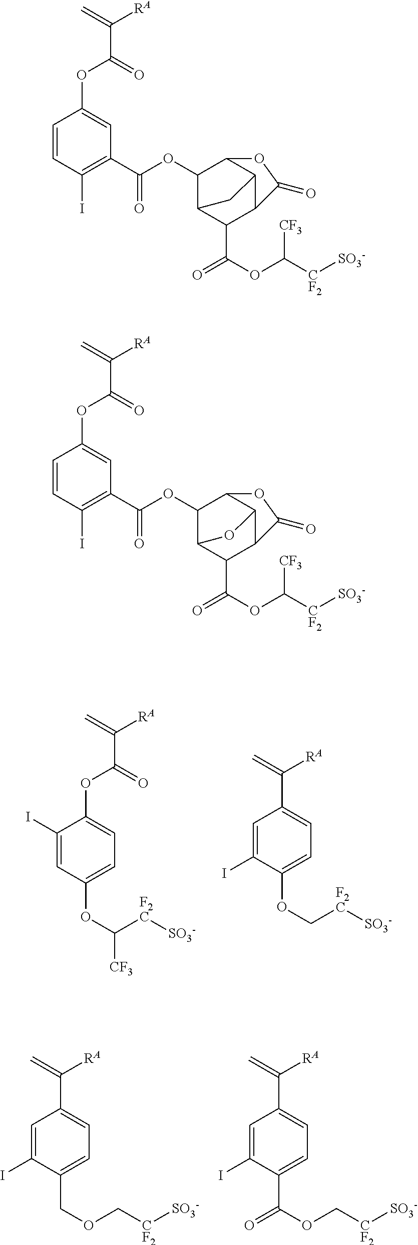

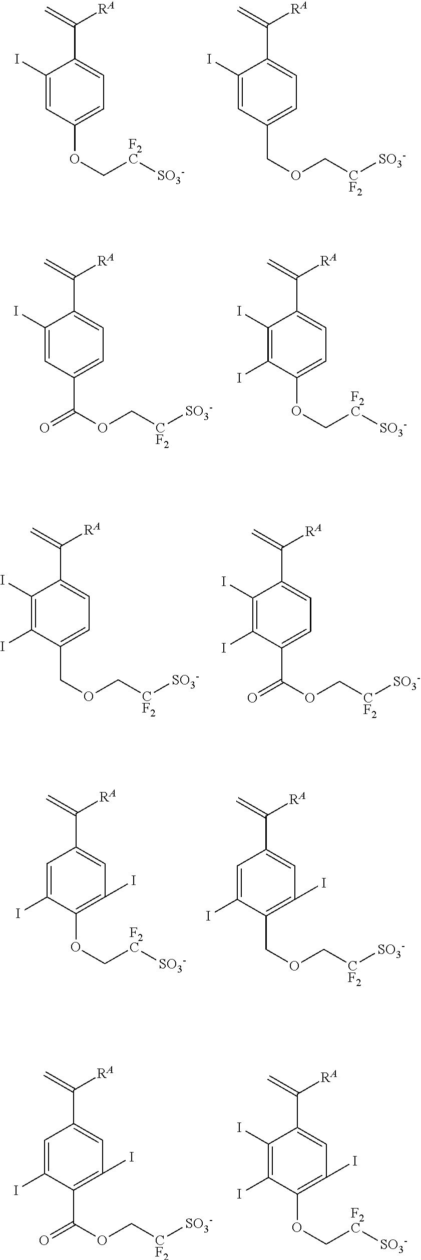

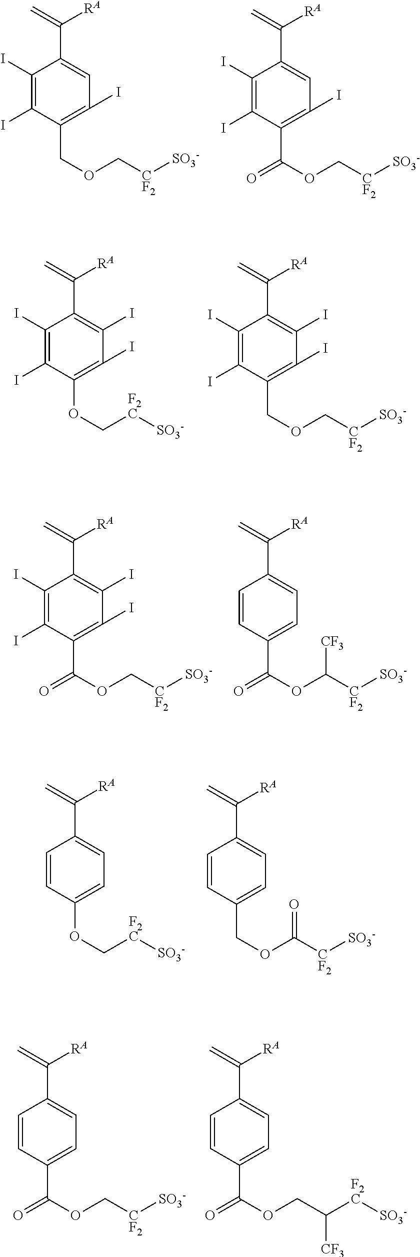

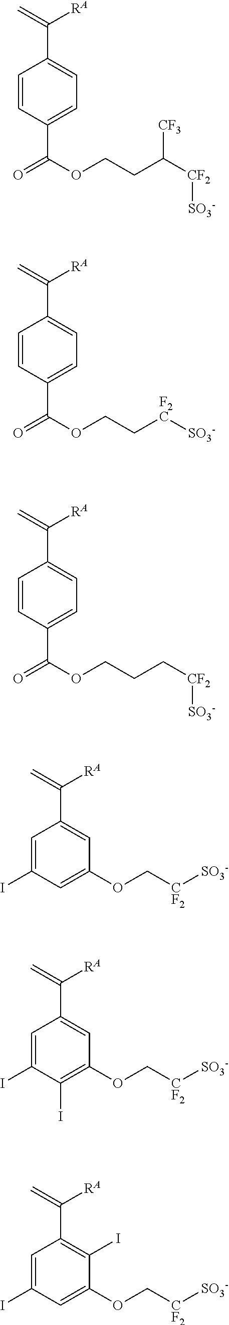

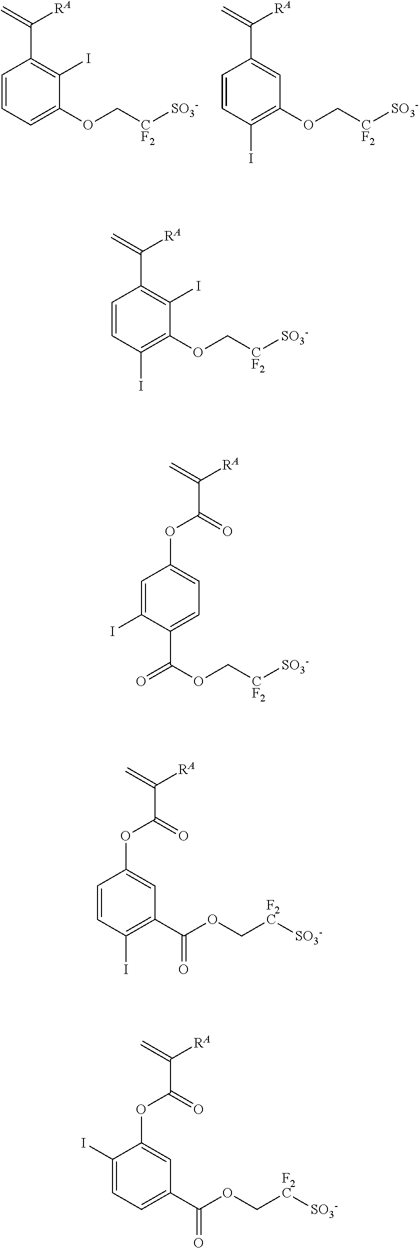

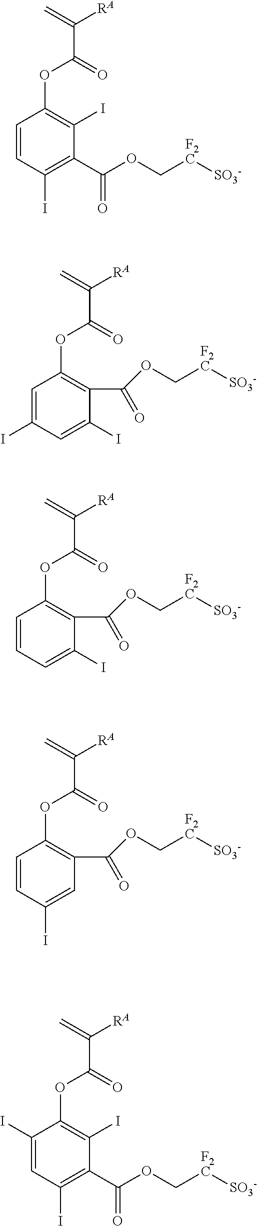

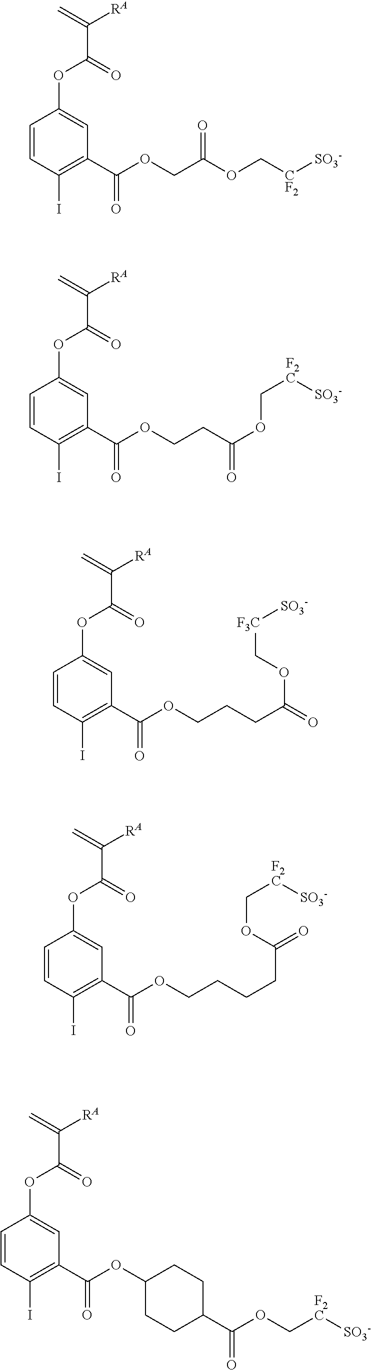

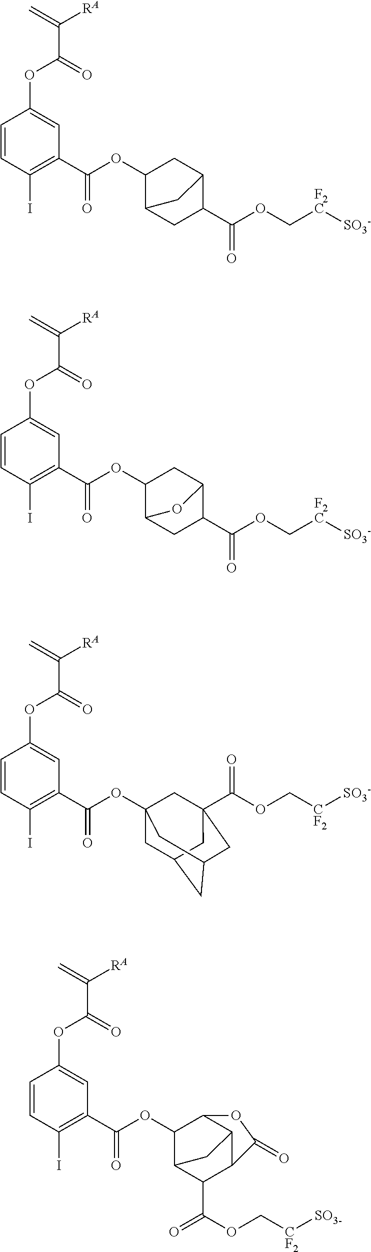

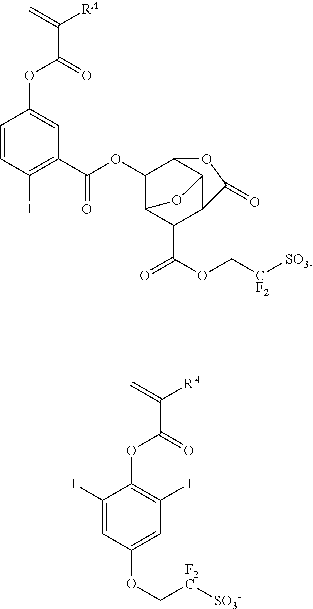

Examples of the anion in the monomer from which repeat unit (c3) is derived are shown below, but not limited thereto. R^{A} is as defined above.

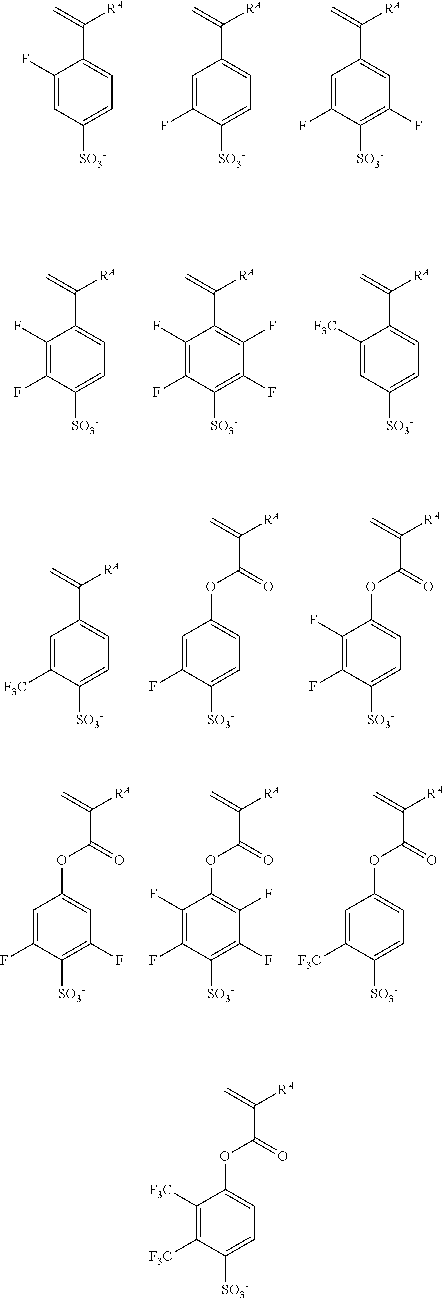

Besides the above-described repeat units, the base polymer may further include repeat units (d), which are derived from styrene, acenaphthylene, indene, coumarin, coumarone, and derivatives thereof.

In the base polymer comprising repeat units (a1), (a2), (b), (c1), (c2), (c3), and (d), a fraction of these units is:

preferably 0≤a1<1.0, 0≤a2<1.0, 0.1≤a1+a2<1.0, 0.1≤b≤0.9, 0≤c1≤0.6, 0≤c2≤0.6, 0≤c3≤0.6, 0≤c1+c2+c3≤0.6, and 0≤d≤0.5;
more preferably 0≤a1≤0.8, 0≤a2≤0.8, 0.2≤a1+a2≤0.8, 0.2≤b≤0.8, 0≤c1≤0.5, 0≤c2≤0.5, 0≤c3≤0.5, 0≤c1+c2+c3≤0.5, and 0≤d≤0.4; and
even more preferably 0≤a1≤0.7, 0≤a2≤0.7, 0.3≤a1+a2≤0.7, 0.25≤b≤0.7, 0≤c1≤0.4, 0≤c2≤0.4, 0≤c3≤0.4, 0≤c1+c2+c3≤0.4, and 0≤d≤0.3. Notably, a1+a2+b+c1+c2+c3+d=1.0.

The base polymer may be synthesized by any desired methods, for example, by dissolving suitable monomers selected from the monomers corresponding to the foregoing repeat units in an organic solvent, adding a radical polymerization initiator thereto, and heating for polymerization. Examples of the organic solvent which can be used for polymerization include toluene, benzene, tetrahydrofuran (THF), diethyl ether, and dioxane. Examples of the polymerization initiator used herein include 2,2′-azobisisobutyronitrile (AIBN), 2,2′-azobis(2,4-dimethylvaleronitrile), dimethyl 2,2-azobis(2-methylpropionate), benzoyl peroxide, and lauroyl peroxide. Preferably the reaction temperature is 50 to 80° C., and the reaction time is 2 to 100 hours, more preferably 5 to 20 hours.

In the case of a monomer having a hydroxy group, the hydroxy group may be replaced by an acetal group susceptible to deprotection with acid, typically ethoxyethoxy, prior to polymerization, and the polymerization be followed by deprotection with weak acid and water. Alternatively, the hydroxy group may be replaced by an acetyl, formyl, pivaloyl or similar group prior to polymerization, and the polymerization be followed by alkaline hydrolysis.

When hydroxystyrene or hydroxyvinylnaphthalene is copolymerized, an alternative method is possible. Specifically, acetoxystyrene or acetoxyvinylnaphthalene is used instead of hydroxystyrene or hydroxyvinylnaphthalene, and after polymerization, the acetoxy group is deprotected by alkaline hydrolysis, for thereby converting the polymer product to hydroxystyrene or hydroxyvinylnaphthalene. For alkaline hydrolysis, a base such as aqueous ammonia or triethylamine may be used. Preferably the reaction temperature is −20° C. to 100° C., more preferably 0° C. to 60° C., and the reaction time is 0.2 to 100 hours, more preferably 0.5 to 20 hours.

The base polymer should preferably have a weight average molecular weight (Mw) in the range of 1,000 to 500,000, and more preferably 2,000 to 30,000, as measured by GPC versus polystyrene standards using tetrahydrofuran (THF) solvent. With too low a Mw, the resist composition may become less heat resistant. A polymer with too high a Mw may lose alkaline solubility and give rise to a footing phenomenon after pattern formation.

If a base polymer has a wide molecular weight distribution or dispersity (Mw/Mn), which indicates the presence of lower and higher molecular weight polymer fractions, there is a possibility that foreign matter is left on the pattern or the pattern profile is degraded. The influences of Mw and Mw/Mn become stronger as the pattern rule becomes finer. Therefore, the base polymer should preferably have a narrow dispersity (Mw/Mn) of 1.0 to 2.0, especially 1.0 to 1.5, in order to provide a resist composition suitable for micropatterning to a small feature size.

The base polymer may be a blend of two or more polymers which differ in compositional ratio, Mw or Mw/Mn.

### (D) Organic Solvent

The positive resist composition may contain (D) an organic solvent. The organic solvent is not particularly limited as long as the foregoing components and other components are dissolvable therein. Examples of the organic solvent used herein are described in U.S. Pat. No. 7,537,880 (JP-A 2008-111103, paragraphs [0144]40145D. Exemplary solvents include ketones such as cyclohexanone, cyclopentanone, methyl-2-n-pentyl ketone, and 2-heptanone; alcohols such as 3-methoxybutanol, 3-methyl-3-methoxybutanol, 1-methoxy-2-propanol, 1-ethoxy-2-propanol, and diacetone alcohol (DAA); ethers such as propylene glycol monomethyl ether, ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as γ-butyrolactone, and mixtures thereof.

The organic solvent is preferably added in an amount of 100 to 10,000 parts, and more preferably 200 to 8,000 parts by weight per 100 parts by weight of the base polymer (C).

### (E) Surfactant

The resist composition may further comprise (E) a surfactant. Exemplary surfactants are described in JP-A 2008-111103, paragraphs [0165]-[0166]. Inclusion of a surfactant may improve or control the coating characteristics of the resist composition. When used, the surfactant is preferably added in an amount of 0.0001 to 10 parts by weight per 100 parts by weight of the base polymer (C). The surfactant may be used alone or in admixture.

### (F) Other Acid Generator

The resist composition may further comprise (F) an acid generator other than component (B) (referred to as other acid generator, hereinafter). The acid generator is capable of generating a strong acid. As used herein, the term “strong acid” refers to a compound having a sufficient acidity to induce deprotection reaction of an acid labile group on the base polymer. The acid generator is typically a compound (PAG) capable of generating an acid upon exposure to actinic ray or radiation. Although the PAG used herein may be any compound capable of generating an acid upon exposure to high-energy radiation, those compounds capable of generating sulfonic acid, imide acid (imidic acid) or methide acid are preferred. Suitable PAGs include sulfonium salts, iodonium salts, sulfonyldiazomethane, N-sulfonyloxyimide, and oxime-O-sulfonate acid generators. Exemplary PAGs are described in JP-A 2008-111103, paragraphs [0122]-[0142] (U.S. Pat. No. 7,537,880).

In the resist composition containing the other acid generator (F), its content is preferably 0.1 to 30 parts by weight, and more preferably 0.2 to 20 parts by weight per 100 parts by weight of the base polymer (C) although the content is not particularly limited as long as the benefits of the invention are not impaired.

### (G) Other Quencher

The resist composition may further comprise (G) a quencher other than component (A) (referred to as other quencher, hereinafter).

The other quencher is typically selected from conventional basic compounds. Conventional basic compounds include primary, secondary, and tertiary aliphatic amines, mixed amines, aromatic amines, heterocyclic amines, nitrogen-containing compounds with carboxy group, nitrogen-containing compounds with sulfonyl group, nitrogen-containing compounds with hydroxy group, nitrogen-containing compounds with hydroxyphenyl group, alcoholic nitrogen-containing compounds, amide derivatives, imide derivatives, and carbamate derivatives. Also included are primary, secondary, and tertiary amine compounds, specifically amine compounds having a hydroxy group, ether bond, ester bond, lactone ring, cyano group, or sulfonic acid ester bond as described in JP-A 2008-111103, paragraphs [0146]-[0164], and compounds having a carbamate group as described in JP 3790649. Addition of such a basic compound is effective for further suppressing the diffusion rate of acid in the resist film or correcting the pattern profile.

Onium salts such as sulfonium salts, iodonium salts and ammonium salts of sulfonic acids which are not fluorinated at α-position and carboxylic acids as described in U.S. Pat. No. 8,795,942 (JP-A 2008-158339) (exclusive of sulfonium salts containing fluorine in both anion and cation) may also be used as the other quencher. While an α-fluorinated sulfonic acid, imide acid, and methide acid are necessary to deprotect the acid labile group of carboxylic acid ester, an α-non-fluorinated sulfonic acid or carboxylic acid are released by salt exchange with an α-non-fluorinated onium salt. An α-non-fluorinated sulfonic acid or carboxylic acid function as a quencher because they do not induce deprotection reaction.

Suitable other quenchers include sulfonium salts having an iodized phenyl group (exclusive of those salts containing fluorine in both anion and cation) as described in JP-A 2017-219836. Since iodine is highly absorptive to EUV of wavelength 13.5 nm, it generates secondary electrons upon EUV exposure. The energy of secondary electrons is transferred to the acid generator to promote its decomposition, contributing to a higher sensitivity.

Also useful are quenchers of polymer type as described in U.S. Pat. No. 7,598,016 (JP-A 2008-239918). The polymeric quencher segregates at the resist film surface and thus enhances the rectangularity of resist pattern. When a protective film is applied as is often the case in the immersion lithography, the polymeric quencher is also effective for preventing a film thickness loss of resist pattern or rounding of pattern top.

The other quencher (G) is preferably added in an amount of 0.001 to 20 parts, more preferably 0.01 to 10 parts by weight per 100 parts by weight of the base polymer (C) although the content is not particularly limited as long as the benefits of the invention are not impaired. The other quencher may be used alone or in admixture.

### Other Components

With the foregoing components, the resist composition may further include other components such as a dissolution inhibitor, water repellency improver, and acetylene alcohol.

The inclusion of a dissolution inhibitor may lead to an increased difference in dissolution rate between exposed and unexposed areas and a further improvement in resolution. The dissolution inhibitor which can be used herein is a compound having at least two phenolic hydroxy groups on the molecule, in which an average of from 0 to 100 mol % of all the hydrogen atoms on the phenolic hydroxy groups are replaced by acid labile groups or a compound having at least one carboxy group on the molecule, in which an average of 50 to 100 mol % of all the hydrogen atoms on the carboxy groups are replaced by acid labile groups, both the compounds having a molecular weight of 100 to 1,000, and preferably 150 to 800. Typical are bisphenol A, trisphenol, phenolphthalein, cresol novolac, naphthalenecarboxylic acid, adamantanecarboxylic acid, and cholic acid derivatives in which the hydrogen atom on the hydroxy or carboxy group is replaced by an acid labile group, as described in U.S. Pat. No. 7,771,914 (JP-A 2008-122932, paragraphs [0155]40178D.

In the resist composition, the dissolution inhibitor is preferably added in an amount of 0 to 50 parts, more preferably 5 to 40 parts by weight per 100 parts by weight of the base polymer (C). The dissolution inhibitor may be used alone or in admixture.

The water repellency improver is added for improving the water repellency on surface of a resist film. The water repellency improver may be used in the topcoatless immersion lithography. Suitable water repellency improvers include polymers having a fluoroalkyl group and polymers having a specific structure with a 1,1,1,3,3,3-hexafluoro-2-propanol residue and are described in JP-A 2007-297590 and JP-A 2008-111103, for example. The water repellency improver to be added to the resist composition should be soluble in the alkaline developer and organic solvent developer. The water repellency improver of specific structure with a 1,1,1,3,3,3-hexafluoro-2-propanol residue is well soluble in the developer. A polymer having an amino group or amine salt copolymerized as repeat units may serve as the water repellent additive and is effective for preventing evaporation of acid during PEB, thus preventing any hole pattern opening failure after development.

The water repellency improver is not only effective for use in the immersion lithography requiring a resist film with higher water repellency, but also effective for reducing outgassing from a resist film upon EB or EUV exposure in a vacuum environment, for resolving hole or trench patterns of small size, and for minimizing blob defects by turning hydrophilic in contact with alkaline developer.

An appropriate amount of the water repellency improver is 0 to 20 parts, more preferably 0.5 to 10 parts by weight per 100 parts by weight of the base polymer (C). The water repellency improver may be used alone or in admixture.

Suitable acetylene alcohols are described in JP-A 2008-122932, paragraphs [0179]-[0182]. An appropriate amount of the acetylene alcohol blended is 0 to 5 parts by weight per 100 parts by weight of the base polymer (C). The acetylene alcohol may be used alone or in admixture.

### Pattern Forming Process

The positive resist composition is used in the fabrication of various integrated circuits. Pattern formation using the resist composition may be performed by well-known lithography processes. The process generally involves the steps of applying the resist composition to form a resist film on a substrate, exposing the resist film to high-energy radiation, and developing the exposed resist film in a developer. If necessary, any additional steps may be added.

Specifically, the positive resist composition is first applied onto a substrate on which an integrated circuit is to be formed (e.g., Si, SiO₂, SiN, SiON, TiN, WSi, BPSG, SOG, or organic antireflective coating) or a substrate on which a mask circuit is to be formed (e.g., Cr, CrO, CrON, MoSi₂, or SiO₂) by a suitable coating technique such as spin coating, roll coating, flow coating, dipping, spraying or doctor coating. The coating is prebaked on a hot plate at a temperature of 60 to 150° C. for 10 seconds to 30 minutes, preferably at 80 to 120° C. for 30 seconds to 20 minutes. The resulting resist film is generally 0.01 to 2 nm thick.

The resist film is then exposed to a desired pattern of high-energy radiation such as UV, deep-UV, EB, EUV of wavelength 3 to 15 nm, x-ray, soft x-ray, excimer laser light, γ-ray or synchrotron radiation. When UV, deep-UV, EUV, x-ray, soft x-ray, excimer laser light, γ-ray or synchrotron radiation is used as the high-energy radiation, the resist film is exposed thereto directly or through a mask having a desired pattern in a dose of preferably about 1 to 200 mJ/cm², more preferably about 10 to 100 mJ/cm². When EB is used as the high-energy radiation, the resist film is exposed thereto directly or through a mask having a desired pattern in a dose of preferably about 0.1 to 100 μC/cm², more preferably about 0.5 to 50 μC/cm². It is appreciated that the inventive resist composition is suited in micropatterning using i-line of wavelength 365 nm, KrF excimer laser, ArF excimer laser, EB, EUV, x-ray, soft x-ray, γ-ray or synchrotron radiation, especially in micropatterning using EB or EUV.

After the exposure, the resist film may be baked (PEB) on a hot plate or in an oven at 50 to 150° C. for 10 seconds to 30 minutes, preferably at 60 to 120° C. for 30 seconds to 20 minutes.

After the exposure or PEB, the resist film is developed in a developer in the form of an aqueous base solution for 3 seconds to 3 minutes, preferably 5 seconds to 2 minutes by conventional techniques such as dip, puddle and spray techniques. A typical developer is a 0.1 to 10 wt %, preferably 2 to 5 wt % aqueous solution of tetramethylammonium hydroxide (TMAH), tetraethylammonium hydroxide (TEAH), tetrapropylammonium hydroxide (TPAH), or tetrabutylammonium hydroxide (TBAH). The resist film in the exposed area is dissolved in the developer whereas the resist film in the unexposed area is not dissolved. In this way, the desired positive pattern is formed on the substrate.

In an alternative embodiment, a negative pattern may be formed from the positive resist composition via organic solvent development. The developer used herein is preferably selected from among 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate, and mixtures thereof.

At the end of development, the resist film is rinsed. As the rinsing liquid, a solvent which is miscible with the developer and does not dissolve the resist film is preferred. Suitable solvents include alcohols of 3 to 10 carbon atoms, ether compounds of 8 to 12 carbon atoms, alkanes, alkenes, and alkynes of 6 to 12 carbon atoms, and aromatic solvents. Specifically, suitable alcohols of 3 to 10 carbon atoms include n-propyl alcohol, isopropyl alcohol, 1-butyl alcohol, 2-butyl alcohol, isobutyl alcohol, tert-butyl alcohol, 1-pentanol, 2-pentanol, 3-pentanol, tert-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, and 1-octanol. Suitable ether compounds of 8 to 12 carbon atoms include di-n-butyl ether, diisobutyl ether, di-sec-butyl ether, di-n-pentyl ether, diisopentyl ether, di-sec-pentyl ether, di-tert-pentyl ether, and di-n-hexyl ether. Suitable alkanes of 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, and cyclononane. Suitable alkenes of 6 to 12 carbon atoms include hexene, heptene, octene, cyclohexene, methylcyclohexene, dimethylcyclohexene, cycloheptene, and cyclooctene. Suitable alkynes of 6 to 12 carbon atoms include hexyne, heptyne, and octyne. Suitable aromatic solvents include toluene, xylene, ethylbenzene, isopropylbenzene, tert-butylbenzene and mesitylene. The solvents may be used alone or in admixture.

Rinsing is effective for minimizing the risks of resist pattern collapse and defect formation. However, rinsing is not essential. If rinsing is omitted, the amount of solvent used may be reduced.

A hole or trench pattern after development may be shrunk by the thermal flow, RELACS® or DSA process. A hole pattern is shrunk by coating a shrink agent thereto, and baking such that the shrink agent may undergo crosslinking at the resist surface as a result of the acid catalyst diffusing from the resist layer during bake, and the shrink agent may attach to the sidewall of the hole pattern. The bake is preferably at a temperature of 70 to 180° C., more preferably 80 to 170° C., for a time of 10 to 300 seconds. The extra shrink agent is stripped and the hole pattern is shrunk.

### EXAMPLES

Examples of the invention are given below by way of illustration and not by way of limitation. The abbreviation “pbw” is parts by weight.

### Synthesis Example

Synthesis of Base Polymers P-1 to P-4

Each of base polymers P-1 to P-4 was prepared by combining suitable monomers, effecting copolymerization reaction thereof in tetrahydrofuran (THF) solvent, pouring the reaction solution into methanol for crystallization, washing the precipitate with hexane, isolation, and drying. The resulting polymer was analyzed for composition by ¹H-NMR spectroscopy, and for Mw and Mw/Mn by GPC versus polystyrene standards using THF solvent.

| | | | | |
|---|---|---|---|---|
| P-1 Mw = 5,200 Mw/Mn = 1.43 | 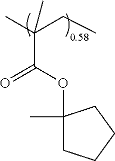 | 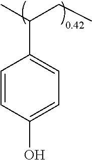 | | |
| P-2 Mw = 4,600 Mw/Mn = 1.41 | 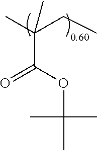 | 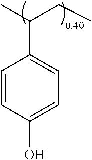 | | |
| P-3 Mw = 4,900 Mw/Mn = 1.44 | 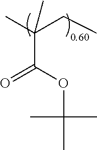 | 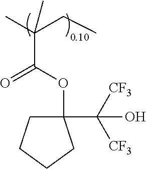 | 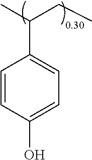 | |
| P-4 Mw = 6,600 Mw/Mn = 1.71 | 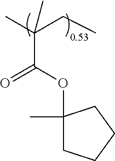 | 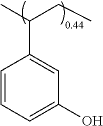 | 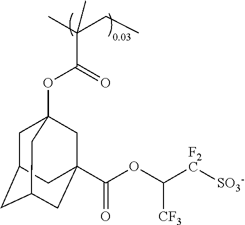 | 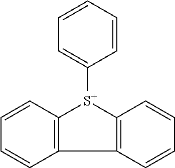 [SMILES: c1ccc(-[s+]2c3ccccc3c3ccccc32)cc1] |

### Examples 1 to 29 and Comparative Examples 1 to 3

Preparation and Evaluation of Positive Resist Compositions

### (1) Preparation of Positive Resist Composition

Positive resist compositions were prepared by dissolving components in an organic solvent containing 50 ppm of surfactant PolyFox PF-636 (Omnova Solutions Inc.) in accordance with the formulation shown in Tables 1 to 3, and filtering through a filter with a pore size of 0.2 μm.

The components in Tables 1 to 3 are identified below.

### Organic Solvent:

PGMEA: propylene glycol monomethyl ether acetate

DAA: diacetone alcohol

EL: ethyl lactate

### Acid Generators: PAG-1 to PAG-8

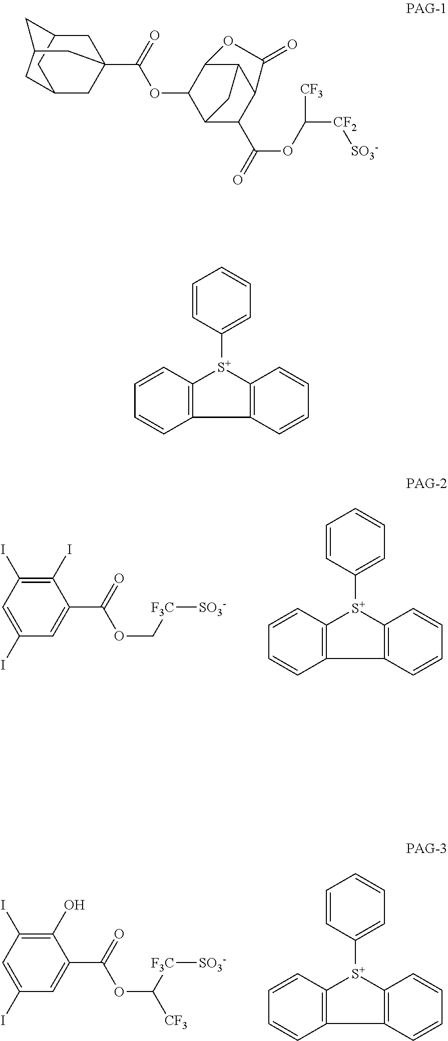

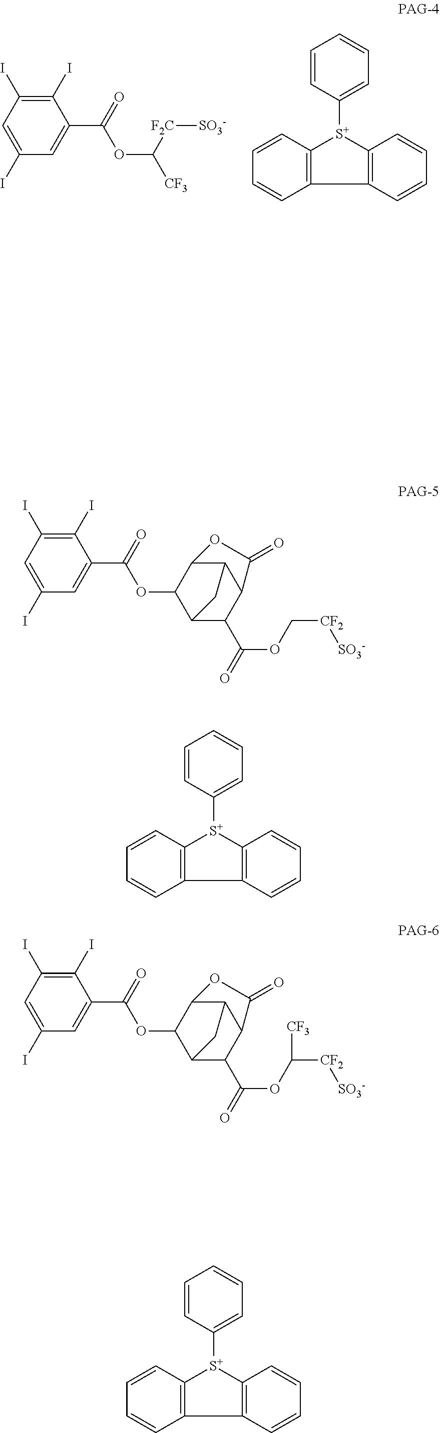

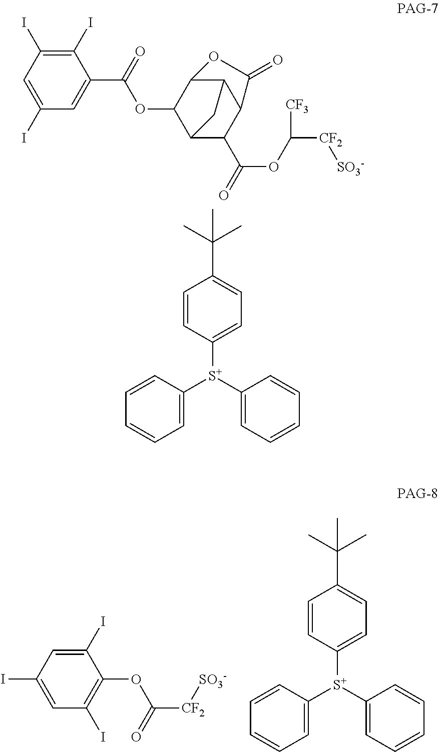

### Quenchers: Q-1 to Q-19

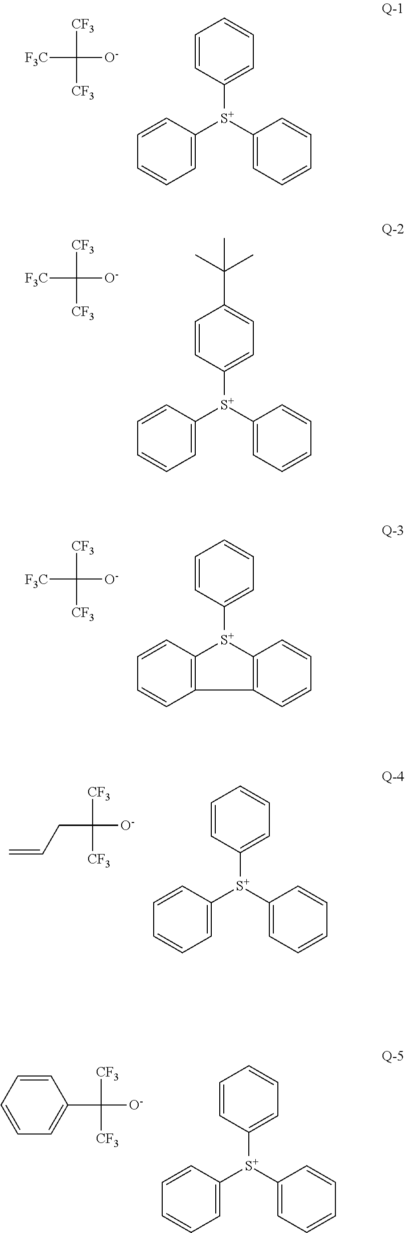

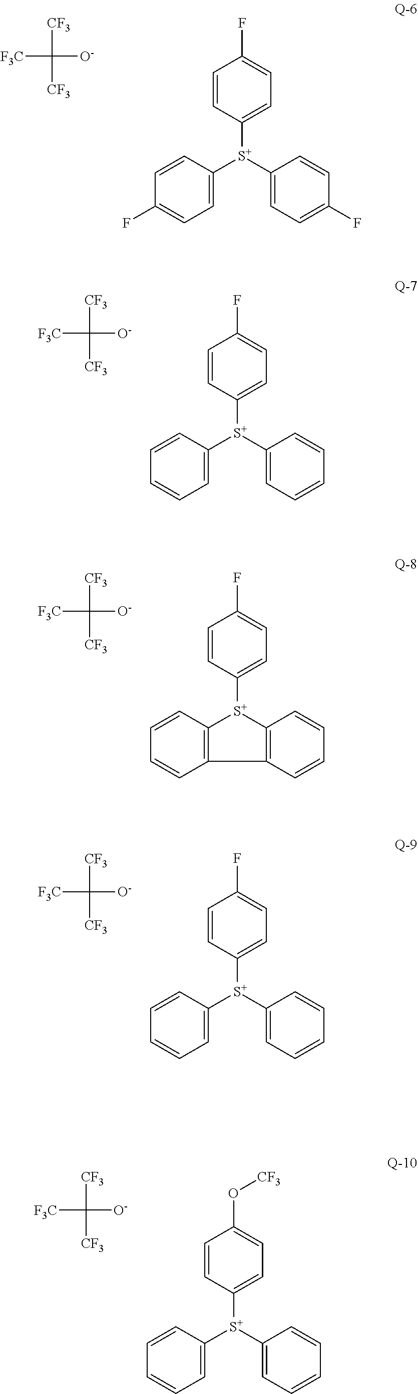

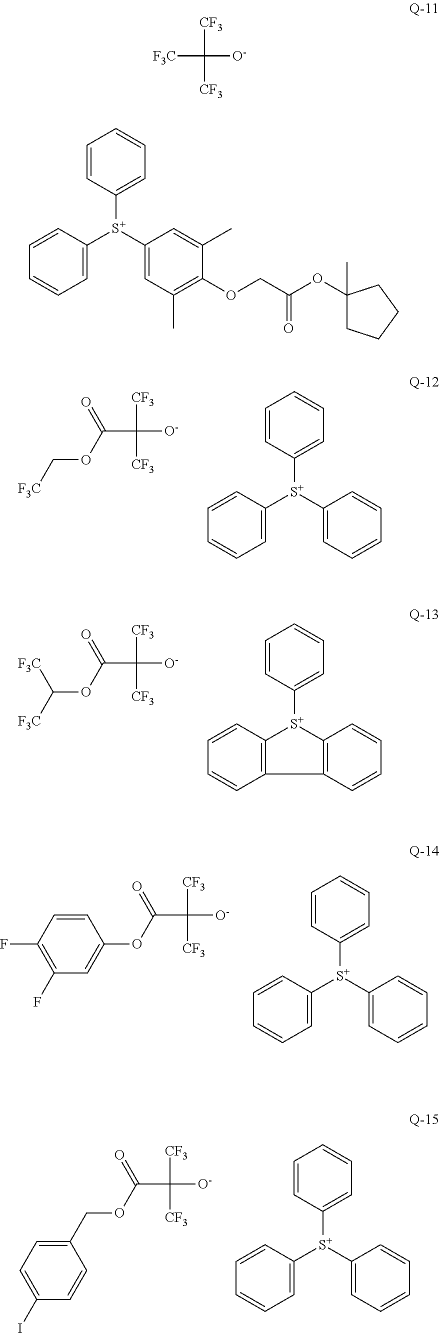

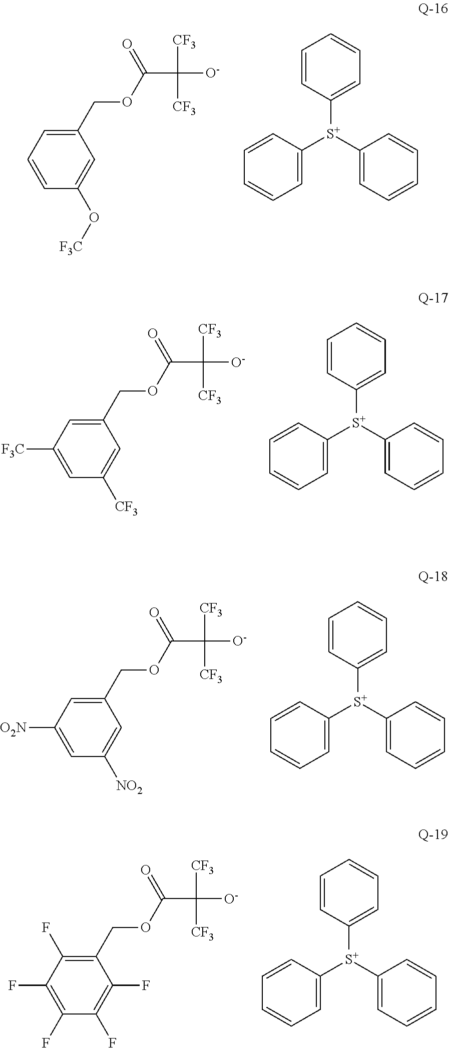

Comparative Quenchers: cQ-1 to cQ-3

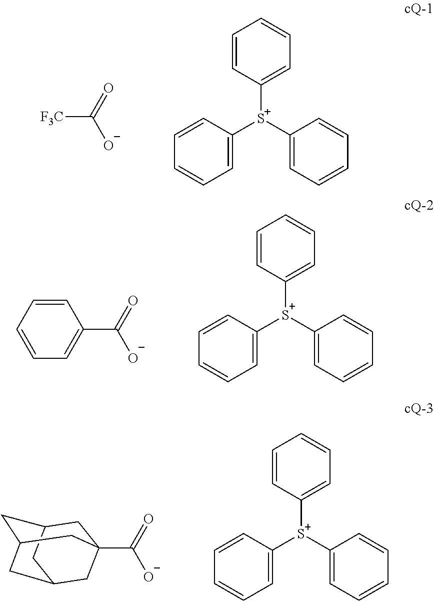 [SMILES: O=C([O-])C(F)(F)F ; c1ccc([S+](c2ccccc2)c2ccccc2)cc1 ; O=C([O-])c1ccccc1 ; c1ccc([S+](c2ccccc2)c2ccccc2)cc1 ; O=C([O-])C12CC3CC(CC(C3)C1)C2 ; c1ccc([S+](c2ccccc2)c2ccccc2)cc1]

### Water Repellency Improver: Polymer FP-1

| | | |
|---|---|---|
| FP-1 Mw = 11,200 Mw/Mn = 1.65 | 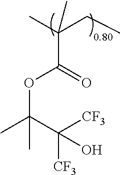 | 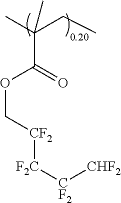 |

### (2) Evaluation by EUV Lithography

Each of the resist compositions in Tables 1 to 3 was spin coated on a silicon substrate having a 20-nm coating of silicon-containing spin-on hard mask SHB-A940 (Shin-Etsu Chemical Co., Ltd., Si content 43 wt %) and prebaked on a hotplate at 105° C. for 60 seconds to form a resist film of 35 nm thick. Using an EUV scanner NXE3400 (ASML, NA 0.33, a 0.9/0.6, 90° dipole illumination), the resist film was exposed to EUV through a mask bearing a 1:1 line-and-space pattern at a pitch 32 nm (on-wafer size). The resist film was baked (PEB) on a hotplate at the temperature shown in Tables 1 to 3 for 60 seconds and developed in a 2.38 wt % TMAH aqueous solution for 30 seconds to form a LS pattern having a line size of 16 nm.

The exposure dose that provides a LS pattern having a line size of 16 nm is reported as sensitivity. The LS pattern was observed under CD-SEM (CG6300, Hitachi High-Tech Corp.) to determine a roughness (LWR). A value of [the thickest line size at which no threading bridges form between lines at a dose smaller than the sensitivity of a resist film] minus [the thinnest line size at which neither pattern collapse nor resist film thickness loss occurs at a dose larger than the sensitivity of a resist film] is computed and reported as process window (PW).

The resist composition is shown in Tables 1 to 3 together with the sensitivity, LWR and PW of EUV lithography.

TABLE 1

| | | | Acid | | | | | | | |
|---|---|---|---|---|---|---|---|---|---|---|
| | | Polymer | generator | Additive | Quencher | Organic solvent | PEB temp. | Sensitivity | LWR | PW |
| | | (pbw) | (pbw) | (pbw) | (pbw) | (pbw) | (° C.) | (mJ/cm²) | (nm) | (nm) |
| Example | 1 | P-1 | PAG-1 | FP-1 | Q-1 | PGMEA (4,000) | 80 | 59 | 2.93 | 3.3 |
| | | (100) | (16.64) | (3.0) | (12.45) | DAA (1,000) | | | | |
| | 2 | P-1 | PAG-2 | FP-1 | Q-1 | PGMEA (4,000) | 80 | 50 | 2.31 | 4.2 |
| | | (100) | (18.08) | (3.0) | (12.45) | DAA (1,000) | | | | |
| | 3 | P-1 | PAG-3 | FP-1 | Q-1 | PGMEA (4,000) | 80 | 55 | 2.16 | 4.2 |
| | | (100) | (17.24) | (3.0) | (12.45) | DAA (1,000) | | | | |
| | 4 | P-1 | PAG-4 | FP-1 | Q-1 | PGMEA (4,000) | 80 | 52 | 2.41 | 4.4 |
| | | (100) | (19.46) | (3.0) | (12.45) | DAA (1,000) | | | | |
| | 5 | P-1 | PAG-5 | FP-1 | Q-1 | PGMEA (4,000) | 80 | 54 | 2.31 | 4.5 |
| | | (100) | (21.68) | (3.0) | (12.45) | DAA (1,000) | | | | |
| | 6 | P-1 | PAG-6 | FP-1 | Q-1 | PGMEA (4,000) | 80 | 55 | 2.16 | 4.6 |
| | | (100) | (23.04) | (3.0) | (12.45) | DAA (1,000) | | | | |
| | 7 | P-1 | PAG-7 | FP-1 | Q-1 | PGMEA (4,000) | 80 | 56 | 2.08 | 4.5 |
| | | (100) | (24.09) | (3.0) | (12.45) | DAA (1,000) | | | | |
| | 8 | P-1 | PAG-8 | FP-1 | Q-1 | PGMEA (4,000) | 80 | 53 | 2.28 | 3.9 |
| | | (100) | (18.96) | (3.0) | (12.45) | DAA (1,000) | | | | |
| | 9 | P-1 | PAG-6 | FP-1 | Q-2 | PGMEA (4,000) | 80 | 56 | 2.2 | 4.5 |
| | | (100) | (23.04) | (3.0) | (13.85) | DAA (1,000) | | | | |
| | 10 | P-1 | PAG-6 | FP-1 | Q-3 | PGMEA (4,000) | 80 | 53 | 2.19 | 4.4 |
| | | (100) | (23.04) | (3.0) | (12.40) | DAA (1,000) | | | | |
| | 11 | P-1 | PAG-6 | FP-1 | Q-4 | PGMEA (4,000) | 80 | 53 | 2.29 | 4.0 |
| | | (100) | (23.04) | (3.0) | (11.75) | DAA (1,000) | | | | |
| | 12 | P-1 | PAG-6 | FP-1 | Q-5 | PGMEA (4,000) | 80 | 56 | 2.30 | 4.0 |
| | | (100) | (23.04) | (3.0) | (12.65) | DAA (1,000) | | | | |
| | 13 | P-1 | PAG-6 | FP-1 | Q-6 | PGMEA (4,000) | 80 | 53 | 2.07 | 4.8 |
| | | (100) | (23.04) | (3.0) | (13.80) | DAA (1,000) | | | | |
| | 14 | P-1 | PAG-6 | FP-1 | Q-7 | PGMEA (3,500) | 80 | 54 | 2.18 | 4.6 |
| | | (100) | (23.04) | (3.0) | (12.90) | DAA (500) | | | | |
| | | | | | | EL (1,000) | | | | |
| | 15 | P-1 | PAG-6 | FP-1 | Q-8 | EL (4,000) | 80 | 53 | 2.17 | 4.5 |
| | | (100) | (23.04) | (3.0) | (12.85) | DAA (1,000) | | | | |
| | 16 | P-1 | PAG-6 | FP-1 | Q-9 | EL (4,000) | 80 | 52 | 2.37 | 4.3 |
| | | (100) | (23.04) | (3.0) | (14.15) | DAA (1,000) | | | | |
| | 17 | P-1 | PAG-6 | FP-1 | Q-10 | EL (4,000) | 80 | 53 | 2.27 | 4.2 |
| | | (100) | (23.04) | (3.0) | (14.55) | DAA (1,000) | | | | |
| | 18 | P-1 | PAG-6 | FP-1 | Q-11 | EL (4,000) | 80 | 54 | 2.31 | 3.8 |
| | | (100) | (23.04) | (3.0) | (17.05) | DAA (1,000) | | | | |
| | 19 | P-2 | PAG-6 | FP-1 | Q-1 | EL (4,000) | 90 | 55 | 2.03 | 4.5 |
| | | (100) | (23.04) | (3.0) | (12.45) | DAA (1,000) | | | | |
| | 20 | P-3 | PAG-6 | FP-1 | Q-1 | EL (4,000) | 85 | 56 | 2.07 | 4.7 |
| | | (100) | (23.04) | (3.0) | (12.45) | DAA (1,000) | | | | |
| | 21 | P-4 | PAG-6 | FP-1 | Q-1 | EL (4,000) | 80 | 58 | 2.17 | 4.3 |
| | | (100) | (12.04) | (3.0) | (12.45) | DAA (1,000) | | | | |

TABLE 2

| | | | Acid | | | | | | | |
|---|---|---|---|---|---|---|---|---|---|---|
| | | Polymer | generator | Additive | Quencher | Organic solvent | PEB temp. | Sensitivity | LWR | PW |
| | | (pbw) | (pbw) | (pbw) | (pbw) | (pbw) | (° C.) | (mJ/cm²) | (nm) | (nm) |
| Example | 22 | P-1 | PAG-4 | FP-1 | Q-12 | EL (4,000) | 80 | 56 | 2.21 | 4.1 |
| | | (100) | (19.46) | (3.0) | (13.90) | DAA (1,000) | | | | |
| | 23 | P-1 | PAG-4 | FP-1 | Q-13 | EL (4,000) | 80 | 53 | 2.26 | 4.0 |
| | | (100) | (19.46) | (3.0) | (15.60) | DAA (1,000) | | | | |
| | 24 | P-1 | PAG-4 | FP-1 | Q-14 | EL (4,000) | 80 | 58 | 2.30 | 3.8 |
| | | (100) | (19.46) | (3.0) | (14.65) | DAA (1,000) | | | | |
| | 25 | P-1 | PAG-4 | FP-1 | Q-15 | EL (4,000) | 80 | 56 | 2.27 | 3.9 |
| | | (100) | (19.46) | (3.0) | (17.25) | DAA (1,000) | | | | |
| | 26 | P-1 | PAG-4 | FP-1 | Q-16 | EL (4,000) | 80 | 51 | 2.22 | 3.6 |
| | | (100) | (19.46) | (3.0) | (16.20) | DAA (1,000) | | | | |
| | 27 | P-1 | PAG-4 | FP-1 | Q-17 | EL (4,000) | 80 | 58 | 2.10 | 3.8 |
| | | (100) | (19.46) | (3.0) | (17.50) | DAA (1,000) | | | | |
| | 28 | P-1 | PAG-4 | FP-1 | Q-18 | EL (4,000) | 80 | 57 | 2.28 | 3.7 |
| | | (100) | (19.46) | (3.0) | (16.35) | DAA (1,000) | | | | |
| | 29 | P-1 | PAG-4 | FP-1 | Q-19 | EL (4,000) | 80 | 56 | 2.17 | 4.0 |
| | | (100) | (19.46) | (3.0) | (13.10) | DAA (1,000) | | | | |
| | | | | | cQ1 | | | | | |
| | | | | | (3.20) | | | | | |

TABLE 3

| | | | Acid | | | | | | | |
|---|---|---|---|---|---|---|---|---|---|---|
| | | Polymer | generator | Additive | Quencher | Organic solvent | PEB temp. | Sensitivity | LWR | PW |
| | | (pbw) | (pbw) | (pbw) | (pbw) | (pbw) | (° C.) | (mJ/cm²) | (nm) | (nm) |
| Comparative | 1 | P-1 | PAG-2 | FP-1 | cQ-1 | PGMEA (4,000) | 80 | 63 | 2.61 | 0.3 |
| Example | | (100) | (18.08) | (3.0) | (9.40) | DAA (1,000) | | | | |
| | 2 | P-1 | PAG-2 | FP-1 | cQ-2 | PGMEA (4,000) | 80 | 62 | 2.81 | 0.6 |
| | | (100) | (18.08) | (3.0) | (9.60) | DAA (1,000) | | | | |
| | 3 | P-1 | PAG-2 | FP-1 | cQ-3 | PGMEA (4,000) | 80 | 65 | 2.96 | 0.8 |
| | | (100) | (18.08) | (3.0) | (11.05) | DAA (1,000) | | | | |

It is evident from Tables 1 to 3 that the positive resist compositions comprising a sulfonium salt containing an anion of specific structure, hexafluoroalkoxide anion as the quencher and a sulfonium salt containing a sulfonate anion having fluorine on the carbon atom at α- and/or β-position of the sulfo group as the acid generator exhibit a high sensitivity, reduced LWR, and broad process window.

Japanese Patent Application No. 2021-102960 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. A positive resist composition comprising
(A) a quencher in the form of a sulfonium salt having the following formula (1): 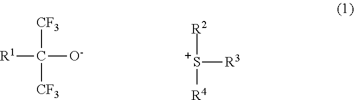
wherein R¹ is fluorine, a C₁-C₄ alkyl group, C₁-C₄ alkyloxy group, C₂-C₄ alkenyl group, C₂-C₄ alkynyl group, phenyl group, or C₁-C₂₀ hydrocarbyloxycarbonyl group, some or all of the hydrogen atoms in the alkyl, alkyloxy, alkenyl, alkynyl, and hydrocarbyloxycarbonyl groups may be substituted by fluorine, chlorine, bromine, iodine, trifluoromethyl, trifluoromethoxy, trifluorothio, hydroxy, cyano, nitro or sulfonyl moiety, some constituent —CH₂— in the alkyl, alkyloxy, alkenyl, alkynyl, and hydrocarbyloxycarbonyl groups may be replaced by an ester bond or ether bond, and some or all of the hydrogen atoms in the phenyl group may be substituted by fluorine, C₁-C₄ fluoroalkyl, C₁-C₄ fluoroalkyloxy, C₁-C₄ fluoroalkylthio, cyano or nitro moiety,
R² to R⁴ are each independently halogen or a C₁-C₂₀ hydrocarbyl group which may contain at least one atom selected from oxygen, sulfur, nitrogen and halogen, R² and R³ may bond together to form a ring with the sulfur atom to which they are attached,
(B) an acid generator in the form of a sulfonium salt consisting of a sulfonate anion having fluorine on the carbon atom at α- and/or β-position of the sulfo group and a sulfonium cation, and
(C) a base polymer comprising repeat units of at least one type selected from repeat units (a1) having a carboxy group whose hydrogen is substituted by an acid labile group and repeat units (a2) having a phenolic hydroxy group whose hydrogen is substituted by an acid labile group.

2. The resist composition of claim 1 wherein the sulfonate anion in the sulfonium salt (B) has the formula (2-1) or (2-2): 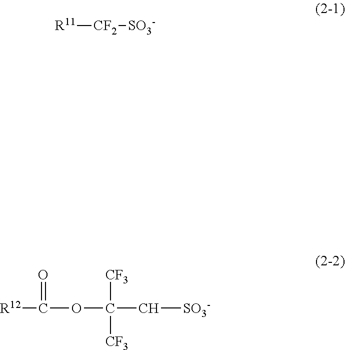
wherein R¹¹ is fluorine or a C₁-C₂₀ hydrocarbyl group which may contain a heteroatom, and R¹² is a C₁-C₂₀ hydrocarbyl group which may contain a heteroatom.

3. The resist composition of claim 1 wherein the sulfonate anion in the sulfonium salt (B) is an iodized sulfonate anion.

4. The resist composition of claim 3 wherein the iodized sulfonate anion has the formula (2-3): 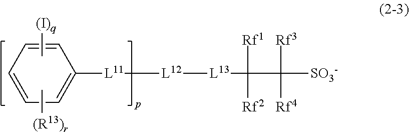
wherein p is an integer of 1 to 3, q is an integer of 1 to 5, r is an integer of 0 to 3, and 1≤q+r≤5,
L¹¹ is a single bond, ether bond, ester bond, amide bond, imide bond, or C₁-C₆ saturated hydrocarbylene group in which some constituent —CH₂— may be replaced by an ether bond or ester bond,
L¹² is a single bond or a C₁-C₂₀ hydrocarbylene group when p is 1, and a C₁-C₂₀ (p+1)-valent hydrocarbon group when p is 2 or 3, the hydrocarbylene group and (p+1)-valent hydrocarbon group may contain at least one atom selected from oxygen, sulfur and nitrogen,
L¹³ is a single bond, ether bond or ester bond,
R¹³ is a hydroxy group, carboxy group, fluorine, chlorine, bromine or amino group, or a C₁-C₂₀ hydrocarbyl group, C₁-C₂₀ hydrocarbyloxy group, C₂-C₂₀ hydrocarbylcarbonyl group, C₂-C₂₀ hydrocarbyloxycarbonyl group, C₂-C₂₀ hydrocarbylcarbonyloxy group, or C₁-C₂₀ hydrocarbylsulfonyloxy group, which may contain fluorine, chlorine, bromine, hydroxy, amino or ether bond, or —N(R^{13A})(R^{13B}), —N(R^{13C})—C(═O)—R^{13D}, or —N(R^{13C})—C(═O)—O—R^{13D}, R^{13A} and R^{13B} are each independently hydrogen or a C₁-C₆ saturated hydrocarbyl group, R^{13C} is hydrogen or a C₁-C₆ saturated hydrocarbyl group in which some or all of the hydrogen atoms may be substituted by halogen, hydroxy, C₁-C₆ saturated hydrocarbyloxy, C₂-C₆ saturated hydrocarbylcarbonyl or C₂-C₆ saturated hydrocarbylcarbonyloxy moiety, R^{13D} is a C₁-C₁₆ aliphatic hydrocarbyl group, C₆-C₁₂ aryl group or C₇-C₁₅ aralkyl group, in which some or all of the hydrogen atoms may be substituted by halogen, hydroxy, C₁-C₆ saturated hydrocarbyloxy, C₂-C₆ saturated hydrocarbylcarbonyl or C₂-C₆ saturated hydrocarbylcarbonyloxy moiety,
Rf¹ to Rf⁴ are each independently hydrogen, fluorine or trifluoromethyl, at least one of Rf¹ to Rf⁴ is fluorine or trifluoromethyl, and Rf¹ and Rf², taken together, may form a carbonyl group.

5. The resist composition of claim 1 wherein repeat units (a1) have the formula (a1) and repeat units (a2) have the formula (a2): 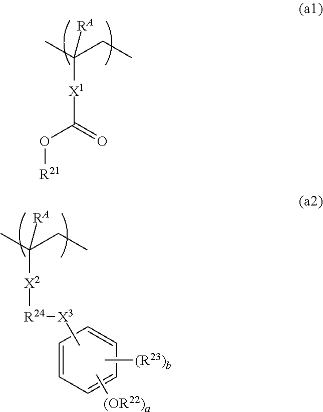
wherein R^{A} is each independently hydrogen or methyl,
X¹ is a single bond, phenylene, naphthylene, or a C₁-C₁₂ linking group containing at least one moiety selected from ether bond, ester bond and lactone ring,
X² is a single bond, ester bond or amide bond,
X³ is a single bond, ether bond or ester bond,
R²¹ and R²² are each independently an acid labile group,
R²³ is fluorine, trifluoromethyl, cyano or a C₁-C₆ saturated hydrocarbyl group,
R²⁴ is a single bond or a C₁-C₆ alkanediyl group in which some carbon may be replaced by an ether bond or ester bond,
a is 1 or 2, b is an integer of 0 to 4, and 1≤a+b≤5.

6. The resist composition of claim 1 wherein the base polymer further comprises repeat units having an adhesive group selected from hydroxy, carboxy, lactone ring, carbonate, thiocarbonate, carbonyl, cyclic acetal, ether bond, ester bond, sulfonic ester bond, cyano, amide bond, —O—C(═O)—S—, and —O—C(═O)—NH—.

7. The resist composition of claim 1, further comprising (D) an organic solvent.

8. The resist composition of claim 1, further comprising (E) a surfactant.

9. A pattern forming process comprising the steps of applying the positive resist composition of claim 1 onto a substrate to form a resist film thereon, exposing the resist film to high-energy radiation, and developing the exposed resist film in a developer.

10. The process of claim 9 wherein the high-energy radiation is i-line, KrF excimer laser, ArF excimer laser, EB, or EUV of wavelength 3 to 15 nm.
